(19) United States
(12) Patent Application Publication
(10) Pub. No.: US 2024/0174649 A1
(43) Pub. Date: May 30, 2024

(54) CULLIN-RING E3 UBIQUITIN LIGASE 4 INHIBITOR COMPOUNDS AND METHODS OF THEIR USE

(71) Applicant: ICAHN SCHOOL OF MEDICINE AT MOUNT SINAI, New York, NY (US)

(72) Inventors: Robert J. DEVITA, New York, NY (US); Zhen-Qiang PAN, New York, NY (US); Khoi Q. HUYNH, New York, NY (US); Moses MOUSTAKIM, New York, NY (US); Kenneth WU, New York, NY (US); Chalada SUEBSUWONG, New York, NY (US); Benjamin D. HOPKINS, New York, NY (US); Peng-Jen CHEN, New York, NY (US); Michael Block LAZARUS, New York, NY (US)

(21) Appl. No.: 18/275,287

(22) PCT Filed: Feb. 1, 2022
(86) PCT No.: PCT/US2022/014774

§ 371 Date: Aug. 1, 2023

Related U.S. Application Data
(60) Provisional application No. 63/144,358, filed on Feb. 1, 2021.

(51) Int. Cl. C07D 405/04; A61P 35/00; C07D 311/22; C07D 405/14; C07D 493/04

(52) U.S. Cl. CPC C07D 405/04; A61P 35/00; C07D 311/22; C07D 405/14; C07D 493/04

(57) ABSTRACT

Disclosed herein are compounds that inhibit cullin-RING E3 ubiquitin ligase 4, a method of inhibiting the catalytic activity of a Cullin-RING E3 Ubiquitin (Ub) Ligase (CRL) in a cell, compositions comprising the inhibitor compounds, a method of treating a tumor, and a method of treating a subject for cancer.

This application claims the priority benefit of U.S. Provisional Patent Application Ser. No. 63/144,358, filed Feb. 1, 2021, which is hereby incorporated by reference in its entirety.

This invention was made with government support under grant numbers GM122751, 1R01CA251425-01, R01GM074830, and R01GM130144 awarded by the National Institutes of Health. The government has certain rights in the invention.

### FIELD OF THE INVENTION

Disclosed herein are compounds that inhibit cullin-RING E3 ubiquitin ligase 4 and methods of their use.

### BACKGROUND OF THE INVENTION

Cullin-RING E3 Ubiquitin (Ub) Ligases (“CRLs”) are the largest RING-type E3 family consisting of ˜300 members, ˜50% of the E3s identified in humans (Petroski and Deshaies, “Function and Regulation of Cullin-RING Ubiquitin Ligases,” Nat. Rev. Mol. Cell Biol. 6:9-20 (2005); Sarikas et al., “The Cullin Protein Family,” Genome Biol. 12(4):220 (2011)). CRLs target many critical regulators of cell division and signaling. Canonical CRLs are modular complexes, in which a cullin (“CUL”) subunit's N-terminal domain assembles interchangeably with CUL-specific substrate receptors capable of binding a substrate. On the other hand, a CUL's C-terminus (CTD) binds a RING finger protein, ROC1/RBX1 for CUL1-4 or ROC2 for CUL5, respectively, to form a core ligase complex. CRL's core ligase collaborates with E2 Ub conjugating enzymes for transferring Ub(s) to the bound substrate, or an Ub moiety on a growing Ub chain.

A selective small-molecule modulator of CRL's function allows one to address mechanistic and phenotypic questions about its targets in biochemical, cell-based, and animal studies. To date, there is only one FDA-approved E3 drug class that targets the substrate receptor cereblon (thalidomide/lenalidomide) (Wertz and Wang, “From Discovery to Bedside: Targeting the Ubiquitin System,” Cell Chem. Biol. 26(2):156-177 (2019)). Current drug/probe discovery efforts against the Ub-proteasome system depend heavily on traditional methods that exploit the ability of small molecule agents to disable an enzyme's catalytic pocket. However, RING-type E3s are atypical enzymes and contribute to ubiquitination by mediating protein-protein interactions (PPI) with substrate, E2 and Ub (Petroski and Deshaies, “Function and Regulation of Cullin-RING Ubiquitin Ligases,” Nat. Rev. Mol. Cell Biol. 6:9-20 (2005)). High resolution structure studies have shown that interactions involving E3's RING domain, E2 and Ub are characterized by large, relatively flat interfaces (Plechanovovi et al., “Structure of a RING E3 Ligase and Ubiquitin-loaded E2 Primed for Catalysis,” Nature 489(7414):115-120 (2012)). Such perceived “undruggable” features impose a significant barrier to structure-based ligand search using either virtual or fragment-based physical screening.

To date, there are no reported small molecule lead compounds targeting the catalytic activity of any CRL.

The present disclosure is directed to overcoming deficiencies in the art.

### SUMMARY OF THE INVENTION

One aspect of the present disclosure relates to a compound of formula (I) having the following structure:

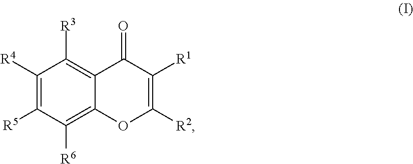

or a stereoisomer, pharmaceutically acceptable salt, oxide, or solvate thereof, wherein
R¹ is H,

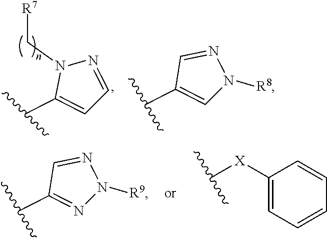

wherein the benzene ring of

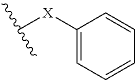

is optionally substituted with NO₂ or halogen;
R² is H or CF₃;
R³, R⁴, R⁵, and R⁶ are each independently selected from the group consisting of H, OH, OAc, halogen, alkoxy, OTf, and ONa;
R⁷, R⁸, and R⁹ are each independently selected from H, C₁-C₆ alkyl, pyridine, or

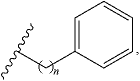 [SMILES: *Cc1ccccc1]

wherein the benzene ring of

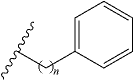 [SMILES: *Cc1ccccc1]

is optionally substituted one or more times with C₁-C₆ alkyl, NH₂, CF₃, halogen, CN, C(O)NH₂, and alkoxy;
X is either present or absent, and when present is CH₂ or O; and
n is 0 or 1;
   with the proviso that when R³ and R⁴ are H, R⁵ and R⁶ are OH, and R² is CF₃, R¹ is not

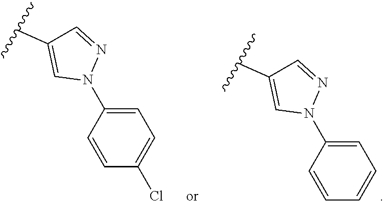 [SMILES: *c1cnn(-c2ccc(Cl)cc2)c1 ; *c1cnn(-c2ccccc2)c1]

Another aspect of the present disclosure relates to a compound of formula (I) having the following structure:

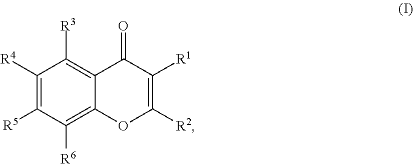

or a stereoisomer, pharmaceutically acceptable salt, oxide, or solvate thereof, wherein
R¹ is H,

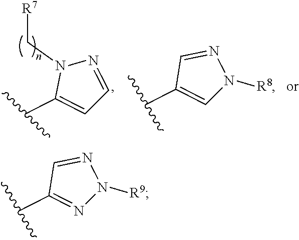

R² is H or CF₃;
R³, R⁴, R⁵, and R⁶ are each independently selected from the group consisting of H, OH, OAc, alkoxy, OTf, and ONa;
R⁷, R⁸, and R⁹ are each independently selected from H, C₁-C₆ alkyl, pyridine, or

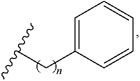

wherein the benzene ring of

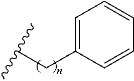

is optionally substituted one or more times with C₁-C₆ alkyl, NH₂, CF₃, halogen, CN, C(O)NH₂, and alkoxy; and
n is 0 or 1;
   with the proviso that when R³ and R⁴ are H, R⁵ and R⁶ are OH, and R² is CF₃, R¹ is not

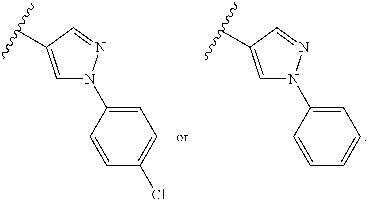 [SMILES: *c1cnn(-c2ccc(Cl)cc2)c1 ; *c1cnn(-c2ccccc2)c1]

Another aspect of the present disclosure relates to a method of inhibiting the catalytic activity of a Cullin-RING E3 Ubiquitin (Ub) Ligase (CRL) in a cell. This method involves contacting a cell with a compound of formula (II) having the following structure:

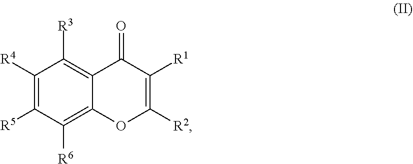

or a stereoisomer, pharmaceutically acceptable salt, oxide, or solvate thereof, wherein
R¹ and R² are each independently H, CF₃,

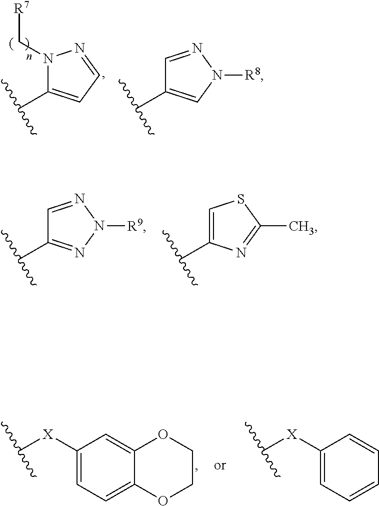

wherein the benzene ring of

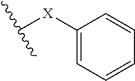

is optionally substituted with NO₂ or halogen;
R³, R⁴, R⁵, and R⁶ are each independently selected from the group consisting of H, halogen, OH, C₁-C₆ alkyl, OAc, NaO, OTf, and alkoxy, wherein two adjacent alkoxy groups may be taken together to form a heterocycle;
R⁷, R⁸, and R⁹ are each independently selected from H, C₁-C₆ alkyl, pyridine, or

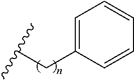

wherein the benzene ring of

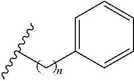

is optionally substituted one or more times with C₁-C₆ alkyl, NH₂, CF₃, halogen, CN, C(O)NH₂, and alkoxy;
X is either present or absent, and when present is CH₂ or O; and
n is 0 or 1;
   under conditions effective to inhibit activity of the CRL in the cell.

Yet another aspect of the present disclosure relates to a method of inhibiting the catalytic activity of a Cullin-RING E3 Ubiquitin (Ub) Ligase (CRL) in a cell. This method involves contacting a cell with a compound of formula (II) having the following structure:

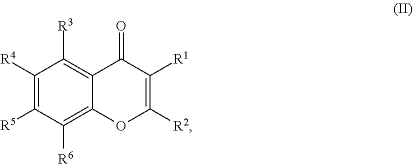

or a stereoisomer, pharmaceutically acceptable salt, oxide, or solvate thereof, wherein

R¹ and R² are each independently H, CF₃,

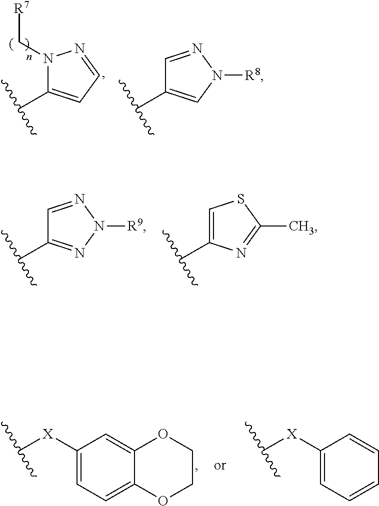

where the benzene ring of

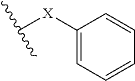

is optionally substituted with NO₂ or halogen;
R³, R⁴, R⁵, and R⁶ are each independently selected from the group consisting of H, OH, C₁-C₆ alkyl, —OAc, NaO—, OTf, and alkoxy, wherein two adjacent alkoxy groups may be taken together to form a heterocycle;
R⁷, R⁸, and R⁹ are each independently selected from H, C₁-C₆ alkyl, pyridine, or

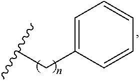

wherein the benzene ring of

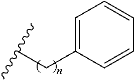

is optionally substituted one or more times with C₁-C₆ alkyl, NH₂, CF₃, halogen, CN, C(O)NH₂, and alkoxy;
X is either present or absent, and when present is C or O; and
n is 0 or 1;
   under conditions effective to inhibit activity of the CRL in the cell.

Another aspect of the disclosure relates to a composition comprising a compound of formula (II) as described herein and a carrier.

A further aspect of the disclosure relates to a method of treating a tumor. This method involves contacting a tumor with a compound of formula (II) under conditions effective to treat the tumor.

Another aspect of the disclosure relates to a method of treating a subject for cancer. This method involves administering to a subject in need of treatment for cancer a compound of formula (II) as described herein under conditions effective to treat the subject for cancer.

A further aspect of the disclosure relates to a compound having a structure selected from

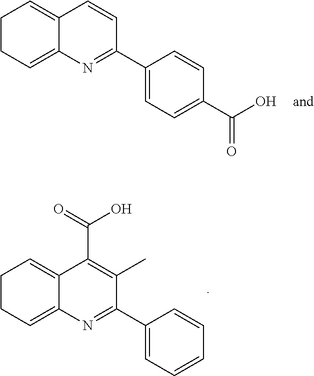 [SMILES: O=C(O)c1ccc(-c2ccc3c(n2)=CCCC=3)cc1 ; Cc1c(-c2ccccc2)nc2c(c1C(=O)O)=CCCC=2]

Another aspect of the disclosure relates to a method of inhibiting the catalytic activity of a Cullin-RING E3 Ubiquitin (Ub) Ligase (CRL) in a cell. This method includes contacting a cell with a compound selected from the group consisting of

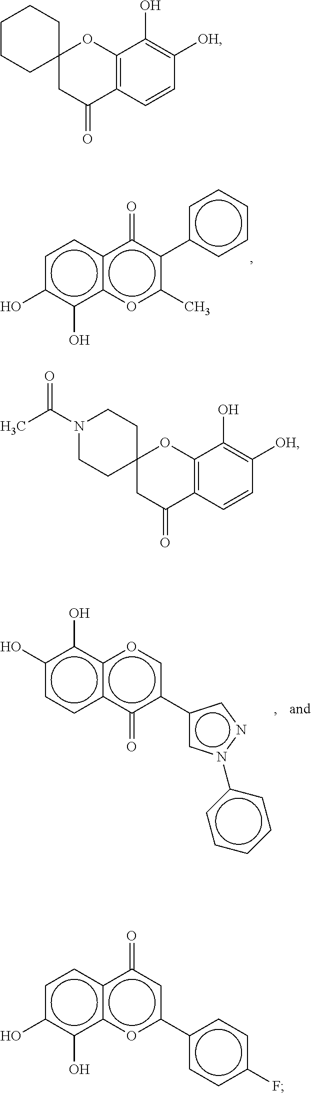

under conditions effective to inhibit activity of the CRL in the cell.

Another aspect of the disclosure relates to a method of treating a subject for cancer. This method involves administering to a subject in need of treatment for cancer a compound selected from the group consisting of

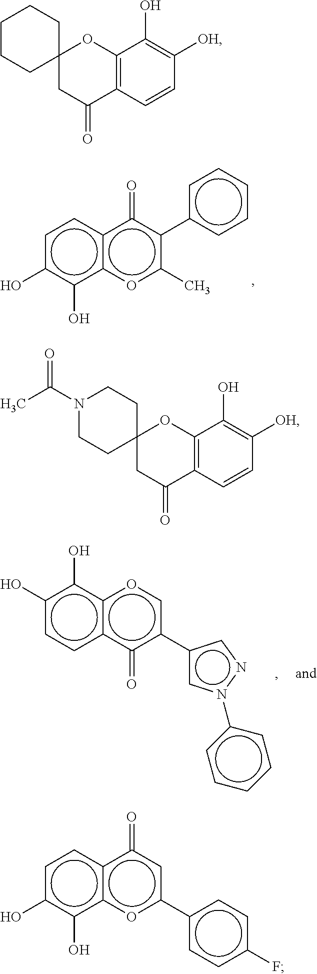

under conditions effective to treat the subject for cancer.

To address the need for small molecule lead compounds targeting the catalytic activity of any CRL, a novel high throughput screen (HTS) platform was created using the “fluorescence (Forster) resonance energy transfer (FRET) K48 di-Ub assay” (Wu et al., “Suramin Inhibits Cullin-RING E3 Ubiquitin Ligases,” PNAS 113(14):E2011-2018 (2016)). In this system, a FRET signal is generated as a result of covalent conjugation of two Ub molecules carrying a pair of matching fluorophores in a reaction that requires E1, E2 Cdc34, and an E3 CRL1 sub-complex (ROC1-CUL1 CTD). Fully functional Ub variants were created to allow only one nucleophilic attack that produces a single Ub-Ub isopeptide bond, thereby eliminating the complexity associated with polyubiquitin chain assembly to ensure a high degree of reproducibility for effective HTS. Each fluorophore was uniquely engineered to either donor or receptor Ub at specific site. A pilot HTS identified a small molecule compound, suramin (an anti-trypansomal drug), that can inhibit E3 CRL1 activity by disrupting its ability to recruit E2 Cdc34 (Wu et al., “Suramin Inhibits Cullin-RING E3 Ubiquitin Ligases,” PNAS 113(14):E2011-2018 (2016)). These observations have provided proof-of-principle evidence that an E2-E3 interface can be perturbed through small-molecule modulators. The present disclosure describes a large scale HTS and extensive follow up hit-to-lead studies, which identified a new class of small molecule inhibitors against E3 CRLs.

Cullin-RING E3 ubiquitin ligases (CRL) are the largest E3 family and direct numerous protein substrates for proteasomal degradation, thereby impacting a myriad of physiological and pathological processes including cancer. To date, there are no reported small molecule inhibitors of the catalytic activity of the CRLs. The present disclosure describes high throughput screening and medicinal chemistry optimization efforts that led to discovery of two compounds, #33-11 and KH-4-43, which inhibit E3 CRL4 and exhibit anti-tumor potential. These first-in-kind compounds bind to CRL4's core catalytic complex, inhibit CRL4 mediated ubiquitination and cause stabilization of CRL4's substrate CDT1 in cells. Treatment with #33-11 or KH-4-43 in a panel of 36 tumor cell lines revealed cytotoxicity. The anti-tumor activity was validated by the ability of the compounds to suppress the growth of human tumor xenografts in mice. Mechanistically, the compound's cytotoxicity was linked to aberrant accumulation of CDT1 that is known to trigger apoptosis. Moreover, a subset of tumor cells was found to express cullin4 proteins at levels as much as 70-fold lower than those in other tumor lines. The low-cullin4-expressing tumor cells appeared to exhibit increased sensitivity to #33-11/KH-4-43.

Cullin-RING E3 ubiquitin ligases (CRL) direct protein degradation to impact a myriad of physiological and pathological processes including cancer. This work reports the discovery of small molecule compounds, #33-11 and KH-4-43, as first-in-kind inhibitors of E3 CRL4 with anti-cancer potential. These compounds have provided opportunity for developing tool compounds to address mechanistic and phenotypic questions about CRL4 in biochemical, cell-based, and animal studies. The results of correlation studies between cullin4 protein level and drug sensitivity as well as cullin4 depletion experiments suggest a role for low E3 abundance in sensitizing tumor cells for apoptosis. Collectively the #33-11/KH-4-43-based CRL4 inhibitors may provide new exploitable therapeutic opportunities to target against a subset of tumor lines that are characterized by low CUL4 expression.

### BRIEF DESCRIPTION OF THE DRAWINGS

FIGS. 1A-C show cytotoxicity of compounds #33-11/KH-4-43 against a panel of tumor lines. The ability of #33-11 to induce apoptosis was measured by Annexin V flow cytometry (FIG. 1A). EC₅₀/EC₉₀ for each cell line are indicated. EC₅₀ or EC₉₀ valued at 100 μM is the upper limit, representing those with EC₅₀ or EC₉₀≥100 μM. Toxic response of a panel of cancer cells to #33-11/KH-4-43, as determined by viability assay (Cell Titerglo) (FIG. 1). Mouse pancreatic cancer cell lines K8484 and DT8082 were isolated from the KPC mice (Olive et al., “Inhibition of Hedgehog Signaling Enhances Delivery of Chemotherapy in a Mouse Model of Pancreatic Cancer,” Science 324(5933):1457-1461 (2009), which is hereby incorporated by reference in its entirety), the rest (34 in total) are all human cancer cell lines. Washout: MV4-11 cells at 10⁵ cells/ml were treated with compound at concentrations as indicated (FIG. 1C). At specified hours post compound treatment, the cells were washed once with fresh complete media without compound and allowed to grow for additional 48 hours followed by viability test as in FIG. 1B.

FIGS. 2A-E show effects of compounds on the ubiquitination of CK1α by E3 CRL4^{CRBN} in vitro. Lenalidomide-dependent ubiquitination of CK1α by E3 CRL4^{CRBN} driven by E2 UbcH5c and Cdc34b (FIG. 2A) (reaction scheme is shown on the top of FIG. 2A). F-Ub=Fluorescein-Ub (colored green). I-Ub-K48R=iFluor555-Ub-Q31C-K48R (colored red). The reaction products were analyzed by both merged fluorescence imaging and immunoblot using anti-CK1α antibody. KH-4-43 inhibits the ubiquitination of CK1α by CRL4^{CRBN} with Ub-K48R (FIG. 2B). KH-4-43 inhibits the ubiquitination of CK1α by CRL4^{CRBN} with the wild type Ub (FIG. 2C). Effects of #33-11 and #33 in the ubiquitination of CK1α by CRL4^{CRBN} (FIGS. 2D-E). The effects of #33-11 and #33 shown in panels D and E were determined using assays similar to B and C, respectively.

FIGS. 3A-D show effects of compound #33 on elongation of the IκBα-Ub fusion substrate by E2 Cdc34 and E3 Nedd8-SCF^{βTrCP2} or SCF^{βTrCP2}. Time course is shown in FIG. 3A. 30 nM Nedd8-SCF^{βTrCP} was used. Effects of #33 on the ubiquitination of IκBα-Ub with the wild type Ub (FIG. 3B) or Ub-K0 (FIG. 3C) by Nedd8-SCF^{βTrCP2} or SCF^{βTrCP2}. 100 nM E3 was used. The incubation was performed at 37° C. for 10 min. In FIG. 3C, the levels of ubiquitination were measured and shown for comparison. FIG. 3D shows effects of #33 on the ubiquitination of IκBα-Ub by Cdc34a or Cdc34b. 100 nM E3 was used. The incubation was performed at 37° C. for 10 min.

FIGS. 4A-B show comparison of compound #33 with #33-11/KH-4-43 on elongation of the IκBα-Ub fusion substrate by E2 Cdc34b and E3 Nedd8-SCF^{βTrCP2} Comparison of compound #33 with #33-11/KH-4-43 on elongation of IκBα-Ub with the wild type Ub (FIG. 4A) or Ub-K0 (FIG. 4B) by Cdc34b and Nedd8-SCF^{βTrCP2}.

FIGS. 5A-C show effects of compound #33 on ubiquitination of β-catenin by E3 Nedd8-SCF^{βTrCP2} and E2 Cdc34b or UbcH5c. Time course is shown in FIG. 5A. Effects of #33 on ubiquitination of β-catenin with Ub-K0 by Nedd8-SCF^{βTrCP2} and UbcH5c are shown in FIG. 5B. The incubation was performed at 37° C. for 1 min. Effects of #33 on ubiquitination of 3-catenin with Ub-K0 (lanes 1-6) or the wild type Ub (lanes 7-12) by Nedd8-SCF^{βTrCP2} and Cdc34b are shown in FIG. 5C. The incubation was performed at 37° C. for 10 min. The results showed that the ubiquitination of β-catenin by UbcH5c was significantly less affected than that by Cdc34b.

FIGS. 6A-B show comparison of compound #33 with #33-11/KH-4-43 on ubiquitination of β-catenin by E3 Nedd8-SCF^{βTrCP2} and E2 Cdc34b or UbcH5c. Comparison of compound #33 with #33-11/KH-4-43 on ubiquitination of β-catenin by Nedd8-SCF^{βTrCP2} and Cdc34b (FIG. 6A) or UbcH5c (FIG. 6B).

FIG. 7 shows effects of compound #33 on CUL1-dependent di-Ub synthesis. #33 inhibits the ability of ROC1-CUL1 CTD to discharge the Cdc34-Ub thiol ester complex. In this reaction, a preformed E2 Cdc34-S-Ub complex was chased with excess receptor Ub in the presence of the E3 ROC1-CUL1 CTD complex. In the absence of compound, Cdc34-S-Ub was discharged, forming di-Ub. Compound #33 was able to block the discharge and prevent the formation of di-Ub (lane 5). However, increasing the concentration of ROC1-CUL1 CTD by 3-fold largely reversed the inhibition (lanes 6 and 7). These results suggest that compound #33 directly targets ROC1-CUL1 CTD. Collectively, the results shown in FIGS. 3A-D, FIGS. 5A-C, and FIG. 7 conclude that #33 is an inhibitor of E3 SCF by targeting the ROC1-CUL1 core ligase.

FIGS. 8A-C show comparison of #33-11/KH-4-43 on gel-based, CUL1-dependent di-Ub synthesis reactions. Compound #33 showed inhibition as it blocked the ability of E3 subcomplex ROC1-CUL1 to discharge Cdc34-Ub and to produce Ub₂ (FIG. 8A). By contrast, no effect was observed with KH-4-43 (FIG. 8B). Straight di-Ub synthesis reaction was run using a previously established di-Ub synthesis assay (Wu et al., “Priming and Extending: an UbcH5/Cdc34 E2 Handoff Mechanism for Polyubiquitination on a SCF Substrate,” Mol. Cell 37:784-796 (2010), which is hereby incorporated by reference in its entirety) (FIG. 8C). As shown, the level of di-Ub product remained almost unchanged in the presence #33-11 up to 150 μM. The addition of 150 μM of #33-11 to the reaction brings DMSO to a concentration of 2.3%, which is close to the threshold of DMSO that would cause inhibition of ubiquitination under the reaction conditions used.

FIG. 9 shows that compound #33-11 exhibits little effect on ubiquitination by E3 complex ROC1-CUL2 or ROC1-CUL3. To determine the effects of #33-11 on other E3 core ligase complexes including ROC1-CUL2 or ROC1-CUL3, a previously established di-Ub synthesis assay (Wu et al., “Priming and Extending: an UbcH5/Cdc34 E2 Handoff Mechanism for Polyubiquitination on a SCF Substrate,” Mol. Cell 37:784-796 (2010), which is hereby incorporated by reference in its entirety), was used. The reaction formed di-Ub product with ROC1-CUL2 (Left, lane 2) or ROC1-CUL3 (right, lane 2). This reaction required E1/E2/E3 (data not shown, see Wu et al., “Priming and Extending: an UbcH5/Cdc34 E2 Handoff Mechanism for Polyubiquitination on a SCF Substrate,” Mol. Cell 37:784-796 (2010), which is hereby incorporated by reference in its entirety). As shown, the level of di-Ub product remained almost unchanged in the presence #33-11 up to 150 μM. The addition of 150 μM of #33-11 to the reaction brought DMSO to a concentration of 2.3%, which is close to the threshold of DMSO that would cause inhibition of ubiquitination under the reaction conditions used.

FIGS. 10A-C show reversal of the inhibitory effects of compound #33 by washing the assembly of substrate-E3-inhibitor complex. Ubiquitination of immobilized GST-IκBα-Ub by Nedd8-SCF^{βTrCP2} is shown in FIG. 10A. The ubiquitination of immobilized GST-IκBα-Ub was inhibited by increasing concentrations of #33 (FIG. 10B). Washing reversed the inhibitory effects of #33 (FIG. 10C). After assembling immobilized GST-IκBα-Ub/Nedd8-SCF^{βTrCP2}/#33 complex, the assembly was gently washed followed by addition of preformed Cdc34b-Ub. Under this condition, low levels of GST-IκBα-Ub ubiquitination were observed (lane 2), indicating that small amount of GST-IκBα-Ub/Nedd8-SCF^{βTrCP2} complex remained after washing and supported ubiquitination. However, no inhibitory effects by #33 were detected (lanes 3 and 4), suggesting that the compound was removed by the washing step. These observations are consistent with compound #33 acting in a reversible manner.

FIGS. 11A-B are graphs showing a comparison of #33/#33-11/KH-4-43 on binding to E3 ROC1-CUL1 CTD and on FRET K48 di-Ub synthesis. Ligand binding to ROC1-CUL1 CTD is shown in FIG. 11A. Multiple Micro-Scale Thermophoresis (MST) binding experiments were performed and the representative binding curves are shown. K_{d} value for each experiment as well as the average are shown. FRET K48 di-Ub synthesis and determination of IC₅₀ is shown in FIG. 11B. Multiple synthesis reactions with each inhibitor were performed and the representative inhibition curves are shown. IC₅₀ value for each experiment as well as the average are shown. FRET assay run without inhibition and time point with which the inhibition experiments were performed is indicated by arrows (FIG. 11B, right).

FIGS. 12A-B are graphs showing the influence of Nedd8 modification on binding of compound #33 to ROC1-CUL1 and on CUL1-dependent di-Ub synthesis. Binding of compound #33 to ROC1-CUL1 and ROC1-Nedd8-CUL1 are shown in FIG. 12A. FRET K48 di-Ub synthesis and IC₅₀ determination are show in FIG. 12B. The time course experiment without inhibitor was run (Top) and linear range time points were picked to run experiments with the inhibitor.

FIGS. 13A-B show effects of compounds #33-11 and KH-4-43 on ubiquitination by E3 ROC1-CUL4A in vitro. Compound #33-11 inhibits ubiquitination by E3 complex ROC1-CUL4A (FIG. 13A). The reaction scheme is shown in FIG. 13A top. RC4=ROC1-CUL4A. F-Ub=Fluorescein-Ub (colored green). R-Ub=Rhodamine-Ub (colored red). The merged imaging shows robust R-Ub chain formation on CUL4A (lane 4), which was blocked by #33-11 (merged, lanes 5-8). Imaging F-Ub alone shows disappearance of CUL4A-F-Ub in the chase (green only, compare lanes 2 and 4), due to its conversion to poly-ubiquitinated forms in elongation. Compound #33-11 blocked the utilization of CUL4A-F-Ub by Cdc34 (lanes 7 and 8). Imaging R-Ub alone shows the utilization of R-Ub in the chase, which was inhibited by #33-11 (red only, lanes 3-8). Side-by-side comparison between KH-4-43 and #33-11 in ubiquitination inhibition is shown in FIG. 13B. The reaction was identical to the one in FIG. 13A with indicated compounds of increasing concentrations.

FIGS. 14A-B show reversal of the inhibitory effects of KH-4-43 by washing the assembly of E3-inhibitor complex. Primed ubiquitination of immobilized GST-ROC1-CUL4A is shown in FIG. 14A. The reaction scheme is shown on top of FIG. 14A. Immobilized GST-ROC1-CUL4A was ubiquitinated in a time-dependent fashion, which was stimulated by priming ubiquitination catalyzed by UbcH5c. Washing reversed the inhibitory effects of KH-4-43 (FIG. 14B). Compound KH-4-43 inhibited the ubiquitination of immobilized GST-ROC1-CUL4A (compare lane 2 with lanes 3 and 5). However, if the beads were washed after inhibitor addition, the inhibitory effects of KH-4-43 were reduced significantly (lanes 4 and 6), suggesting that the compound were removed by the washing step. There observations are consistent with the KH-4-43 being a reversible manner.

FIGS. 15A-C show effects of #33-11 on ³²P-Ub chain elongation by ROC1-CUL4A. To specifically determine the effects of #33-11 on Ub chains assembled on cullins, the “Cullin-³²P-Ub elongation assay” was employed. In this two-step reaction, CUL4A or CUL1 was first mono-ubiquitinated by incubating ROC1-CUL4A or ROC1-CUL1 with E1 and E2 UbcH5c to form CUL4A (or CUL1)-³²P-Ub (lane 2). In step 2, CUL4A-Ub or CUL1-Ub was elongated by chasing with preformed Cdc34b˜Ub in the presence or absence of #33-11. As shown, CUL4A-³²P-Ub disappeared in chase, concomitant with accumulation of higher molecular weight Ub conjugates (Left, lane 4). Compound #33-11 inhibited the elongation of CUL4A-³²P-Ub with an IC₅₀ of 21 μM, similar to that determined using the fluorescence assay. By contrast, little effects were observed on the levels of CUL1-³²P-Ub with as much as 150 μM of #33-11 (Right, lanes 5-8; also see quantification shown below the gel). The apparent discordance between high IC₅₀ (21 μM) and low Kd (0.22 μM) is because the “Cullin-Ub elongation assay” requires high levels of ROC1-CUL4A (0.9 μM). Lowering E3 diminished the reaction. Compound #33-11 inhibited elongation by 50% when the compound/E3 ratio approached 30:1.

FIG. 16 shows effects of #33-11 on Ub chain assembly by Nedd8-ROC1-CUL1 or Nedd8-ROC1-CUL4A. The effects of #33-11 on Ub chain assembly by Nedd8-ROC1-CUL1 (Top) or Nedd8-ROC1-CUL4A (Bottom) were compared. The use of Coomassie stain for measurements allowed tracking of all the reaction components in reactions with or without the compound. The results showed that nearly all of free Ub was utilized by either Nedd8-ROC1-CUL1 (Top) or Nedd8 ROC1-CUL4A (Bottom) to assemble Ub chains (compare lanes 1 and 2). In the absence of Nedd8-ROC1-CUL1, the levels of free Ub remained unchanged (compare Top, lanes 1-3 and 7), demonstrating the requirement of E3 for the reaction. In the presence of Nedd8-ROC1-CUL1, #33-11 at 10 or 100 μM had little effect on the reaction, while this compound at 1 mM blocked the utilization of Ub (Top, lanes 4-6). However, #33-11 at 10 μM was able to block the utilization of free Ub in the presence of Nedd8-ROC1-CUL4A (Bottom, lanes 3-6). This difference demonstrates that #33-11 inhibits Nddd8-ROC1-CUL4A more potently than Nedd8-ROC1-CUL1.

FIG. 17 is a schematic showing the discovery of E3 CRL inhibitors. The E3 inhibitor discovery program comprised multiple phases including HTS, hit-to-lead characterization, SAR-by-catalog, and SAR by medicinal chemistry. Compound #33 was identified as a hit by HTS using the FRET K48 di-Ub assay (Wu et al., “Suramin Inhibits Cullin-RING E3 Ubiquitin Ligases,” PNAS 113(14):E2011-2018(2016), which is hereby incorporated by reference in its entirety). Subsequent hit characterization has revealed the ability of #33 to inhibit E3 SCF/CRL1 (FIGS. 3A-D, FIGS. 5A-C, and FIG. 7). Substructure searches of commercially available compound libraries identified 21 analogs of #33. The SAR analyses of these 21 analogs, including E3 binding (FIGS. 19A-C), E3 activity inhibition (FIGS. 4A-B and FIGS. 6A-B), and substrate stabilization (FIGS. 20A-C), have led to the conclusion that #33-11, a #33 analog, selectively targets E3 CRL4. 56 analogs of #33-11 were synthesized, resulting in identification of an improved analog KH-4-43 (FIGS. 2A-E, FIGS. 19A-C, and FIGS. 20A-C).

FIG. 18 shows effects of compounds on Ub thiol ester complex formation with E1/E2 Cdc34. Reaction mixture (15p) contained 33 mM Tris-HCl (pH 7.4), 1.7 mM MgCl₂, 0.33 mM DTT, BSA (0.07 mg/ml), I-Ub-Q31C/K48R (1.62 μM), Ub E1 (14 nM), with or without E2 Cdc34 (124 nM), in the presence or absence of compound. The reaction was initiated by addition of ATP (0.66 mM) and the incubation was at 37° C. for 15 min. The reaction was stopped by EDTA (12.5 mM). The reaction products were separated by 4-20% SDS-PAGE followed by detection with a scanner (Typhoon 9500). The levels of thiol esters formed are shown.

FIGS. 19A-C show a new class of E3 CRL inhibitors and their selective interactions with ROC1-CUL4A CTD. Chemical structures are shown in FIG. 19A and ligand-E3 binding measured by MST are shown in FIG. 19B-C. Purified ROC1-CUL4A CTD was mixed with increasing amounts of KH-4-43 and the resulting mixtures were analyzed by MST. The fitting binding curve was generated with calculated K_{d} (FIG. 19B). Similar binding experiments were performed with various complexes or single protein agent along with indicated compounds. The binding K_{d} is indicated in FIG. 19C. With respect to ROC1-CUL1 CTD, average K_{d} is shown for each compound based on results of multiple MST experiments as detailed in FIG. 11A. CUL4A CTD=CUL4A amino acids 400-759; CUL1 CTD=CUL1 amino acids 411-776.

FIGS. 20A-C show that treatment of cells with compounds KH-4-43 and #33-11 caused accumulation of E3 CRL4 substrate CDT1. KH-4-43 or #33-11 caused accumulation of CDT1 in AML MV4-11 cells in a dose-dependent manner (FIG. 20A). The abundance of CDT1 in cells treated with compounds were analyzed by immunoblot. The graph shows the quantification of three independent experiments with error bars indicating experimental variations. Comparison of #33-11 with the Nedd8 inhibitor MLN4924 in MV4-11 cells is shown in FIG. 20B. Comparison of #33-11 and #33 in the ability to cause accumulation of CDT1 in AML MV4-11 and NB-4 cells is shown in FIG. 20C. Loading controls: GAPDH, Cyclophilin B, and Tubulin.

FIGS. 21A-C show low expression of CUL4A and CUL4B in a subset of tumor cells. The abundance of CUL4A or CUL4B in a panel of 10 tumor lines were analyzed by immunoblot (FIG. 21A). FIG. 21B is a pair of graphs showing the relative abundance of CUL4A (left) or CUL4B (right). The numbers of biological replicates are: 7 (MV4-11), 4 (NB-4), 4 (MOLT-4), 2 (ML-2), 2 (K562), 3 (THP1), 3 (U2OS), 3 (MDA-MB-231), 3 (HCT116), and 2 (H1299). Insets are close-up view of the low-CUL4 expressing lines. A scatter plot of CUL4 abundance vs. drug sensitivity to #33-11 is shown in FIG. 21C. The CUL4 concentration and #33-11 sensitivity (EC₅₀) for indicated cell lines were obtained from Table 4.

FIG. 22 shows Immunoblot analysis of CUL1, 2, 3, or 5 from a panel of tumor lines. The same subset of tumor lines analyzed in FIG. 5A was subjected to immunoblotting for the abundance of CUL1-3 and CUL5. Relative level of CUL1, 2, 3, or 5 is shown below each cullin blot. Quantification of GAPDH and Cyclophilin B, both of which are loading controls, revealed that variation of these standards between the cell lines examined is less than 30%.

FIG. 23 is a graph showing cytotoxicity of compounds KH-4-43 against a panel of tumor lines. The ability of KH-4-43 to induce apoptosis was measured by Annexin V flow cytometry.

FIGS. 24A-D show that #33-11 targets CRL4/CDT1 specifically. FIGS. 24A-B show depletion of CUL4 sensitized U2OS cells to #33-11 for apoptosis. Immunoblots confirmed depletion of CUL4A or CUL4B in U2OS cells treated with siRNAs (FIG. 24A). siRNA-exposed U2OS cells were treated with #33-11, followed by Annexin V flow cytometry to quantify apoptosis (FIG. 24B). The graph represents three independent experiments with error bars indicating experimental variations. IC₅₀ was calculated using SigmaPlot and two-sided t test was performed using Microsoft Excel to determine P value of 0.0047. FIGS. 24 C-D show that the CDT1 knockdown diminishes cytotoxic effects of #33-11. An AML MV4-11 cell line was created to induce expression of shRNA by Doxycycline (DOX) to deplete CDT1. Western analysis confirms depletion of CDT1 by DOX. HSP90/GAPDH/Cyclophilin B are loading controls (FIG. 24C). Apoptotic response with or without DOX is shown in FIG. 24D. The Annexin V positive apoptotic cells in response to #33-11 were reduced by DOX treatment. The graph incorporates two technical repeats.

FIGS. 25A-E show results of a mouse study. Tolerance/Toxicity of #33-11 is shown in FIG. 25A. Effects of KH-4-43 in the AML MV4-11 subcutaneous xenograft are shown in FIGS. 25B-E. Body weight changes are shown in FIG. 25B. Changes of tumor growth in weight in groups G1-G5 were determined by statistical analysis using one-way ANOVA (FIG. 25C). End-of-study PK: concentration of KH-4-43 in the plasma in G3 is shown in FIG. 25D. Concentration of KH-4-43 in the tumor in the G4 group is shown in FIG. 25E.

FIG. 26 is a graph showing effects of KH-4-43 in the AML MV4-11 subcutaneous xenograft. 10 mice were used in each group (n=10). The changes of tumor volume in groups G1-G5 at indicated time points after tumor inoculation were subjected to statistical analysis using two-way ANOVA, respectively.

FIG. 27 is a graph showing characterization of 6-Bromo-7,8-dihydroxy-3-phenoxy-2-(trifluoromethyl)-4H-chromen-4-one.

FIG. 28 is a graph showing characterization of 6-Bromo-3-(4-bromophenoxy)-7,8-dihydroxy-2-(trifluoromethyl)-4H-chromen-4-one.

FIG. 29 is a graph showing characterization of 2-(4-Bromophenoxy)-1-(2,3,4-trihydroxyphenyl)ethan-1-one.

FIG. 30 is a graph showing characterization of 2-(3-Bromophenoxy)-1-(2,3,4-trihydroxyphenyl)ethan-1-one.

FIG. 31 is a graph showing characterization of 2-(2-Bromophenoxy)-1-(2,3,4-trihydroxyphenyl)ethan-1-one.

FIG. 32 is a graph showing characterization of 3-(4-Bromophenoxy)-7,8-dihydroxy-2-(trifluoromethyl)-4H-chromen-4-one.

FIG. 33 is a graph showing characterization of 3-(3-Bromophenoxy)-7,8-dihydroxy-2-(trifluoromethyl)-4H-chromen-4-one.

FIG. 34 is a graph showing characterization of 3-(2-Bromophenoxy)-7,8-dihydroxy-2-(trifluoromethyl)-4H-chromen-4-one.

FIGS. 35A-C show an inverse screen that assessed the impact of KH-4-43 and 33-11 on a panel of cell lines from 14 tumor types, highlighting the sensitivity of ovarian cancer cell lines to CRL4 inhibition. FIG. 35A shows the tumor types evaluated in dose response assays.

FIG. 35B shows the sensitivity of these lines to #33-11. FIG. 35C shows the sensitivity of these lines to KH-4-43.

FIGS. 36A-B show the sensitivity of an expanded panel of ovarian cancer cell lines to CRL4 inhibition by KH-4-43 via dose response analysis in a six point dose response assay (10 μM-125 nM) (FIG. 36A) and the quantification of these curves as area under the curve and effective concentration 30/50 values (FIG. 36B).

FIG. 37 shows the results of a survival study of ovarian cancer OVCAR8 xenografts showing the impact of KH-4-43 (50 mg/kg) as compared to standard of care Cisplatin or Vehicle controls.

FIG. 38 is a graph showing KH-4-43 dose response in murine PDAC KPC cell lines.

### DETAILED DESCRIPTION OF THE INVENTION

The present disclosure relates to inhibitors of cullin-RING E3 ubiquitin ligase 4 and methods of their use.

One aspect of the present disclosure relates to a compound of formula (I) having the following structure:

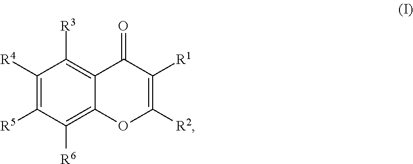

or a stereoisomer, pharmaceutically acceptable salt, oxide, or solvate thereof, wherein
R¹ is H,

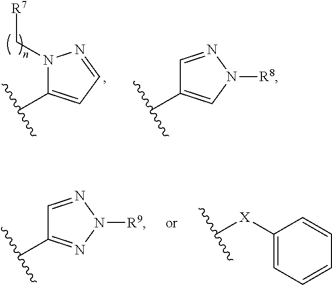

wherein the benzene ring of

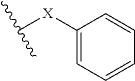

is optionally substituted with NO₂ or halogen;
R² is H or CF₃;
R³, R⁴, R⁵, and R⁶ are each independently selected from the group consisting of H, OH, OAc, halogen, alkoxy, OTf, and ONa;
R⁷, R⁸, and R⁹ are each independently selected from H, C₁-C₆ alkyl, pyridine, or

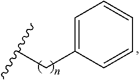

wherein the benzene ring of

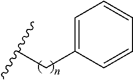

is optionally substituted one or more times with C₁-C₆ alkyl, NH₂, CF₃, halogen, CN, C(O)NH₂, and alkoxy;
X is either present or absent, and when present is CH₂ or O; and
n is 0 or 1;
   with the proviso that when R³ and R⁴ are H, R⁵ and R⁶ are OH, and R² is CF₃, R¹ is not

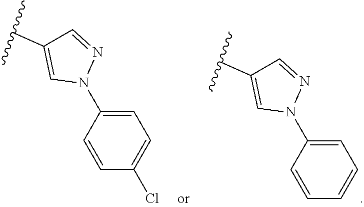 [SMILES: *c1cnn(-c2ccc(Cl)cc2)c1 ; *c1cnn(-c2ccccc2)c1 ; "or"]

Another aspect of the disclosure relates to a compound of formula (I) having the following structure:

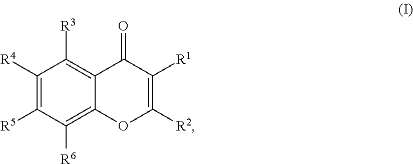

or a stereoisomer, pharmaceutically acceptable salt, oxide, or solvate thereof, wherein
R¹ is H,

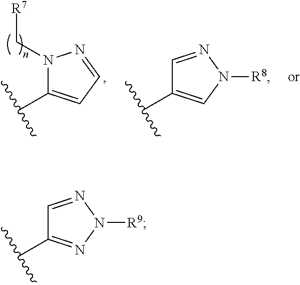

R² is H or CF₃;
R³, R⁴, R⁵, and R⁶ are each independently selected from the group consisting of H, OH, OAc, alkoxy, OTf, and ONa;
R⁷, R⁸, and R⁹ are each independently selected from H, C₁-C₆ alkyl, pyridine, or

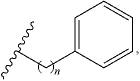

wherein the benzene ring of

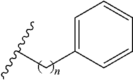

is optionally substituted one or more times with C₁-C₆ alkyl, NH₂, CF₃, halogen, CN, C(O)NH₂, and alkoxy; and
n is 0 or 1;
   with the proviso that when R³ and R⁴ are H, R⁵ and R⁶ are OH, and R² is CF₃, R¹ is not

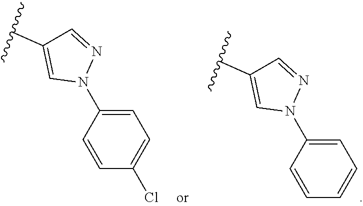 [SMILES: *c1cnn(-c2ccc(Cl)cc2)c1 ; *c1cnn(-c2ccccc2)c1 ; "or"]

As used above, and throughout the description herein, the following terms, unless otherwise indicated, shall be understood to have the following meanings. If not defined otherwise herein, all technical and scientific terms used herein have the same meaning as is commonly understood by one of ordinary skill in the art to which this technology belongs.

As used herein, the term “halogen” means fluoro, chloro, bromo, or iodo.

The term “alkyl” means an aliphatic hydrocarbon group which may be straight or branched having about 1 to about 6 carbon atoms in the chain (or the number of carbons designated by “Cₙ-Cₙ”, where n is the numerical range of carbon atoms). Branched means that one or more lower alkyl groups such as methyl, ethyl, or propyl are attached to a linear alkyl chain. Exemplary alkyl groups include methyl, ethyl, n-propyl, i-propyl, n-butyl, t-butyl, n-pentyl, and 3-pentyl.

The term “alkoxy” means groups of from 1 to 6 carbon atoms of a straight, branched, or cyclic configuration and combinations thereof attached to the parent structure through an oxygen. Examples include methoxy, ethoxy, propoxy, isopropoxy, butoxy, cyclopropyloxy, cyclohexyloxy, and the like. Alkoxy also includes methylenedioxy and ethylenedioxy in which each oxygen atom is bonded to the atom, chain, or ring from which the methylenedioxy or ethylenedioxy group is pendant so as to form a ring. Thus, for example, phenyl substituted by alkoxy may be, for example,

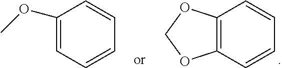

As used herein, “heterocycle” refers to a stable 3- to 18-membered ring (radical) of carbon atoms and from one to five heteroatoms selected from nitrogen, oxygen, and sulfur. The heterocycle may be a monocyclic or a polycyclic ring system, which may include fused, bridged, or spiro ring systems; and the nitrogen, carbon, or sulfur atoms in the heterocycle may be optionally oxidized; the nitrogen atom may be optionally quaternized; and the ring may be partially or fully saturated.

The phrases “substituted or unsubstituted” and “optionally substituted” mean a group may (but does not necessarily) have a substituent at each substitutable atom of the group (including more than one substituent on a single atom), and the identity of each substituent is independent of the others.

The term “substituted” means that one or more hydrogen on a designated atom is replaced with a selection from the indicated group, provided that the designated atom's normal valency is not exceeded. “Unsubstituted” atoms bear all of the hydrogen atoms dictated by their valency. When a substituent is oxo (i.e., ═O), then 2 hydrogens on the atom are replaced. Combinations of substituents and/or variables are permissible only if such combinations result in stable compounds. By “stable compound” it is meant a compound that is sufficiently robust to survive isolation to a useful degree of purity from a reaction mixture and formulation into an efficacious therapeutic agent.

By “compound(s)” of the disclosure and equivalent expressions, it is meant compounds herein described, which expression includes the prodrugs, the pharmaceutically acceptable salts, the oxides, and the solvates, e.g. hydrates, where the context so permits.

Compounds described herein may contain one or more asymmetric centers and may thus give rise to enantiomers, diastereomers, and other stereoisomeric forms. Each chiral center may be defined, in terms of absolute stereochemistry, as (R)- or (S)-. The present disclosure is meant to include all such possible isomers, as well as mixtures thereof, including racemic and optically pure forms. Optically active (R)- and (S)-, (−)- and (+)-, or (D)- and (L)-isomers may be prepared using chiral synthons or chiral reagents, or resolved using conventional techniques. All tautomeric forms are also intended to be included.

As would be understood by a person of ordinary skill in the art, the recitation of “a compound” is intended to include salts, solvates, oxides, and inclusion complexes of that compound as well as any stereoisomeric form, or a mixture of any such forms of that compound in any ratio. Thus, in accordance with some embodiments of the present disclosure, a compound as described herein, including in the contexts of pharmaceutical compositions, methods of treatment, and compounds per se, is provided as the salt form.

The term “solvate” refers to a compound in the solid state, where molecules of a suitable solvent are incorporated in the crystal lattice. A suitable solvent for therapeutic administration is physiologically tolerable at the dosage administered. Examples of suitable solvents for therapeutic administration are ethanol and water. When water is the solvent, the solvate is referred to as a hydrate. In general, solvates are formed by dissolving the compound in the appropriate solvent and isolating the solvate by cooling or using an antisolvent. The solvate is typically dried or azeotroped under ambient conditions.

Inclusion complexes are described in Remington, The Science and Practice of Pharmacy, 19th Ed. 1:176-177 (1995), which is hereby incorporated by reference in its entirety. The most commonly employed inclusion complexes are those with cyclodextrins, and all cyclodextrin complexes, natural and synthetic, are specifically encompassed by the present invention.

The term “pharmaceutically acceptable salt” refers to salts prepared from pharmaceutically acceptable non-toxic acids or bases including inorganic acids and bases and organic acids and bases.

The term “pharmaceutically acceptable” means it is, within the scope of sound medical judgment, suitable for use in contact with the cells of humans and lower animals without undue toxicity, irritation, allergic response and the like, and are commensurate with a reasonable benefit/risk ratio.

In some embodiments of the compound of formula (I), R¹ is H.

In some embodiments of the compound of formula (I), R¹ is

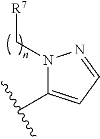

### and R⁷ is

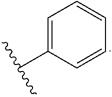 [SMILES: *c1ccccc1]

In some embodiments of the compound of formula (I), R¹ is

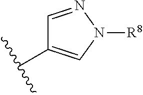

and R⁸ is substituted

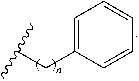

In some embodiments of the compound of formula (I), R¹ is

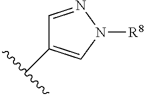

and R⁸ is C₁-C₆ alkyl.

In some embodiments of the compound of formula (I), R¹ is

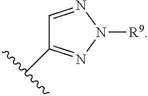

In some embodiments of the compound of formula (I), R² is H.

In some embodiments of the compound of formula (I), R² is CF₃.

In some embodiments of the compound of formula (I), R³ and R⁴ are both H.

In some embodiments of the compound of formula (I), R⁵ and R⁶ are both OH, OAc, ONa, or alkoxy. In some embodiments, R⁵ and R⁶ are both OH. Exemplary compounds may include, but are not limited to,

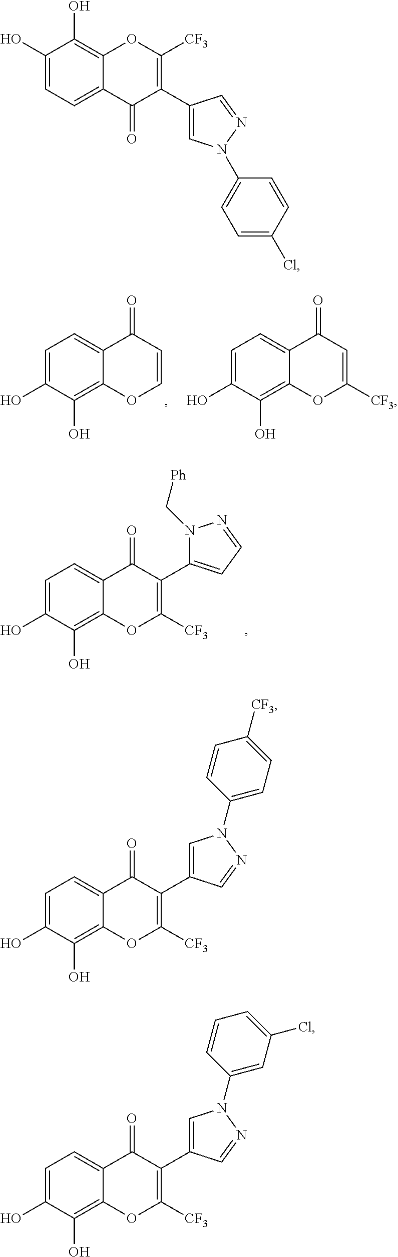

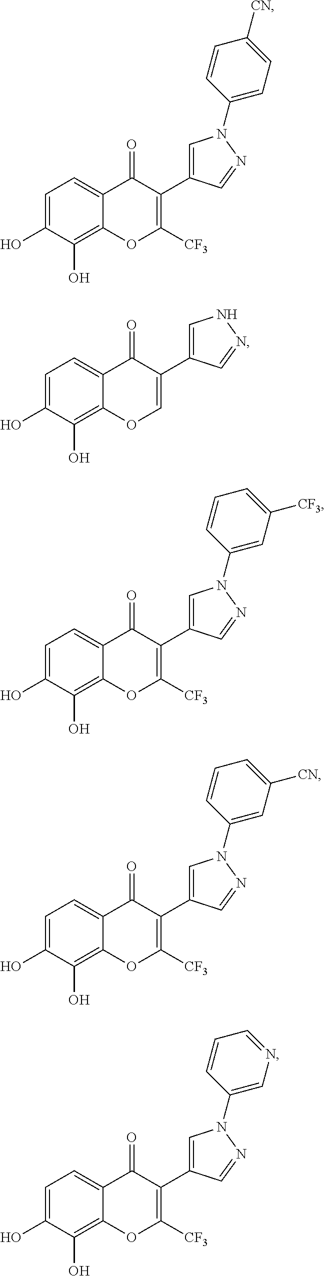 [SMILES: N#Cc1ccc(-n2cc(-c3c(C(F)(F)F)oc4c(O)c(O)ccc4c3=O)cn2)cc1 ; O=c1c(-c2cn[nH]c2)coc2c(O)c(O)ccc12 ; O=c1c(-c2cnn(-c3cccc(C(F)(F)F)c3)c2)c(C(F)(F)F)oc2c(O)c(O)ccc12 ; N#Cc1cccc(-n2cc(-c3c(C(F)(F)F)oc4c(O)c(O)ccc4c3=O)cn2)c1 ; O=c1c(-c2cnn(-c3cccnc3)c2)c(C(F)(F)F)oc2c(O)c(O)ccc12]

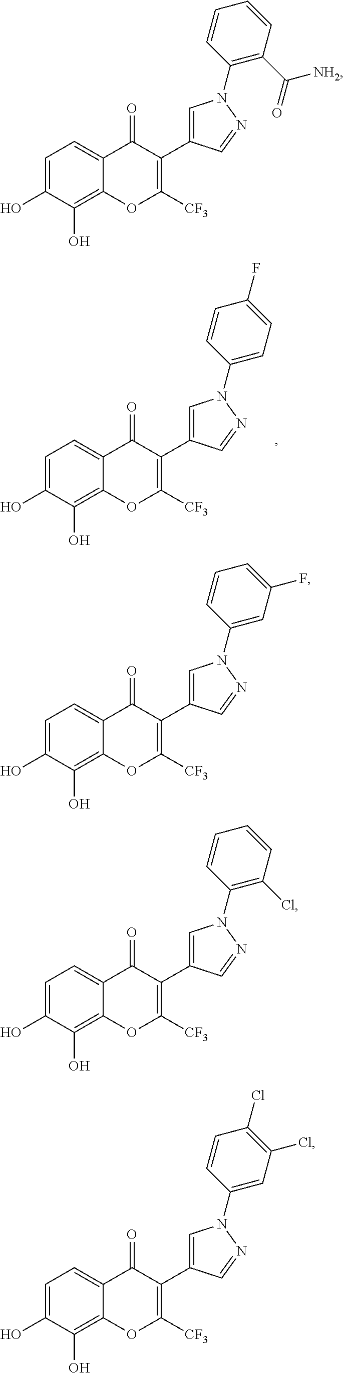 [SMILES: NC(=O)c1ccccc1-n1cc(-c2c(C(F)(F)F)oc3c(O)c(O)ccc3c2=O)cn1 ; O=c1c(-c2cnn(-c3ccc(F)cc3)c2)c(C(F)(F)F)oc2c(O)c(O)ccc12 ; O=c1c(-c2cnn(-c3cccc(F)c3)c2)c(C(F)(F)F)oc2c(O)c(O)ccc12 ; O=c1c(-c2cnn(-c3ccccc3Cl)c2)c(C(F)(F)F)oc2c(O)c(O)ccc12 ; O=c1c(-c2cnn(-c3ccc(Cl)c(Cl)c3)c2)c(C(F)(F)F)oc2c(O)c(O)ccc12]

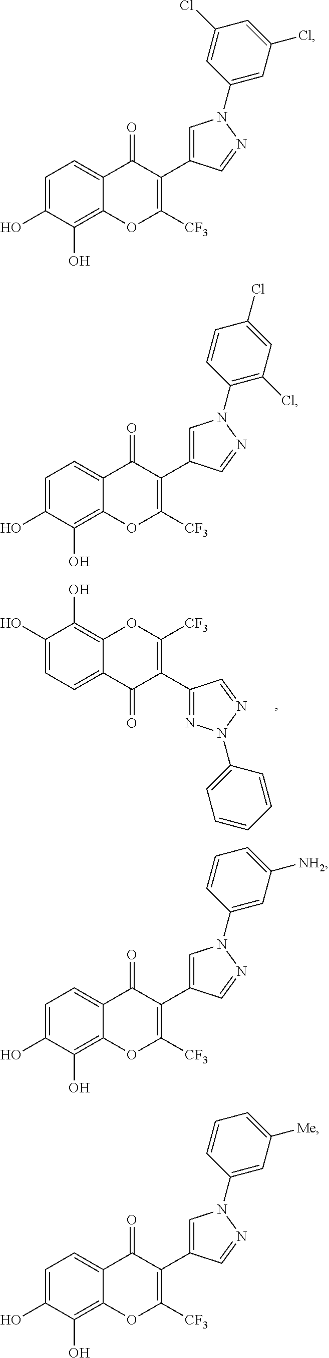

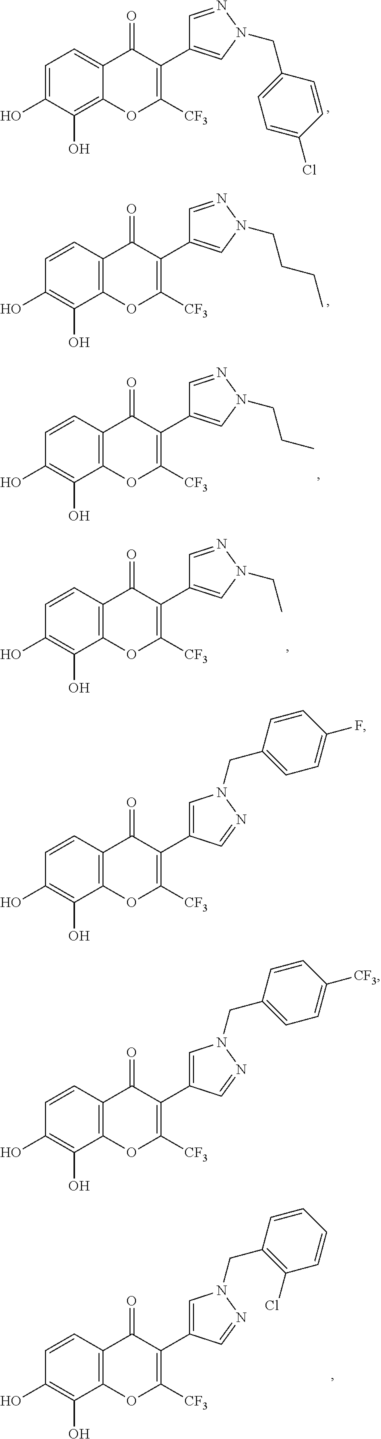

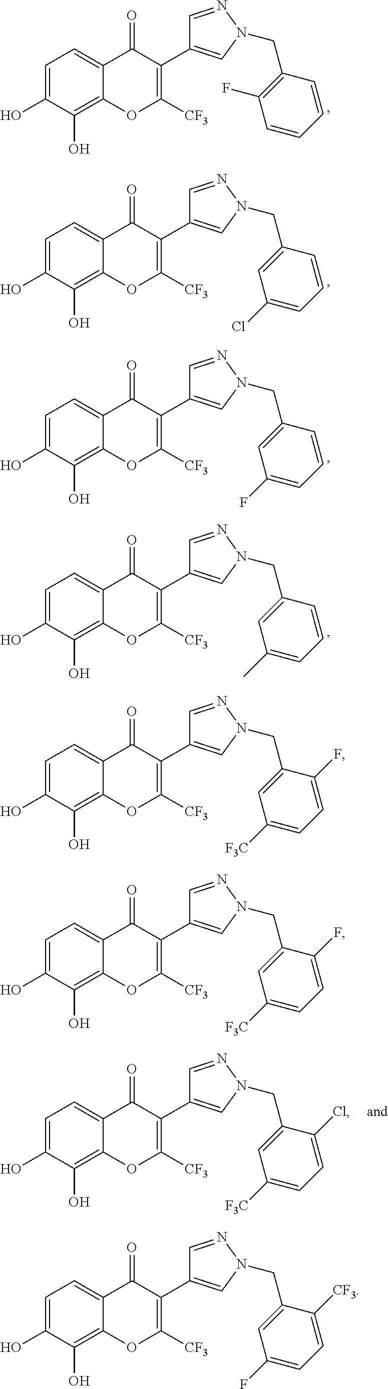

In some embodiments of the compound of formula (I), the compound is selected from the group consisting of

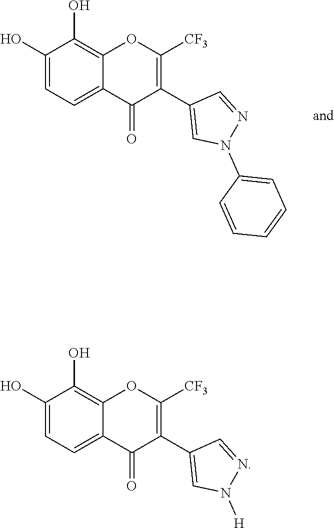

In some embodiments of the compound of formula (I), R⁵ and R⁶ are both OAc. An exemplary compound of these embodiments is

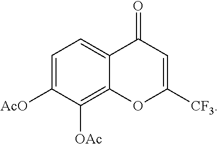 [SMILES: CC(=O)Oc1ccc2c(=O)cc(C(F)(F)F)oc2c1OC(C)=O]

In some embodiments of the compound of formula (I), R⁵ and R⁶ are both alkoxy. Exemplary compounds may include, but are not limited to,

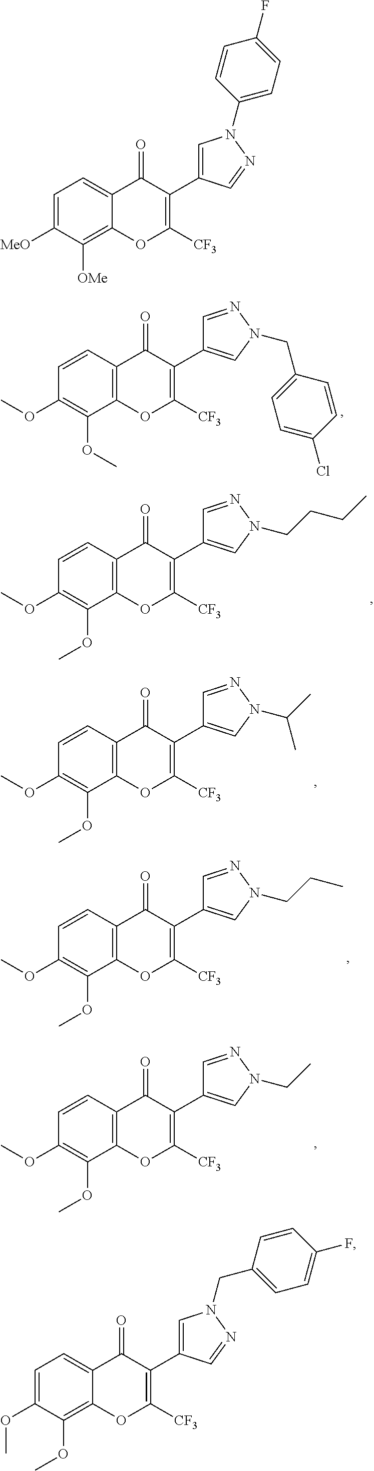

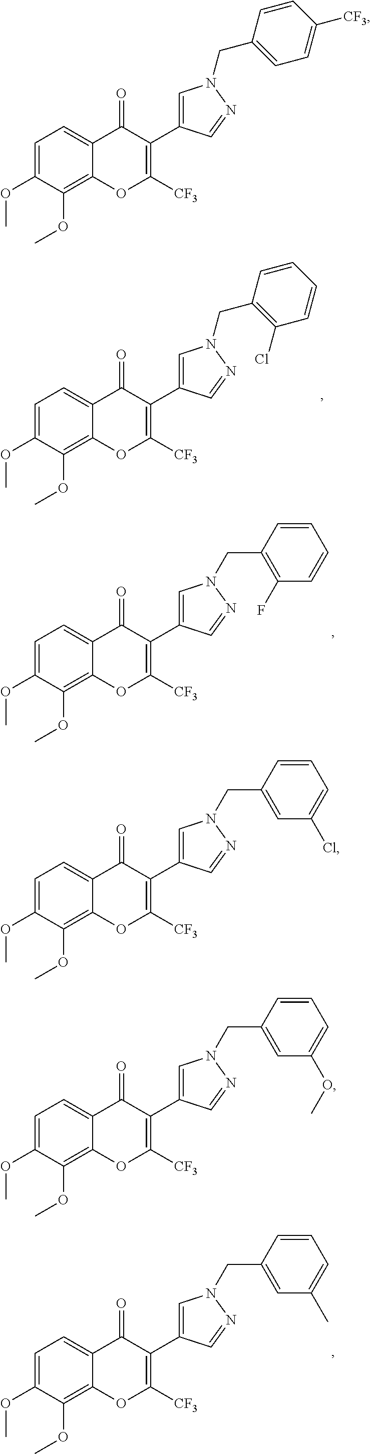 [SMILES: COc1ccc2c(=O)c(-c3cnn(Cc4ccc(C(F)(F)F)cc4)c3)c(C(F)(F)F)oc2c1OC ; COc1ccc2c(=O)c(-c3cnn(Cc4ccccc4Cl)c3)c(C(F)(F)F)oc2c1OC ; COc1ccc2c(=O)c(-c3cnn(Cc4ccccc4F)c3)c(C(F)(F)F)oc2c1OC ; COc1ccc2c(=O)c(-c3cnn(Cc4cccc(Cl)c4)c3)c(C(F)(F)F)oc2c1OC ; COc1cccc(Cn2cc(-c3c(C(F)(F)F)oc4c(OC)c(OC)ccc4c3=O)cn2)c1 ; COc1ccc2c(=O)c(-c3cnn(Cc4cccc(C)c4)c3)c(C(F)(F)F)oc2c1OC]

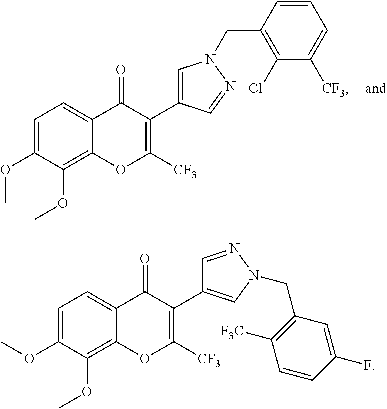 [SMILES: COc1ccc2c(=O)c(-c3cnn(Cc4cccc(C(F)(F)F)c4Cl)c3)c(C(F)(F)F)oc2c1OC ; "and" ; COc1ccc2c(=O)c(-c3cnn(Cc4cc(F)ccc4C(F)(F)F)c3)c(C(F)(F)F)oc2c1OC]

In some embodiments of the compound of formula (I), the compound is

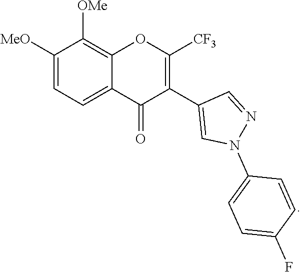 [SMILES: COc1ccc2c(=O)c(-c3cnn(-c4ccc(F)cc4)c3)c(C(F)(F)F)oc2c1OC]

In some embodiments of the compound of formula (I), the compound is selected from the group consisting of

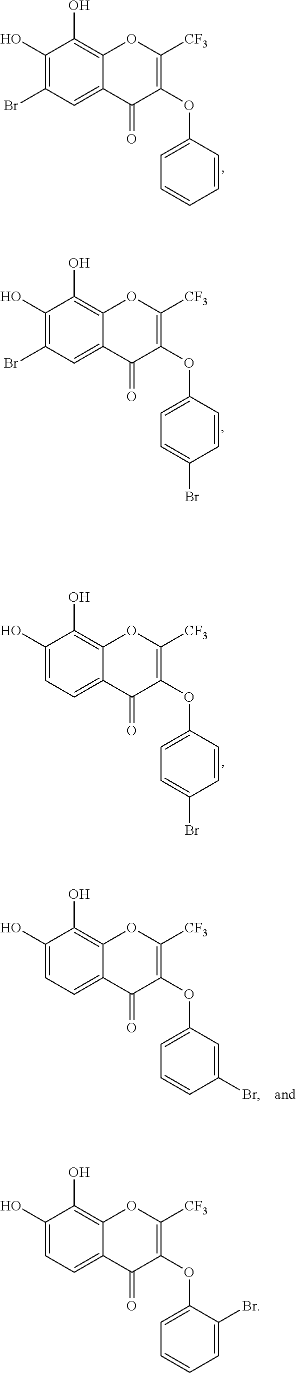 [SMILES: O=c1c(Oc2ccccc2)c(C(F)(F)F)oc2c(O)c(O)c(Br)cc12 ; O=c1c(Oc2ccc(Br)cc2)c(C(F)(F)F)oc2c(O)c(O)c(Br)cc12 ; O=c1c(Oc2ccc(Br)cc2)c(C(F)(F)F)oc2c(O)c(O)ccc12 ; O=c1c(Oc2cccc(Br)c2)c(C(F)(F)F)oc2c(O)c(O)ccc12 ; O=c1c(Oc2ccccc2Br)c(C(F)(F)F)oc2c(O)c(O)ccc12]

In some embodiments of the compound of formula (I), R⁵ and R⁶ are both ONa. An exemplary compound is

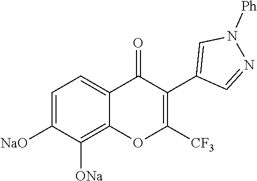

In some embodiments of the compound of formula (I), R⁵ and R⁶ are different substituents. Exemplary compounds include, but are not limited to,

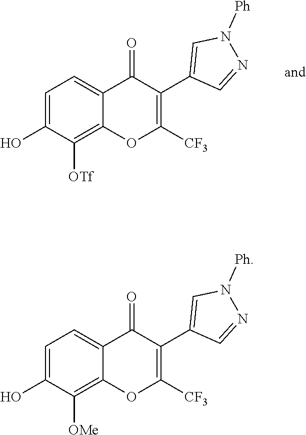 [SMILES: O=c1c(-c2cnn(-c3ccccc3)c2)c(C(F)(F)F)oc2c(OS(=O)(=O)C(F)(F)F)c(O)ccc12 ; COc1c(O)ccc2c(=O)c(-c3cnn(-c4ccccc4)c3)c(C(F)(F)F)oc12]

In some embodiments of the compound of formula (I), R² is H or CF₃. In some embodiments, R² is CF₃.

Specific compounds of formula (I) include, without limitation, the compounds in the following Table 1.

TABLE 1
Selected Compounds of Formula (I)

| | | | E3 (CUL1) | E3 (CUL4) |
|---|---|---|---|---|
| Compound | | Apoptosis | Inhibition | Inhibition |
| Code | Compound Structure | EC50 (μm) | EC50 (μm) | EC50 (μm) |
| KH-4-43 | 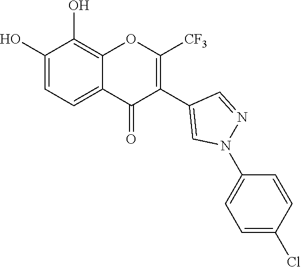 [SMILES: O=c1c(-c2cnn(-c3ccc(Cl)cc3)c2)c(C(F)(F)F)oc2c(O)c(O)ccc12] | 4 | 7 | 10 |
| KH-3-49 | 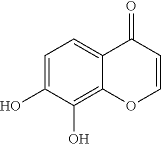 [SMILES: O=c1ccoc2c(O)c(O)ccc12] | ND | 11 | >>75 |
| KH-3-50 | 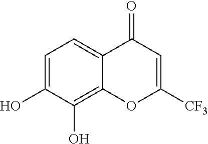 [SMILES: O=c1cc(C(F)(F)F)oc2c(O)c(O)ccc12] | ND | 4.9 | Inactive |
| KH-3-12 | 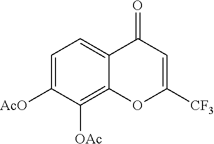 [SMILES: CC(=O)Oc1ccc2c(=O)cc(C(F)(F)F)oc2c1OC(C)=O] | ND | 32 | >>75 |
| KH-3-160A | 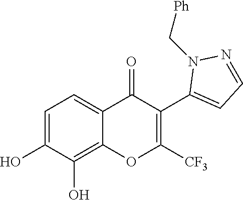 [SMILES: O=c1c(-c2ccnn2Cc2ccccc2)c(C(F)(F)F)oc2c(O)c(O)ccc12] | 21 | 3.4 | ~20 |
| KH-3-150 | 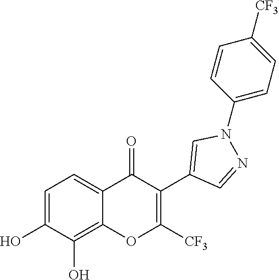 [SMILES: O=c1c(-c2cnn(-c3ccc(C(F)(F)F)cc3)c2)c(C(F)(F)F)oc2c(O)c(O)ccc12] | 6 | 9.3 | >14 |
| KH-3-151 | 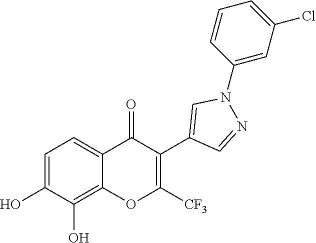 [SMILES: O=c1c(-c2cnn(-c3cccc(Cl)c3)c2)c(C(F)(F)F)oc2c(O)c(O)ccc12] | 5 | 13 | ~14 |
| KH-3-152 | 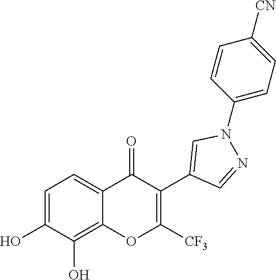 [SMILES: N#Cc1ccc(-n2cc(-c3c(C(F)(F)F)oc4c(O)c(O)ccc4c3=O)cn2)cc1] | 30 | 9 | >14 |
| KH-3-141 | 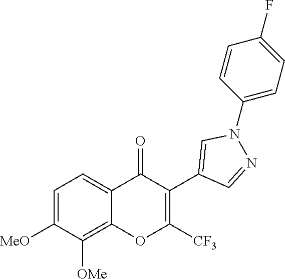 [SMILES: COc1ccc2c(=O)c(-c3cnn(-c4ccc(F)cc4)c3)c(C(F)(F)F)oc2c1OC] | Inactive | Inactive | Inactive |
| KH-3-134 | 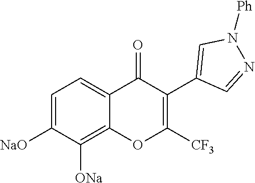 | Inactive | 21 | Inactive |
| KH-3-115 | 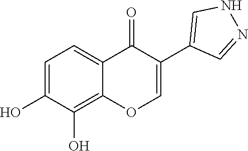 [SMILES: O=c1c(-c2cn[nH]c2)coc2c(O)c(O)ccc12] | ND | 3.8 | Inactive |
| KH-4-65 | 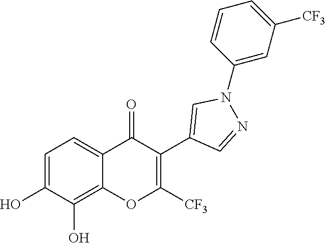 [SMILES: O=c1c(-c2cnn(-c3cccc(C(F)(F)F)c3)c2)c(C(F)(F)F)oc2c(O)c(O)ccc12] | ND | 8 | >14 |
| KH-4-66 | 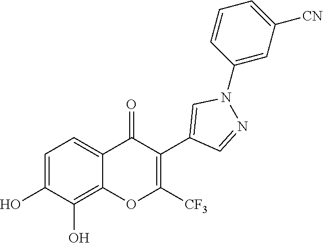 [SMILES: N#Cc1cccc(-n2cc(-c3c(C(F)(F)F)oc4c(O)c(O)ccc4c3=O)cn2)c1] | ND | 9 | >14 |
| KH-4-67 | 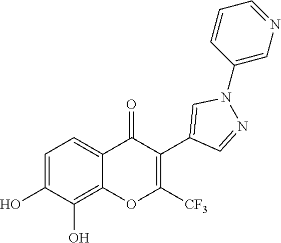 [SMILES: O=c1c(-c2cnn(-c3cccnc3)c2)c(C(F)(F)F)oc2c(O)c(O)ccc12] | ND | Inactive | Inactive |
| KH-4-44 | 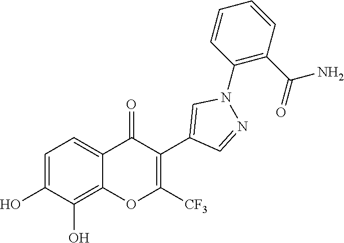 [SMILES: NC(=O)c1ccccc1-n1cc(-c2c(C(F)(F)F)oc3c(O)c(O)ccc3c2=O)cn1] | ND | 10 | Inactive |
| KH-3-174 | 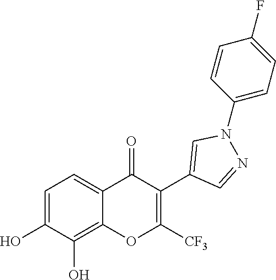 [SMILES: O=c1c(-c2cnn(-c3ccc(F)cc3)c2)c(C(F)(F)F)oc2c(O)c(O)ccc12] | ND | 8 | >14 |
| KH-4-41 | 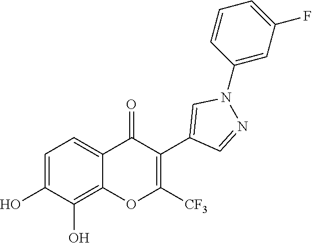 [SMILES: O=c1c(-c2cnn(-c3cccc(F)c3)c2)c(C(F)(F)F)oc2c(O)c(O)ccc12] | 9 | 5 | ~14 |
| KH-4-88 | 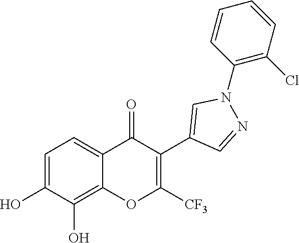 [SMILES: O=c1c(-c2cnn(-c3ccccc3Cl)c2)c(C(F)(F)F)oc2c(O)c(O)ccc12] | 26 | ND | >>14 |
| KH-4-99 | 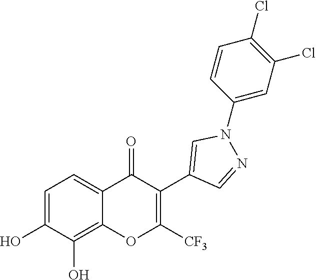 [SMILES: O=c1c(-c2cnn(-c3ccc(Cl)c(Cl)c3)c2)c(C(F)(F)F)oc2c(O)c(O)ccc12] | 9 | ND | ~10 |
| KH-4-116 | 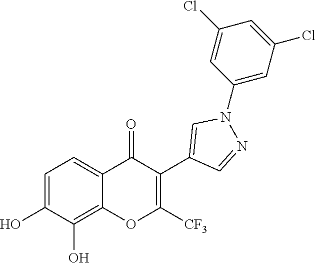 [SMILES: O=c1c(-c2cnn(-c3cc(Cl)cc(Cl)c3)c2)c(C(F)(F)F)oc2c(O)c(O)ccc12] | 6 | ND | ~10 |
| KH-4-98 | 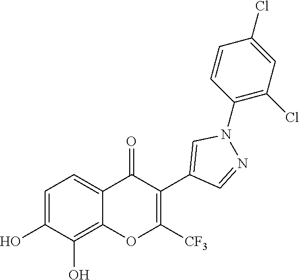 [SMILES: O=c1c(-c2cnn(-c3ccc(Cl)cc3Cl)c2)c(C(F)(F)F)oc2c(O)c(O)ccc12] | 9 | ND | ~10 |
| KH-4-136 | 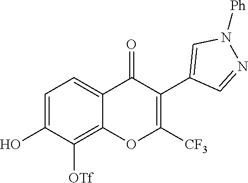 [SMILES: O=c1c(-c2cnn(-c3ccccc3)c2)c(C(F)(F)F)oc2c(OS(=O)(=O)C(F)(F)F)c(O)ccc12] | Inactive | ND | Inactive |
| KH-5-09 | 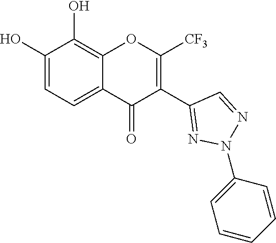 [SMILES: O=c1c(-c2cnn(-c3ccccc3)n2)c(C(F)(F)F)oc2c(O)c(O)ccc12] | ND | ND | >>14 |
| KH-4-119 | 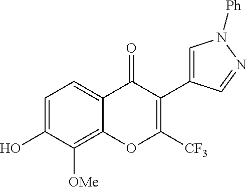 [SMILES: COc1c(O)ccc2c(=O)c(-c3cnn(-c4ccccc4)c3)c(C(F)(F)F)oc12] | Inactive | ND | Inactive |
| KH-4-78 | 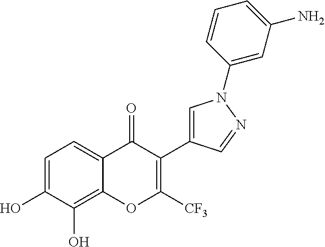 [SMILES: Nc1cccc(-n2cc(-c3c(C(F)(F)F)oc4c(O)c(O)ccc4c3=O)cn2)c1] | Inactive | ND | >>14 |
| KH-4-79 | 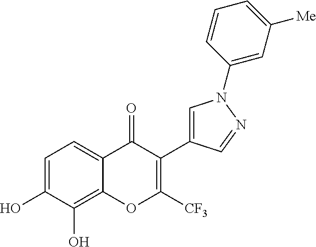 [SMILES: Cc1cccc(-n2cc(-c3c(C(F)(F)F)oc4c(O)c(O)ccc4c3=O)cn2)c1] | 19 | ND | ~14 |
| MSSM-RJD- MM-001 | 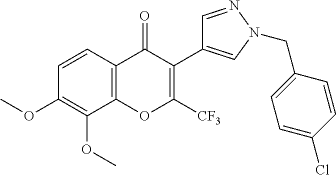 [SMILES: COc1ccc2c(=O)c(-c3cnn(Cc4ccc(Cl)cc4)c3)c(C(F)(F)F)oc2c1OC] | >>20 | >100 | >10 |
| MSSM-RJD- MM-002 | 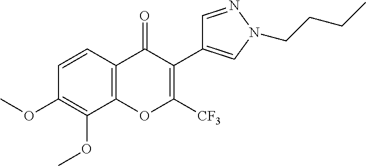 [SMILES: CCCCn1cc(-c2c(C(F)(F)F)oc3c(OC)c(OC)ccc3c2=O)cn1] | >>20 | 96 | >>10 |
| MSSM-RJD- MM-003 | 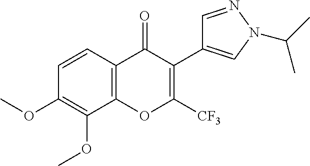 [SMILES: COc1ccc2c(=O)c(-c3cnn(C(C)C)c3)c(C(F)(F)F)oc2c1OC] | >>20 | >100 | >>10 |
| MSSM-RJD- MM-004 | 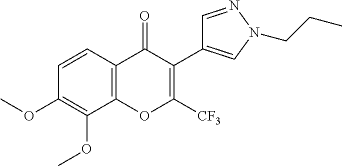 [SMILES: CCCn1cc(-c2c(C(F)(F)F)oc3c(OC)c(OC)ccc3c2=O)cn1] | >>20 | >100 | >>10 |
| MSSM-RJD- MM-005 | 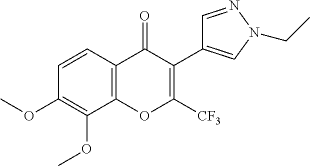 [SMILES: CCn1cc(-c2c(C(F)(F)F)oc3c(OC)c(OC)ccc3c2=O)cn1] | >>20 | >100 | >>10 |
| MSSM-RJD- MM-006 | 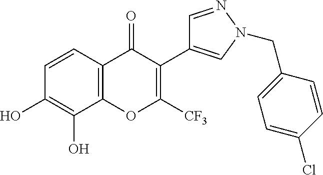 [SMILES: O=c1c(-c2cnn(Cc3ccc(Cl)cc3)c2)c(C(F)(F)F)oc2c(O)c(O)ccc12] | >20 | 7.6 | >10 |
| MSSM-RJD- MM-007 | 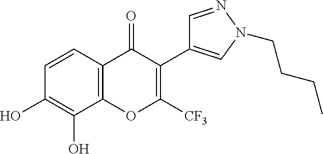 [SMILES: CCCCn1cc(-c2c(C(F)(F)F)oc3c(O)c(O)ccc3c2=O)cn1] | >>20 | 3.8 | >>10 |
| MSSM-RJD- MM-008 | 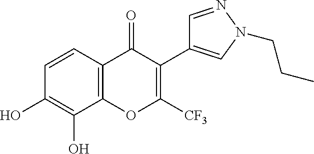 [SMILES: CCCn1cc(-c2c(C(F)(F)F)oc3c(O)c(O)ccc3c2=O)cn1] | >>20 | 4 | >>10 |
| MSSM-RJD- MM-009 | 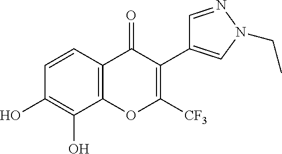 [SMILES: CCn1cc(-c2c(C(F)(F)F)oc3c(O)c(O)ccc3c2=O)cn1] | >>20 | 6 | >>10 |
| MSSM-RJD- MM-010 | 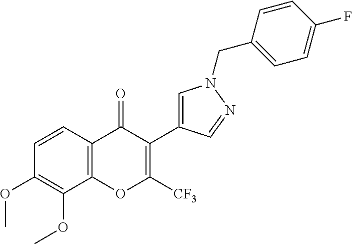 [SMILES: COc1ccc2c(=O)c(-c3cnn(Cc4ccc(F)cc4)c3)c(C(F)(F)F)oc2c1OC] | >>20 | >100 | >>10 |
| MSSM-RJD- MM-011 | 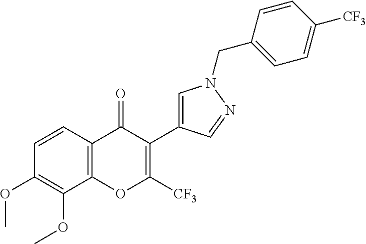 [SMILES: COc1ccc2c(=O)c(-c3cnn(Cc4ccc(C(F)(F)F)cc4)c3)c(C(F)(F)F)oc2c1OC] | >>20 | >100 | >>10 |
| MSSM-RJD- MM-012 | 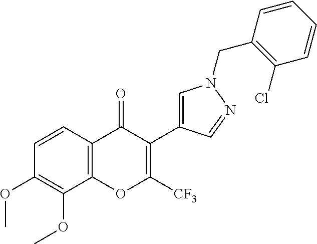 [SMILES: COc1ccc2c(=O)c(-c3cnn(Cc4ccccc4Cl)c3)c(C(F)(F)F)oc2c1OC] | >>20 | >100 | >>10 |
| MSSM-RJD- MM-013 | 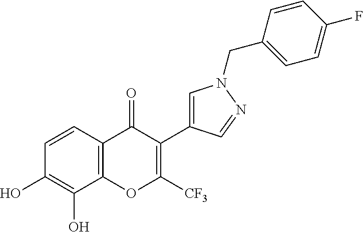 [SMILES: O=c1c(-c2cnn(Cc3ccc(F)cc3)c2)c(C(F)(F)F)oc2c(O)c(O)ccc12] | >>20 | 7.2 | >30 |
| MSSM-RJD- MM-014 | 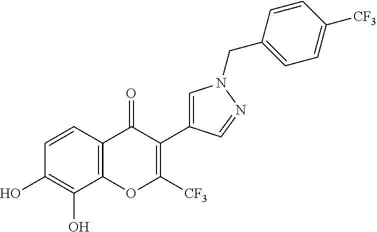 [SMILES: O=c1c(-c2cnn(Cc3ccc(C(F)(F)F)cc3)c2)c(C(F)(F)F)oc2c(O)c(O)ccc12] | ~8 | 5.4 | ~10 |
| MSSM-RJD- MM-015 | 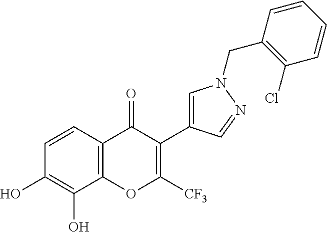 [SMILES: O=c1c(-c2cnn(Cc3ccccc3Cl)c2)c(C(F)(F)F)oc2c(O)c(O)ccc12] | >20 | 8.2 | >>10 |
| MSSM-RJD- MM-016 | 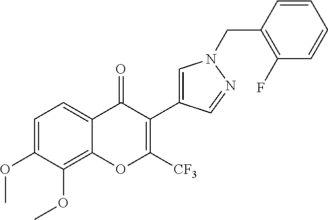 [SMILES: COc1ccc2c(=O)c(-c3cnn(Cc4ccccc4F)c3)c(C(F)(F)F)oc2c1OC] | >>20 | >100 | >>10 |
| MSSM-RJD- MM-017 | 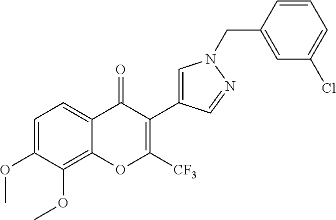 [SMILES: COc1ccc2c(=O)c(-c3cnn(Cc4cccc(Cl)c4)c3)c(C(F)(F)F)oc2c1OC] | ND | 82 | >>10 |
| MSSM-RJD- MM-018 | 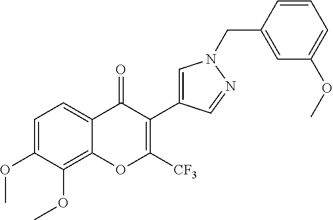 [SMILES: COc1cccc(Cn2cc(-c3c(C(F)(F)F)oc4c(OC)c(OC)ccc4c3=O)cn2)c1] | ND | >100 | >>10 |
| MSSM-RJD- MM-019 | 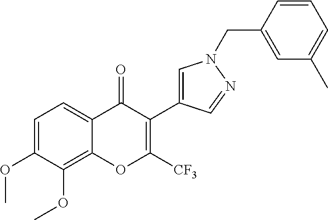 [SMILES: COc1ccc2c(=O)c(-c3cnn(Cc4cccc(C)c4)c3)c(C(F)(F)F)oc2c1OC] | ND | 92 | >>10 |
| MSSM-RJD- MM-020 | 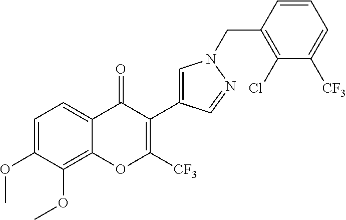 [SMILES: COc1ccc2c(=O)c(-c3cnn(Cc4cccc(C(F)(F)F)c4Cl)c3)c(C(F)(F)F)oc2c1OC] | >20 | 53 | >>10 |
| MSSM-RJD- MM-021 | 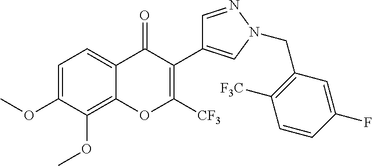 [SMILES: COc1ccc2c(=O)c(-c3cnn(Cc4cc(F)ccc4C(F)(F)F)c3)c(C(F)(F)F)oc2c1OC] | ~20 | 66 | >>10 |
| MSSM-RJD- MM-022 | 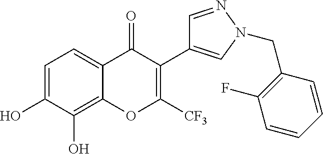 [SMILES: O=c1c(-c2cnn(Cc3ccccc3F)c2)c(C(F)(F)F)oc2c(O)c(O)ccc12] | >20 | 30 | >>10 |
| MSSM-RJD- MM-023 | 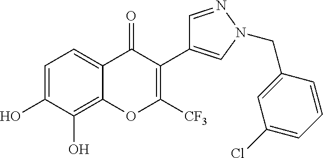 [SMILES: O=c1c(-c2cnn(Cc3cccc(Cl)c3)c2)c(C(F)(F)F)oc2c(O)c(O)ccc12] | <20, >10 | 8.4 | >10 |
| MSSM-RJD- MM-024 | 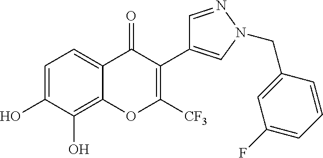 [SMILES: O=c1c(-c2cnn(Cc3cccc(F)c3)c2)c(C(F)(F)F)oc2c(O)c(O)ccc12] | <20, >10 | 7.9 | >10 |
| MSSM-RJD- MM-025 | 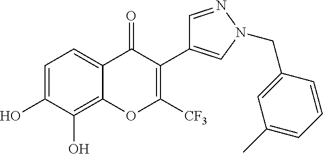 [SMILES: Cc1cccc(Cn2cc(-c3c(C(F)(F)F)oc4c(O)c(O)ccc4c3=O)cn2)c1] | <20, >10 | 3.9 | >10 |
| MSSM-RJD- MM-026 | 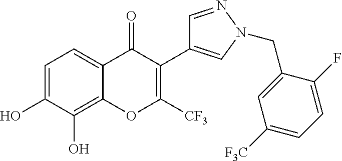 [SMILES: O=c1c(-c2cnn(Cc3cc(C(F)(F)F)ccc3F)c2)c(C(F)(F)F)oc2c(O)c(O)ccc12] | >20 | 6.5 | >10 |
| MSSM-RJD- MM-027 | 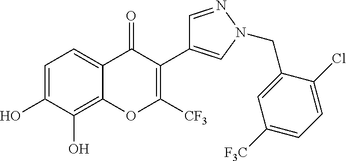 [SMILES: O=c1c(-c2cnn(Cc3cc(C(F)(F)F)ccc3Cl)c2)c(C(F)(F)F)oc2c(O)c(O)ccc12] | <20, >10 | 5.6 | >10 |
| MSSM-RJD- MM-028 | 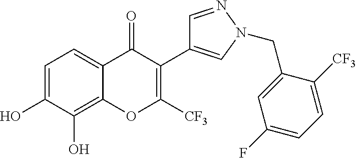 [SMILES: O=c1c(-c2cnn(Cc3cc(F)ccc3C(F)(F)F)c2)c(C(F)(F)F)oc2c(O)c(O)ccc12] | ~20 | 7.3 | ~10 |

Another aspect of the present disclosure relates to compounds having the following structure:

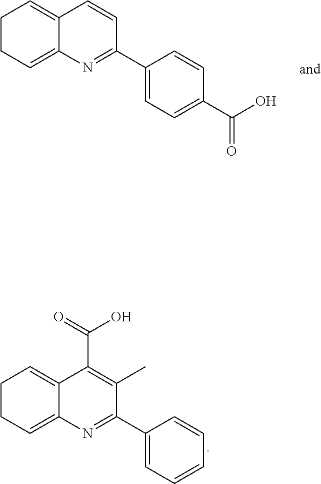 [SMILES: O=C(O)c1ccc(-c2ccc3c(n2)=CCCC=3)cc1 ; Cc1c(-c2ccccc2)nc2c(c1C(=O)O)=CCCC=2]

Yet another aspect of the disclosure relates to compounds having the following structure:

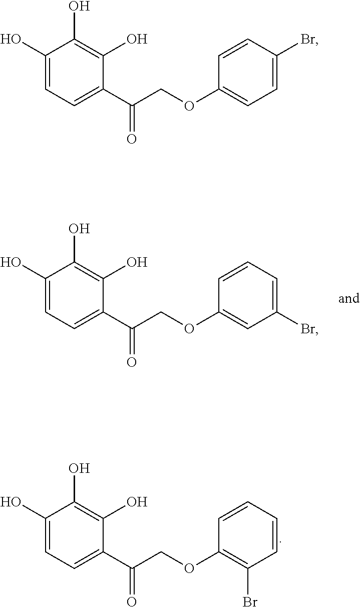 [SMILES: O=C(COc1ccc(Br)cc1)c1ccc(O)c(O)c1O ; O=C(COc1cccc(Br)c1)c1ccc(O)c(O)c1O ; O=C(COc1ccccc1Br)c1ccc(O)c(O)c1O]

Yet another aspect of the present disclosure relates to compounds having the following structure:

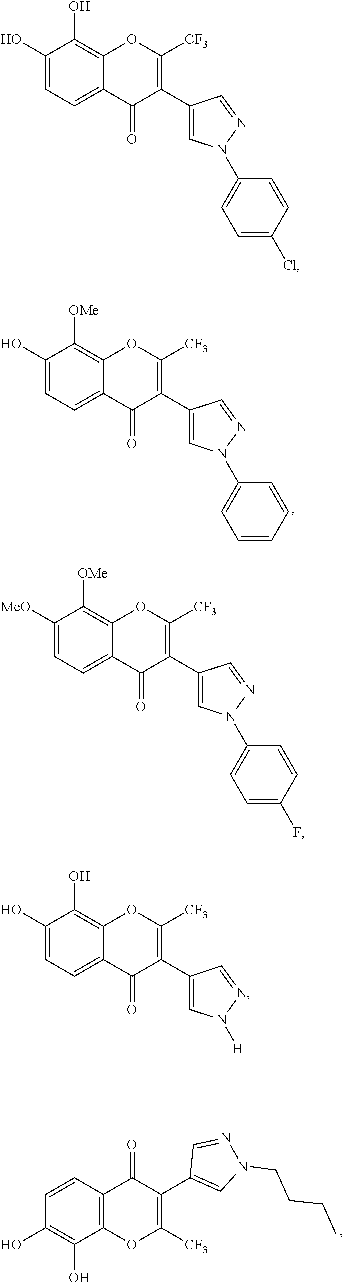 [SMILES: O=c1c(-c2cnn(-c3ccc(Cl)cc3)c2)c(C(F)(F)F)oc2c(O)c(O)ccc12 ; COc1c(O)ccc2c(=O)c(-c3cnn(-c4ccccc4)c3)c(C(F)(F)F)oc12 ; COc1ccc2c(=O)c(-c3cnn(-c4ccc(F)cc4)c3)c(C(F)(F)F)oc2c1OC ; O=c1c(-c2cn[nH]c2)c(C(F)(F)F)oc2c(O)c(O)ccc12 ; CCCCn1cc(-c2c(C(F)(F)F)oc3c(O)c(O)ccc3c2=O)cn1]

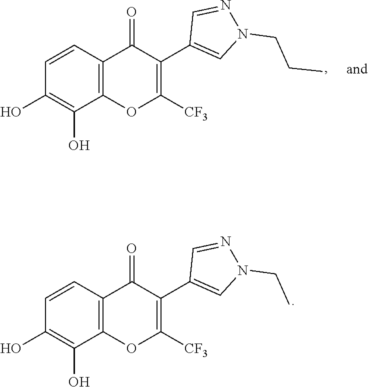 [SMILES: CCCn1cc(-c2c(C(F)(F)F)oc3c(O)c(O)ccc3c2=O)cn1 ; CCn1cc(-c2c(C(F)(F)F)oc3c(O)c(O)ccc3c2=O)cn1]

Compounds of the present disclosure can be made according to known methods. Some methods of making compounds of the present disclosure are described infra in the Examples. In some embodiments, compounds of the present disclosure where R³ is CF₃ can be prepared according to Scheme 1 outlined below.

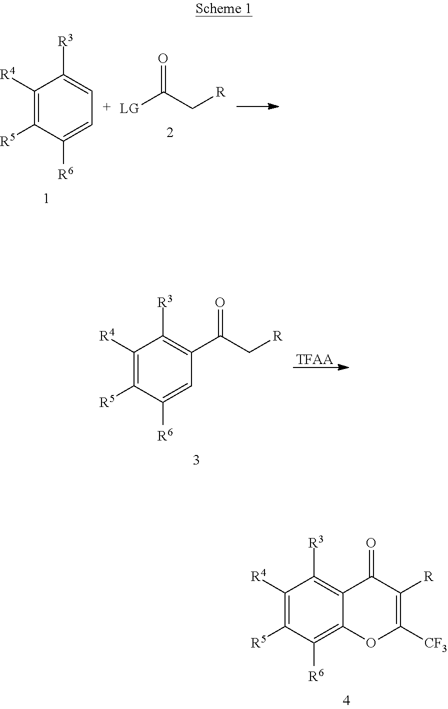

LG is a leaving group

### R is H, CF₃,

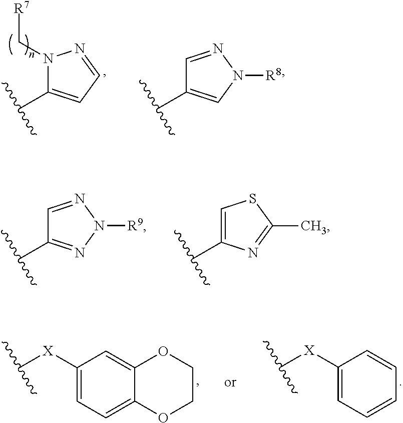

Acylation of compound (1) with compound (2) leads to formation of the compound (3) in the presence of boron trifluoride etherate. The reaction can be carried out neat or in a variety of solvents. The reaction can be carried out in a presence of a catalyst. Suitable catalysts that can be used include boron trifluoride etherate. This reaction can be carried out at a room temperature or at elevated temperatures. Reaction of compound (3) with 2,2,2-trifluoroacetic anhydride (TFAA) in a presence of a base leads to formation of the final product (4). The reaction can be carried out neat or in a variety of solvents. Suitable bases that can be used include pyridine and triethyl amine. During the reaction process, the one or more hydroxyl groups on the compound (1) fragments can be protected by a suitable protecting group which can be selectively removed at a later time if desired. A detailed description of these groups and their selection and chemistry is contained in “The Peptides, Vol. 3”, Gross and Meinenhofer, Eds., Academic Press, New York, 1981; Wuts et al., “Greene's Protective Groups in Organic Synthesis,” Fourth Edition, John Wiley & Sons, Inc., Hoboken, New Jersey, 2007, which are hereby incorporated by reference in their entirety.

Compound (4), where one of the R⁴ is H, can be halogenated according to Scheme 2 outlined below.

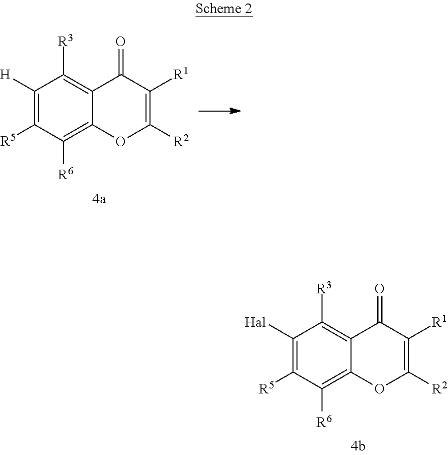

This reaction can be carried out in a variety of solvents, for example dimethyl formamide (DMF), dimethoxyethane (DME), and acetonitrile. Suitable halogenated agents that can be used include N-bromosuccinimide (NBS), KBr, Br₂, I₂, and HBr. This reaction can be carried out at a room temperature or at elevated temperatures.

Another aspect of the present disclosure relates to a method of inhibiting the catalytic activity of a Cullin-RING E3 Ubiquitin (Ub) Ligase (CRL) in a cell. This method involves contacting a cell with a compound described in the present disclosure under conditions effective to inhibit activity of the CRL in the cell.

A further aspect of the present disclosure relates to a method of inhibiting the catalytic activity of a Cullin-RING E3 Ubiquitin (Ub) Ligase (CRL) in a cell. This method involves contacting a cell with a compound of formula (II) having the following structure:

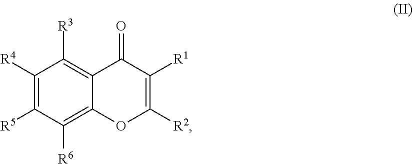

or a stereoisomer, pharmaceutically acceptable salt, oxide, or solvate thereof, wherein
R¹ and R² are each independently H, CF₃,

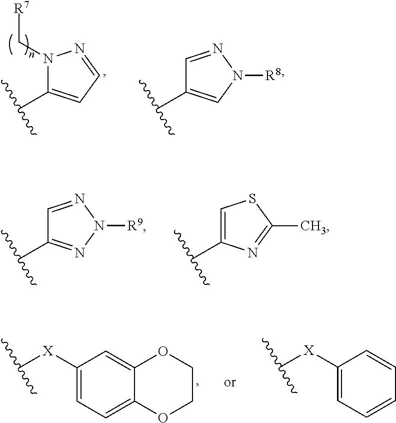

wherein the benzene ring of

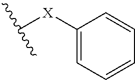

is optionally substituted with NO₂ or halogen;
R³, R⁴, R⁵, and R⁶ are each independently selected from the group consisting of H, halogen, OH, C₁-C₆ alkyl, OAc, NaO, OTf, and alkoxy, wherein two adjacent alkoxy groups may be taken together to form a heterocycle;
R⁷, R⁸, and R⁹ are each independently selected from H, C₁-C₆ alkyl, pyridine, or

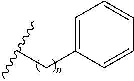

wherein the benzene ring of

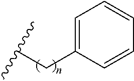

is optionally substituted one or more times with C₁-C₆ alkyl, NH₂, CF₃, halogen, CN, C(O)NH₂, and alkoxy;
X is either present or absent, and when present is CH₂ or O; and
n is 0 or 1;
   under conditions effective to inhibit activity of the CRL in the cell.

In some embodiments, the cell is contacted with a compound of formula (II), where R³, R⁴, R⁵, and R⁶ are each independently selected from the group consisting of H, OH, C₁-C₆ alkyl, OAc, NaO, OTf, and alkoxy, wherein two adjacent alkoxy groups may be taken together to form a heterocycle.

A further aspect of the present disclosure relates to a method of inhibiting the catalytic activity of a Cullin-RING E3 Ubiquitin (Ub) Ligase (CRL) in a cell. This method involves contacting a cell with a compound of formula (II) having the following structure:

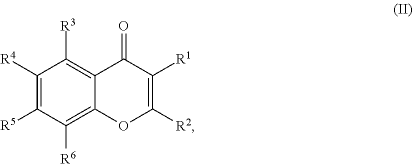

or a stereoisomer, pharmaceutically acceptable salt, oxide, or solvate thereof, wherein
R¹ and R² are each independently H, CF₃,

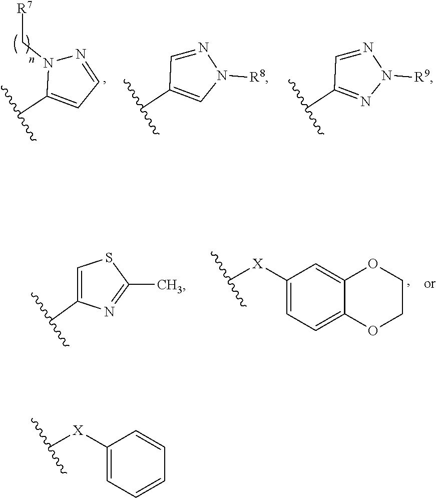

where the benzene ring of

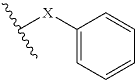

is optionally substituted with NO₂ or halogen;
R³, R⁴, R⁵, and R⁶ are each independently selected from the group consisting of H, OH, C₁-C₆ alkyl, —OAc, NaO—, OTf, and alkoxy, wherein two adjacent alkoxy groups may be taken together to form a heterocycle;
R⁷, R⁸, and R⁹ are each independently selected from H, C₁-C₆ alkyl, pyridine, or

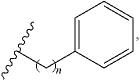

wherein the benzene ring of

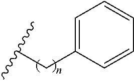

is optionally substituted one or more times with C₁-C₆ alkyl, NH₂, CF₃, halogen, CN, C(O)NH₂, and alkoxy;
X is either present or absent, and when present is CH₂ or O; and
n is 0 or 1;
   under conditions effective to inhibit activity of the CRL in the cell.

In some embodiments, the cell is contacted with a compound of formula (II), where R¹ is

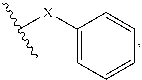

where the benzene ring is optionally substituted, and where X is O.

In some embodiments, the cell is contacted with a compound selected from the group consisting of

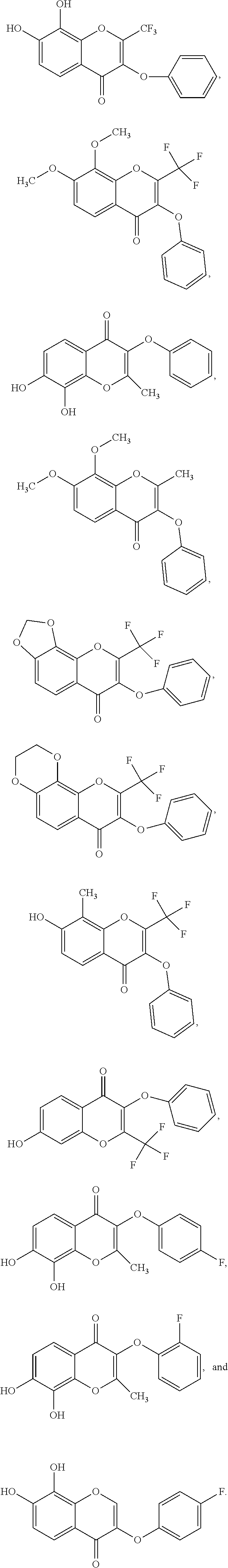

In some embodiments, the cell is contacted with a compound selected from the group consisting of

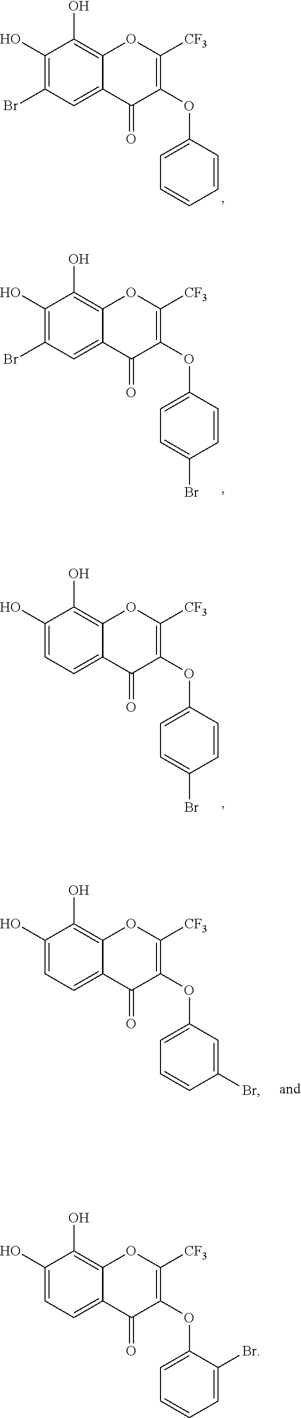 [SMILES: O=c1c(Oc2ccccc2)c(C(F)(F)F)oc2c(O)c(O)c(Br)cc12 ; O=c1c(Oc2ccc(Br)cc2)c(C(F)(F)F)oc2c(O)c(O)c(Br)cc12 ; O=c1c(Oc2ccc(Br)cc2)c(C(F)(F)F)oc2c(O)c(O)ccc12 ; O=c1c(Oc2cccc(Br)c2)c(C(F)(F)F)oc2c(O)c(O)ccc12 ; O=c1c(Oc2ccccc2Br)c(C(F)(F)F)oc2c(O)c(O)ccc12]

In some embodiments, the cell is contacted with a compound of formula (II), where R² is CF₃.

In some embodiments, the cell is contacted with a compound of formula (II), where R³ and R⁴ are both H. Exemplary compounds include, but are not limited to,

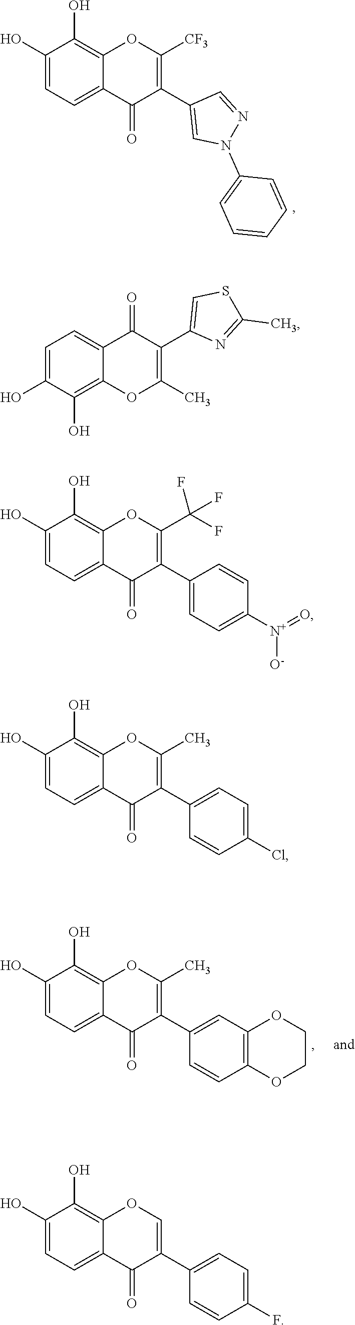 [SMILES: O=c1c(-c2cnn(-c3ccccc3)c2)c(C(F)(F)F)oc2c(O)c(O)ccc12 ; Cc1nc(-c2c(C)oc3c(O)c(O)ccc3c2=O)cs1 ; O=c1c(-c2ccc([N+](=O)[O-])cc2)c(C(F)(F)F)oc2c(O)c(O)ccc12 ; Cc1oc2c(O)c(O)ccc2c(=O)c1-c1ccc(Cl)cc1 ; Cc1oc2c(O)c(O)ccc2c(=O)c1-c1ccc2c(c1)OCCO2 ; O=c1c(-c2ccc(F)cc2)coc2c(O)c(O)ccc12]

In some embodiments, the cell is contacted with a compound selected from the group consisting of

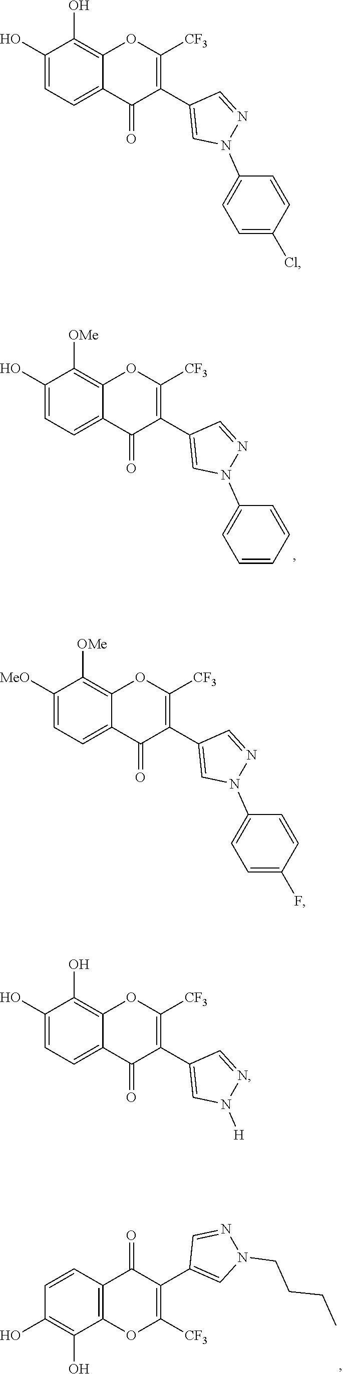 [SMILES: O=c1c(-c2cnn(-c3ccc(Cl)cc3)c2)c(C(F)(F)F)oc2c(O)c(O)ccc12 ; COc1c(O)ccc2c(=O)c(-c3cnn(-c4ccccc4)c3)c(C(F)(F)F)oc12 ; COc1ccc2c(=O)c(-c3cnn(-c4ccc(F)cc4)c3)c(C(F)(F)F)oc2c1OC ; O=c1c(-c2cn[nH]c2)c(C(F)(F)F)oc2c(O)c(O)ccc12 ; CCCCn1cc(-c2c(C(F)(F)F)oc3c(O)c(O)ccc3c2=O)cn1]

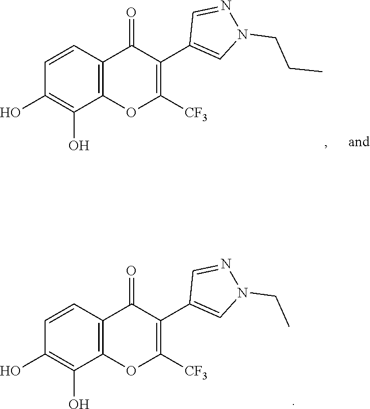 [SMILES: CCCn1cc(-c2c(C(F)(F)F)oc3c(O)c(O)ccc3c2=O)cn1 ; CCn1cc(-c2c(C(F)(F)F)oc3c(O)c(O)ccc3c2=O)cn1]

In some embodiments, the cell is contacted with a compound of formula (II), where R⁵ and R⁶ are both OH.

In some embodiments, the cell is contacted with a compound of formula (II), where R¹ is

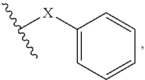

and where the benzene ring is optionally substituted, and where X is O. Exemplary compounds of this embodiment include, but are not limited to,

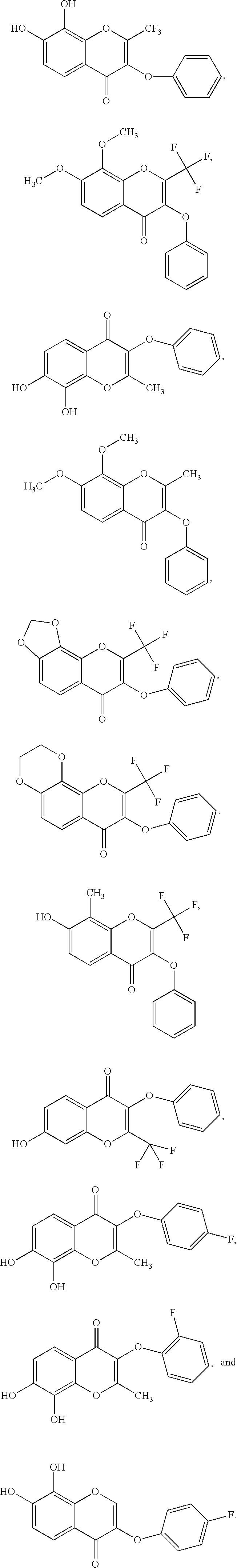

Specific compounds of formula (II) include, without limitation, the compounds in the above Table 1 and the compounds in the following Table 2.

TABLE 2
Selected Compounds of Formula (II)

| Compound Code | Compound Structure |
|---|---|
| #33 | 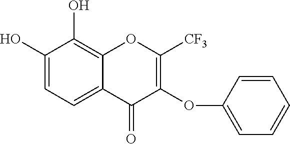 [SMILES: O=c1c(Oc2ccccc2)c(C(F)(F)F)oc2c(O)c(O)ccc12] |
| #33-11 | 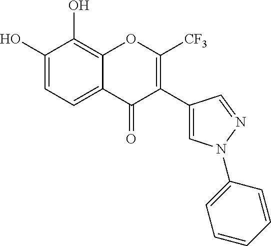 [SMILES: O=c1c(-c2cnn(-c3ccccc3)c2)c(C(F)(F)F)oc2c(O)c(O)ccc12] |
| #33-1 | 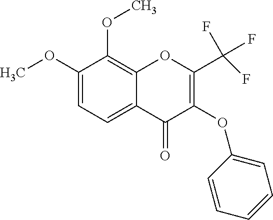 [SMILES: COc1ccc2c(=O)c(Oc3ccccc3)c(C(F)(F)F)oc2c1OC] |
| #33-2 | 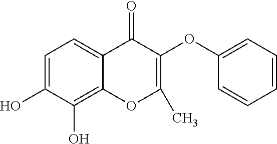 [SMILES: Cc1oc2c(O)c(O)ccc2c(=O)c1Oc1ccccc1] |
| #33-3 | 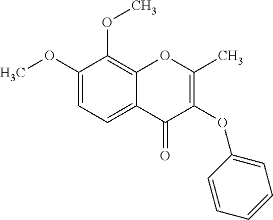 [SMILES: COc1ccc2c(=O)c(Oc3ccccc3)c(C)oc2c1OC] |
| #33-4 | 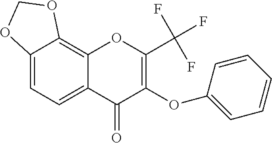 [SMILES: O=c1c(Oc2ccccc2)c(C(F)(F)F)oc2c3c(ccc12)OCO3] |
| #33-5 | 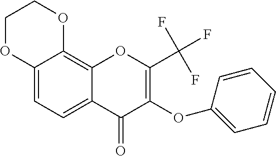 [SMILES: O=c1c(Oc2ccccc2)c(C(F)(F)F)oc2c3c(ccc12)OCCO3] |
| #33-6 | 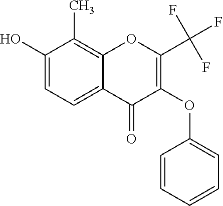 [SMILES: Cc1c(O)ccc2c(=O)c(Oc3ccccc3)c(C(F)(F)F)oc12] |
| #33-7 | 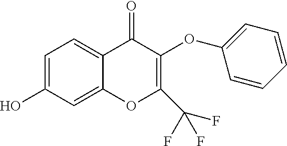 [SMILES: O=c1c(Oc2ccccc2)c(C(F)(F)F)oc2cc(O)ccc12] |
| #33-9 | 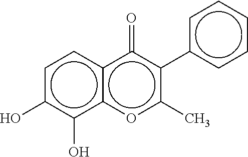 |
| #33-12 | 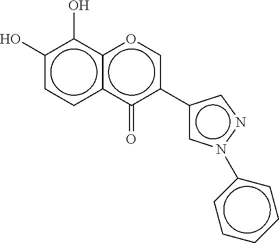 |
| #33-13 | 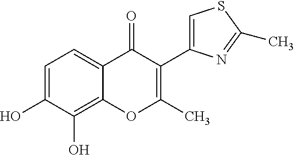 [SMILES: Cc1nc(-c2c(C)oc3c(O)c(O)ccc3c2=O)cs1] |
| #33-14 | 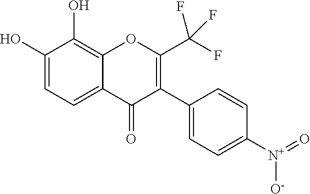 [SMILES: O=c1c(-c2ccc([N+](=O)[O-])cc2)c(C(F)(F)F)oc2c(O)c(O)ccc12] |
| #33-15 | 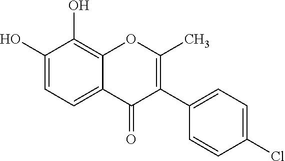 [SMILES: Cc1oc2c(O)c(O)ccc2c(=O)c1-c1ccc(Cl)cc1] |
| #33-16 | 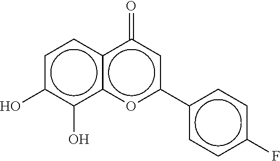 |
| #33-17 | 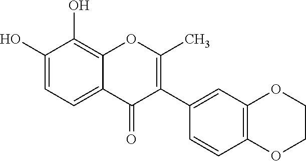 [SMILES: Cc1oc2c(O)c(O)ccc2c(=O)c1-c1ccc2c(c1)OCCO2] |
| #33-18 | 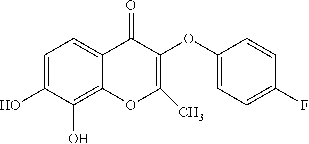 [SMILES: Cc1oc2c(O)c(O)ccc2c(=O)c1Oc1ccc(F)cc1] |
| #33-19 | 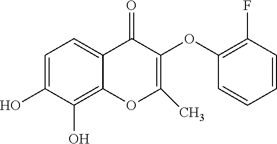 [SMILES: Cc1oc2c(O)c(O)ccc2c(=O)c1Oc1ccccc1F] |
| #33-20 | 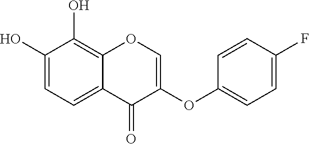 [SMILES: O=c1c(Oc2ccc(F)cc2)coc2c(O)c(O)ccc12] |
| #33-21 | 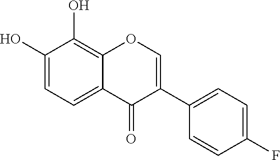 [SMILES: O=c1c(-c2ccc(F)cc2)coc2c(O)c(O)ccc12] |

Another aspect of the present disclosure relates to a method of inhibiting the catalytic activity of a Cullin-RING E3 Ubiquitin (Ub) Ligase (CRL) in a cell. This method involves contacting a cell with a compound selected from the group consisting of

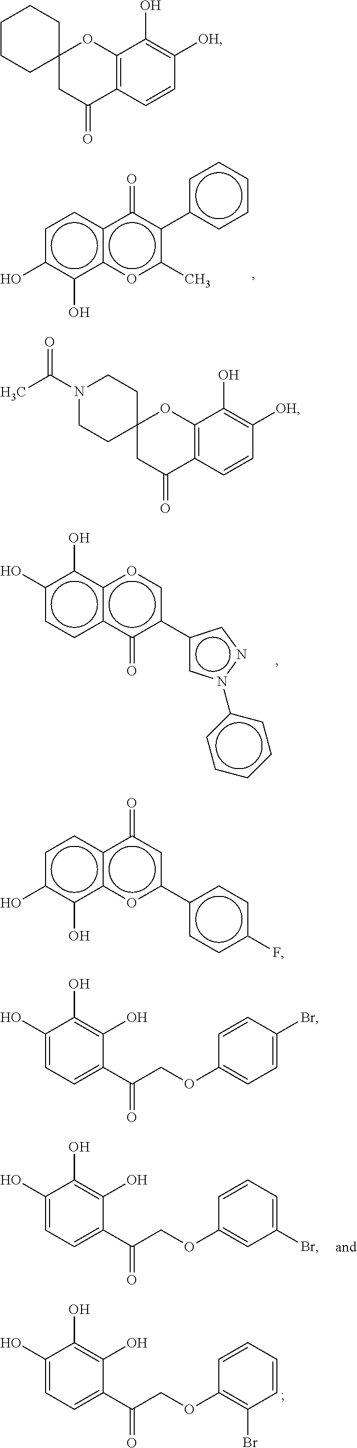

under conditions effective to inhibit activity of the CRL in the cell.

Another aspect of the present disclosure relates to a method of inhibiting the catalytic activity of a Cullin-RING E3 Ubiquitin (Ub) Ligase (CRL) in a cell. This method involves contacting a cell with a compound selected from the group consisting of

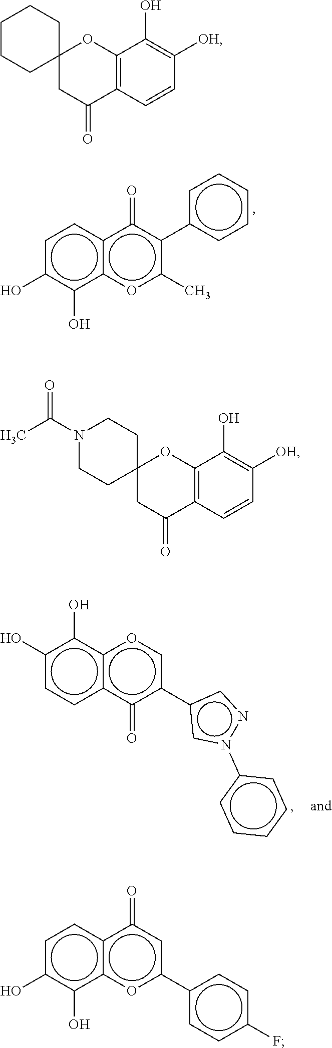

under conditions effective to inhibit activity of the CRL in the cell.

In carrying out this and other methods of the disclosure, the cell may be a mammalian cell. Mammalian cells include cells from, for example, mice, hamsters, rats, cows, sheep, pigs, goats, and horses, monkeys, dogs (e.g., Canis familiaris), cats, rabbits, guinea pigs, and primates, including humans. In one particular embodiment, the cell is a human cell.

In another embodiment, the cell is a cancer cell.

This and other methods described herein may be carried out ex vivo or in vivo. When carried out ex vivo, a population of cells may be, according to one embodiment, provided by obtaining cells from a subject and culturing the cells in a liquid medium suitable for the in vitro or ex vivo culture of mammalian cells, in particular human cells.

A further aspect of the present disclosure relates to a composition comprising a compound according to formula (I) or formula (II) described herein and a carrier.

In one embodiment, the carrier is a pharmaceutically-acceptable carrier.

While it may be possible for the compounds described herein (i.e., compounds of formula (I) and compounds of formula (II)) to be administered as the raw chemical, it may be preferable to present them as a pharmaceutical composition. Thus, in some embodiments, there is provided a pharmaceutical composition comprising a compound of formula (I) or formula (II), or a pharmaceutically acceptable salt or solvate thereof, together with one or more pharmaceutically carriers thereof and optionally one or more other therapeutic ingredients.

The carrier(s) must be “acceptable” in the sense of being compatible with the other ingredients of the formulation and not deleterious to the recipient thereof. Furthermore, notwithstanding the statements herein regarding the term “compound” including salts thereof as well, so that independent claims reciting “a compound” will be understood as referring to salts thereof as well, if in an independent claim reference is made to a compound or a pharmaceutically acceptable salt thereof, it will be understood that claims which depend from that independent claim which refer to such a compound also include pharmaceutically acceptable salts of the compound, even if explicit reference is not made to the salts in the dependent claim.

Formulations include those suitable for oral, parenteral (including subcutaneous, intradermal, intramuscular, intravenous, and intraarticular), rectal, and topical (including dermal, buccal, sublingual, and intraocular) administration. The most suitable route may depend upon the condition and disorder of the recipient. The formulations may conveniently be presented in unit dosage form and may be prepared by any of the methods well known in the art of pharmacy. Such methods include the step of bringing into association a compound of formula (I) or formula (II) or a pharmaceutically acceptable salt or solvate thereof (“active ingredient”) with the carrier, which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations suitable for oral administration may be presented as discrete units such as capsules, cachets, or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary, or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide sustained, delayed or controlled release of the active ingredient therein.

The pharmaceutical compositions may include a “pharmaceutically acceptable inert carrier,” and this expression is intended to include one or more inert excipients, which include, for example and without limitation, starches, polyols, granulating agents, microcrystalline cellulose, diluents, lubricants, binders, disintegrating agents, and the like. If desired, tablet dosages of the disclosed compositions may be coated by standard aqueous or non-aqueous techniques. “Pharmaceutically acceptable carrier” also encompasses controlled release means.

Pharmaceutical compositions may also optionally include other therapeutic ingredients, anti-caking agents, preservatives, sweetening agents, colorants, flavors, desiccants, plasticizers, dyes, and the like. Any such optional ingredient must be compatible with the compound of formula (I) or formula (II) to insure the stability of the formulation. The composition may contain other additives as needed including, for example, lactose, glucose, fructose, galactose, trehalose, sucrose, maltose, raffinose, maltitol, melezitose, stachyose, lactitol, palatinite, starch, xylitol, mannitol, myoinositol, and the like, and hydrates thereof, and amino acids, for example alanine, glycine and betaine, and peptides and proteins, for example albumen.

Examples of excipients for use as the pharmaceutically acceptable carriers and the pharmaceutically acceptable inert carriers and the aforementioned additional ingredients include, but are not limited to, binders, fillers, disintegrants, lubricants, anti-microbial agents, and coating agents.

Dose ranges for adult humans vary, but may generally be from about 0.005 mg to 10 g/day orally. Tablets or other forms of presentation provided in discrete units may conveniently contain an amount of compound of formula (I) which is effective at such dosage or as a multiple of the same, for instance, units containing 5 mg to 500 mg, or around 10 mg to 200 mg. The precise amount of compound administered to a patient will be the responsibility of the attendant physician. However, the dose employed will depend on a number of factors, including the age and sex of the patient, the precise disorder being treated, and its severity.

A dosage unit (e.g., an oral dosage unit) can include from, for example, 1 to 30 mg, 1 to 40 mg, 1 to 100 mg, 1 to 300 mg, 1 to 500 mg, 2 to 500 mg, 3 to 100 mg, 5 to 20 mg, 5 to 100 mg (e.g., 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, 500 mg) of a compound described herein.

Additional information about pharmaceutical compositions and their formulation is described in Remington: The Science and Practice of Pharmacy, 20^{th} Edition, 2000, which is hereby incorporated by reference in its entirety.

The agents can be administered, e.g., by intravenous injection, intramuscular injection, subcutaneous injection, intraperitoneal injection, topical, sublingual, intraarticular (in the joints), intradermal, buccal, ophthalmic (including intraocular), intranasaly (including using a cannula), or by other routes. The agents can be administered orally, e.g., as a tablet or cachet containing a predetermined amount of the active ingredient, gel, pellet, paste, syrup, bolus, electuary, slurry, capsule, powder, granules, as a solution or a suspension in an aqueous liquid or a non-aqueous liquid, as an oil-in-water liquid emulsion or a water-in-oil liquid emulsion, via a micellar formulation (see, e.g., PCT Publication No. WO 97/11682, which is hereby incorporated by reference in its entirety) via a liposomal formulation (see, e.g., EP Patent No. 736299, PCT Publication No. WO 99/59550, and PCT Publication No. WO 97/13500, which are hereby incorporated by reference in their entirety), via formulations described in PCT Publication No. WO 03/094886 (which is hereby incorporated by reference in its entirety) or in some other form. The agents can also be administered transdermally (i.e., via reservoir-type or matrix-type patches, microneedles, thermal poration, hypodermic needles, iontophoresis, electroporation, ultrasound, or other forms of sonophoresis, jet injection, or a combination of any of the preceding methods (Prausnitz et al., “Current Status and Future Potential of Transdermal Drug Delivery,” Nature Reviews Drug Discovery 3:115 (2004), which is hereby incorporated by reference in its entirety). The agents can be administered locally.

The agents can be administered in the form a suppository or by other vaginal or rectal means. The agents can be administered in a transmembrane formulation as described in PCT Publication No. WO 90/07923, which is hereby incorporated by reference in its entirety. The agents can be administered non-invasively via the dehydrated particles described in U.S. Pat. No. 6,485,706, which is hereby incorporated by reference in its entirety. The agents can be administered in an enteric-coated drug formulation as described in PCT Publication No. WO 02/49621, which is hereby incorporated by reference in its entirety. The agents can be administered intranasaly using the formulation described in U.S. Pat. No. 5,179,079, which is hereby incorporated by reference in its entirety. Formulations suitable for parenteral injection are described in PCT Publication No. WO 00/62759, which is hereby incorporated by reference in its entirety. The agents can be administered using the casein formulation described in U.S. Patent Application Publication No. 2003/0206939 and PCT Publication No. WO 00/06108, which are hereby incorporated by reference in their entirety. The agents can be administered using the particulate formulations described in U.S. Patent Application Publication No. 20020034536, which is hereby incorporated by reference in its entirety.

The agents, alone or in combination with other suitable components, can be administered by pulmonary route utilizing several techniques including, but not limited to, intratracheal instillation (delivery of solution into the lungs by syringe), intratracheal delivery of liposomes, insufflation (administration of powder formulation by syringe or any other similar device into the lungs), and aerosol inhalation. Aerosols (e.g., jet or ultrasonic nebulizers, metered-dose inhalers (“MDIs”), and dry-Powder inhalers (“DPIs”)) can also be used in intranasal applications. Aerosol formulations are stable dispersions or suspensions of solid material and liquid droplets in a gaseous medium and can be placed into pressurized acceptable propellants, such as hydrofluoroalkanes (HFAs, i.e., HFA-134a and HFA-227, or a mixture thereof), dichlorodifluoromethane (or other chlorofluorocarbon propellants such as a mixture of Propellants 11, 12, and/or 114), propane, nitrogen, and the like. Pulmonary formulations may include permeation enhancers such as fatty acids, and saccharides, chelating agents, enzyme inhibitors (e.g., protease inhibitors), adjuvants (e.g., glycocholate, surfactin, span 85, and nafamostat), preservatives (e.g., benzalkonium chloride or chlorobutanol), and ethanol (normally up to 5% but possibly up to 20%, by weight). Ethanol is commonly included in aerosol compositions as it can improve the function of the metering valve and in some cases also improve the stability of the dispersion.

Pulmonary formulations may also include surfactants which include, but are not limited to, bile salts and those described in U.S. Pat. No. 6,524,557 and references therein, which are hereby incorporated by reference in their entirety. The surfactants described in U.S. Pat. No. 6,524,557, e.g., a C₈-C₁₆ fatty acid salt, a bile salt, a phospholipid, or alkyl saccharide are advantageous in that some of them also reportedly enhance absorption of the compound in the formulation.

Also suitable are dry powder formulations comprising a therapeutically effective amount of active compound blended with an appropriate carrier and adapted for use in connection with a dry-powder inhaler. Absorption enhancers that can be added to dry powder formulations include those described in U.S. Pat. No. 6,632,456, which is hereby incorporated by reference in its entirety. PCT Publication No. WO 02/080884, which is hereby incorporated by reference in its entirety, describes new methods for the surface modification of powders. Aerosol formulations may include those described in U.S. Pat. Nos. 5,230,884 and 5,292,499; PCT Publication Nos. WO 017/8694 and 01/78696; and U.S. Patent Application Publication No. 2003/019437, 2003/0165436; and PCT Publication No. WO 96/40089 (which includes vegetable oil), which are hereby incorporated by reference in their entirety. Sustained release formulations suitable for inhalation are described in U.S. Patent Application Publication Nos. 2001/0036481, 2003/0232019, and 2004/0018243 as well as in PCT Publication Nos. WO 01/13891, 02/067902, 03/072080, and 03/079885, which are hereby incorporated by reference in their entirety.

Pulmonary formulations containing microparticles are described in PCT Publication No. WO 03/015750, U.S. Patent Application Publication No. 2003/0008013, and PCT Publication No. WO 00/00176, which are hereby incorporated by reference in their entirety. Pulmonary formulations containing stable glassy state powder are described in U.S. Patent Application Publication No. 2002/0141945 and U.S. Pat. No. 6,309,671, which are hereby incorporated by reference in their entirety. Other aerosol formulations are described in EP Patent No. 1338272, PCT Publication No. WO 90/09781, U.S. Pat. Nos. 5,348,730 and 6,436,367, PCT Publication No. WO 91/04011, and U.S. Pat. Nos. 6,294,153 and 6,290,987, which are hereby incorporated by reference in their entirety, which describe a liposomal based formulation that can be administered via aerosol or other means.

Powder formulations for inhalation are described in U.S. Patent Application Publication No. 2003/0053960 and PCT Publication No. WO 01/60341, which are hereby incorporated by reference in their entirety. The agents can be administered intranasally as described in U.S. Patent Application Publication No. 2001/0038824, which is hereby incorporated by reference in its entirety.

Solutions of medicament in buffered saline and similar vehicles are commonly employed to generate an aerosol in a nebulizer. Simple nebulizers operate on Bernoulli's principle and employ a stream of air or oxygen to generate the spray particles. More complex nebulizers employ ultrasound to create the spray particles. Both types are well known in the art and are described in standard textbooks of pharmacy such as Sprowls' American Pharmacy and Remington's The Science and Practice of Pharmacy.

Other devices for generating aerosols employ compressed gases, usually hydrofluorocarbons and chlorofluorocarbons, which are mixed with the medicament and any necessary excipients in a pressurized container. These devices are likewise described in standard textbooks such as Sprowls and Remington.

The agent can be incorporated into a liposome to improve half-life. The agent can also be conjugated to polyethylene glycol (“PEG”) chains. Methods for pegylation and additional formulations containing PEG-conjugates (i.e., PEG-based hydrogels, PEG modified liposomes) can be found in Harris and Chess, Nature Reviews Drug Discovery 2:214-221, which is hereby incorporated by reference in its entirety, and the references therein. The agent can be administered via a nanocochleate or cochleate delivery vehicle (BioDelivery Sciences International). The agents can be delivered transmucosally (i.e., across a mucosal surface such as the vagina, eye or nose) using formulations such as that described in U.S. Pat. No. 5,204,108, which is hereby incorporated by reference in its entirety. The agents can be formulated in microcapsules as described in PCT Publication No. WO 88/01165, which is hereby incorporated by reference in its entirety. The agent can be administered intra-orally using the formulations described in U.S. Patent Application Publication No. 2002/0055496, PCT Publication No. WO 00/47203, and U.S. Pat. No. 6,495,120, which are hereby incorporated by reference in their entirety. The agent can be delivered using nanoemulsion formulations described in PCT Publication No. WO 01/91728, which is hereby incorporated by reference in its entirety.

Another aspect of the present disclosure relates to a method of treating a tumor. This method involves contacting a tumor with a compound described herein under conditions effective to treat the tumor.

Another aspect of the present disclosure relates to a method of treating a tumor. This method involves contacting a tumor with a compound of formula (II) as described herein under conditions effective to treat the tumor.

In some embodiments of carrying out this and other methods of the present disclosure, the method is carried out ex vivo.

In some embodiments of carrying out this and other methods of the present disclosure, the method is carried out in vivo.

In some embodiments, the tumor comprises cells that are low-CUL4-expressing cells.

A further aspect of the present disclosure relates to a method of treating a subject for cancer. This method involves administering to a subject in need of treatment for cancer a compound described herein under conditions effective to treat the subject for cancer.

Yet another aspect of the present disclosure relates to a method of treating a subject for cancer. This method involves administering to a subject in need of treatment for cancer a compound of formula (II) described herein under conditions effective to treat the subject for cancer.

In some embodiments, the cancer treatment method described herein is carried out by administering a compound of formula (II) orally, transdermally, parenterally, subcutaneously, intravenously, intramuscularly, or intraperitoneally.

In carrying out the treatment methods described herein, administering of compounds to a subject may involve administering pharmaceutical compositions containing the compound(s) (i.e., a compound of formula (II)) in therapeutically effective amounts, which means an amount of compound effective in treating the stated conditions and/or disorders in the subject. Such amounts generally vary according to a number of factors well within the purview of ordinarily skilled artisans. These include, without limitation, the particular subject, as well as its age, weight, height, general physical condition, and medical history, the particular compound used, as well as the carrier in which it is formulated and the route of administration selected for it; the length or duration of treatment; and the nature and severity of the condition being treated.

Administering typically involves administering pharmaceutically acceptable dosage forms, which means dosage forms of compounds described herein and includes, for example, tablets, dragees, powders, elixirs, syrups, liquid preparations, including suspensions, sprays, inhalants tablets, lozenges, emulsions, solutions, granules, capsules, and suppositories, as well as liquid preparations for injections, including liposome preparations. Techniques and formulations generally may be found in Remington's Pharmaceutical Sciences, Mack Publishing Co., Easton, Pa., latest edition, which is hereby incorporated by reference in its entirety.

In carrying out treatment methods, the drug (i.e., a compound of formula (II)) may be contained, in any appropriate amount, in any suitable carrier substance. The drug may be present in an amount of up to 99% by weight of the total weight of the composition. The composition may be provided in a dosage form that is suitable for the oral, parenteral (e.g., intravenously, intramuscularly), rectal, cutaneous, nasal, vaginal, inhalant, skin (patch), or ocular administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols.

Pharmaceutical compositions according to the present disclosure may be formulated to release the active drug substantially immediately upon administration or at any predetermined time or time period after administration.

Controlled release formulations include (i) formulations that create a substantially constant concentration of the drug(s) within the body over an extended period of time; (ii) formulations that after a predetermined lag time create a substantially constant concentration of the drug(s) within the body over an extended period of time; (iii) formulations that sustain drug(s) action during a predetermined time period by maintaining a relatively, constant, effective drug level in the body with concomitant minimization of undesirable side effects associated with fluctuations in the plasma level of the active drug substance; (iv) formulations that localize drug(s) action by, e.g., spatial placement of a controlled release composition adjacent to or in the diseased tissue or organ; and (v) formulations that target drug(s) action by using carriers or chemical derivatives to deliver the drug to a particular target cell type.

Administration of drugs in the form of a controlled release formulation is especially preferred in cases in which the drug has (i) a narrow therapeutic index (i.e., the difference between the plasma concentration leading to harmful side effects or toxic reactions and the plasma concentration leading to a therapeutic effect is small; in general, the therapeutic index (“TI”) is defined as the ratio of median lethal dose (LD₅₀) to median effective dose (ED₅₀)); (ii) a narrow absorption window in the gastro-intestinal tract; or (iii) a very short biological half-life so that frequent dosing during a day is required in order to sustain the plasma level at a therapeutic level.

Any of a number of strategies can be pursued to obtain controlled release in which the rate of release outweighs the rate of metabolism of the drug in question. Controlled release may be obtained by appropriate selection of various formulation parameters and ingredients, including, e.g., various types of controlled release compositions and coatings. Thus, the drug is formulated with appropriate excipients into a pharmaceutical composition that, upon administration, releases the drug in a controlled manner (single or multiple unit tablet or capsule compositions, oil solutions, suspensions, emulsions, microcapsules, microspheres, nanoparticles, patches, and liposomes).

Thus, administering may be carried out orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes. Compounds may be administered alone or with suitable pharmaceutical carriers, and can be in solid or liquid form, such as tablets, capsules, powders, solutions, suspensions, or emulsions.

The subject may be a mammalian subject. In some embodiments, the subject is a human subject. Suitable human subjects include, without limitation, children, adults, and elderly subjects having a beta-cell and/or insulin deficiency.

In some embodiments, the subject may be bovine, ovine, porcine, feline, equine, murine, canine, lapine, etc.

In some embodiments of this and other aspects of the present disclosure, the designation of a compound is meant to designate the compound per se, as well as any pharmaceutically acceptable salt, hydrate, isomer, racemate, ester, or ether thereof. The designation of a compound is meant to designate the compound as specifically designated per se, as well as any pharmaceutically acceptable salt thereof.

Within the context of the present disclosure, by “treating” it is meant preventive or curative treatment.

The terms “treat” and “treating” in the context of the administration of a therapeutically effective amount of a combination of agents refers to a treatment/therapy from which a subject in need of treatment for a disease receives a beneficial effect, such as the reduction, decrease, attenuation, diminishment, stabilization, remission, suppression, inhibition or arrest of the development or progression of the disease, or a symptom thereof. In some embodiments, the treatment/therapy that a subject receives does not cure the disease, but prevents the progression or worsening of the disease. In certain embodiments, the treatment/therapy that a subject receives does not prevent the onset/development of disease, but may prevent the onset of disease symptoms.

The terms “subject” or “patient” are used interchangeably. As used herein, the terms “subject” and “subjects” refer to an animal. For example, the subject may be a mammal. Suitable mammals include non-human mammals (e.g., a camel, donkey, zebra, cow, horse, horse, cat, dog, rat, and mouse, etc.), non-human primates (e.g., a monkey, chimpanzee, etc.), and a human. In some embodiments of the methods according to the present disclosure, the subject is a non-human mammal. In certain embodiments of the methods according to the present disclosure, the subject is a pet (e.g., dog or cat) or farm animal (e.g., a horse, pig or cow). In other specific embodiments of the methods according to the present disclosure, the subject is a human.

In some embodiments of the methods disclosed herein, the subject treated in accordance with the methods described herein has been diagnosed with cancer. Techniques for diagnosing cancer are known to one of skill in the art and include, without limitation, biopsy (e.g., fine needle biopsy), magnetic resonance imaging (MRI), computation tomography (CT or CAT scan), positron emission tomography (PET) or PET-CT scan, etc.

In some embodiments of the methods disclosed herein, the method further involves selecting a subject in need of cancer treatment prior to said administering.

In some embodiments of the methods according to the present disclosure, said administering results in one, two, three or more of the following effects: complete response, partial response, increase in overall survival, increase in disease free survival, increase in objective response rate, increase in time to progression, increase in progression-free survival, increase in time-to-treatment failure, and improvement or elimination of one or more symptoms of the disease.

In some embodiments, said administering is effective to prolong overall survival and/or progression-free survival in the subject. In some embodiments, a method of treating disease as described herein results in an increase in overall survival. In other embodiments, a method of treating the disease as described herein results in an increase in progression-free survival. In other specific embodiments, a method of treating disease as described herein results in an increase in overall survival and an increase in progression-free survival.

The term “complete response” refers to an absence of clinically detectable disease with normalization of any previously abnormal imaging or serum studies.

The term “partial response” refers to at least about a 10%, 20%, 30%, 40%, 50%, 60%, 70%, 80%, or 90% decrease in all measurable tumor burden (i.e., the number of cancer cells present in the subject, or the measured bulk of tumor masses or the quantity of abnormal monoclonal protein) in the absence of new lesions.

The term “overall survival” is defined as the time from randomization until death from any cause, and is measured in the intent-to-treat population. Overall survival should be evaluated in randomized controlled studies. Demonstration of a statistically significant improvement in overall survival can be considered to be clinically significant if the toxicity profile is acceptable, and has often supported new drug approval

Several endpoints are based on tumor assessments. These endpoints include disease free survival (DFS), objective response rate (ORR), time to progression (TTP), progression-free survival (PFS), and time-to-treatment failure (TTF). The collection and analysis of data on these time-dependent endpoints are based on indirect assessments, calculations, and estimates (e.g., tumor measurements).

Generally, “disease free survival” or “DFS” is defined as the time from randomization until recurrence of tumor or death from any cause. Although overall survival is a conventional endpoint for most adjuvant settings, DFS can be an important endpoint in situations where survival may be prolonged, making a survival endpoint impractical. DFS can be a surrogate for clinical benefit or it can provide direct evidence of clinical benefit. This determination is based on the magnitude of the effect, its risk-benefit relationship, and the disease setting. The definition of DFS can be complicated, particularly when deaths are noted without prior tumor progression documentation. These events can be scored either as disease recurrences or as censored events. Although all methods for statistical analysis of deaths have some limitations, considering all deaths (deaths from all causes) as recurrences can minimize bias. DFS can be overestimated using this definition, especially in patients who die after a long period without observation. Bias can be introduced if the frequency of long-term follow-up visits is dissimilar between the study arms or if dropouts are not random because of toxicity.

As used herein, “objective response rate” or “ORR” is defined as the proportion of patients with tumor size reduction of a predefined amount and for a minimum time period. Response duration usually is measured from the time of initial response until documented tumor progression. Generally, the FDA has defined ORR as the sum of partial responses plus complete responses. When defined in this manner, ORR is a direct measure of drug antitumor activity, which can be evaluated in a single-arm study. If available, standardized criteria should be used to ascertain response. A variety of response criteria have been considered appropriate (e.g., RECIST criteria) (Therasse et al., “New Guidelines to Evaluate the Response to Treatment in Solid Tumors. European Organization for Research and Treatment of Cancer, National Cancer Institute of the United States, National Cancer Institute of Canada,” J. Natl. Cancer Inst. 92:205-216 (2000), which is hereby incorporated by reference in its entirety). The significance of ORR is assessed by its magnitude and duration, and the percentage of complete responses (no detectable evidence of tumor).

As used herein, “time to progression” or “TTP” and “progression-free survival” or “PFS” have served as primary endpoints for drug approval. TTP is defined as the time from randomization until objective tumor progression; TTP does not include deaths. PFS is defined as the time from randomization until objective tumor progression or death. Compared with TTP, PFS is the preferred regulatory endpoint. PFS includes deaths and thus can be a better correlate to overall survival. PFS assumes patient deaths are randomly related to tumor progression. However, in situations where the majority of deaths are unrelated to cancer, TTP can be an acceptable endpoint.

As an endpoint to support drug approval, PFS can reflect tumor growth and be assessed before the determination of a survival benefit. Its determination is not confounded by subsequent therapy. For a given sample size, the magnitude of effect on PFS can be larger than the effect on overall survival. However, the formal validation of PFS as a surrogate for survival for the many different malignancies that exist can be difficult. Data are sometimes insufficient to allow a robust evaluation of the correlation between effects on survival and PFS. Cancer trials are often small, and proven survival benefits of existing drugs are generally modest. The role of PFS as an endpoint to support licensing approval varies in different cancer settings. Whether an improvement in PFS represents a direct clinical benefit or a surrogate for clinical benefit depends on the magnitude of the effect and the risk-benefit of the new treatment compared to available therapies.

As used herein, “time-to-treatment failure” or “TTF” is defined as a composite endpoint measuring time from randomization to discontinuation of treatment for any reason, including disease progression, treatment toxicity, and death. TTF is not recommended as a regulatory endpoint for drug approval. TTF does not adequately distinguish efficacy from these additional variables. A regulatory endpoint should clearly distinguish the efficacy of the drug from toxicity, patient, or physician withdrawal, or patient intolerance.

In some embodiments, treating a subject comprises an improvement in and/or the elimination of one or more symptoms of cancer in the subject. The one or more symptoms of cancer may include, but are not limited to, a lump on the roof of the mouth, under the tongue, or in the bottom of the mouth; an abnormal area on the lining of the mouth; numbness of the upper jaw, palate, face, or tongue; difficulty swallowing or chewing; hoarseness; dull pain; a bump or nodule in front of the ear or underneath the jaw; paralysis of a facial nerve; fatigue; pain; weakness; loss of appetite; swollen lymph nodes; persistent bad breath; and unintended weight loss.

In some embodiments, said administering is effective to induce regression of a primary tumor and/or a metastatic tumor in the subject. Techniques for evaluating tumor regression and/or metastatic disease are known to one of skill in the art and include, without limitation, biopsy (e.g., fine needle biopsy), magnetic resonance imaging (MRI), computation tomography (CT or CAT scan), positron emission tomography (PET) or PET-CT scan, etc.

Yet another aspect of the present disclosure relates to a method of treating a subject for cancer. This method involves administering to a subject in need of treatment for cancer a compound selected from the group consisting of

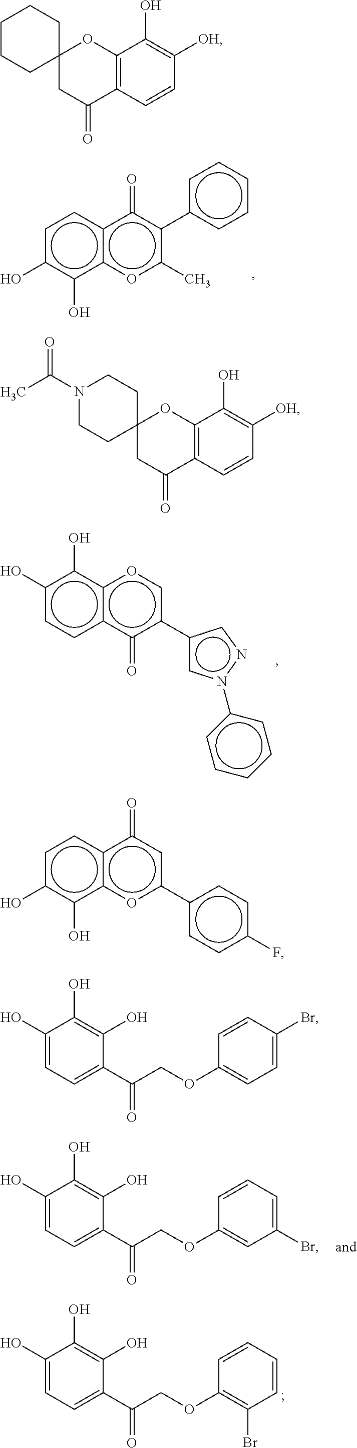

under conditions effective to treat the subject for cancer.

Yet another aspect of the present disclosure relates to a method of treating a subject for cancer. This method involves administering to a subject in need of treatment for cancer a compound selected from the group consisting of

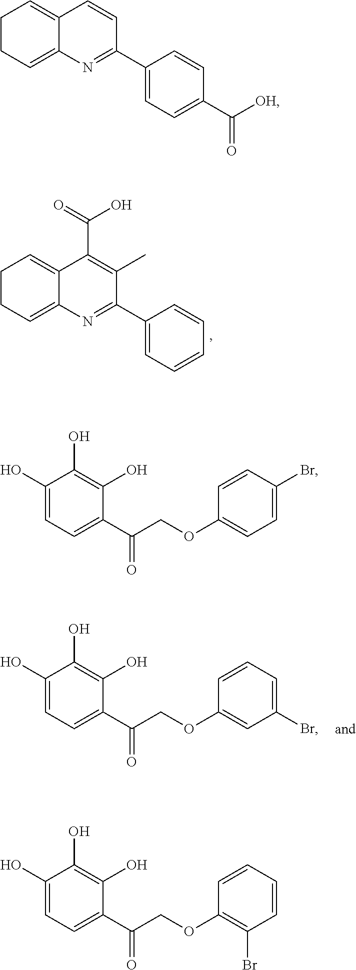 [SMILES: O=C(O)c1ccc(-c2ccc3c(n2)=CCCC=3)cc1 ; Cc1c(-c2ccccc2)nc2c(c1C(=O)O)=CCCC=2 ; O=C(COc1ccc(Br)cc1)c1ccc(O)c(O)c1O ; O=C(COc1cccc(Br)c1)c1ccc(O)c(O)c1O ; "and" ; O=C(COc1ccccc1Br)c1ccc(O)c(O)c1O]

under conditions effective to treat the subject for cancer.

Cancers amenable to the treatment include, without limitation, Acute Lymphoblastic Leukemia (ALL), Acute Myeloid Leukemia (AML), Adrenocortical Carcinoma, Adrenal Cortex Cancer, Anal Cancer, Appendix Cancer, Astrocytomas, Atypical Teratoid/Rhabdoid Tumor, Basal Cell Carcinoma, Extrahepatic Bile Duct Cancer, Bladder Cancer, Bone Cancer, Brain Tumors, Breast Cancer, Bronchial Tumors, Burkitt Lymphoma, Carcinoid Tumor, Cardiac (Heart) Tumors, Cervical Cancer, Cholangiocarcinoma, Chronic Lymphocytic Leukemia (CLL), Chronic Myelogenous Leukemia (CML), Chronic Myeloproliferative Neoplasms, Colorectal Cancer, Cutaneous T-Cell Lymphoma, Ductal Carcinoma In Situ (DCIS), Endometrial Cancer, Ependymoma, Esophageal, Esthesioneuroblastoma, Ewing Sarcoma, Intraocular Melanoma, Retinoblastoma, Malignant Fibrous Histiocytoma of Bone, Osteosarcoma, Gallbladder Cancer, Gastric (Stomach) Cancer, Gastrointestinal Carcinoid Tumor, Gastrointestinal Stromal Tumors (GIST), Gestational Trophoblastic Disease, Gliomas, Hairy Cell Leukemia, Head and Neck Cancer, Hepatocellular (Liver) Cancer, Langerhans Cell Histiocytosis, Hodgkin Lymphoma, Hypopharyngeal Cancer, Intraocular Melanoma, Kaposi Sarcoma, Kidney cancer, Langerhans Cell Histiocytosis, Leukemia, Lung Cancer, Lymphoma, Medullary Thyroid Carcinoma, Melanoma, Intraocular (Eye) Melanoma, Merkel Cell Carcinoma, Malignant Mesothelioma, Metastatic Squamous Neck Cancer with Occult Primary, Multiple Endocrine Neoplasia Syndromes, Multiple Myeloma/Plasma Cell Neoplasms, 5 Myelodysplastic Syndromes, Myelodysplastic/Myeloproliferative Neoplasms, and Chronic Myeloproliferative Neoplasms, Chronic Myelogenous Leukemia (CML), Acute Myeloid Leukemia (AML), Nasopharyngeal Cancer, Neuroblastoma, Non-Hodgkin Lymphoma, Non-Small Cell Lung Cancer, Oral Cancer, Lip and Oral Cavity Cancer, Oropharyngeal Cancer, Ovarian Cancer, Pancreatic Cancer and Pancreatic Neuroendocrine Tumors (Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Plasma Cell Neoplasm/Multiple Myeloma, Primary Central Nervous System (CNS) Lymphoma, Prostate Cancer, Rectal Cancer, Renal Cell (Kidney) Cancer, Retinoblastoma, Rhabdomyosarcoma, Salivary Gland Cancer, Sarcoma, Sezary Syndrome, Small Cell Lung Cancer, Small Intestine Cancer, Soft Tissue Sarcoma, Squamous Cell Carcinoma, Squamous Neck Cancer, Stomach (Gastric) Cancer, T-Cell Lymphoma, Testicular Cancer, Throat Cancer, Thymoma and Thymic Carcinoma, Thyroid Cancer, Transitional Cell Cancer of the Renal Pelvis and Ureter, Urethral Cancer, Uterine Cancer, Endometrial and Uterine Sarcoma, Vaginal Cancer, Vulvar Cancer, Waldenstrom Macroglobulinemia, and Wilms Tumor.

In some embodiments, the treatment method is carried out to treat leukemia.

### EXAMPLES

### Example 1—Materials and Methods

Commercial Compounds

The following compounds were purchased from ChemBridge: #33 (ID #6664054; 7,8-dihydroxy-3-phenoxy-2-(trifluoromethyl)-4H-chromen-4-one), #33-11 (ID #6655693; 7,8-dihydroxy-3-(1-phenyl-1H-pyrazol-4-yl)-2-(trifluoromethyl)-4H-chromen-4-one), #33-2 (ID #6664007; 7,8-dihydroxy-2-methyl-3-phenoxy-4H-chromen-4-one), #33-3 (ID #6687390; 7,8-dimethoxy-2-methyl-3-phenoxy-4H-chromen-4-one), #33-4 (ID #6679857; 7-phenoxy-8-(trifluoromethyl)-6H-[1,3]dioxolo[4,5-h]chromen-6-one), #33-5 (ID #6651985; 8-phenoxy-9-(trifluoromethyl)-2,3-dihydro-7H-[1,4]dioxino[2,3-h]chromen-7-one), and #33-7 (ID #5314926; 7-hydroxy-3-phenoxy-2-(trifluoromethyl)-4H-chromen-4-one).

Protein Reagents

Previous publications have described the preparation of the following reagents: ROC1-CUL1 and Nedd8-ROC1-CUL1 (Zheng et al., “Structure of the Cul1-Rbx1-Skp1-F boxSkp2 SCF Ubiquitin Ligase Complex,” Nature 416(6882):703-709 (2002), which is hereby incorporated by reference in its entirety); ROC1-CUL4A, Nedd8-ROC1-CUL4A, GST-ROC1-CUL4A (Angers et al., “Molecular Architecture and Assembly of the DDB1-CUL4A Ubiquitin Ligase Machinery,” Nature 443(7111):590-593 (2006), which is hereby incorporated by reference in its entirety); ROC1-CUL1 CTD, ³²P-IκBα-Ub, iFluor555-Ub-Q31C-K48R, Ub-NC-1555, Ub E1, Cdc34a, and UbcH5c (Wu et al., “Suramin Inhibits Cullin-RING E3 Ubiquitin Ligases,” PNAS 113(14):E2011-2018 (2016), which is hereby incorporated by reference in its entirety); Skp1-βTrCP (Kovacev et al., “Priming and Extending: an UbcH5/Cdc34 E2 Handoff Mechanism for Polyubiquitination on a SCF Substrate,” Mol. Cell 37:784-796 (2010), which is hereby incorporated by reference in its entirety); ³²P-β-catenin (Kovacev et al., “A Snapshot at Ubiquitin Chain Elongation: Lysine 48-Tetra-ubiquitin Slows Down Ubiquitination,” J. Biol. Chem. 289(10):7068-7081 (2014), which is hereby incorporated by reference in its entirety); ³²P-PK-Ub (Tan et al., “Recruitment of a ROC1:Cullin1 Ubiquitin Ligase by Skp1 and HOS to Catalyze the Ubiquitination of IkBa,” Mol. Cell 3:527-533 (1999), which is hereby incorporated by reference in its entirety); and PK-Ub (1-74) (Gazdoiu et al., “Human cdc34 Employs Distinct Sites to Coordinate Attachment of Ubiquitin to a Substrate and Assembly of Polyubiquitin Chains,” Mol. Cell Biol. 27(20):7041-7052 (2007), which is hereby incorporated by reference in its entirety). CRL4^{CRBN}, Cdc34b, Fluorescein-Ub, Rhodamine-Ub, and Ub K0 were purchased from Boston Biochem.

Cells

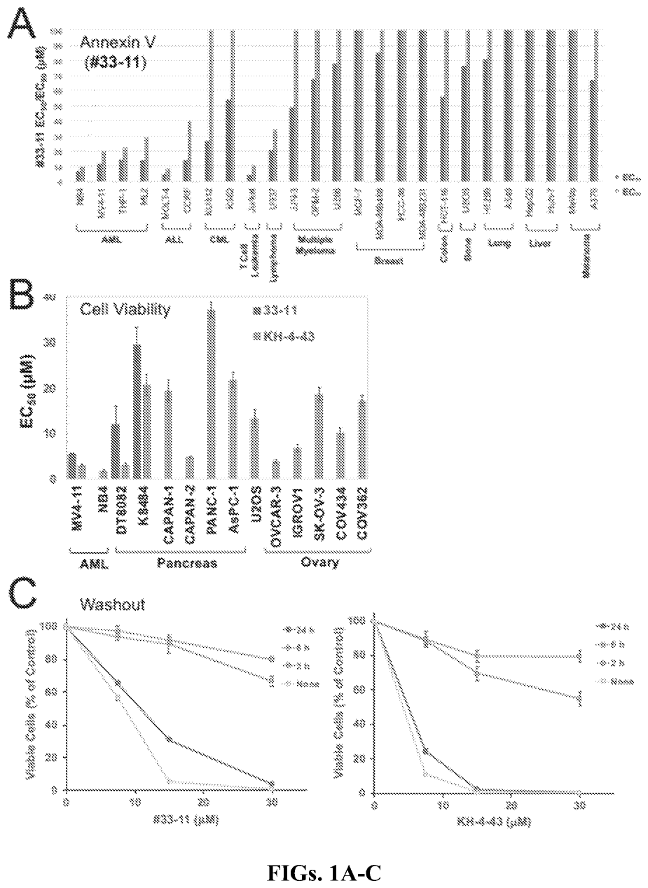

36 tumor cell lines were used for this study (FIG. 1). Human leukemia cells lines (e.g., AML MV4-11) and H1299 cells were maintained in RPMI 1640 media supplemented with 10% heat-inactivated FBS and penicillin (100 U/mL)-streptomycin (100 μg/mL) in a 37° C. humidified incubator with 5% CO₂. All other cell lines were maintained in DMEM instead of RPMI. All cell media reagents were obtained from Thermo Fisher Scientific.

### Example 2—High Throughput Screen, Hits Validation, and Classification

The high throughput screen (“HTS”) employed the FRET K48 di-Ub assay as established previously (Wu et al., “Suramin Inhibits Cullin-RING E3 Ubiquitin Ligases,” PNAS 113(14):E2011-2018 (2016), which is hereby incorporated by reference in its entirety) and was carried out at Memorial Sloan Kettering Cancer Center Core Screening Facility. For HTS, the assay was formatted using three reagent addition steps. Library compounds, stored in 10 mM stock plates (DMSO), were transferred into assay plates and each well contains a single compound at 100 μM in 1 μl 10% DMSO. The complete complement of proteins/enzymes (6.5 μl), containing Ub E1 (14 nM), E2 Cdc34 (124 nM), E3 ROC1-CUL1 CTD (0.2 μM), Ub C31-I555 (donor, 0.93 μM), and Ub C64-I647 (receptor, 1.62 μM), in the presence of 33 mM Tris-HCl (pH 7.4), 1.7 mM MgCl₂, 0.33 mM DTT, and BSA (0.07 mg/ml), was added to each plate. The concentrations of all five proteins have been determined based on Kₘ measurement as described previously (Wu et al., “Suramin Inhibits Cullin-RING E3 Ubiquitin Ligases,” PNAS 113(14):E2011-2018 (2016), which is hereby incorporated by reference in its entirety). To initiate ubiquitination, ATP (2.5 μl in 2.64 mM) was added and its final concentration was 0.66 mM. The concentration of ATP was set high to minimize identification of nucleotide analogues because the priority of the HTS is to find small molecule modulators specific for E3 ROC1-CUL1 CTD. In this setting, the concentration of compound and DMSO in a given well were 10 μM and 1%, respectively. After incubation at room temperature for 45 min, EDTA (30 mM) was added to terminate the reaction. The reaction was quantified based on the ratio of acceptor:donor fluorescence (excitation 515 nm; donor emission 570 nm; acceptor emission 670 nm) using a Victor high-throughput plate reader (Perkin Elmer).

The ChemBridge library of 107,940 compounds, selected based on their structural diversity and drug-like property, were formatted in 77 1,536-well library plates, with 1,408 small molecules distributed in 1-24 & 29-48 columns; 25-28 columns were left empty for controls. Columns 25 and 26 contained all reaction mixture without compound but with 1% DMSO. Columns 27 and 28 contained 5 and 0.8 μM Suramin (an inhibitor of E3 ROC1-CUL1 CTD; Wu et al., “Suramin Inhibits Cullin-RING E3 Ubiquitin Ligases,” PNAS 113(14):E2011-2018 (2016), which is hereby incorporated by reference in its entirety). Under the screening conditions, at 5 and 0.8 μM, Suramin typically inhibits the reaction by 100 and 50%, respectively. These controls were used for plate validation and assay normalization. Data were normalized against plate controls. Screen performance were judged by Z′ score (Zhang et al., “A Simple Statistical Parameter for Use in Evaluation and Validation of High Throughput Screening Assays,” J. Biomol. Screen 4:67-73 (1999), which is hereby incorporated by reference in its entirety). The Z′ score for each of the 77 assay plates was calculated: All values were greater than 0.5, with 74 of 77 plates exhibiting Z′ score of 0.75 or higher. Thus, the screening quality was judged excellent. 136 positive hits were identified based on a cut-off rate with three standard deviations from the controls.

Subsequent gel-based assays were performed to eliminate false positives that are fluorescent in nature, corrupted compounds on assay plates, or poor storage. First, the reaction material retrieved from the select wells in an assay plate was subjected to gel electrophoresis to validate the inhibition of di-Ub synthesis by the compound. Further confirmation of inhibition was carried out by testing the effects of freshly purchased compounds on the di-Ub synthesis reaction. In addition, HTS hits were subject to PAINS screen and preliminary medicinal chemistry evaluation. Nine Hits passed these tests.

The inhibitors identified above were then subjected to follow up assays for classification. These include E1-Ub and E2 Cdc34-Ub thiol ester formation assays (Wu et al., “Suramin Inhibits Cullin-RING E3 Ubiquitin Ligases,” PNAS 113(14):E2011-2018 (2016), which is hereby incorporated by reference in its entirety), as well as E3 discharge assay. Altogether, these analyses led to classification of 9 hits into one E1 inhibitor, one E2 Cdc34 inhibitor, and 7 inhibitors of E3 ROC1-CUL1 CTD. Of the 7 E3 inhibitors, compound #33 was pursued as described in this work.

### Example 3—Chemical Reagents and Methods

Reactions were performed under a nitrogen atmosphere unless otherwise noted. Tetrahydrofuran, acetonitrile, dimethyl sulfoxide, dichloromethane, acetone, and toluene were purchased from Acros Organics. All other solvents used were ACS grade, and all other reagents were used as received, unless otherwise noted. All other commercially available chemicals were purchased from Alfa Aesar (Ward Hill, MA), Sigma-Aldrich (St. Louis, MO), Oakwood Products (West Columbia, SC), Strem Chemicals (Newburport, MA), and TCI America (Portland, OR). Qualitative TLC analysis was performed on 250 mm thick, 60 Å, glass backed, F254 silica (Silicycle, Quebec City, Canada). Visualization was accomplished with UV light and exposure to p-anisaldehyde or KMnO₄ stain solutions followed by heating. Flash chromatography was performed using FlashCombi. ¹H NMR spectra were acquired on a Bruker Ultrashield (at 600 MHz) High resolution mass spectrometry data were acquired on an Agilent 1200 TOF LC/MS.

### Example 4—Synthetic Procedures

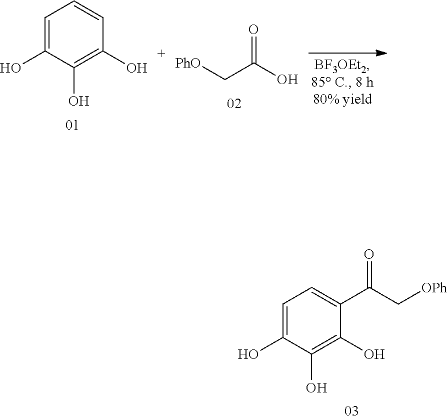 [SMILES: Oc1cccc(O)c1O ; O=C(O)COc1ccccc1 ; O=C(COc1ccccc1)c1ccc(O)c(O)c1O]

Friedel Crafts Procedure (Basha et al., “A Mild and Efficient Protocol to Synthesize Chromones, Isoflavones, and Homoisoflavones Using the Complex 2,4,6-trichloro-1,3,5-triazine/dimethylformamide,” Can. J. Chem. 91:763-768 (2013), which is hereby incorporated by reference in its entirety): Phenol (03): To a solution of pyrogallol 01 (0.174 g, 1.38 mmol) in BF₃OEt₂ (13.8 mL, 0.1 M) at 23° C. was added commercial carboxylic acid 02 (0.199 g, 1.31 mmol) dropwise, and the mixture was stirred for 2.5 hours at 85° C. Upon consumption of starting material, the reaction mixture was quenched with sat. NaOAc (50 mL). The solution was extracted with EtOAc (2×100 mL). The combined organic layers were washed with brine (50 mL) and dried over MgSO₄. After removal of solvent by rotary evaporation, the crude material was purified by flash chromatography (1:1 hexanes/EtOAc eluent), affording phenol 03 (0.274 g, 80% yield) as a colorless solid. ¹H NMR (600 MHz, CDCl₃) δ 7.34 (t, J=8.2 Hz 1H), 7.06 (t, J=7.8 Hz 1H) 6.96 (d, J=8.2 Hz 2H), 6.70 (t, J=7.8 Hz, 1H), 6.51 (d, J=8.2 Hz, 1H), 4.71 (s, 2H); HRMS (TOF LC/MS) m/z cal'd for (M+H)⁺ [C₁₄H₁₂O₅+H]⁺: 260.24, found 261.0292.

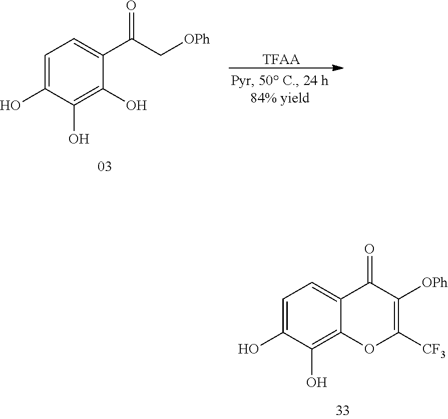 [SMILES: O=C(COc1ccccc1)c1ccc(O)c(O)c1O ; "TFAA" ; O=c1c(Oc2ccccc2)c(C(F)(F)F)oc2c(O)c(O)ccc12]

Cyclization Procedure (Otsalyuk et al., “Synthetic Analogs of Xanthocercin,” Chem. Nat. Compd. 34:284-288 (1998), which is hereby incorporated by reference in its entirety): Compound (33): To a solution of phenol 03 (0.097 g, 0.372 mmol) in pyridine (1.5 mL, 0.3 M) at 23° C. was added TFAA (0.180 mL, 1.29 mmol), and the mixture was stirred for 16 hours at 50° C. Upon consumption of starting material, the solution was diluted with H₂O (10 mL) and then extracted with EtOAc (2×20 mL). The combined organic layers were washed with brine (20 mL) and dried over MgSO₄. After removal of solvent by rotary evaporation, the crude material was purified by flash chromatography (1:1 hexanes/EtOAc eluent), affording compound 33 (0.121 g, 84% yield) as a pale yellow solid. ¹H NMR (600 MHz, MeOD) δ 7.55 (d, J=8.8 Hz, 1H), 7.32 (dd, J=7.5, 8.0 Hz, 2H), 7.08 (dd, J=7.5, 8.0 Hz, 1H), 7.05 (d, J=8.8 Hz, 1H), 6.99 (d, J=8.0 Hz 1H); HRMS (TOF LC/MS) m/z cal'd for (M+H)⁺ [C₁₆H₉F₃O₅+H]⁺: 338.24, found 339.0478.

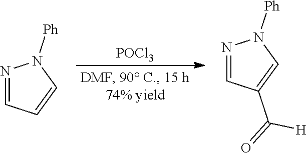

To an anhydrous DMF (10.7 mL, 14.0 mmol) was slowly added POCl₃ (13 mL, 14.0 mmol) at 0° C. under N₂. After 30 min, phenyl pyrazole (4.6 mL, 35.0 mmol) was added. The reaction mixture was stirred at 90° C. for 15 hours. Upon consumption of starting material, the solution was diluted with H₂O (50 mL) and then extracted with EtOAc (2×100 mL). The combined organic layers were washed with brine (50 mL) and dried over MgSO₄. After removal of solvent by rotary evaporation, the residue was recrystallized with 1:4 EtOAc/hexane to afford phenyl pyrazole carbaldehyde (4.44 g, 74% yield) as a colorless solid. ¹H NMR (600 MHz, (CDCl₃) δ 8.00 (s, 1H), 7.67-7.65 (m, 3H), 7.48 (t, J=7.3 Hz, 2H), 7.35 (t, J=7.3 Hz, 1H); HRMS (TOF LC/MS (APCI)) m/z cal'd for (M+H)⁺ [C₁₀H₉N₂O+H]⁺: 173.07, found 173.0705.

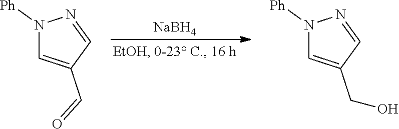 [SMILES: O=Cc1cnn(-c2ccccc2)c1 ; "NaBH4" ; OCc1cnn(-c2ccccc2)c1]

To a solution of phenyl pyrazole carbaldehyde (3.94 g, 20.87 mmol) in EtOH (35 mL, 0.7 M) was added NaBH₄ (2.025 g, 53.54 mmol) portion wise at 0° C. The reaction mixture was stirred at 23° C. for 16 hours. The solution was diluted with H₂O (100 mL) at 0° C. and then extracted with EtOAc (2×150 mL). The combined organic layers were washed with brine (100 mL) and dried over MgSO₄. The solvent was removed by rotary evaporation to afford the benzyl pyrazole alcohol as pale yellow oil. The crude was used in next step. ¹H NMR (600 MHz, CDCl₃) δ 7.97 (s, 1H), 7.75 (s, 1H), 7.70 (d, J=8.6 Hz, 2H), 7.48 (dd, J=7.5. 8.6 Hz, 2H), 7.33 (t, J=7.5 Hz, 1H), 4.71 (d, J=5.2 Hz, 2H); HRMS (TOF LC/MS) m/z cal'd for (M+H)⁺ [C₁₀H₁₀N₂O+H]⁺: 174.20, found 175.4088.

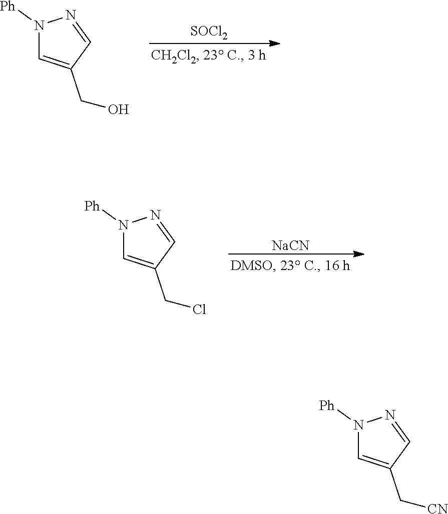 [SMILES: OCc1cnn(-c2ccccc2)c1 ; "SOCl2" ; ClCc1cnn(-c2ccccc2)c1 ; "NaCN" ; N#CCc1cnn(-c2ccccc2)c1]

To a solution of benzyl pyrazole alcohol in CH₂Cl₂ (21 mL, 1 M) was added thionyl chloride (2.00 mL, 26.89 mmol) at 23° C. under N₂. The reactant mixture was stirred for 3 hours. The solvent was removed by rotary evaporation and the residue was filtered through a pad of SiO₂ with EtOAc (100 mL). The solvent was removed by rotary evaporation to afford benzyl pyrazole chloride as brown oil. The crude was used in next step. ¹H NMR (600 MHz, CDCl₃) δ 7.99 (s, 1H), 7.76 (s, 1H), 7.69 (d, J=8.0 Hz, 2H), 7.48 (dd, J=7.5. 8.0 Hz, 2H), 7.33 (t, J=7.5 Hz, 1H), 4.65 (s, 2H);

To a solution of benzyl pyrazole chloride in DMSO (15 mL, 1.4 M) was added NaCN (1.48 g, 30.23 mmol) at 23° C. After the mixture was stirred for 16 hours, it was diluted with H₂O (100 mL) and extracted with EtOAc (2×150 mL). The combined organic layers were washed with brine (100 mL) and dried over MgSO₄. The solvent was removed by rotary evaporation to afford 2-(1-benzyl-1H-pyrazol-4-yl)acetonitrile as a brown oil. The crude was used in next step. ¹H NMR (600 MHz, CDCl₃) δ 7.97 (s, 1H), 7.69 (s, 1H), 7.68 (d, J=8.1 Hz, 2H), 7.49 (dd, J=7.4, 8.1 Hz, 2H), 7.34 (t, J=7.4 Hz, 1H), 3.71 (s, 2H); HRMS (TOF LC/MS) m/z cal'd for (M+H)⁺ [C₁₁H₉N₃+H]⁺: 183.21, found 184.0872.

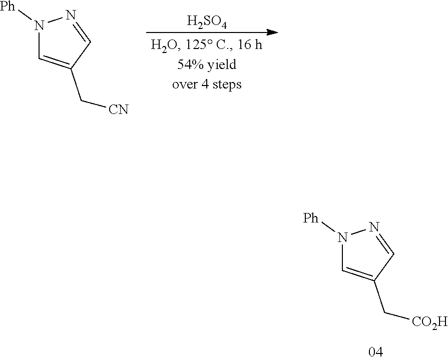 [SMILES: N#CCc1cnn(-c2ccccc2)c1 ; "H2SO4" ; O=C(O)Cc1cnn(-c2ccccc2)c1]

To a solution of pyrazole acetonitrile in H₂O (42 mL, 0.5 M) was added sulfuric acid (42 mL, 0.5 M) at 23° C. The reactant mixture was stirred at 125° C. for 16 hours. The reactant solution was slowly added into sat. NaOH (100 mL) at 0° C. The solution was acidified to pH 5. The mixture was extracted with EtOAC (2×150 ml). The combined organic layers were washed with brine (100 mL) and dried over MgSO₄. The solvent was removed by rotary evaporation, and the residue was recrystallized with (˜10:1 Hexane/EtOAC eluent) to afford benzyl pyrazole carboxylic acid 04 (2.293 g, 54% yield over 4 steps) as a colorless solid. NMR (600 MHz, CDCl₃) δ 7.96 (s, 1H), 7.70 (s, 1H), 7.68 (d, J=8.3 Hz, 2H), 7.47 (dd, J=7.5, 83 Hz, 2H), 7.31 (t, J=7.4 Hz, 1H), 3.67 (s, 2H); HRMS (TOF LC/MS) m/z cal'd for (M+H)⁺ [C₁₁H₁₀N₂O₂+H]⁺: 202.24, found 203.6023.

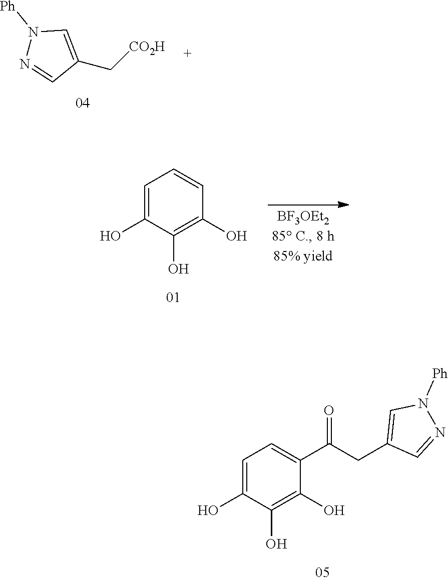 [SMILES: O=C(O)Cc1cnn(-c2ccccc2)c1 ; Oc1cccc(O)c1O ; O=C(Cc1cnn(-c2ccccc2)c1)c1ccc(O)c(O)c1O]

Fridel Crafts Procedure: Phenol 05. To a solution of pyrogallol 01 (0.1858 g, 1.45 mmol) in BF₃OEt₂ (2.6 mL, 0.33 M) at 23° C. was added carboxylic acid 04 (Menozzi et al., “Synthesis of 5-Substituted 1-Aryl-1H-Pyrazole-4-Acetonitriles, 4-Methyl-1-Phenyl-1H-Pyrazole-3-Carbonitriles and Pharmacologically Active 1-Aryl-1H-Pyrazole-4-Acetic Acids,” J. Heterocycl. Chem., 30:997-1002 (1993), which is hereby incorporated by reference in its entirety) (0.259 g, 1.28 mmol) dropwise, and the mixture was stirred for 16 hours at 85° C. Upon consumption of starting material, the reaction mixture was quenched with sat. NaOAc (10 mL). The solution was extracted with EtOAc (2×20 mL). The combined organic layers were washed with brine (20 mL) and dried over MgSO₄. After removal of solvent by rotary evaporation, the crude material was purified by flash chromatography (1:20 MeOH/CH₂Cl₂ eluent), affording phenol 05 (0.339 mg, 85% yield) as a brown oil. ¹H NMR (600 MHz, MeOD) δ 8.18 (s, 1H), 7.73 (d, J=8.9 Hz 1H) 7.72 (s, 1H), 7.52 (d, J=8.9 Hz, 1H), 7.49 (t, J=7.5 Hz, 1H), 7.33 (t, J=7.5 Hz, 1H), 6.51 (d, J=8.1 Hz, 1H), 6.49 (d, J=7.5, 8.1 Hz, 1H), 6.33 (d, J=8.1 Hz, 1H), 4.27 (s, 2H); HRMS (TOF LC/MS) m/z cal'd for (M+H)⁺ [C₁₇H₁₄N₂O₄+H]⁺: 310.31, found 311.1033.

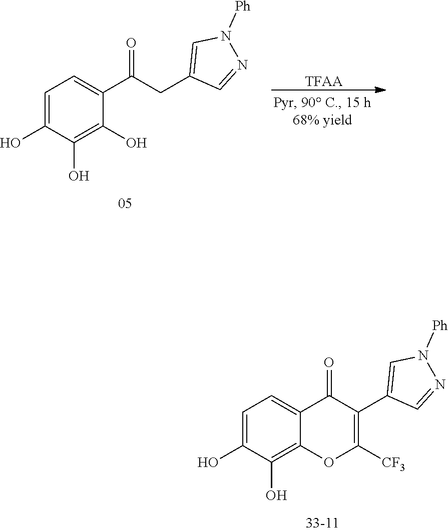 [SMILES: O=C(Cc1cnn(-c2ccccc2)c1)c1ccc(O)c(O)c1O ; "TFAA" ; O=c1c(-c2cnn(-c3ccccc3)c2)c(C(F)(F)F)oc2c(O)c(O)ccc12]

Cyclization Procedure: Compound #33-11. To a solution of phenol 05 (1.140 g, 3.67 mmol) in pyridine (15 mL, 0.06 M) at 23° C. was added TFAA (2.1 mL, 14.98 mmol), and the mixture was stirred for 15 hours at 90° C. Upon consumption of starting material, the solution was diluted with H₂O (30 mL) and then extracted with EtOAc (2×60 mL). The combined organic layers were washed with brine (60 mL) and dried over MgSO₄. After removal of solvent by rotary evaporation, the crude material was purified by flash chromatography (1:20 MeOH/CH₂Cl₂ eluent), affording compound #33-11 (0.972 g, 68% yield) as a colorless solid. ¹H NMR (600 MHz, (CD₃)₂CO) δ 9.41 (s, 1H), 8.89 (s, 1H), 8.52 (s, 1H), 7.94 (d, J=7.5 Hz 2H) 7.81 (s, 1H), 7.62 (d, J=8.7 Hz, 1H), 7.55 (dd, J=7.5, 8.7 Hz, 2H), 7.37 (t, J=7.5 Hz, 1H), 7.14 (d, J=8.7 Hz, 1H); HRMS (TOF LC/MS) m/z cal'd for (M+H)⁺ [C₁₉H₁₁F₃N₂O₄+H]⁺: 388.30, found 389.0907.

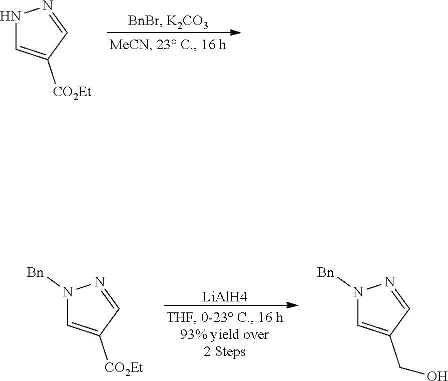

Ethyl 1-benzyl-1H-pyrazole-4-carboxylate: To a solution of the commercial ethyl 1H-pyrazole-4-carboxylate (5.0716 g, 0.0362 mol) in MeCN (34 mL, 0.1 M) was added K₂CO₃ (4.84 g, 0.035 mol) and BnBr (4.00 mL, 0.0382 mml) subsequently at 23° C. under N₂. The reactant solution was stirred for 16 hours and diluted with H₂O (100 mL). The solution was extracted with EtOAc (2×150 mL). The combined organic layers were washed with brine (100 mL) and dried over MgSO₄. The solvent was removed by rotary evaporation to afford ethyl 1-benzyl-1H-pyrazole-4-carboxylate (Kalla et al., “Novel 1,3-Disubstituted 8-(1-benzyl-1H-pyrazol-4-yl) Xanthines: High Affinity and Selective A2B Adenosine Receptor Antagonists,” J. Med. Chem. 49(12):3682-3692 (2006), which is hereby incorporated by reference in its entirety) as pale yellow oil. The crude was directly used to the next step. ¹H NMR (600 MHz, CDCl₃) δ 7.96 (s, 1H), 7.87 (s, 1H), 7.41-7.35 (m, 3H), 7.27 (d, J=6.9 Hz, 2H), 5.33 (s, 2H), 4.30 (q, J=7.1 Hz, 2H), 1.35 (t, J=7.1 Hz, 3H).

(1-Benzyl-1H-pyrazol-4-yl) methanol: To a solution of crude benzyl ester in THF (60 mL, 0.6 M) was added LiAlH₄ (1.34 g, 0.0353 mol) portion wise at 0° C. The reaction mixture was stirred at 23° C. for 16 h. The solution was diluted with EtOAc (150 mL) at 0° C. and H₂O was added drop-wise until gas evolution was stopped. The mixture was stirred for 1 hour and the white precipitate was filtered through MgSO₄ with EtOAc (150 mL). The solvent was removed by rotary evaporation to afford the desired product (5.97 g, 93% over 2 steps) as pale yellow oil. ¹H NMR (600 MHz, CDCl₃) δ 7.57 (s, 1H), 7.41 (s, 1H), 7.37-7.33 (m, 3H), 7.25 (d, J=6.9 Hz, 2H), 5.31 (s, 2H), 4.60 (s, 2H); HRMS (TOF LC/MS (APCI)) m/z cal'd for (M+H)⁺ [C₁₁H₁₂N₂O+H]⁺: 188.23, found 189.4144.

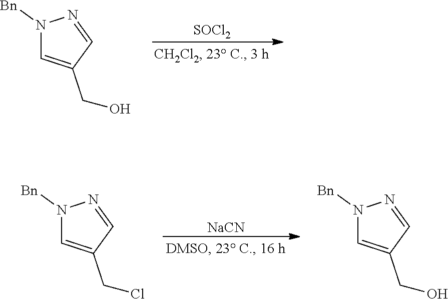 [SMILES: OCc1cnn(Cc2ccccc2)c1 ; "SOCl2" ; ClCc1cnn(Cc2ccccc2)c1 ; "NaCN" ; OCc1cnn(Cc2ccccc2)c1]

1-benzyl-4-(chloromethyl)-1H-pyrazole: To a solution of (1-benzyl-1H-pyrazol-4-yl) methanol (4.04 g, 0.0215 mol) in CH₂Cl₂ (60 mL, 0.35 M) was added thionyl chloride (5.0 mL, 0.0672 mol, 4.3 M) at 23° C. under N₂. The reactant mixture was stirred for 3 hours. The solvent was removed by rotary evaporation and the residue was filtered through a pad of SiO₂ with EtOAc (150 mL). The solvent was removed by rotary evaporation to afford as yellow oil. The crude was directly used to the next step. ¹H NMR (600 MHz, CDCl₃) δ 7.89 (s, 1H), 7.54 (s, 1H), 7.48-7.42 (m, 3H), 7.28 (m, 2H), 5.68 (s, 2H), 4.51 (s, 2H).

2-(1-Benzyl-1H-pyrazol-4-yl) acetonitrile: To a solution of crude chloride in DMSO (50 mL, 0.4 M) was added NaCN (4.24 g, 0.0866 mmol) at 23° C. After the mixture was stirred for 16 hours, it was diluted with H₂O (150 mL) and extracted with EtOAc (2×150 mL). The combined organic layers were washed with brine (100 mL) and dried over MgSO₄. The solvent was removed by rotary evaporation to afford 2-(1-benzyl-1H-pyrazol-4-yl) acetonitrile as a brown oil. The crude was directly used to the next step. ¹H NMR (600 MHz, CDCl₃) δ 7.51 (s, 1H), 7.40 (s, 1H), 7.38-7.33 (m, 3H), 7.25 (d, J=6.7 Hz, 2H), 5.30 (s, 2H), 4.36 (s, 2H); HRMS (TOF LC/MS (APCI)) m/z cal'd for (M+H)⁺ [C₁₂H₁₁N₃+H]⁺: 197.24, found 198.7659.

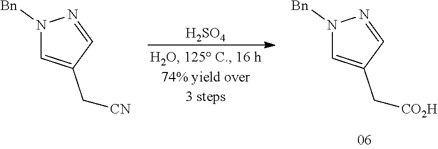 [SMILES: N#CCc1cnn(Cc2ccccc2)c1 ; "H2SO4" ; O=C(O)Cc1cnn(Cc2ccccc2)c1]

2-(1-Benzyl-1H-pyrazol-4-yl) acetic acid (06): To a solution of pyrazole acetonitrile in H₂O (72 mL, 0.3 M) was added sulfuric acid (72 mL, 0.3 M) at 23° C. The reactant mixture was stirred at 125° C. for 16 hours. The reactant solution was slowly added into saturated NaOH (100 mL) at 0° C. The solution was acidified to pH 5. The mixture was extracted with EtOAC (2×150 ml). The combined organic layers were washed with brine (100 mL) and dried over MgSO₄. The solvent was removed by rotary evaporation, and the residue was recrystallized with (˜10:1 Hexane/EtOAC eluent) to afford 2-(1-benzyl-1H-pyrazol-4-yl) acetic acid (06) (3.44 g, 74% yield over 3 steps) as a colorless solid. NMR (600 MHz, CDCl₃) δ 7.67 (s, 1H), 7.44 (s, 1H), 7.40-7.32 (m, 3H), 7.32 (d, J=7.2 Hz, 2H), 5.43 (s, 2H), 3.57 (s, 2H); HRMS (TOF LC/MS (APCI)) m/z cal'd for (M+H)⁺ [C₁₂H₁₂N₂O₂+H]⁺: 216.24, found 217.7009.

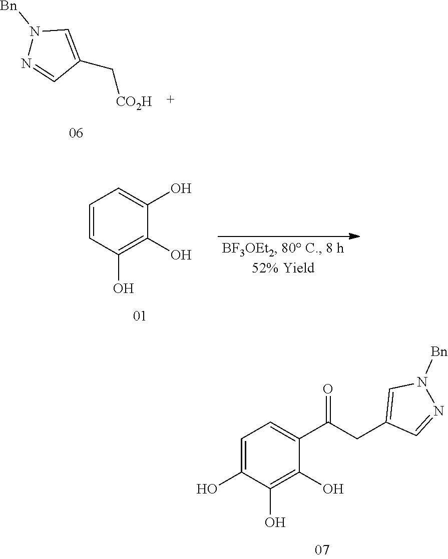 [SMILES: O=C(O)Cc1cnn(Cc2ccccc2)c1 ; Oc1cccc(O)c1O ; O=C(Cc1cnn(Cc2ccccc2)c1)c1ccc(O)c(O)c1O]

Friedel Crafts Procedure (Basha et al., “A Mild and Efficient Protocol to Synthesize Chromones, Isoflavones, and Homoisoflavones Using the Complex 2,4,6-trichloro-1,3,5-triazine/dimethylformamide,” Can. J. Chem. 91:763-768 (2013), which is hereby incorporated by reference in its entirety): 2-(1-benzyl-1H-pyrazol-4-yl)-1-(2,3,4-trihydroxyphenyl) ethan-1-one (07): To a solution of pyrogallol 01 (0.646 g, 5.13 mmol) in BF₃OEt₂ (5.5 mL, 1.0 M) at 23° C. was added carboxylic acid 06 (1.090 g, 5.04 mmol), and the mixture was stirred for 16 hours at 80° C. Upon consumption of starting material, the reaction mixture was poured into sat. NaOAc (30 mL). The solution was extracted with EtOAc (2×100 mL). The combined organic layers were washed with brine (50 mL) and dried over MgSO₄. After removal of solvent by rotary evaporation, the crude material was purified by flash chromatography (1:1 hexanes/EtOAc eluent), affording 2-(1-benzyl-1H-pyrazol-4-yl)-1-(2,3,4-trihydroxyphenyl) ethan-1-one 07 (0.883 mg, 52% yield) as a brown solid. ¹H NMR (600 MHz, CD₃OD) δ 7.64 (s, 1H), 7.47 (d, J=3.0 Hz, 1H), 7.45 (s, 1H), 7.35-7.29 (m, 3H), 7.21 (d, J=7.0 Hz, 2H), 6.45 (d, J=8.9 Hz, 1H), 5.32 (s, 2H), 4.16 (s, 2H); HRMS (TOF LC/MS (APCI)) m/z cal'd for (M+H)⁺ [C₁₈H₁₆N₂O₃+H]⁺: 324.34, found 325.1408.

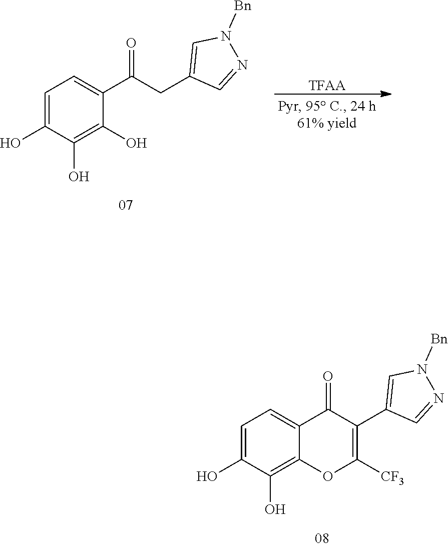 [SMILES: O=C(Cc1cnn(Cc2ccccc2)c1)c1ccc(O)c(O)c1O ; "TFAA" ; O=c1c(-c2cnn(Cc3ccccc3)c2)c(C(F)(F)F)oc2c(O)c(O)ccc12]

Cyclization Procedure (Otsalyuk et al., “Synthetic Analogs of Xanthocercin,” Chem. Nat. Compd. 34:284-288 (1998), which is hereby incorporated by reference in its entirety): 3-(1-Benzyl-1H-pyrazol-4-yl)-7,8-dihydroxy-2-(trifluoromethyl)-4H-chromen-4-one (08): To a solution of phenol 07 (0.296 g, 0.913 mmol) in pyridine (9.0 mL) at 23° C. was added TFAA (0.50 mL, 3.57 mmol), and the mixture was stirred for 19 hours at 95° C. Upon consumption of starting material, the solution was diluted with H₂O (40 mL) and then extracted with EtOAc (2×100 mL). The combined organic layers were washed with brine (30 mL) and dried over MgSO₄. After removal of solvent by rotary evaporation, the crude material was purified by flash chromatography (1:1 hexanes/EtOAc eluent), affording chromenone 08 (0.2256 mg, 61% yield) as a yellow solid. ¹H NMR (600 MHz, (CD3)₂CO) δ 7.94 (s, 1H), 7.58 (d, J=2.0 Hz, 1H), 7.57 (s, 1H), 7.38 (m, 3H), 7.33 (d, J=2.0 Hz, 2H), 7.0 (d, J=8.7 Hz, 1H), 5.46 (s, 2H); HRMS (TOF LC/MS (APCI)) m/z cal'd for (M+H)⁺ [C₂₀H₁₃F₃N₂O₄+H]⁺: 402.33, found 403.1706.

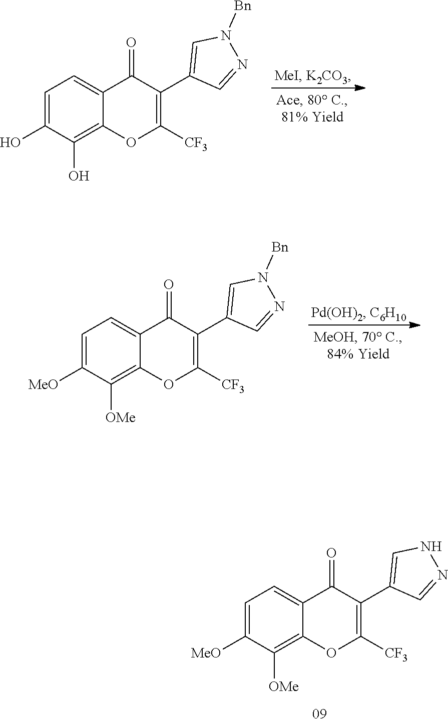

3-(1-Benzyl-1H-pyrazol-4-yl)-7,8-dimethoxy-2-(trifluoromethyl)-4H-chromen-4-one: To a solution of phenol 08 (0.70 g, 1.74 mmol) in anhydrous acetone (9.0 mL, 0.2 M) at 23° C. was added K₂CO₃ (0.597 g. 4.31 mmol) and Mel (3.00 mL, 48.61 mmol) subsequently, and the mixture was stirred for 20 hours at 60° C. Upon consumption of starting material, the solution was diluted with H₂O (50 mL) and then extracted with EtOAc (2×100 mL). The combined organic layers were washed with brine (50 mL) and dried over MgSO₄. After removal of solvent by rotary evaporation, the residue was recrystallized with (˜10:1 Hexane/EtOAC eluent) to afford 3-(1-benzyl-1H-pyrazol-4-yl)-7,8-dimethoxy-2-(trifluoromethyl)-4H-chromen-4-one (0.601 g, 81% yield) as a colorless solid. ¹H NMR (600 MHz, (CD₃)₂CO) δ 7.96 (s, 1H), 7.88 (d, J=9.0 Hz, 1H), 7.59 (s, 1H), 7.40-7.32 (m, 6H), 5.47 (s, 2H), 4.06 (s, 3H), 3.99 (s, 3H); HRMS (TOF LC/MS (APCI)) m/z cal'd for (M+H)⁺ [C₂₂H₁₇F₃N₂O₄+H]⁺: 430.08, found 431.1198.

7,8-Dimethoxy-3-(1H-pyrazol-4-yl)-2-(trifluoromethyl)-4H-chromen-4-one (09): To a solution of 3-(1-benzyl-1H-pyrazol-4-yl)-7,8-dimethoxy-2-(trifluoromethyl)-4H-chromen-4-one (0.301 g, 0.699 mmol) in anhydrous EtOH (7.0 mL, 0.1 M) at 23° C. was added Pd(OH)₂ (0.316 g, 100% w/w) and cyclohexene (7.0 mL, 0.1 M), and the mixture was stirred for 72 hours at 85° C. Upon consumption of starting material, the reaction mixture was filtered through a SiO₂ plug, washing with EtOAc (35 mL). After removal of solvent by rotary evaporation, the residue was recrystallized with (˜10:1 Hexane/EtOAC eluent) to afford pyrazole 09 (0.200 g, 84% yield) as a colorless solid. ¹H NMR (600 MHz, (CD3)₂CO) δ 7.89 (d, J=88 Hz, 2H), 7.36 (d, J=8.8 Hz, 2H), 4.07 (s, 3H), 4.00 (s, 3H); HRMS (TOF LC/MS (APCI)) m/z cal'd for (M+H)+[C₁₅H₁₁F₃N₂O₄+H]⁺: 340.21, found 341.6822.

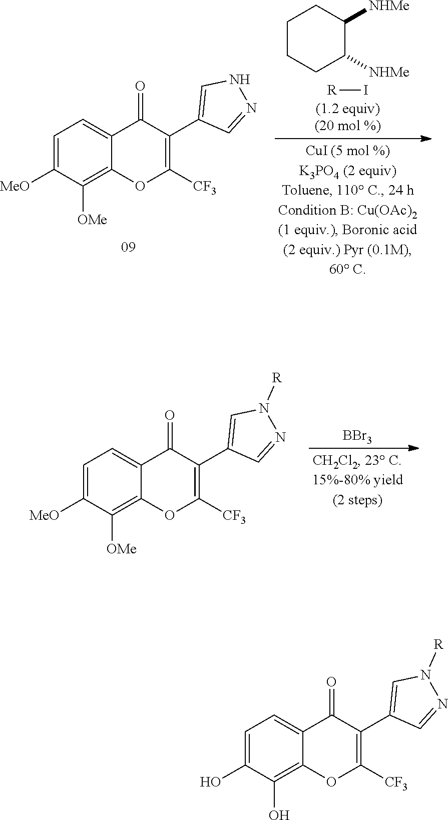

General copper catalyzed cross coupling procedure A (Antilla et al., “Copper-Diamine Catalyzed N-Arylation of Pyrroles, Pyrazoles, Indazoles, Imidazoles, and Triazoles,” J. Org. Chem. 69:5578-5587 (2004), which is hereby incorporated by reference in its entirety): To a mixture of 09 (1 equiv), dimethyl amine (20 mol %), CuI (5 mol %), and K₂CO₃ (2 equiv) in toluene (0.1 M) was added aryl iodide (1.2 equiv). The reaction mixture was stirred at 110° C. and monitored by TLC. Upon completion, the reaction mixture was filtered through a SiO₂ plug, washing with EtOAc (˜4× reaction volume). The solvent was then removed by rotary evaporation, and the resulting residue was used for the next step.

General copper catalyzed cross coupling procedure B (Mukherjee & Sarkar, “Pyrazole-tethered Arylphosphine Ligands for Suzuki Reactions of Aryl Chlorides: How Important is Chelation?” Tet. Lett. 45(52):9525-9528 (2004), which is hereby incorporated by reference in its entirety): To a mixture of 09 (1 equiv.), boronic acid (2 equiv.) in pyridine (0.1 M) was added Cu(OAc) (1.0 equiv.). The reaction mixture was stirred at 60° C. and monitored by TLC. Upon completion, the reaction mixture was filtered through a SiO₂ plug, washing with EtOAc (˜4× reaction volume). The solvent was then removed by rotary evaporation, and the resulting residue was used for the next step.

General demethylation procedure: To the chromenone residue in CH₂Cl₂ (0.1 M) was added BBr₃ (2 equiv) at 0° C. The reaction was slowly warm to 23° C. and stirred for 5 hours. The reaction mixture was stirred at 23° C. and monitored by TLC. Upon completion, the reaction mixture was quenched with MeOH (˜2× reaction volume) and stirred for additional 1 h. The solvent was then removed by rotary evaporation, and the resulting residue was purified by flash chromatography on SiO₂.

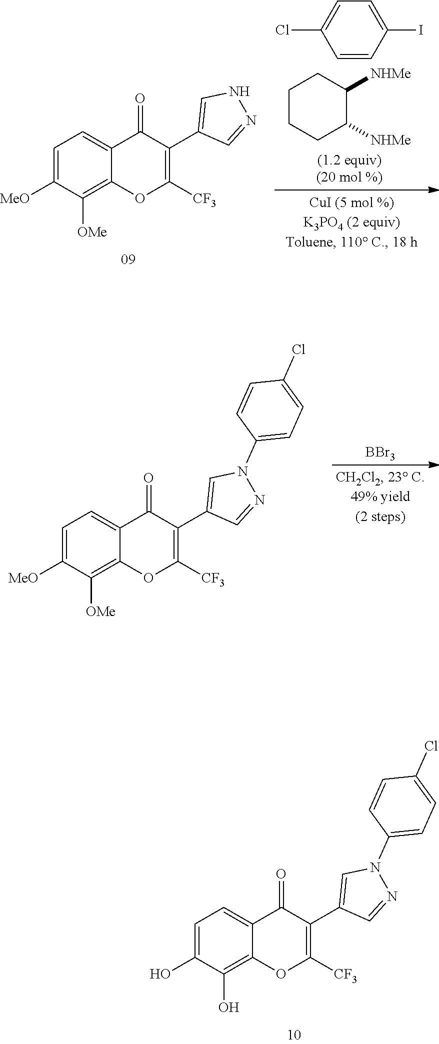

3-(1-(4-chlorophenyl)-1H-pyrazol-4-yl)-7,8-dimethoxy-2-(trifluoromethyl)-4H-chromen-4-one: Following general copper catalyzed cross coupling procedure A: To a mixture of 09 (14.3 mg, 0.0420 mmol), methyl diamine (6.90 mg, 0.0489 mmol), CuI (2.00 mg, 0.0105 mmol), and K₃PO₄ (24.70 mg, 0.116 mmol) in toluene (0.5 mL, 0.1 M) was added aryl iodide (22.5 mg, 0.0943 mmol). The reaction mixture was stirred at 110° C. for 18 hours. Upon completion, the reaction mixture was filtered through a SiO₂ plug, washing with EtOAc (2 mL). The solvent was then removed by rotary evaporation, and the resulting residue was used for the next step.

3-(1-(4-chlorophenyl)-1H-pyrazol-4-yl)-7,8-dihydroxy-2-(trifluoromethyl)-4H-chromen-4-one; KH-4-43 (10): Following general demethylation procedure: To the solution of the resulting residue in CH₂Cl₂ (0.5 mL, 0.1 M) was added BBr₃ (0.10 mL, 0.100 mmol) at 0° C. The reaction was slowly warm to 23° C. and stirred for 3 hours. Upon completion, the reaction mixture was quenched with MeOH (1.0 mL) and stirred for additional 1 hour. The solvent was removed by rotary evaporation, and the resulting residue was purified by flash chromatography (1:1 hexanes/EtOAc eluent) to afford analog 10 (8.7 mg, 49% yield over 2 steps) as a colorless solid. ¹H NMR (600 MHz, CD₃Cl) δ 8.23 (s, 1H), 7.84 (s, 1H), 7.80 (d, J=8.8 Hz, 1H), 7.72 (d, J=8.7 Hz, 2H), 7.48 (d, J=8.7 Hz, 2H), 7.13 (d, J=8.8 Hz, 1H); HRMS (TOF LC/MS (APCI)) m/z cal'd for (M+H)⁺ [C₁₉H₁₀ClF₃N₂O₄+H]⁺: 422.74, found 423.0340.

Large scale synthesis of KH-4-43 (1.1 gram) was carried out at BioDuro using the above procedure with minor modifications. The chemical and biological properties of the large scale preparation were determined to be identical to the small scale materials.

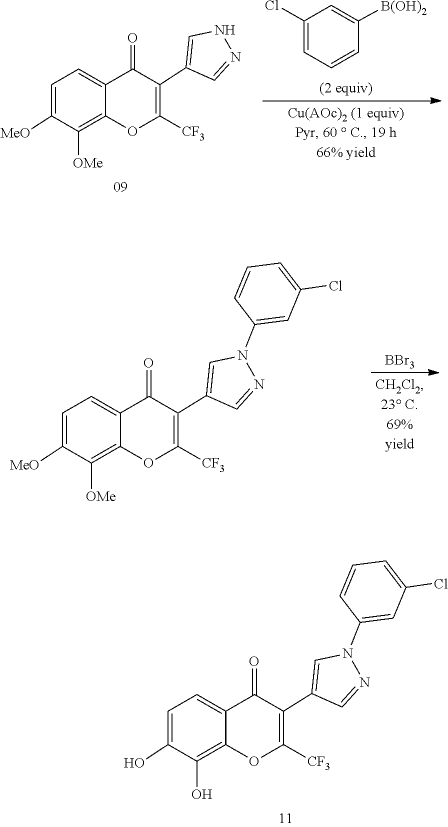 [SMILES: COc1ccc2c(=O)c(-c3cn[nH]c3)c(C(F)(F)F)oc2c1OC ; OB(O)c1cccc(Cl)c1 ; COc1ccc2c(=O)c(-c3cnn(-c4cccc(Cl)c4)c3)c(C(F)(F)F)oc2c1OC ; "BBr3" ; O=c1c(-c2cnn(-c3cccc(Cl)c3)c2)c(C(F)(F)F)oc2c(O)c(O)ccc12]

KH-3-151, Analog 11: Following general copper catalyzed cross coupling procedure B: To a mixture of 09 (15.5 mg, 0.0441 mmol), boronic acid (14.7 mg, 0.105 mmol) in pyridine (0.4 mL, 0.1 M) was added Cu(OAc)₂ (8.6 mg, 0.0473 mmol). The reaction mixture was stirred at 60° C. for 19 hours. Upon completion, the reaction mixture was filtered through a SiO₂ plug, washing with EtOAc (1.6 mL). The solvent was then removed by rotary evaporation, and the resulting residue was purified by flash chromatography (4:1 hexanes/EtOAc eluent) to afford dimethoxyl chromenone (13.0 mg, 66% yield) as a colorless solid. ¹H NMR (600 MHz, CD₃Cl) δ 8.19 (s, 1H), 8.01 (d, J=9.0 Hz, 1H), 7.85 (s, 1H), 7.76-7.70 (m, 2H), 7.20 (t, J=8.7 Hz, 1H), 7.14 (d, J=9.0 Hz, 1H), 7.11 (s, 1H), 4.06 (s, 3H), 4.05 (s, 3H); HRMS (TOF LC/MS (APCI)) m/z cal'd for (M+H)⁺ [C₂₁H₁₄ClF₃N₂O₄+H]⁺: 434.34, found 435.3637.

Following general demethylation procedure: To the solution of the dimethoxyl chromenone (14.5 mg, 0.0322 mol) in CH₂Cl₂ (0.5 mL, 0.1 M) was added BBr₃ (0.13 mL, 0.129 mmol) at 0° C. The reaction was slowly warm to 23° C. and stirred for 3 hours. Upon completion, the reaction mixture was quenched with MeOH (1.0 mL) and stirred for additional 1 hour. The solvent was removed by rotary evaporation, and the resulting residue was purified by flash chromatography (1:1 hexanes/EtOAc eluent) to afford analog 11 (9.4 mg, 69% yield over 2 steps) as a colorless solid. ¹H NMR (600 MHz, (CD₃)₂CO) δ 8.59 (s, 1H), 8.59 (s, 1H), 8.01 (s, 1H), 7.91 (d, J=8.2 Hz, 1H), 7.84 (s, 1H), 7.62-7.53 (m, 2H), 7.39 (d, J=8.2 Hz, 1H), 7.14 (d, J=7.9 Hz, 1H); HRMS (TOF LC/MS (APCI)) m/z cal'd for (M+H)⁺ [C₁₉H₁₀ClF₃N₂O₄+H]⁺: 422.74, found 423.0446.

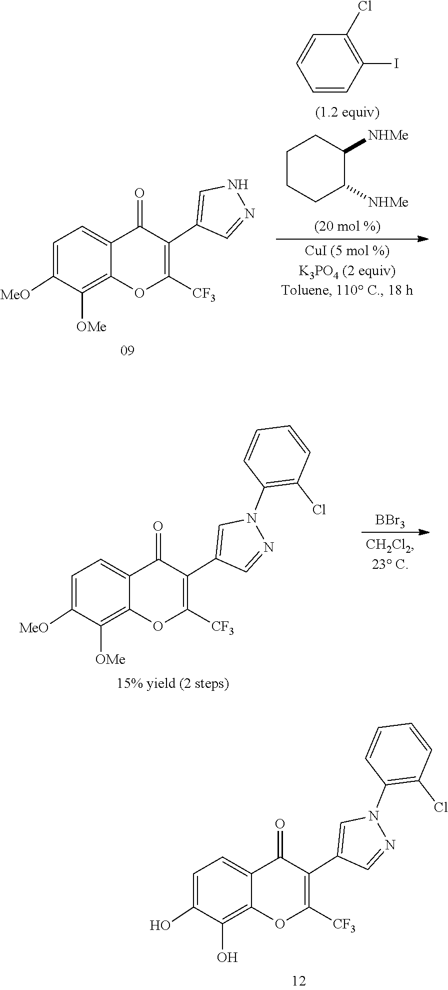

KH-4-88, Analog 12. Following general copper catalyzed cross coupling procedure A: To a mixture of 09 (14.4 mg, 0.0423 mmol), methyl diamine (4.6 mg, 0.0325 mmol), CuI (2.00 mg, 0.0105 mmol), and K₃PO₄ (23.2 mg, 0.116 mmol) in toluene (0.5 mL, 0.1 M) was added aryl iodide (11 μL, 0.0907 mmol). The reaction mixture was stirred at 110° C. for 18 hours. Upon completion, the reaction mixture was filtered through a SiO₂ plug, washing with EtOAc (2 mL). The solvent was then removed by rotary evaporation, and the resulting residue was used for the next step.

Following general demethylation procedure: To the solution of the resulting residue in CH₂Cl₂ (0.5 mL, 0.1 M) was added BBr₃ (0.10 mL, 0.100 mmol M) at 0° C. The reaction was slowly warm to 23° C. and stirred for 3 hours. Upon completion, the reaction mixture was quenched with MeOH (1.0 mL) and stirred for additional 1 hour. The solvent was removed by rotary evaporation, and the resulting residue was purified by flash chromatography (1:1 hexanes/EtOAc eluent) to afford analog 12 (2.7 mg, 15% yield over 2 steps) as a colorless solid. ¹H NMR (600 MHz, (CD₃)₂CO) δ 8.28 (s, 1H), 7.87 (s, 1H), 7.74 (dd, J=1.3, 7.8 Hz, 1H), 7.69 (dd, J=1.3, 7.8 Hz, 2H), 7.61 (d, J=8.7 Hz, 2H), 7.59-7.51 (m, 2H), 7.15 (d, J=8.7 Hz, 1H); HRMS (TOF LC/MS (APCI)) m/z cal'd for (M+H)⁺ [C₁₉H₁₀ClF₃N₂O₄+H]⁺: 422.74, found 423.0325.

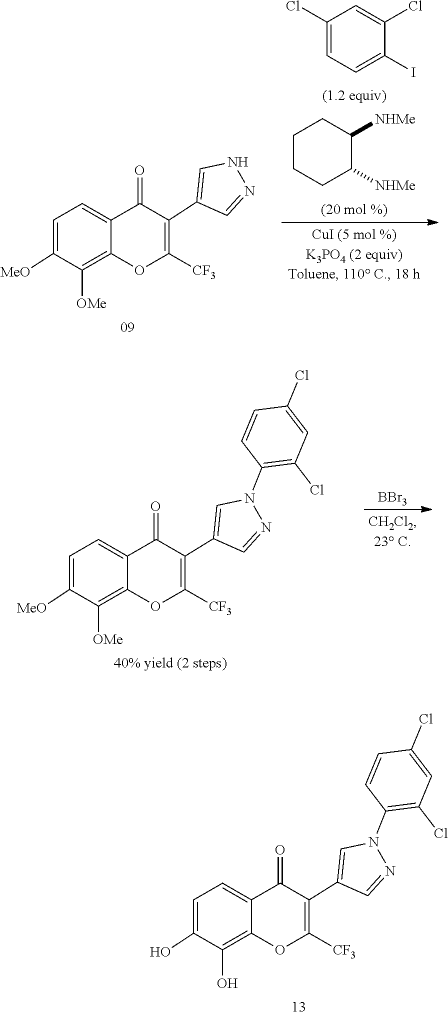

KH-4-98, Analog 13. Following general copper catalyzed cross coupling procedure A: To a mixture of 09 (14.5 mg, 0.0426 mmol), methyl diamine (5.5 mg, 0.0389 mmol), CuI (1.6 mg, 0.0105 mmol), and K₃PO₄ (21.0 mg, 0.116 mmol) in toluene (0.5 mL, 0.1 M) was added aryl iodide (12 μL, 0.0893 mmol). The reaction mixture was stirred at 110° C. for 18 hours. Upon completion, the reaction mixture was filtered through a SiO₂ plug, washing with EtOAc (2 mL). The solvent was then removed by rotary evaporation, and the resulting residue was used for the next step.

Following general demethylation procedure: To the solution of the resulting residue in CH₂Cl₂ (0.5 mL, 0.1 M) was added BBr₃ (0.10 mL, 0.100 mmol) at 0° C. The reaction was slowly warm to 23° C. and stirred for 3 hours. Upon completion, the reaction mixture was quenched with MeOH (1.0 mL) and stirred for additional 1 hour. The solvent was removed by rotary evaporation, and the resulting residue was purified by flash chromatography (1:1 hexanes/EtOAc eluent) to afford analog 13 (7.8 mg, 40% yield over 2 steps) as a colorless solid. ¹H NMR (600 MHz, (CD₃)₂CO) δ 8.30 (s, 1H), 7.86 (s, 1H), 7.77 (d, J=2.3 Hz, 1H), 7.76 (d, J=8.6 Hz, 1H), 7.64-7.59 (m, 2H), 7.13 (d, J=8.7 Hz, 1H); HRMS (TOF LC/MS (APCI)) m z cal'd for (M)⁺ [C₁₉H₉Cl₂F₃N₂O₄]⁺: 457.19, found 456.9931.

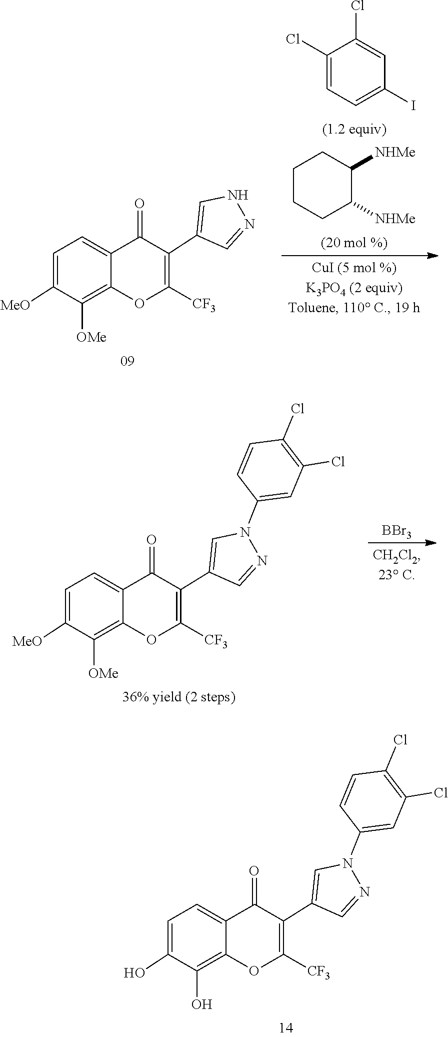 [SMILES: Clc1ccc(I)cc1Cl ; COc1ccc2c(=O)c(-c3cn[nH]c3)c(C(F)(F)F)oc2c1OC ; CN[C@@H]1CCCC[C@H]1NC ; COc1ccc2c(=O)c(-c3cnn(-c4ccc(Cl)c(Cl)c4)c3)c(C(F)(F)F)oc2c1OC ; "BBr3" ; O=c1c(-c2cnn(-c3ccc(Cl)c(Cl)c3)c2)c(C(F)(F)F)oc2c(O)c(O)ccc12]

KH-4-99, Analog 14. Following general copper catalyzed cross coupling procedure A: To a mixture of 09 (13.9 mg, 0.0409 mmol), methyl diamine (6.5 mg, 0.0460 mmol), CuI (1.8 mg, 0.0 mmol), and K₃PO₄ (24.70 mg, 0.116 mmol) in toluene (0.5 mL, 0.1 M) was added aryl iodide (22.5 mg, 0.0945 mmol). The reaction mixture was stirred at 110° C. for 19 hours. Upon completion, the reaction mixture was filtered through a SiO₂ plug, washing with EtOAc (2 mL). The solvent was then removed by rotary evaporation, and the resulting residue was used for the next step.

Following general demethylation procedure: To the solution of the resulting residue in CH₂Cl₂ (0.5 mL, 0.1 M) was added BBr₃ (0.10 mL, 0.100 mmol) at 0° C. The reaction was slowly warm to 23° C. and stirred for 3 hours. Upon completion, the reaction mixture was quenched with MeOH (1.0 mL) and stirred for additional 1 hour. The solvent was removed by rotary evaporation, and the resulting residue was purified by flash chromatography (1:1 hexanes/EtOAc eluent) to afford analog 14 (6.8 mg, 36% yield over 2 steps) as a colorless solid. ¹H NMR (600 MHz, (CD₃)₂CO) δ 8.63 (s, 1H), 8.9 (d, J=2.6 Hz, 1H), 7.96 (dd, J=2.6, 8.8 Hz, 1H), 7.85 (s, 1H), 7.75 (d, J=8.8 Hz, 1H), 7.60 (d, J=7.8 Hz, 1H), 7.13 (d, J=7.8 Hz, 1H); HRMS (TOF LC/MS (APCI)) m/z cal'd for (M+H)⁺ [C₁₉H₉Cl₂F₃N₂O₄+H]⁺: 457.19, found 456.9963.

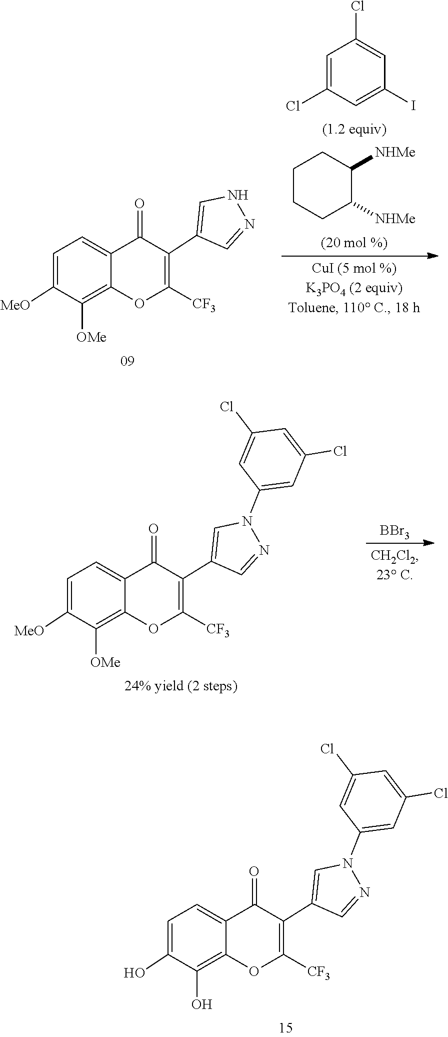 [SMILES: Clc1cc(Cl)cc(I)c1 ; COc1ccc2c(=O)c(-c3cn[nH]c3)c(C(F)(F)F)oc2c1OC ; CN[C@@H]1CCCC[C@H]1NC ; COc1ccc2c(=O)c(-c3cnn(-c4cc(Cl)cc(Cl)c4)c3)c(C(F)(F)F)oc2c1OC ; "BBr3" ; O=c1c(-c2cnn(-c3cc(Cl)cc(Cl)c3)c2)c(C(F)(F)F)oc2c(O)c(O)ccc12]

KH-4-116, Analog 15. Following general copper catalyzed cross coupling procedure A: To a mixture of 09 (13.6 mg, 0.0399 mmol), methyl diamine (6.2 mg, 0.0415 mmol), CuI (1.2 mg, 0.0063 mmol), and K₃PO₄ (22.6 mg, 0.106 mmol) in toluene (0.4 mL, 0.1 M) was added aryl iodide (24.7 mg, 0.0905 mmol). The reaction mixture was stirred at 110° C. for 18 hours. Upon completion, the reaction mixture was filtered through a SiO₂ plug, washing with EtOAc (2 mL). The solvent was then removed by rotary evaporation, and the resulting residue was used for the next step.

Following general demethylation procedure: To the solution of the resulting residue in CH₂Cl₂ (0.5 mL, 0.1 M) was added BBr₃ (0.10 mL, 0.100 mmol M) at 0° C. The reaction was slowly warm to 23° C. and stirred for 3 hours. Upon completion, the reaction mixture was quenched with MeOH (1.0 mL) and stirred for additional 1 hour. The solvent was removed by rotary evaporation, and the resulting residue was purified by flash chromatography (1:1 hexanes/EtOAc eluent) to afford analog 15 (4.6 mg, 24% yield over 2 steps) as a colorless solid. ¹H NMR (600 MHz, CDCl₃) δ 8.24 (s, 1H), 7.84 (s, 1H), 7.79 (d, J=8.8 Hz, 1H), 7.71 (d, J=1.7 Hz, 2H), 7.33 (m, 2H), 7.13 (d, J=8.8 Hz, 1H); HRMS (TOF LC/MS (APCI)) m/z cal'd for (M+H)⁺ [C₁₉H₉Cl₂F₃N₂O₄+H]⁺: 457.19, found 458.9914.

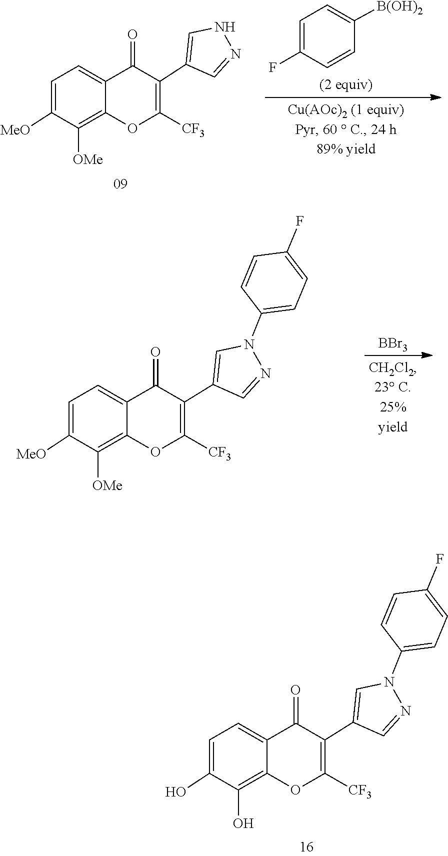

KH-3-174, Analog 16. Following general copper catalyzed cross coupling procedure B: To a mixture of 09 (15.1 mg, 0.0444 mmol), boronic acid (15.2 mg, 0.109 mmol) in pyridine (0.4 mL, 0.1 M) was added Cu(OAc)₂ (9.9 mg, 0.0545 mmol). The reaction mixture was stirred at 60° C. for 24 hours. Upon completion, the reaction mixture was filtered through a SiO₂ plug, washing with EtOAc (1.6 mL). The solvent was then removed by rotary evaporation, and the resulting residue was purified by flash chromatography (4:1 hexanes/EtOAc eluent) to afford dimethoxyl (17.1 mg, 89% yield) as a colorless solid. ¹H NMR (600 MHz, (CD₃Cl) δ 8.19 (s, 1H), 8.00 (d, J=9.0 Hz, 1H), 7.85 (s, 1H), 7.77-7.70 (m, 2H), 7.20 (m, 2H), 7.15 (d, J=9.0 Hz, 1H), 4.06 (s, 3H), 4.05 (s, 3H); HRMS (TOF LC/MS (APCI)) m/z cal'd for (M+H)⁺ [C₂₁H₁₄F₄N₂O₄+H]⁺: 434.34, found 435.2325.

Following general demethylation procedure: To the solution of the dimethoxyl chromenone (17.1 mg, 0.0394 mol) in CH₂Cl₂ (0.4 mL, 0.1 M) was added BBr₃ (0.10 mL, 0.100 mmol) at 0° C. The reaction was slowly warm to 23° C. and stirred for 3 hours. Upon completion, the reaction mixture was quenched with MeOH (1.0 mL) and stirred for additional 1 hour. The solvent was removed by rotary evaporation, and the resulting residue was purified by flash chromatography (1:1 hexanes/EtOAc eluent) to afford analog 16 (4.0 mg, 25% yield) as a colorless solid. ¹H NMR (600 MHz, (CD₃)₂CO) δ 8.66 (s, 1H), 8.12 (s, 1H), 8.07-8.00 (m, 2H), 7.59 (d, J=8.7 Hz, 1H), 7.42-7.35 (m, 2H), 7.19 (d, J=8.7 Hz, 1H); HRMS (TOF LC/MS (APCI)) m/z cal'd for (M+H)⁺ [C₁₉H₁₀F₄N₂O₄+H]⁺: 406.29, found 407.1000.

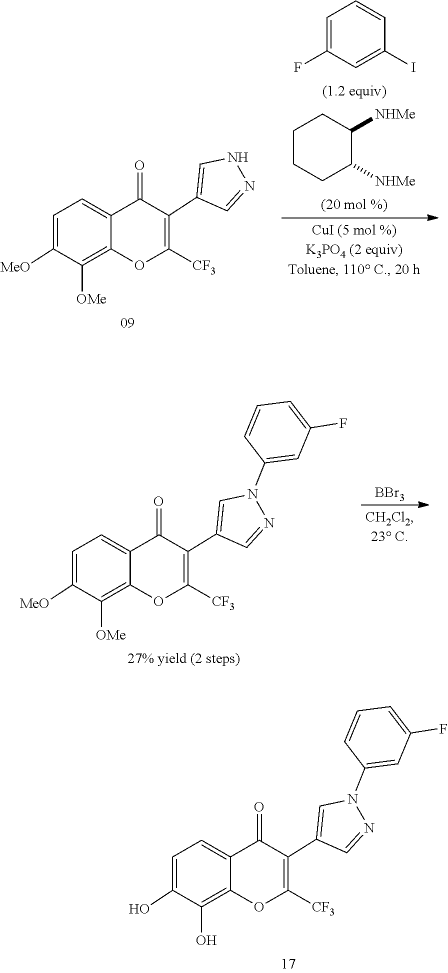

KH-4-41, Analog 17. Following general copper catalyzed cross coupling procedure: To a mixture of 09 (14.7 mg, 0.0432 mmol), methyl diamine (4.8 mg, 0.0337 mmol), CuI (1.9 mg, 0.0998 mmol), and K₃PO₄ (24.9 mg, 0.117 mmol) in toluene (0.45 mL, 0.1 M) was added aryl iodide (11.0 μL, 0.0941 mmol). The reaction mixture was stirred at 110° C. for 20 hours. Upon completion, the reaction mixture was filtered through a SiO₂ plug, washing with EtOAc (2 mL). The solvent was then removed by rotary evaporation, and the resulting residue was used for the next step.

Following general demethylation procedure A: To the solution of the resulting residue in CH₂Cl₂ (0.45 mL, 0.1 M) was added BBr₃ (0.10 mL, 0.100 mmol) at 0° C. The reaction was slowly warm to 23° C. and stirred for 3 hours. Upon completion, the reaction mixture was quenched with MeOH (1.0 mL) and stirred for additional 1 hour. The solvent was removed by rotary evaporation, and the resulting residue was purified by flash chromatography (1:1 hexanes/EtOAc eluent) to afford analog 17 (4.7 mg, 27% yield over 2 steps) as a colorless solid. ¹H NMR (600 MHz, (CD₃)₂CO) δ 8.58 (s, 1H), 7.83 (s, 1H), 7.81-7.75 (m, 2H), 7.62-7.55 (m, 2H), 7.13 (d, J=8.6 Hz, 2H); HRMS (TOF LC/MS (APCI)) m/z cal'd for (M+H)⁺ [C₁₉H₁₀F₄N₂O₄+H]⁺: 406.29, found 407.0868.

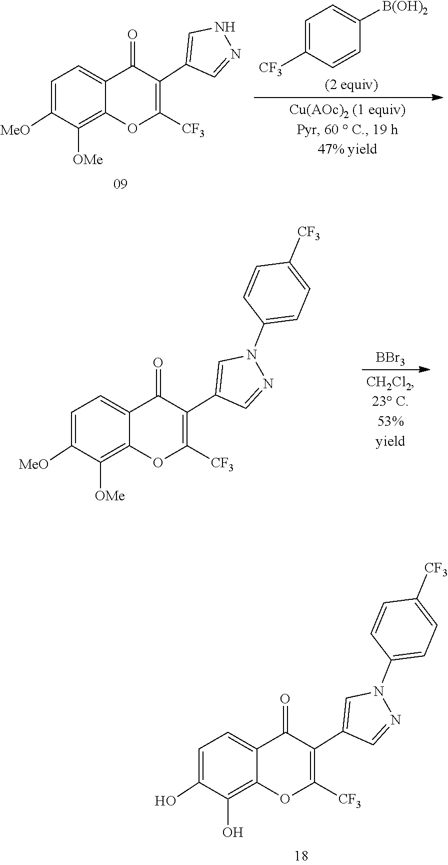

KH-3-150, Analog 18. Following general copper catalyzed cross coupling procedure B: To a mixture of 06 (15.3 mg, 0.0497 mmol), boronic acid (17.4 mg, 0.0916 mmol) in pyridine (0.5 mL, 0.1 M) was added Cu(OAc)₂ (8.1 mg, 0.0446 mmol). The reaction mixture was stirred at 60° C. for 19 hours. Upon completion, the reaction mixture was filtered through a SiO₂ plug, washing with EtOAc (2.0 mL). The solvent was then removed by rotary evaporation, and the resulting residue was purified by flash chromatography (4:1 hexanes/EtOAc eluent) to afford dimethoxyl chromenone (17.1 mg, 47% yield) as a colorless solid. ¹H NMR (600 MHz, CD₃Cl) δ 8.32 (s, 1H), 8.01 (d, J=9.0 Hz, 1H), 7.94-7.87 (m, 2H), 7.78 (d, J=7.6 Hz, 2H), 7.15 (d, J=9.0 Hz, 1H), 7.10 (s, 1H), 4.06 (s, 3H), 4.05 (s, 3H); HRMS (TOF LC/MS (APCI)) m/z cal'd for (M+H)⁺ [C₂₂H₁₄F₆N₂O₄+H]⁺: 484.35, found 485.1874.

Following general demethylation procedure: To the solution of the dimethoxyl chromenone (10.3 m, 0.0213 mol) in CH₂Cl₂ (2.1 mL, 0.01 M) was added BBr₃ (90 μL, 0.0934 mmol) at 0° C. The reaction was slowly warm to 23° C. and stirred for 3 hours. Upon completion, the reaction mixture was quenched with MeOH (2.0 mL) and stirred for additional 1 hour. The solvent was removed by rotary evaporation, and the resulting residue was purified by flash chromatography (1:1 hexanes/EtOAc eluent) to afford analog 18 (5.1 mg, 53% yield) as a colorless solid. ¹H NMR (600 MHz, (CD₃)₂CO) δ 8.92 (s, 1H), 8.66 (s, 1H), 8.19 (d, J=8.3 Hz, 2H), 7.94-7.86 (m, 2H), 7.61 (d, J=8.7 Hz, 1H), 7.15 (d, J=8.7 Hz, 1H); HRMS (TOF LC/MS (APCI)) m/z cal'd for (M+H)⁺ [C₂₀H₁₀F₆N₂O₄+H]⁺: 456.29, found 457.0802.

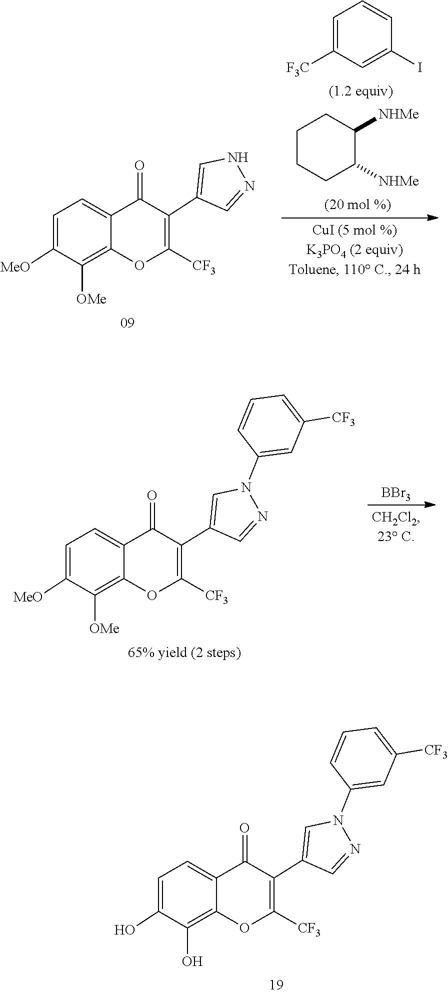 [SMILES: FC(F)(F)c1cccc(I)c1 ; COc1ccc2c(=O)c(-c3cn[nH]c3)c(C(F)(F)F)oc2c1OC ; CN[C@@H]1CCCC[C@H]1NC ; COc1ccc2c(=O)c(-c3cnn(-c4cccc(C(F)(F)F)c4)c3)c(C(F)(F)F)oc2c1OC ; "BBr3" ; O=c1c(-c2cnn(-c3cccc(C(F)(F)F)c3)c2)c(C(F)(F)F)oc2c(O)c(O)ccc12]

KH-4-65, Analog 19. Following general copper catalyzed cross coupling procedure A: To a mixture of 09 (13.8 mg, 0.0406 mmol), methyl diamine (4.3 mg, 0.0302 mmol), CuI (1.4 mg, 0.00735 mmol), and K₃PO₄ (21.4 mg, 0.101 mmol) in toluene (0.4 mL, 0.1 M) was added aryl iodide (21.4 mg, 0.0787 mmol). The reaction mixture was stirred at 110° C. for 24 hours. Upon completion, the reaction mixture was filtered through a SiO₂ plug, washing with EtOAc (2 mL). The solvent was then removed by rotary evaporation, and the resulting residue was used for the next step.

Following general demethylation procedure: To the solution of the resulting residue in CH₂Cl₂ (0.5 mL, 0.1 M) was added BBr₃ (0.10 mL, 0.100 mmol) at 0° C. The reaction was slowly warm to 23° C. and stirred for 3 hours. Upon completion, the reaction mixture was quenched with MeOH (1.0 mL) and stirred for additional 1 hour. The solvent was removed by rotary evaporation, and the resulting residue was purified by flash chromatography (1:1 hexanes/EtOAc eluent) to afford analog 19 (12.0 mg, 65% yield over 2 steps) as a colorless solid. ¹H NMR (600 MHz, (CD₃)₂CO) δ 8.70 (s, 1H), 8.30 (s, 1H), 8.26 (d, J=8.2 Hz, 1H), 7.87 (s, 1H), 7.81 (dd, J=7.9, 8.2 Hz, 1H), 7.71 (d, J=7.9 Hz, 1H), 7.61 (d, J=8.6 Hz, 1H), 7.14 (d, J=8.6 Hz, 1H); HRMS (TOF LC/MS (APCI)) m/z cal'd for (M+H)⁺ [C₂₀H₁₀F₆N₂O₄+H]⁺: 456.29, found 457.0664.

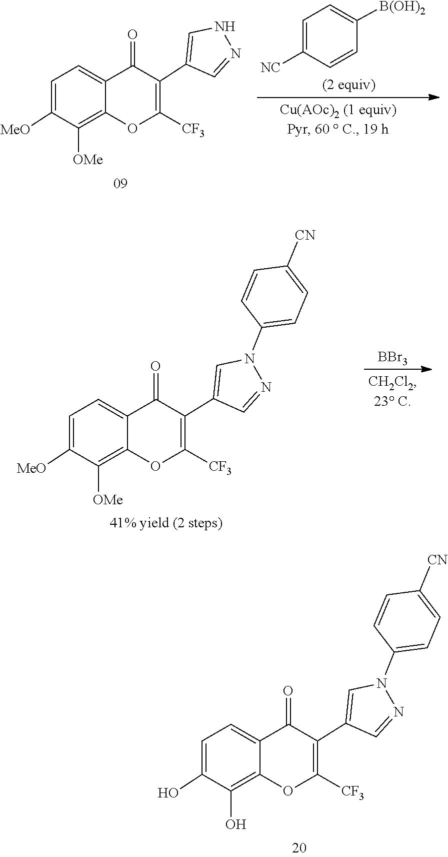

KH-3-152, Analog 20. Following general copper catalyzed cross coupling procedure B: To a mixture of 09 (14.8 mg, 0.0435 mmol), boronic acid (13.7 mg, 0.0932 mmol) in pyridine (0.4 mL, 0.1 M) was added Cu(OAc)₂ (9.6 mg, 0.0529 mmol). The reaction mixture was stirred at 60° C. for 19 hours. Upon completion, the reaction mixture was filtered through a SiO₂ plug, washing with EtOAc (2.0 mL). The solvent was then removed by rotary evaporation, and the resulting residue was used for the next step.

Following general demethylation procedure: To the solution of the dimethoxyl chromenone (6.0 m, 0.0136 mol) in CH₂Cl₂ (1.3 mL, 0.01 M) was added BBr₃ (55 μL, 0.0544 mmol) at 0° C. The reaction was slowly warm to 23° C. and stirred for 3 hours. Upon completion, the reaction mixture was quenched with MeOH (2.0 mL) and stirred for additional 1 hour. The solvent was removed by rotary evaporation, and the resulting residue was purified by flash chromatography (1:1 hexanes/EtOAc eluent) to afford analog 20 (2.3 mg, 41% yield) as a colorless solid. ¹H NMR (600 MHz(CD₃)₂CO) δ 9.49 (s, 1H), 868 (s, 1H), 8.19 (d, J=8.7 Hz, 1H), 7.98 (d, J=8.7 Hz, 1H), 7.66-7.58 (m, 2H), 7.01 (d, J=8.7 Hz, 1H); HRMS (TOF LC/MS (APCI)) m/z cal'd for (M+H)⁺ [C₂₀H₁₀F₃N₃O₄+H]⁺: 413.31, found 414.0698.

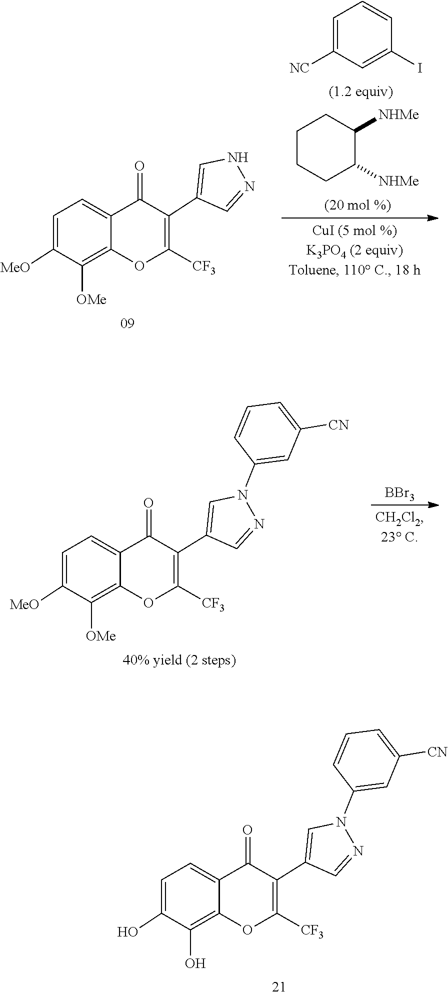 [SMILES: N#Cc1cccc(I)c1 ; COc1ccc2c(=O)c(-c3cn[nH]c3)c(C(F)(F)F)oc2c1OC ; CN[C@@H]1CCCC[C@H]1NC ; COc1ccc2c(=O)c(-c3cnn(-c4cccc(C#N)c4)c3)c(C(F)(F)F)oc2c1OC ; "BBr3" ; N#Cc1cccc(-n2cc(-c3c(C(F)(F)F)oc4c(O)c(O)ccc4c3=O)cn2)c1]

KH-4-66, Analog 21. Following general copper catalyzed cross coupling procedure A: To a mixture of 09 (15.0 mg, 0.041 mmol), methyl diamine (4.2 mg, 0.0295 mmol), CuI (1.8 mg, 0.00945 mmol), and K₃PO₄ (22.5 mg, 0.106 mmol) in toluene (0.4 mL, 0.1 M) was added aryl iodide (23.2 mg, 0.101 mmol). The reaction mixture was stirred at 110° C. for 18 hours. Upon completion, the reaction mixture was filtered through a SiO₂ plug, washing with EtOAc (2 mL). The solvent was then removed by rotary evaporation, and the resulting residue was used for the next step.

Following general demethylation procedure: To the solution of the resulting residue in CH₂Cl₂ (0.5 mL, 0.1 M) was added BBr₃ (0.10 mL, 0.100 mmol) at 0° C. The reaction was slowly warm to 23° C. and stirred for 3 hours. Upon completion, the reaction mixture was quenched with MeOH (1.0 mL) and stirred for additional 1 hour. The solvent was removed by rotary evaporation, and the resulting residue was purified by flash chromatography (1:1 hexanes/EtOAc eluent) to afford analog 21 (7.3 mg, 40% yield over 2 steps) as a colorless solid. ¹H NMR (600 MHz, (CD₃)₂CO) δ 9.69 (s, 1H), 9.04 (s, 1H), 8.67 (s, 1H), 8.6 (s, 1H), 8.33-8.28 (d, 1H), 7.88 (s, 1H), 7.82-7.56 (m, 2H), 7.59 (d, J=8.7 Hz, 1H), 7.14 (d, J=8.7 Hz, 1H); HRMS (TOF LC/MS (APCI)) m/z cal'd for (M+H)⁺ [C₂₀H₁₀F₃N₃O₄+H]⁺: 413.31, found 414.0718.

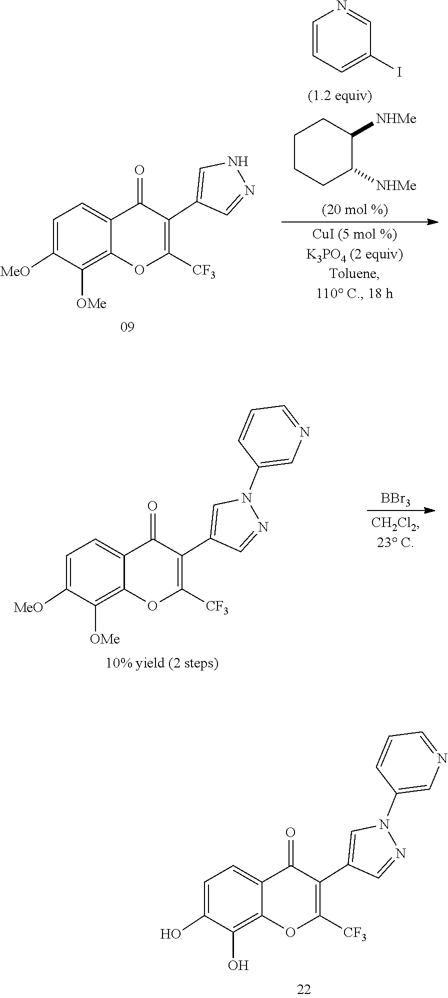 [SMILES: Ic1cccnc1 ; COc1ccc2c(=O)c(-c3cn[nH]c3)c(C(F)(F)F)oc2c1OC ; CN[C@@H]1CCCC[C@H]1NC ; COc1ccc2c(=O)c(-c3cnn(-c4cccnc4)c3)c(C(F)(F)F)oc2c1OC ; "BBr3" ; O=c1c(-c2cnn(-c3cccnc3)c2)c(C(F)(F)F)oc2c(O)c(O)ccc12]

KH-4-67, Analog 22. Following general copper catalyzed cross coupling procedure A: To a mixture of 09 (15.1 mg, 0.0444 mmol), methyl diamine (6.0 mg, 0.0422 mmol), CuI (1.8 mg, 0.00945 mmol), and K₃PO₄ (23.0 mg, 0.108 mmol) in toluene (0.5 mL, 0.1 M) was added aryl iodide (23.9 mg, 0.117 mmol). The reaction mixture was stirred at 110° C. for 18 hours. Upon completion, the reaction mixture was filtered through a SiO₂ plug, washing with EtOAc (2 mL). The solvent was then removed by rotary evaporation, and the resulting residue was used for the next step.

Following general demethylation procedure: To the solution of the resulting residue in CH₂Cl₂ (0.5 mL, 0.1 M) was added BBr₃ (0.10 mL, 0.100 mmol) at 0° C. The reaction was slowly warm to 23° C. and stirred for 3 hours. Upon completion, the reaction mixture was quenched with MeOH (1.0 mL) and stirred for additional 1 hour. The solvent was removed by rotary evaporation, and the resulting residue was purified by flash chromatography (1:1 hexanes/EtOAc eluent) to afford analog 22 (1.8 mg, 10% yield over 2 steps) as a colorless solid. ¹H NMR (600 MHz, (CD₃)₂CO) δ 9.50 (s, 1H), 9.10 (d, J=8.1 Hz, 1H), 9.10 (d, J=5.6 Hz, 1H), 8.86 (s, 1H), 8.23 (5, J=5.6, 8.1 Hz, 1H), 7.98 (s, 1H), 7.62 (d, J=8.7 Hz, 1H), 7.07 (d, J=8.7 Hz, 1H); HRMS (TOF LC/MS (APCI)) m/z cal'd for (M+H)⁺ [C₁₈H₁₀F₃N₃O₄+H]⁺: 389.28, found 390.0744.

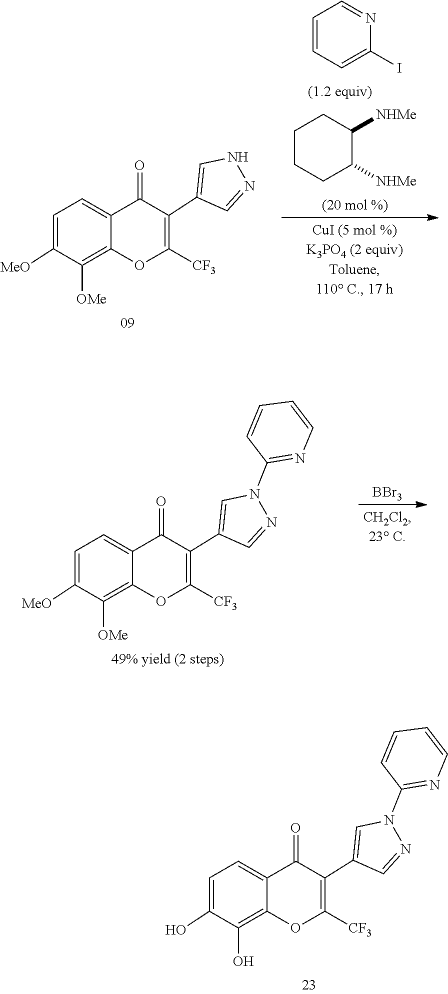 [SMILES: Ic1ccccn1 ; COc1ccc2c(=O)c(-c3cn[nH]c3)c(C(F)(F)F)oc2c1OC ; CN[C@@H]1CCCC[C@H]1NC ; COc1ccc2c(=O)c(-c3cnn(-c4ccccn4)c3)c(C(F)(F)F)oc2c1OC ; "BBr3" ; O=c1c(-c2cnn(-c3ccccn3)c2)c(C(F)(F)F)oc2c(O)c(O)ccc12]

Analog 23. Following general copper catalyzed cross coupling procedure A: To a mixture of 09 (15.5 mg, 0.0456 mmol), methyl diamine (4.0 mg, 0.0281 mmol), CuI (1.2 mg, 0.00630 mmol), and K₃PO₄ (21.7 mg, 0.102 mmol) in toluene (0.45 mL, 0.1 M) was added aryl iodide (9.5 μL, 0.0894 mmol). The reaction mixture was stirred at 110° C. for 17 hours. Upon completion, the reaction mixture was filtered through a SiO₂ plug, washing with EtOAc (2 mL). The solvent was then removed by rotary evaporation, and the resulting residue was used for the next step.

Following general demethylation procedure: To the solution of the resulting residue in CH₂Cl₂ (0.5 mL, 0.1 M) was added BBr₃ (0.10 mL, 0.100 mmol) at 0° C. The reaction was slowly warm to 23° C. and stirred for 3 hours. Upon completion, the reaction mixture was quenched with MeOH (1.0 mL) and stirred for additional 1 hour. The solvent was removed by rotary evaporation, and the resulting residue was purified by flash chromatography (1:1 hexanes/EtOAc eluent) to afford analog 23 (8.7 mg, 49% yield over 2 steps) as a colorless solid. ¹H NMR (600 MHz(CD₃)₂CO) δ 8.82 (s, 1H), 8.50 (d, J=4.6 Hz, 1H), 8.10-8.01 (m, 2H), 7.87 (s, 1H), 7.61 (d, J=8.7 Hz, 1H), 7.41-7.35 (m, 1H), 7.61 (d, J=8.7 Hz, 1H); HRMS (TOF LC/MS (APCI)) m/z cal'd for (M+H)⁺ [C₁₈H₁₀F₃N₃O₄+H]⁺: 389.28, found 390.0700.

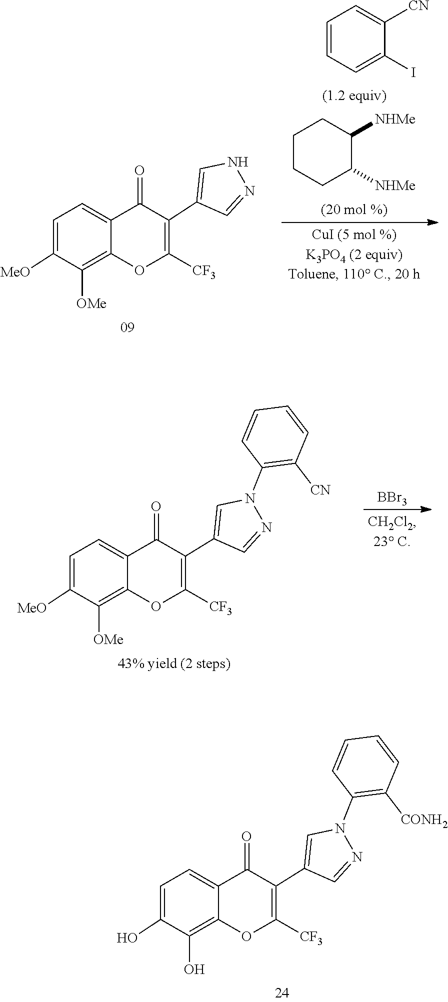

KH-4-44, Analog 24. Following general copper catalyzed cross coupling procedure A: To a mixture of 09 (12.9 mg, 0.0379 mmol), methyl diamine (12.9 mg, 0.0907 mmol), CuI (2.5 mg, 0.0131 mmol), and K₃PO₄ (27.9 mg, 0.131 mmol) in toluene (0.4 mL, 0.1 M) was added aryl iodide (20.6 mg, 0.0899 mmol). The reaction mixture was stirred at 110° C. for 20 hours. Upon completion, the reaction mixture was filtered through a SiO₂ plug, washing with EtOAc (2 mL). The solvent was then removed by rotary evaporation, and the resulting residue was used for the next step.

Following general demethylation procedure: To the solution of the resulting residue in CH₂Cl₂ (0.5 mL, 0.1 M) was added BBr₃ (0.10 mL, 0.100 mmol) at 0° C. The reaction was slowly warm to 23° C. and stirred for 3 hours. Upon completion, the reaction mixture was quenched with MeOH (1.0 mL) and stirred for additional 1 hour. The solvent was removed by rotary evaporation, and the resulting residue was purified by flash chromatography (1:1 hexanes/EtOAc eluent) to afford analog 24 (7.1 mg, 43% yield over 2 steps) as a colorless solid. ¹H NMR (600 MHz, (CD₃)₂CO) δ 8.47 (s, 1H), 8.12 (s, 1H), 7.85 (dt, J=1.4, 7.7 Hz, 2H), 7.79 (dt, J=1.4, 7.7 Hz, 1H), 7.67 (dt, J=1.4, 7.7 Hz, 1H), 7.59 (d, J=8.7 Hz, 1H), 7.16 (d, J=Hz, 1H); HRMS (TOF LC/MS (APCI)) m/z cal'd for (M+H)⁺ [C₂₀H₁₂F₃N₃O₅+H]⁺: 431.32, found 432.0796.

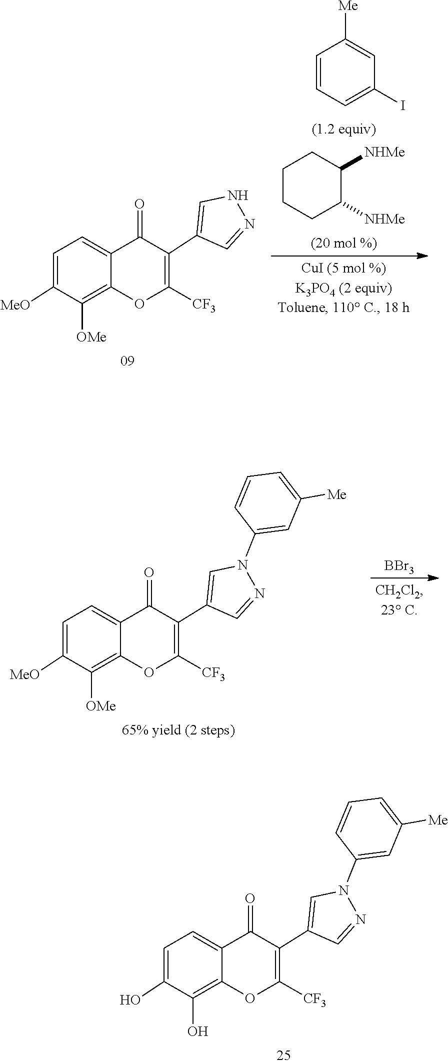

KH-4-79, Analog 25. Following general copper catalyzed cross coupling procedure A: To a mixture of 09 (15.3 mg, 0.0449 mmol), methyl diamine (6.6 mg, 0.0464 mmol), CuI (2.0 mg, 0.0105 mmol), and K₃PO₄ (27.4 mg, 0.129 mmol) in toluene (0.45 mL, 0.1 M) was added aryl iodide (11.5 μL, 0.113 mmol). The reaction mixture was stirred at 110° C. for 18 hours. Upon completion, the reaction mixture was filtered through a SiO₂ plug, washing with EtOAc (2 mL). The solvent was then removed by rotary evaporation, and the resulting residue was used for the next step.

Following general demethylation procedure: To the solution of the resulting residue in CH₂Cl₂ (0.45 mL, 0.1 M) was added BBr₃ (0.10 mL, 0.100 mmol) at 0° C. The reaction was slowly warm to 23° C. and stirred for 3 hours. Upon completion, the reaction mixture was quenched with MeOH (1.0 mL) and stirred for additional 1 hour. The solvent was removed by rotary evaporation, and the resulting residue was purified by flash chromatography (1:1 hexanes/EtOAc eluent) to afford analog 25 (11.8 mg, 65% yield over 2 steps) as a colorless solid. ¹H NMR (600 MHz, (CD₃)₂CO) δ 8.63 (s, 1H), 8.03 (s, 1H), 7.83 (s, 1H), 7.77 (d, J=7.8 Hz, 1H), 7.61 (d, J=8.7 Hz, 1H), 7.46 (t, J=7.8 Hz, 1H), 7.27 (d, J=7.8 Hz, 1H), 7.16 (d, J=8.7 Hz, 1H), 2.46 (s, 3H); HRMS (TOF LC/MS (APCI)) m/z cal'd for (M+H)⁺ [C₂₀H₁₃F₃N₂O₄+H]⁺: 402.32, found 403.1663.

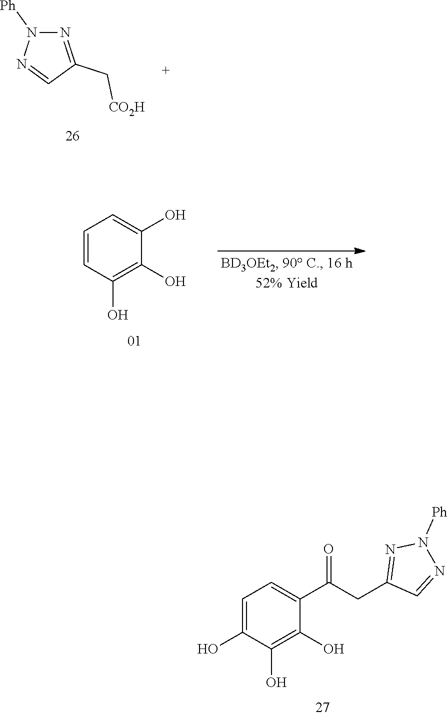 [SMILES: O=C(O)Cc1cnn(-c2ccccc2)n1 ; Oc1cccc(O)c1O ; O=C(Cc1cnn(-c2ccccc2)n1)c1ccc(O)c(O)c1O]

Phenol 27. Following general Fridel Crafts Procedure: To a solution of pyrogallol 01 (0.638 mg, 0.506 mmol) in BF₃OEt₂ (0.5 mL, 1.0 M) at 23° C. was added commercial carboxylic acid 26 (10.793 mL, 0.390 mmol), and the mixture was stirred for 22 hours at 90° C. Upon consumption of starting material, the reaction mixture was quenched with sat. NaOAc (10 mL). The solution was extracted with EtOAc (2×50 mL). The combined organic layers were washed with brine (30 mL) and dried over MgSO₄. After removal of solvent by rotary evaporation, the crude material was purified by flash chromatography (1:1 hexanes/EtOAc eluent), affording phenol 27 (0.0735 mg, 61% yield) as a yellow solid. ¹H NMR (600 MHz, CDCl₃) δ 12.47 (s, 1H), 8.06 (d, J=8.4 Hz, 2H), 7.82 (s, 1H), 7.52-7.47 (m, 3H), 7.36 (t, J=7.2 Hz, 1H), 6.61 (d, J=8.4 Hz, 1H), 5.93 (s, 1H), 5.48 (s, 1H), 4.48 (s, 2H); HRMS (TOF LC/MS (APCI)) m/z cal'd for (M+H)⁺ [C₁₆H₁₃N₃O₄+H]⁺: 311.29, found 312.0996.

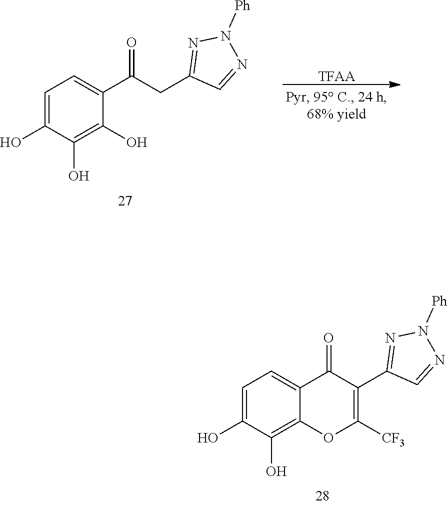 [SMILES: O=C(Cc1cnn(-c2ccccc2)n1)c1ccc(O)c(O)c1O ; "TFAA" ; O=c1c(-c2cnn(-c3ccccc3)n2)c(C(F)(F)F)oc2c(O)c(O)ccc12]

KH-5-09, Compound 28. To a solution of phenol 27 (0.0735 g, 0.236 mmol) in pyridine (2.4 mL, 0.1 M) at 23° C. was added TFAA (0.13 mL, 0.927 mmol), and the mixture was stirred for 20 hours at 90° C. Upon consumption of starting material, the solution was diluted with H₂O (15 mL) and then extracted with EtOAc (2×30 mL). The combined organic layers were washed with brine (30 mL) and dried over MgSO₄. After removal of solvent by rotary evaporation, the crude material was purified by flash chromatography (1:1 hexanes/EtOAc eluent), affording compound 28 (0.0533 mg, 58% yield) as a colorless solid. ¹H NMR (600 MHz, (CD₃)₂CO) δ 9.54 (s, 1H), 9.02 (s, 1H), 8.20 (s, 1H), 8.15 (d, J=8.1 Hz, 2H), 7.66-7.58 (m, 3H), 7.47 (t, J=7.4 Hz, 1H), 7.18 (d, J=8.1 Hz, 1H); HRMS (TOF LC/MS (APCI)) m z cal'd for (M+H)⁺ [C₁₈H₁₀F₃N₃O₄+H]⁺:389.28, found 390.0810.

In the following chemical syntheses all solvents were purchased from commercial sources and used without purification (HPLC or analytical grade). Anhydrous solvents were purchased from Acros Organics stored under a nitrogen atmosphere with activated molecular sieves. Standard vacuum line techniques were used, and glassware was flame dried prior to use. Deionised water was sourced using an HYDRO® Picopure Ultra-Pure water system. Organic solvents were dried during workup using anhydrous Na2SO4. Thin Layer Chromatography (TLC) was carried out using aluminum plates coated with 60 F254 silica gel. Plates were visualised using UV light (254 or 365 nm) or staining with Ninhydrin (1 M, EtOH) or 1% aq. KMnO₄. Normal-phase silica gel chromatography was carried out using a CombiFlash®Rf+ Teledyne ISCO flash column chromatography system (LPLC). NMR spectra were recorded using a Bruker Avance 600 MHz spectrometer using the deuterated solvent stated. Chemical shifts (6) quoted in parts per million (ppm) and referenced to the residual solvent peak. Multiplicities are denoted as s—singlet, d—doublet, t—triplet, q—quartet, and quin—quintet and derivatives thereof (br denotes a broad resonance peak). Coupling constants recorded as Hz and round to the nearest 0.1 Hz. Two-dimensional NMR experiments (COSY, HSQC, HMBC) were used to aid the assignment of ¹H and ¹³C spectra. Low Resolution mass spectra were recorded on a Waters SQ Detector 2 (LC-MS). Compound names were generated using ChemBioDraw Ultra v 14 systematic naming. Atom numbering in structures is purely for the purposes of assignment and does not reflect IUPAC numbering conventions.

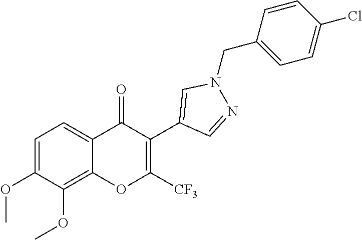 [SMILES: COc1ccc2c(=O)c(-c3cnn(Cc4ccc(Cl)cc4)c3)c(C(F)(F)F)oc2c1OC]

3-(1-(4-chlorobenzyl)-1H-pyrazol-4-yl)-7,8-dimethoxy-2-(trifluoromethyl)-4H-chromen-4-one, MSSM-RJD-MM-001. Initially, 7,8-dimethoxy-3-(1H-pyrazol-4-yl)-2-(trifluoromethyl)-4H-chromen-4-one (100 mg, 0.294 mmol, 1 eq) was dissolved in MeCN (3 mL, anhydrous) before potassium carbonate (90 mg, 0.647 mmol, 2.2 eq) and 4-chlorobenzyl bromide (66 mg, 0.323 mmol, 1.1 eq) were added sequentially and the reaction mixture was stirred at room temperature for 16 hours. Upon reaction completion, the reaction mixture was concentrated to a residue which was purified via LPLC (Hex:EA 1:0 to 3:2) to give a white powder (116 mg, 0.250 mmol, 85%). LR-ESI-MS: C₂₂H₁₇ClF₃N₂O₄ [M+H]⁺ m/z found 465.2, cal'd 465.1.

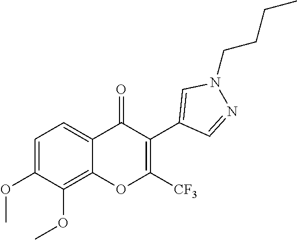 [SMILES: CCCCn1cc(-c2c(C(F)(F)F)oc3c(OC)c(OC)ccc3c2=O)cn1]

3-(1-butyl-1H-pyrazol-4-yl)-7,8-dimethoxy-2-(trifluoromethyl)-4H-chromen-4-one, MSSM-RJD-MM-002. Initially, 7,8-dimethoxy-3-(1H-pyrazol-4-yl)-2-(trifluoromethyl)-4H-chromen-4-one (100 mg, 0.294 mmol, 1 eq) was dissolved in MeCN (3 mL, anhydrous) before potassium carbonate (90 mg, 0.647 mmol, 2.2 eq) and n-butyl bromide (35 μL, 0.323 mmol, 1.1 eq) were added sequentially and the reaction mixture was stirred at room temperature for 16 hours. Upon reaction completion, the reaction mixture was concentrated to a residue which was purified via LPLC (Hex:EA 1:0 to 3:2) to give a white powder (67 mg, 0.17 mmol, 58%). LR-ESI-MS: C₁₉H₂₀F₃N₂O₄[M+H]⁺ m/z found 397.3, cal'd 397.1.

3-(1-propyl-1H-pyrazol-4-yl)-7,8-dimethoxy-2-(trifluoromethyl)-4H-chromen-4-one was prepared according to the above procedure using n-propyl iodide. 3-(1-ethyl-1H-pyrazol-4-yl)-7,8-dimethoxy-2-(trifluoromethyl)-4H-chromen-4-one was prepared according to the above procedure using ethyl iodide.

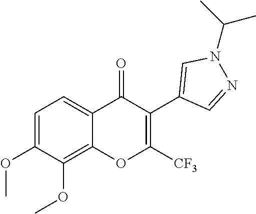 [SMILES: COc1ccc2c(=O)c(-c3cnn(C(C)C)c3)c(C(F)(F)F)oc2c1OC]

3-(1-isopropyl-1H-pyrazol-4-yl)-7,8-dimethoxy-2-(trifluoromethyl)-4H-chromen-4-one, MSSM-RJD-MM-003. Initially, 7,8-dimethoxy-3-(1H-pyrazol-4-yl)-2-(trifluoromethyl)-4H-chromen-4-one (100 mg, 0.294 mmol, 1 eq) was dissolved in MeCN (3 mL, anhydrous) before potassium carbonate (90 mg, 0.647 mmol, 2.2 eq) and 2-iodopropane (32 μL, 0.323 mmol, 1.1 eq) were added sequentially and the reaction mixture was stirred at room temperature for 16 hours. Upon reaction completion, the reaction mixture was concentrated to a residue which was purified via LPLC (Hex:EA 1:0 to 3:2) to give a white powder (17 mg, 0.044 mmol, 15%). LR-ESI-MS: C₁₈H₁₈F₃N₂O₄ [M+H]+m/z found 383.2, cal'd 383.1.

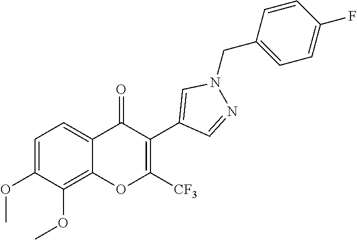 [SMILES: COc1ccc2c(=O)c(-c3cnn(Cc4ccc(F)cc4)c3)c(C(F)(F)F)oc2c1OC]

3-(1-(4-fluorobenzyl)-1H-pyrazol-4-yl)-7,8-dimethoxy-2-(trifluoromethyl)-4H-chromen-4-one, MSSM-RJD-MM-010. Initially, 7,8-dimethoxy-3-(1H-pyrazol-4-yl)-2-(trifluoromethyl)-4H-chromen-4-one (100 mg, 0.294 mmol, 1 eq) was dissolved in MeCN (3 mL, anhydrous) before potassium carbonate (90 mg, 0.647 mmol, 2.2 eq) and 4-fluorobenzyl bromide (40 μL, 0.323 mmol, 1.1 eq) were added sequentially and the reaction mixture was stirred at room temperature for 16 hours. Upon reaction completion, the reaction mixture was concentrated to a residue which was purified via LPLC (Hex:EA 1:0 to 3:2) to give a white powder (104 mg, 0.232 mmol, 79%). LR-ESI-MS: C₂₂H₁₇F₄N₂O₄[M+H]⁺ m/z found 449.2, cal'd 449.1.

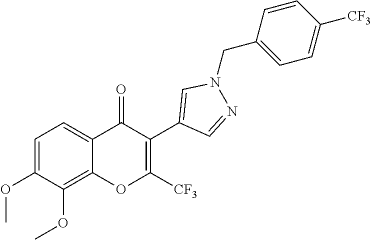 [SMILES: COc1ccc2c(=O)c(-c3cnn(Cc4ccc(C(F)(F)F)cc4)c3)c(C(F)(F)F)oc2c1OC]

7,8-dimethoxy-2-(trifluoromethyl)-3-(1-(4-(trifluoromethyl)benzyl)-1H-pyrazol-4-yl)-4H-chromen-4-one, MSSM-RJD-MM-011. Initially, 7,8-dimethoxy-3-(1H-pyrazol-4-yl)-2-(trifluoromethyl)-4H-chromen-4-one (100 mg, 0.294 mmol, 1 eq) was dissolved in MeCN (3 mL, anhydrous) before potassium carbonate (90 mg, 0.647 mmol, 2.2 eq) and 4-trifluoromethylbenzyl bromide (50 μL, 0.323 mmol, 1.1 eq) were added sequentially and the reaction mixture was stirred at room temperature for 16 hours. Upon reaction completion, the reaction mixture was concentrated to a residue which was purified via LPLC (Hex:EA 1:0 to 3:2) to give a white powder (112 mg, 0.225 mmol, 77%). LR-ESI-MS: C₂₃H₁₇F₆N₂O₄[M+H]⁺ m z found 499.2, cal'd 499.1.

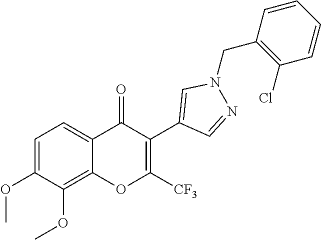 [SMILES: COc1ccc2c(=O)c(-c3cnn(Cc4ccccc4Cl)c3)c(C(F)(F)F)oc2c1OC]

3-(1-(2-chlorobenzyl)-1H-pyrazol-4-yl)-7,8-dimethoxy-2-(trifluoromethyl)-4H-chromen-4-one, MSSM-RJD-MM-012. Initially, 7,8-dimethoxy-3-(1H-pyrazol-4-yl)-2-(trifluoromethyl)-4H-chromen-4-one (100 mg, 0.294 mmol, 1 eq) was dissolved in MeCN (3 mL, anhydrous) before potassium carbonate (90 mg, 0.647 mmol, 2.2 eq) and 2-chlorobenzyl bromide (42 μL, 0.323 mmol, 1.1 eq) were added sequentially and the reaction mixture was stirred at room temperature for 16 hours. Upon reaction completion, the reaction mixture was concentrated to a residue which was purified via LPLC (Hex:EA 1:0 to 3:2) to give a white powder (101 mg, 0.217 mmol, 74%). LR-ESI-MS: C₂₂H₁₇ClF₃N₂O₄ [M+H]⁺ m/z found 465.2, cal'd 465.1.

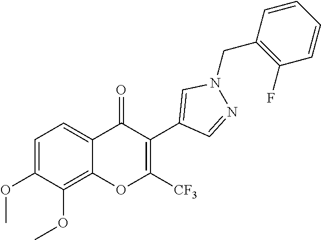 [SMILES: COc1ccc2c(=O)c(-c3cnn(Cc4ccccc4F)c3)c(C(F)(F)F)oc2c1OC]

3-(1-(2-fluorobenzyl)-1H-pyrazol-4-yl)-7,8-dimethoxy-2-(trifluoromethyl)-4H-chromen-4-one, MSSM-RJD-MM-016. Initially, 7,8-dimethoxy-3-(1H-pyrazol-4-yl)-2-(trifluoromethyl)-4H-chromen-4-one (100 mg, 0.294 mmol, 1 eq) was dissolved in MeCN (3 mL, anhydrous) before potassium carbonate (90 mg, 0.647 mmol, 2.2 eq) and 2-fluorobenzyl bromide (39 μL, 0.323 mmol, 1.1 eq) were added sequentially and the reaction mixture was stirred at room temperature for 16 hours. Upon reaction completion, the reaction mixture was concentrated to a residue which was purified via LPLC (Hex:EA 1:0 to 3:2) to give a white powder (86 mg, 0.191 mmol, 65%). LR-ESI-MS: C₂₂H₁₇F₄N₂O₄[M+H]⁺ m/z found 449.1, cal'd 449.1.

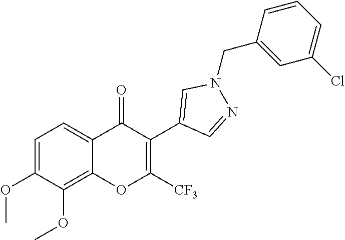 [SMILES: COc1ccc2c(=O)c(-c3cnn(Cc4cccc(Cl)c4)c3)c(C(F)(F)F)oc2c1OC]

3-(1-(3-chlorobenzyl)-1H-pyrazol-4-yl)-7,8-dimethoxy-2-(trifluoromethyl)-4H-chromen-4-one, MSSM-RJD-MM-017. Initially, 7,8-dimethoxy-3-(1H-pyrazol-4-yl)-2-(trifluoromethyl)-4H-chromen-4-one (100 mg, 0.294 mmol, 1 eq) was dissolved in MeCN (3 mL, anhydrous) before potassium carbonate (90 mg, 0.647 mmol, 2.2 eq) and 3-chlorobenzyl bromide (42 μL, 0.323 mmol, 1.1 eq) were added sequentially and the reaction mixture was stirred at room temperature for 16 hours. Upon reaction completion, the reaction mixture was concentrated to a residue which was purified via LPLC (Hex:EA 1:0 to 3:2) to give a white powder (61 mg, 0.131 mmol, 45%). LR-ESI-MS: C₂₂H₁₇ClF₃N₂O₄ [M+H]⁺ m/z found 465.2, cal'd 465.1.

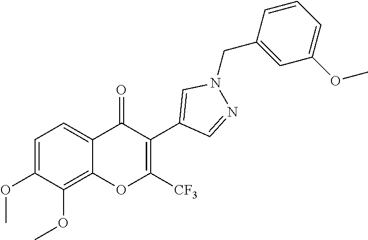 [SMILES: COc1cccc(Cn2cc(-c3c(C(F)(F)F)oc4c(OC)c(OC)ccc4c3=O)cn2)c1]

7,8-dimethoxy-3-(1-(3-methoxybenzyl)-1H-pyrazol-4-yl)-2-(trifluoromethyl)-4H-chromen-4-one, MSSM-RJD-MM-018. Initially, 7,8-dimethoxy-3-(1H-pyrazol-4-yl)-2-(trifluoromethyl)-4H-chromen-4-one (100 mg, 0.294 mmol, 1 eq) was dissolved in MeCN (3 mL, anhydrous) before potassium carbonate (90 mg, 0.647 mmol, 2.2 eq) and 3-methoxybenzyl bromide (45 μL, 0.323 mmol, 1.1 eq) were added sequentially and the reaction mixture was stirred at room temperature for 16 hours. Upon reaction completion, the reaction mixture was concentrated to a residue which was purified via LPLC (Hex:EA 1:0 to 3:2) to give a white powder (54 mg, 0.116 mmol, 40%). LR-ESI-MS: C₂₃H₂₀F₃N₂O₅[M+H]⁺ m/z found 461.2, cal'd 461.1.

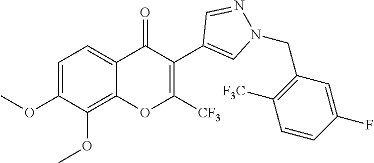 [SMILES: COc1ccc2c(=O)c(-c3cnn(Cc4cc(F)ccc4C(F)(F)F)c3)c(C(F)(F)F)oc2c1OC]

3-(1-(5-fluoro-2-(trifluoromethyl)benzyl)-1H-pyrazol-4-yl)-7,8-dimethoxy-2-(trifluoromethyl)-4H-chromen-4-one, MSSM-RJD-MM-021. Initially, 7,8-dimethoxy-3-(1H-pyrazol-4-yl)-2-(trifluoromethyl)-4H-chromen-4-one (100 mg, 0.294 mmol, 1 eq) was dissolved in MeCN (3 mL, anhydrous) before potassium carbonate (90 mg, 0.647 mmol, 2.2 eq) and 2-trifluoromethyl-5-fluorobenzyl bromide (55 μL, 0.323 mmol, 1.1 eq) were added sequentially and the reaction mixture was stirred at room temperature for 16 hours. Upon reaction completion, the reaction mixture was concentrated to a residue which was purified via LPLC (Hex:EA 1:0 to 3:2) to give a white powder (66 mg, 0.127 mmol, 43%). LR-ESI-MS: C₂₃H₁₆F₇N₂O₄[M+H]⁺ m/z found 517.2, cal'd 517.1.

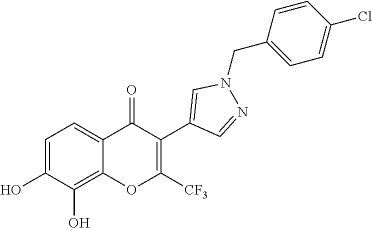 [SMILES: O=c1c(-c2cnn(Cc3ccc(Cl)cc3)c2)c(C(F)(F)F)oc2c(O)c(O)ccc12]

3-(1-(4-chlorobenzyl)-1H-pyrazol-4-yl)-7,8-dihydroxy-2-(trifluoromethyl)-4H-chromen-4-one, MSSM-RJD-MM-006. Initially, 3-(1-(4-chlorobenzyl)-1H-pyrazol-4-yl)-7,8-dimethoxy-2-(trifluoromethyl)-4H-chromen-4-one (50 mg, 0.11 mmol, 1 eq) was dissolved in DCM (2 mL, anhydrous) under an argon atmosphere. The solution was cooled to 0° C. before boron tribromide (200 μL, 0.23 mmol, 1 M, 2.1 eq) was added dropwise and the mixture was stirred for 16 hours at ambient temperature. Upon reaction completion, the mixture was quenched through the addition of cold water. The organic layer was washed with water (×3), brine (×1) and dried over Na₂SO₄ before being filtered and concentrated to a residue that was purified via LPLC (Hex:EA 1:0 to 0:1) to give a white powder (13 mg, 0.031 mmol, 29%). LR-ESI-MS: C₂₀H₁₃ClF₃N₂O₄ [M+H]⁺ m/z found 437.1, cal'd 437.1.

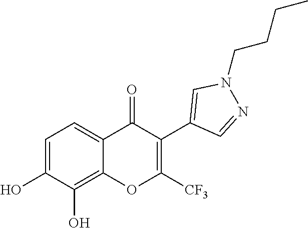 [SMILES: CCCCn1cc(-c2c(C(F)(F)F)oc3c(O)c(O)ccc3c2=O)cn1]

3-(1-butyl-1H-pyrazol-4-yl)-7,8-dihydroxy-2-(trifluoromethyl)-4H-chromen-4-one, MSSM-RJD-MM-007. Initially, 3-(1-butyl-1H-pyrazol-4-yl)-7,8-dimethoxy-2-(trifluoromethyl)-4H-chromen-4-one (20 mg, 0.05 mmol, 1 eq) was dissolved in DCM (2 mL, anhydrous) under an argon atmosphere. The solution was cooled to 0° C. before boron tribromide (106 μL, 0.1 mmol, 1 M, 2.1 eq) was added dropwise and the mixture was stirred for 16 hours at ambient temperature. Upon reaction completion, the mixture was quenched through the addition of cold water. The organic layer was washed with water (×3), brine (×1) and dried over Na₂SO₄ before being filtered and concentrated to a residue that was purified via LPLC (Hex:EA 1:0 to 0:1) to give a white powder (6.2 mg, 0.017 mmol, 33%). LR-ESI-MS: C₁₇H₁₆F₃N₂O₄[M+H]⁺ m/z found 369.2, cal'd 369.1.

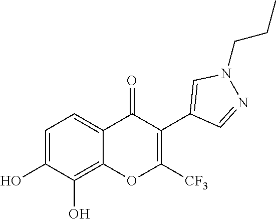 [SMILES: CCCn1cc(-c2c(C(F)(F)F)oc3c(O)c(O)ccc3c2=O)cn1]

7,8-dihydroxy-3-(1-propyl-1H-pyrazol-4-yl)-2-(trifluoromethyl)-4H-chromen-4-one, MSSM-RJD-MM-008. Initially, 7,8-dimethoxy-3-(1-propyl-1H-pyrazol-4-yl)-2-(trifluoromethyl)-4H-chromen-4-one (32 mg, 0.08 mmol, 1 eq) was dissolved in DCM (2 mL, anhydrous) under an argon atmosphere. The solution was cooled to 0° C. before boron tribromide (180 μL, 0.18 mmol, 1 M, 2.1 eq) was added dropwise and the mixture was stirred for 16 hours at ambient temperature. Upon reaction completion, the mixture was quenched through the addition of cold water. The organic layer was washed with water (×3), brine (×1) and dried over Na₂SO₄ before being filtered and concentrated to a residue that was purified via LPLC (Hex:EA 1:0 to 0:1) to give a white powder (8.3 mg, 0.023 mmol, 28%). LR-ESI-MS: C₁₆H₁₄F₃N₂O₄ [M+H]⁺ m/z found 355.1, cal'd 355.1.

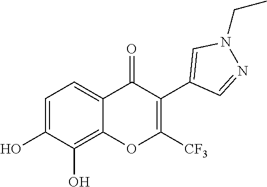 [SMILES: CCn1cc(-c2c(C(F)(F)F)oc3c(O)c(O)ccc3c2=O)cn1]

3-(1-ethyl-1H-pyrazol-4-yl)-7,8-dihydroxy-2-(trifluoromethyl)-4H-chromen-4-one, MSSM-RJD-MM-009. Initially, 3-(1-ethyl-1H-pyrazol-4-yl)-7,8-dimethoxy-2-(trifluoromethyl)-4H-chromen-4-one (22 mg, 0.06 mmol, 1 eq) was dissolved in DCM (2 mL, anhydrous) under an argon atmosphere. The solution was cooled to 0° C. before boron tribromide (130 μL, 0.13 mmol, 1 M, 2.1 eq) was added dropwise and the mixture was stirred for 16 hours at ambient temperature. Upon reaction completion, the mixture was quenched through the addition of cold water. The organic layer was washed with water (×3), brine (×1) and dried over Na₂SO₄ before being filtered and concentrated to a residue that was purified via LPLC (Hex:EA 1:0 to 0:1) to give a white powder (4 mg, 0.01 mmol, 20%). LR-ESI-MS: C₁₅H₁₂F₃N₂O₄ [M+H]⁺ m/z found 341.1, cal'd 341.1.

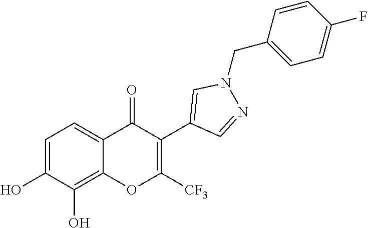 [SMILES: O=c1c(-c2cnn(Cc3ccc(F)cc3)c2)c(C(F)(F)F)oc2c(O)c(O)ccc12]

3-(1-(4-fluorobenzyl)-1H-pyrazol-4-yl)-7,8-dihydroxy-2-(trifluoromethyl)-4H-chromen-4-one, MSSM-RJD-MM-013. Initially, 3-(1-(4-fluorobenzyl)-1H-pyrazol-4-yl)-7,8-dimethoxy-2-(trifluoromethyl)-4H-chromen-4-one (54 mg, 0.12 mmol, 1 eq) was dissolved in DCM (2 mL, anhydrous) under an argon atmosphere. The solution was cooled to 0° C. before boron tribromide (253 μL, 0.25 mmol, 1 M, 2.1 eq) was added dropwise and the mixture was stirred for 16 hours at ambient temperature. Upon reaction completion, the mixture was quenched through the addition of cold water. The organic layer was washed with water (×3), brine (×1) and dried over Na₂SO₄ before being filtered and concentrated to a residue that was purified via LPLC (Hex:EA 1:0 to 0:1) to give a white powder (34 mg, 0.12 mmol, 66%). LR-ESI-MS: C₂₀H₁₃F₄N₂O₄[M+H]⁺ m/z found 421.2, cal'd 421.1.

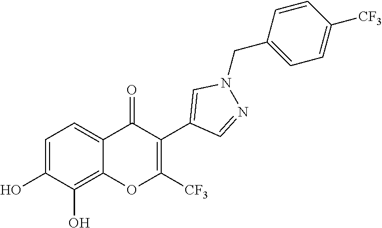 [SMILES: O=c1c(-c2cnn(Cc3ccc(C(F)(F)F)cc3)c2)c(C(F)(F)F)oc2c(O)c(O)ccc12]

7,8-dihydroxy-2-(trifluoromethyl)-3-(1-(4-(trifluoromethyl)benzyl)-1H-pyrazol-4-yl)-4H-chromen-4-one, MSSM-RJD-MM-014. Initially, 7,8-dimethoxy-2-(trifluoromethyl)-3-(1-(4-(trifluoromethyl)benzyl)-1H-pyrazol-4-yl)-4H-chromen-4-one (52 mg, 0.1 mmol, 1 eq) was dissolved in DCM (2 mL, anhydrous) under an argon atmosphere. The solution was cooled to 0° C. before boron tribromide (219 μL, 0.22 mmol, 1 M, 2.1 eq) was added dropwise and the mixture was stirred for 16 hours at ambient temperature. Upon reaction completion, the mixture was quenched through the addition of cold water. The organic layer was washed with water (×3), brine (×1) and dried over Na₂SO₄ before being filtered and concentrated to a residue that was purified via LPLC (Hex:EA 1:0 to 0:1) to give a white powder (29 mg, 0.1 mmol, 60%). LR-ESI-MS: C₂₁H₁₃F₆N₂O₄[M+H]⁺ m/z found 471.2, cal'd 471.1.

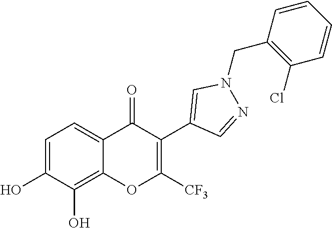 [SMILES: O=c1c(-c2cnn(Cc3ccccc3Cl)c2)c(C(F)(F)F)oc2c(O)c(O)ccc12]

3-(1-(2-chlorobenzyl)-1H-pyrazol-4-yl)-7,8-dihydroxy-2-(trifluoromethyl)-4H-chromen-4-one, MSSM-RJD-MM-015. Initially, 3-(1-(2-chlorobenzyl)-1H-pyrazol-4-yl)-7,8-dimethoxy-2-(trifluoromethyl)-4H-chromen-4-one (52 mg, 0.1 mmol, 1 eq) was dissolved in DCM (2 mL, anhydrous) under an argon atmosphere. The solution was cooled to 0° C. before boron tribromide (235 μL, 0.24 mmol, 1 M, 2.1 eq) was added dropwise and the mixture was stirred for 16 hours at ambient temperature. Upon reaction completion, the mixture was quenched through the addition of cold water. The organic layer was washed with water (×3), brine (×1) and dried over Na₂SO₄ before being filtered and concentrated to a residue that was purified via LPLC (Hex:EA 1:0 to 0:1) to give a white powder (31 mg, 0.1 mmol, 64%). LR-ESI-MS: C₂₀H₁₃ClF₃N₂O₄ [M+H]⁺ m/z found 437.2, cal'd 437.1.

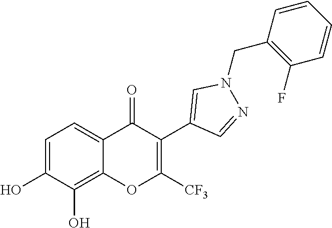 [SMILES: O=c1c(-c2cnn(Cc3ccccc3F)c2)c(C(F)(F)F)oc2c(O)c(O)ccc12]

3-(1-(2-fluorobenzyl)-1H-pyrazol-4-yl)-7,8-dihydroxy-2-(trifluoromethyl)-4H-chromen-4-one, MSSM-RJD-MM-022. Initially, 3-(1-(2-fluorobenzyl)-1H-pyrazol-4-yl)-7,8-dimethoxy-2-(trifluoromethyl)-4H-chromen-4-one (47 mg, 0.1 mmol, 1 eq) was dissolved in DCM (2 mL, anhydrous) under an argon atmosphere. The solution was cooled to 0° C. before boron tribromide (230 μL, 0.23 mmol, 1 M, 2.1 eq) was added dropwise and the mixture was stirred for 16 hours at ambient temperature. Upon reaction completion, the mixture was quenched through the addition of cold water. The organic layer was washed with water (×3), brine (×1) and dried over Na₂SO₄ before being filtered and concentrated to a residue that was purified via LPLC (Hex:EA 1:0 to 0:1) to give a white powder (16 mg, 0.04 mmol, 37%). LR-ESI-MS: C₂₀H₁₃F₄N₂O₄[M+H]⁺ m/z found 421.2, cal'd 421.1.

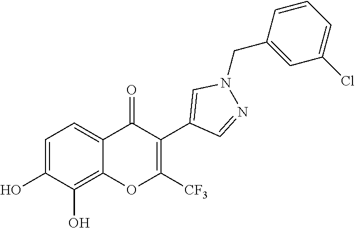 [SMILES: O=c1c(-c2cnn(Cc3cccc(Cl)c3)c2)c(C(F)(F)F)oc2c(O)c(O)ccc12]

3-(1-(3-chlorobenzyl)-1H-pyrazol-4-yl)-7,8-dihydroxy-2-(trifluoromethyl)-4H-chromen-4-one, MSSM-RJD-MM-023. Initially, 3-(1-(3-chlorobenzyl)-1H-pyrazol-4-yl)-7,8-dimethoxy-2-(trifluoromethyl)-4H-chromen-4-one (37 mg, 0.08 mmol, 1 eq) was dissolved in DCM (2 mL, anhydrous) under an argon atmosphere. The solution was cooled to 0° C. before boron tribromide (180 μL, 0.18 mmol, 1 M, 2.1 eq) was added dropwise and the mixture was stirred for 16 hours at ambient temperature. Upon reaction completion, the mixture was quenched through the addition of cold water. The organic layer was washed with water (×3), brine (×1) and dried over Na₂SO₄ before being filtered and concentrated to a residue that was purified via LPLC (Hex:EA 1:0 to 0:1) to give a white powder (13 mg, 0.03 mmol, 37%). LR-ESI-MS: C₂₀H₁₃ClF₃N₂O₄ [M+H]⁺ m/z found 437.1, cal'd 437.1.

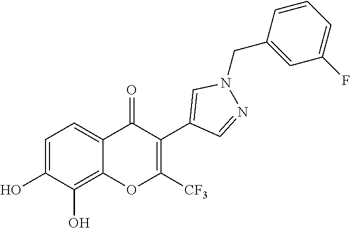 [SMILES: O=c1c(-c2cnn(Cc3cccc(F)c3)c2)c(C(F)(F)F)oc2c(O)c(O)ccc12]

3-(1-(3-fluorobenzyl)-1H-pyrazol-4-yl)-7,8-dihydroxy-2-(trifluoromethyl)-4H-chromen-4-one, MSSM-RJD-MM-024. Initially, 3-(1-(3-fluorobenzyl)-1H-pyrazol-4-yl)-7,8-dimethoxy-2-(trifluoromethyl)-4H-chromen-4-one (43 mg, 0.1 mmol, 1 eq) was dissolved in DCM (2 mL, anhydrous) under an argon atmosphere. The solution was cooled to 0° C. before boron tribromide (210 μL, 0.21 mmol, 1 M, 2.1 eq) was added dropwise and the mixture was stirred for 16 hours at ambient temperature. Upon reaction completion, the mixture was quenched through the addition of cold water. The organic layer was washed with water (×3), brine (×1) and dried over Na₂SO₄ before being filtered and concentrated to a residue that was purified via LPLC (Hex:EA 1:0 to 0:1) to give a white powder (20 mg, 0.05 mmol, 50%). LR-ESI-MS: C₂₀H₁₃F₄N₂O₄[M+H]⁺ m/z found 421.2, cald 421.1.

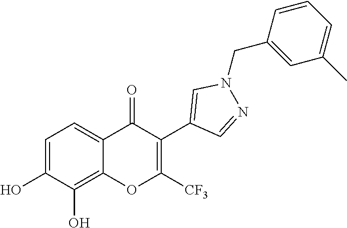 [SMILES: Cc1cccc(Cn2cc(-c3c(C(F)(F)F)oc4c(O)c(O)ccc4c3=O)cn2)c1]

7,8-dihydroxy-3-(1-(3-methylbenzyl)-1H-pyrazol-4-yl)-2-(trifluoromethyl)-4H-chromen-4-one, MSSM-RJD-MM-025. Initially, 7,8-dimethoxy-3-(1-(3-methylbenzyl)-1H-pyrazol-4-yl)-2-(trifluoromethyl)-4H-chromen-4-one (47 mg, 0.11 mmol, 1 eq) was dissolved in DCM (2 mL, anhydrous) under an argon atmosphere. The solution was cooled to 0° C. before boron tribromide (230 μL, 0.23 mmol, 1 M, 2.1 eq) was added dropwise and the mixture was stirred for 16 hours at ambient temperature. Upon reaction completion, the mixture was quenched through the addition of cold water. The organic layer was washed with water (×3), brine (×1) and dried over Na₂SO₄ before being filtered and concentrated to a residue that was purified via LPLC (Hex:EA 1:0 to 0:1) to give a white powder (22 mg, 0.05 mmol, 50%). LR-ESI-MS: C₂₁H₁₆F₃N₂O₄[M+H]⁺ m/z found 417.2, cal'd 417.1.

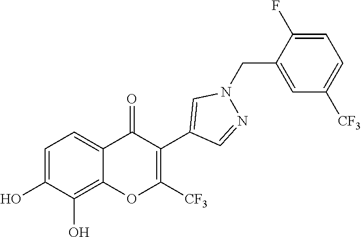 [SMILES: O=c1c(-c2cnn(Cc3cc(C(F)(F)F)ccc3F)c2)c(C(F)(F)F)oc2c(O)c(O)ccc12]

3-(1-(2-fluoro-5-(trifluoromethyl)benzyl)-1H-pyrazol-4-yl)-7,8-dihydroxy-2-(trifluoromethyl)-4H-chromen-4-one, MSSM-RJD-MM-026. Initially, 3-(1-(2-fluoro-5-(trifluoromethyl)benzyl)-1H-pyrazol-4-yl)-7,8-dimethoxy-2-(trifluoromethyl)-4H-chromen-4-one (41 mg, 0.08 mmol, 1 eq) was dissolved in DCM (2 mL, anhydrous) under an argon atmosphere. The solution was cooled to 0° C. before boron tribromide (170 μL, 0.17 mmol, 1 M, 2.1 eq) was added dropwise and the mixture was stirred for 16 hours at ambient temperature. Upon reaction completion, the mixture was quenched through the addition of cold water. The organic layer was washed with water (×3), brine (×1) and dried over Na₂SO₄ before being filtered and concentrated to a residue that was purified via LPLC (Hex:EA 1:0 to 0:1) to give a white powder (26 mg, 0.05 mmol, 67%). LR-ESI-MS: C₂₁H₁₂F₇N₂O₄ [M+H]⁺ m/z found 489.1, cal'd 489.1.

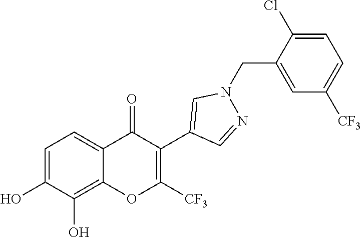 [SMILES: O=c1c(-c2cnn(Cc3cc(C(F)(F)F)ccc3Cl)c2)c(C(F)(F)F)oc2c(O)c(O)ccc12]

3-(1-(2-chloro-5-(trifluoromethyl)benzyl)-1H-pyrazol-4-yl)-7,8-dihydroxy-2-(trifluoromethyl)-4H-chromen-4-one, MSSM-RJD-MM-027. Initially, 3-(1-(2-chloro-5-(trifluoromethyl)benzyl)-1H-pyrazol-4-yl)-7,8-dimethoxy-2-(trifluoromethyl)-4H-chromen-4-one (46 mg, 0.09 mmol, 1 eq) was dissolved in DCM (2 mL, anhydrous) under an argon atmosphere. The solution was cooled to 0° C. before boron tribromide (190 μL, 0.19 mmol, 1 M, 2.1 eq) was added dropwise and the mixture was stirred for 16 hours at ambient temperature. Upon reaction completion, the mixture was quenched through the addition of cold water. The organic layer was washed with water (×3), brine (×1) and dried over Na₂SO₄ before being filtered and concentrated to a residue that was purified via LPLC (Hex:EA 1:0 to 0:1) to give a white powder (14 mg, 0.03 mmol, 33%). LR-ESI-MS: C₂₁H₁₂ClF₆N₂O₄ [M+H]⁺ m/z found 505.1, cal'd 505.0.

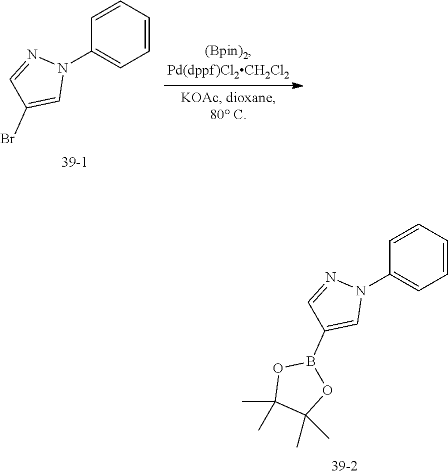 [SMILES: Brc1cnn(-c2ccccc2)c1 ; CC1(C)OB(c2cnn(-c3ccccc3)c2)OC1(C)C]

1-Phenyl-4-(4,4,5,5-tetramethyl-1,3,2-dioxaborolan-2-yl)-1H-pyrazole (intermediate 39-2): To a solution of 4-bromo-1-phenyl-1H-pyrazole (intermediate 39-1) (150.0 mg, 0.67 mmol), bis(pinacolato)diboron (256.1 mg, 1.01 mmol) and Pd(dppf)Cl₂·CH₂Cl₂ (57.7 mg, 0.07 mmol) in 1,4-dioxane (4 mL) was added KOAc (197.8 mg, 2.02 mmol) under nitrogen. The resulting mixture was stirred at 80° C. for 16 hours. After being quenched with H₂O (3 mL), the aqueous layer was extracted with EtOAc (2×20 mL). The combined organic extracts were washed with brine, dried over anhydrous Na₂SO₄, filtered, and concentrated. The residue was purified by column chromatography on silica gel (EtOAc/hexane, 5:95 to 10:90) to afford intermediate 39-2 (132.8 mg, 73%) as a white solid; ¹H NMR (CDCl₃, 600 MHz) 8.22 (1H, s), 7.96 (1H, s), 7.68 (2H, d, J=8.5 Hz), 7.43 (2H, t, J=8.5 Hz), 7.27 (1H, t, J=7.3 Hz), 1.33 (12H, s); HRMS (ESI-TOF) m/z: [M+H]⁺ calculated for C₁₅H₂₀BN₂O₂ 271.1612; found 271.1690.

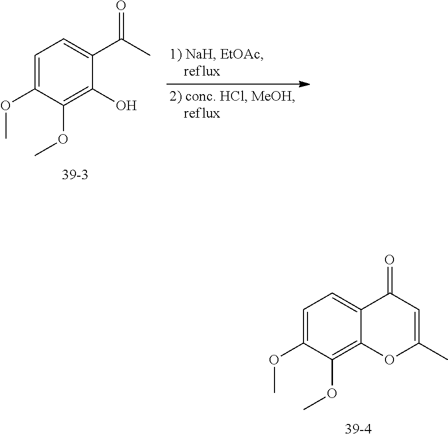 [SMILES: COc1ccc(C(C)=O)c(O)c1OC ; COc1ccc2c(=O)cc(C)oc2c1OC]

7,8-Dimethoxy-2-methyl-4H-chromen-4-one (intermediate 39-4): To a solution of 1-(2-hydroxy-3,4-dimethoxyphenyl)ethenone (intermediate 39-3) (500.0 mg, 2.55 mmol) in anhydrous ethylacetate (15 mL) was added 60% NaH (203.9 mg, 5.10 mmol) under nitrogen. The resulting mixture was refluxed for 16 hours. The reaction mixture was allowed to cool and was concentrated to remove solvent. Methanol (10 mL) was added to the crude product then concentrated HCl (2 mL) was added. The reaction was refluxed for 30 min and methanol was removed in vacuum. After with H₂O (10 mL), the aqueous layer was extracted with EtOAc (2×20 mL). The combined organic extracts were washed with brine, dried over anhydrous Na₂SO₄, filtered, and concentrated. The residue was purified by column chromatography on silica gel (EtOAc/hexane, 20:80 to 40:60) to afford intermediate 39-4 (221.7 mg, 40%) as a pale yellow solid; ¹H NMR (CDCl₃, 600 MHz) 7.87 (1H, d, J=8.5 Hz), 6.98 (1H, d, J=8.5 Hz), 6.07 (1H, s), 3.95-3.94 (6H, m), 2.39 (3H, s); HRMS (ESI-TOF) m/z: [M+H]⁺ calculated for C₁₂H₁₃O₄ 221.0814; found 221.0813.

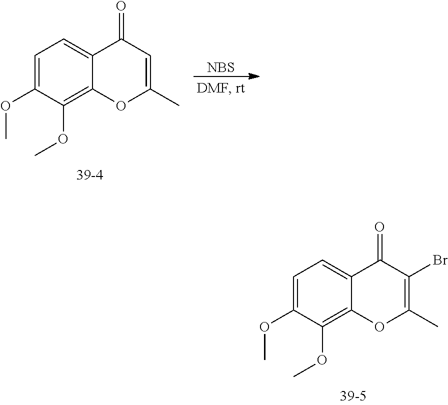

3-Bromo-7,8-dimethoxy-2-methyl-4H-chromen-4-one (intermediate 39-5): To a solution of intermediate 39-4 (160.0 mg, 0.73 mmol) in anhydrous DMF (5 mL) was added NBS (142.2 mg, 0.80 mmol) under nitrogen. The resulting mixture was stirred at room temperature for 2.5 hours. After being quenched with H₂O (3 mL), the aqueous layer was extracted with EtOAc (2×20 mL). The combined organic extracts were washed with brine, dried over anhydrous Na₂SO₄, filtered, and concentrated. The residue was purified by column chromatography on silica gel (EtOAc/hexane, 30:70) to afford intermediate 39-5 (172.6 mg, 80%) as a pale yellow solid; ¹H NMR (CDCl₃, 600 MHz) 7.96 (1H, d, J=8.5 Hz), 7.04 (1H, d, J=8.5 Hz), 3.99 (3H, s), 3.97 (3H, s), 2.69 (2H, s); HRMS (ESI-TOF) m/z: [M+H]⁺ calculated for C₁₂H₁₂⁷⁹BrO₄ and C₁₂H₂⁸¹BrO₄ 298.9913, 300.9893; found 298.9926, 300.9901.

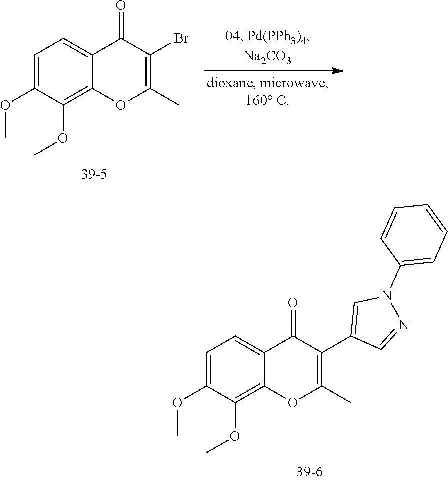 [SMILES: COc1ccc2c(=O)c(Br)c(C)oc2c1OC ; COc1ccc2c(=O)c(-c3cnn(-c4ccccc4)c3)c(C)oc2c1OC]

7,8-Dimethoxy-2-methyl-3-(1-phenyl-1H-pyrazol-4-yl)-4H-chromen-4-one, (intermediate 39-6): To a solution of intermediate 39-5 (25.0 mg, 0.08 mmol), 04 (22.6 mg, 0.08 mmol) and Pd(PPh₃)₄ (9.2 mg, 0.008 mmol) in 1,4-dioxane (1 mL) was 1 M Na₂CO₃ (0.17 mL 0.17 mmol) under nitrogen. The resulting mixture was heated in microwave at 160° C. for 15 min. After being quenched with H₂O (3 mL), the aqueous layer was extracted with EtOAc (2×10 mL). The combined organic extracts were washed with brine, dried over anhydrous Na₂SO₄, filtered, and concentrated. The residue was purified by column chromatography on silica gel (EtOAc/hexane, 20:80 to 40:60) to afford intermediate 39-6 (13.7 mg, 45%) as a white solid; ¹H NMR (CDCl₃, 600 MHz) 8.35 (1H, s), 7.97 (1H, d, J=9.8 Hz), 7.82 (1H, s), 7.75 (2H, d, J=7.3 Hz), 7.46 (2H, t, J=7.3 Hz), 7.30 (1H, t, J=7.3 Hz), 7.04 (1H, d, J=9.8 Hz), 4.01 (3H, s), 4.00 (3H, s), 2.63 (3H, s); HRMS (ESI-TOF) m/z: [M+H]⁺ calculated for C₂₁H₁₉N₂O₄ 363.1339; found 363.1332.

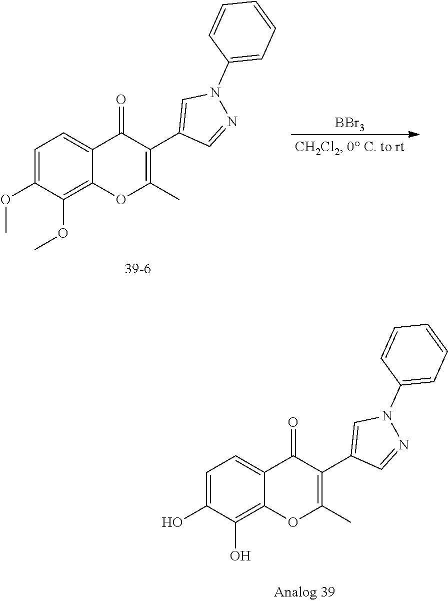 [SMILES: COc1ccc2c(=O)c(-c3cnn(-c4ccccc4)c3)c(C)oc2c1OC ; "BBr3" ; Cc1oc2c(O)c(O)ccc2c(=O)c1-c1cnn(-c2ccccc2)c1]

7,8-Dihydroxy-2-methyl-3-(1-phenyl-1H-pyrazol-4-yl)-4H-chromen-4-one, (Analog 39): To a solution of intermediate 39-6 (11.0 mg, 0.03 mmol) in anhydrous dichloromethane (1 mL) was added 1 M BBr₃ (0.15 mL 0.15 mmol) dropwise at 0° C. The resulting mixture was stirred at 0° C. to room temperature for 5 hours then saturated NaHCO₃ (1 mL) and EtOAc (1 mL) were added. The reaction was stirred at room temperature for 30 min. Water (5 mL) was added and the aqueous layer was extracted with EtOAc (2×10 mL). The combined organic extracts were washed with brine, dried over anhydrous Na₂SO₄, filtered, and concentrated. The residue was purified by column chromatography on silica gel (MeOH/CH₂Cl₂, 5:95) to afford Analog 39 (7.6 mg, 76%) as a white solid; ¹H NMR (CD₃OD, 600 MHz) 8.39 (1H, s), 7.85 (1H, s), 7.78 (2H, d, J=8.5 Hz), 7.52-7.48 (3H, m), 7.33 (1H, t, J=7.3 Hz), 6.92 (1H, d, J=9.8 Hz), 2.57 (3H, s); HRMS (ESI-TOF) m/z: [M+H]⁺ calculated for C₁₉H₁₅N₂O₄ 335.1026; found 335.1030.

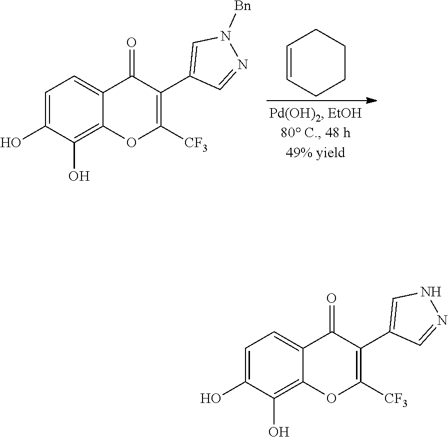 [SMILES: O=c1c(-c2cnn(Cc3ccccc3)c2)c(C(F)(F)F)oc2c(O)c(O)ccc12 ; C1=CCCCC1 ; O=c1c(-c2cn[nH]c2)c(C(F)(F)F)oc2c(O)c(O)ccc12]

7,8-Dihydroxy-3-(1H-pyrazol-4-yl)-2-(trifluoromethyl)-4H-chromen-4-one: To a mixture of 3-(1-benzyl-1H-pyrazol-4-yl)-7,8-dihydroxy-2-(trifluoromethyl)-4H-chromen-4-one (10 mg, 0.02 mmol) in EtOH (0.5 mL) was added cyclohexene (0.24 mL) and Pd(OH)₂ (10 mg), respectively. The reaction mixture was refluxed for 48 hours. Upon completion, the reaction mixture was filtered through a Celite pad, washing with EtOAc (5 mL). The solvent was then removed by rotary evaporation, and the resulting residue was purified by flash chromatography (1:3 Hexane/EtOAc eluent) to afford 7,8-dihydroxy-3-(1H-pyrazol-4-yl)-2-(trifluoromethyl)-4H-chromen-4-one (3.5 mg, 49% yield). 1H NMR (600 MHz, (CD₃OD) δ 7.70 (br, 2H), 7.55 (d, J=8.5 Hz, 1H), 7.00 (d, J=8.5 Hz, 1H), 2.64 (s, 3H); HRMS (TOF LC/MS (APCI)) m/z cal'd for (M+H)⁺ [C₁₃H₇F₃N₂O₄+H]⁺: 313.04, found 313.2883.

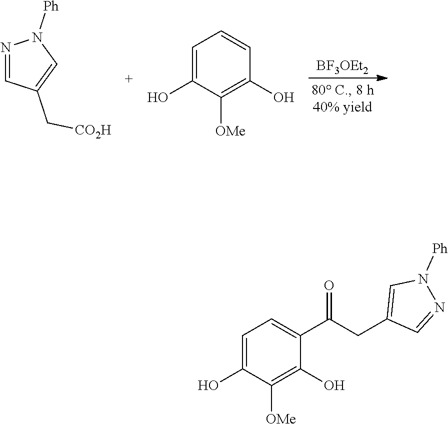 [SMILES: O=C(O)Cc1cnn(-c2ccccc2)c1 ; COc1c(O)cccc1O ; COc1c(O)ccc(C(=O)Cc2cnn(-c3ccccc3)c2)c1O ; "BF3OEt2"]

1-(2,4-Dihydroxy-3-methoxyphenyl)-2-(1-phenyl-1H-pyrazol-4-yl)ethenone was prepared from 2-(1-benzyl-1H-pyrazol-4-yl) acetic acid and 2-methoxybenzene-1,3-diol and purified by column chromatography on silica gel (95:5 CH₂Cl₂/MeOH eluent) to afford 1-(2,4-dihydroxy-3-methoxyphenyl)-2-(1-phenyl-1H-pyrazol-4-yl)ethenone (1.95 g, 40% yield). The crude product was used in next step; HRMS (TOF LC/MS (APCI)) m/z cal'd for (M+H)+[C₁₈H₁₇N₂O₄+H]⁺: 325.1183, found 325.1528.

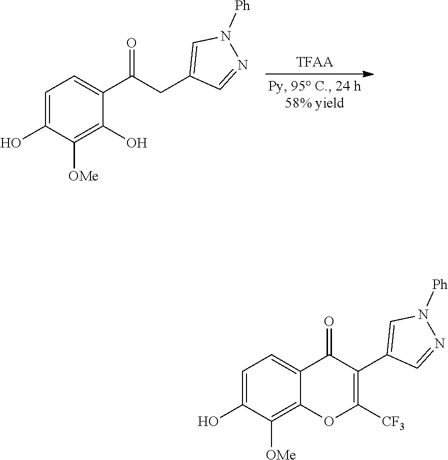

7-Hydroxy-8-methoxy-3-(1-phenyl-1H-pyrazol-4-yl)-2-(trifluoromethyl)-4H-chromen-4-one was prepared from 1-(2,4-dihydroxy-3-methoxyphenyl)-2-(1-phenyl-1H-pyrazol-4-yl)ethenone and purified by column chromatography on silica gel (80:20 to 65:35 Hexane/EtOAc eluent) (94.5 mg, 58% yield). The crude product was used in next step. HRMS (TOF LC/MS (APCI)) m/z cal'd for (M+H)⁺ [C₂₀H₁₄F₃N₂O₄+H]⁺: 403.09, found 403.1116.

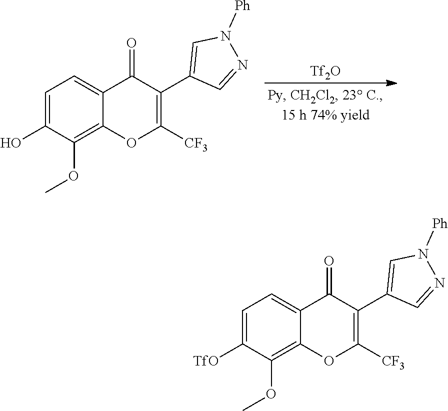 [SMILES: "Tf2O" ; COc1c(O)ccc2c(=O)c(-c3cnn(-c4ccccc4)c3)c(C(F)(F)F)oc12 ; COc1c(OS(=O)(=O)C(F)(F)F)ccc2c(=O)c(-c3cnn(-c4ccccc4)c3)c(C(F)(F)F)oc12]

8-Methoxy-4-oxo-3-(1-phenyl-1H-pyrazol-4-yl)-2-(trifluoromethyl)-4H-chromen-7-yl trifluoromethanesulfonate: To a mixture of 7-hydroxy-8-methoxy-3-(1-phenyl-1H-pyrazol-4-yl)-2-(trifluoromethyl)-4H-chromen-4-one (94.5 mg, 0.24 mmol) in CH₂Cl₂ (2.5 mL) was added pyridine (0.03 mL) and trifluoromethanesulfonic anhydride (0.06 mL) at 0° C. under N₂. The reaction mixture was warmed to room temperature and stirred for 15 hours. The reaction was diluted with H₂O (10 mL) was added then extracted with CH₂Cl₂ (2×10 mL). The combined organic layers were washed with brine (10 mL) and dried over MgSO₄. After removal of solvent by rotary evaporation, the residue was purified by column chromatography on silica gel (1:1 Hexane/EtOAc eluent) to afford 8-methoxy-4-oxo-3-(1-phenyl-1H-pyrazol-4-yl)-2-(trifluoromethyl)-4H-chromen-7-yl trifluoromethanesulfonate (92.8 mg, 74% yield). ¹H NMR (600 MHz, (CD₃Cl) δ 8.26 (s, 1H), 8.02 (d, J=8.5 Hz, 1H), 7.85 (s, 1H), 7.75 (d, J=8.5 Hz, 2H), 7.49 (t, J=8.5 Hz, 2H), 7.36-7.30 (m, 2H), 4.24 (s, 3H); HRMS (TOF LC/MS (APCI)) m z cal'd for (M+H)⁺ [C₂₁H₁₃F₆N₂O₆S+H]⁺: 535.04, found 535.4952.

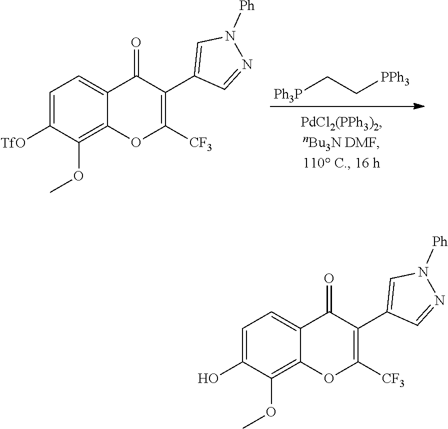

7-Hydroxy-8-methoxy-3-(1-phenyl-1H-pyrazol-4-yl)-2-(trifluoromethyl)-4H-chromen-4-one: To a mixture of 8-methoxy-4-oxo-3-(1-phenyl-1H-pyrazol-4-yl)-2-(trifluoromethyl)-4H-chromen-7-yl trifluoromethanesulfonate (6.8 mg, 0.02 mmol), 1,2-ethanediylbis(triphenylphosphorane) (1.0 mg, 0.002 mmol) and PdCl₂(PPh₃)₂ (1.0 mg, 0.001 mmol) in anhydrous DMF (0.2 mL) was added tributylamine (0.02 mL, 0.07 mmol) under nitrogen. The reaction mixture was stirred at 90° C. for 16 hours. Upon completion, the reaction mixture was filtered through a SiO₂ plug, washing with EtOAc (2 mL). The solvent was then removed by rotary evaporation, and the resulting residue was purified by flash chromatography (3:1 to 1:1 Hexane/EtOAc eluent) to afford 7-hydroxy-8-methoxy-3-(1-phenyl-1H-pyrazol-4-yl)-2-(trifluoromethyl)-4H-chromen-4-one. ¹H NMR (600 MHz, (CD₃Cl) δ 8.25 (s, 1H), 7.91 (d, J=8.5 Hz, 1H), 7.84 (s, 1H), 7.75 (d, J=8.5 Hz, 2H), 7.48 (t, J=7.3 Hz, 2H), 7.33 (t, J=7.3 Hz, 1H), 7.11 (d, J=8.5 Hz, 1H), 4.15 (s, 3H); HRMS (TOF LC/MS (APCI)) m/z cal'd for (M+H)⁺ [C₂₀H₁₄F₃N₂O₄+H]⁺: 403.09, found 403.29.

### Example 5—Preparation of ROC1-CUL4A CTD

Cloning: Plasmid expressing Human Cul4A CTD (residues 400-759)/GST-ROC1 complex was created using Quickchange II XL site-directed mutagenesis kit (Agilent Technologies) according to the manufacturer's protocols. The template plasmid used (Angers et al., “Molecular Architecture and Assembly of the DDB1-CUL4A Ubiquitin Ligase Machinery,” Nature 443(7111):590-3 (2006), which is hereby incorporated by reference in its entirety) contains sequences co-expressing GST-ROC1 (ROC1 sequence starting at aa15) and 6×His-CUL4A (CUL4A sequence starting at residue 39). The sequence coding for the N-terminal Cul4A residues up to aa399 was removed using the following complementary mutagenesis primers: 5′-GCC GCG CGG CAG CCA TAA GAG ACC CAA GCC-3′ (SEQ ID NO:1) and 5′-GGC TTG GGT CTC TTA TGG CTG CCG CGC GGC-3′ (SEQ ID NO:2). The italicized sequences contain part of the N-terminal thrombin site contained in the template sequence and the bold sequences denotes the codon sequence for CUL4A aa400. The resulting construct was verified by DNA sequencing.

Expression and purification: The plasmid expressing CUL4A-CTD/GST-ROC1 complex was transformed into Rosetta™ 2(DE3) (Novagen) cells per the manufacturer's instructions. A single colony was picked from the Ampicillin and Chloramphenicol LB agar plate and inoculated into a starter culture containing LB media, 100 μg/ml carbenicillin, and 34 mg/L chloramphenicol overnight in a 37° C. incubator shaking at 250 rpm. The starter culture was expanded 1:50 into the same media supplemented with 0.5 mM ZnCl₂, grown at 37° C. at 250 rpm until the culture reached an OD600 of 0.7. The culture was cooled to 18° C. in a water bath and protein expression was induced with 0.5 mM Isopropyl-1-thio-b-D-galactopyranoside. The culture was then incubated overnight at 18° C., 250 rpm. Cells were pelleted by centrifugation at 5000×g for 15 min at 4° C. and stored at −80° C. until extraction. For extraction, pellet was re-suspended in lysis buffer (50 mM Tris-HCl, pH 8.0, 1% TRITON X-100, 0.5M NaCl, 2 mM phenylmethylsulfonyl fluoride, 0.4 μg/ml antipain, and 0.2 μg/ml leupeptin, 5 mM DTT) then sonicated for 2 min on pulse mode using an Omni Rupter 4000 Ultrasonic Homogenizer (Omni International) and clarified by centrifugation at 17,000 rpm in an SS-34 rotor for 30 min, 4° C. The extract was incubated with Glutathione Sepharose beads (GE Healthcare) overnight, 4° C. The beads were packed into a chromatography column and washed with 20× column volumes of lysis buffer, followed by 10× column volumes of buffer A (50 mM Tris-HCl, pH 7.5, 300 mM NaCl, 0.01% NP-40, 10% glycerol, 1 mM DTT). The ROC1-CUL4A CTD complex was then eluted with buffer A containing 20 mM glutathione. Fractions containing the ROC1-CUL4A CTD complex, as determined by Coomassie staining of SDS-PAGE gel analysis were pooled, concentrated and buffer exchanged to thrombin cleavage buffer (20 mM Tris-HCl, pH 8.4, 150 mM NaCl, 10% glycerol, 2.5 mM CaCl₂) using an Amicon Ultra centrifugal filter. The concentrated material was bound to Ni-NTA beads (Qiagen) 4° C. overnight on a shaker. The beads were then washed with cleavage buffer then incubated with biotinylated thrombin (Novagen) at 4° C. to cleave off the GST and 6×His tags. Supernatant containing the cleaved complex was collected after overnight incubation. The concentration of ROC1-CUL4A CTD was 0.33 mg/ml. The yield was 0.66 mg per 1 liter of induced bacterial culture.

### Example 6—siRNA Transfection

SMARTpool ON-TARGETplus small interfering RNAs (siRNAs) for CUL4A, CUL4B, and Non-targeting control were purchased from Dharmacon. CUL4A SMARTpool targeting sequences were: GCACAGAUCCUUCCGUUUA (SEQ ID NO:3), GAACAGCGAUCGUAAUCAA (SEQ ID NO:4), GCAUGUGGAUUCAAAGUUA (SEQ ID NO:5), and GCGAGUACAUCAAGACUUU (SEQ ID NO:6). CUL4B SMARTpool targeting sequences were: UAAAUAACCUCCUUGAUGA (SEQ ID NO:7), CAGAAGUCAUUAAUUGCUA (SEQ ID NO:8), CGGAAAGAGUGCAUCUGUA (SEQ ID NO:9), and GCUAUUGGCCGACAUAUGU (SEQ ID NO:10). Transfections were done using Lipofectamine RNAIMAX (ThermoFisher) according to the manufacturer's protocols. Cells were grown to ˜60% confluence prior to siRNA transfection.

### Example 7—Stable Cell Line Generation

The SMARTvector Inducible Lentiviral shRNA system (Dharmacon) was used to create stable inducible AML cell lines, enabling the regulation of the abundance of CDT1 by Doxycycline (DOX). Lentiviral particles targeting human CDT1 (targeting sequence GATGCTGGGGAGTCCTGCA (SEQ ID NO:11)), GAPDH, and Non-targeting controls were purchased from the manufacturer and used to transduce MV4-11 AML. Transduction and stable cell line selection were performed according to the manufacturer's instructions for suspension cells.

### Example 8—Ligand Binding (MST)

E3 ligase complex (˜2 nmol) was labeled with fluorescent dye using Monolith NT Protein Labeling Kit Red-NHS (Nanotemper) in the presence of 20 mM MES pH 6.0 and 150 mM NaCl. Excess dye was removed using the included gravity flow column and eluted in storage buffer containing 20 mM Tris-HCl, pH 7.4, 150 mM NaCl, and 10% glycerol. The fluorescent E3 complex (˜0.01-0.06 μM) was mixed with increasing amount of compound in a buffer (20 μl) that contains phosphate-buffered saline, 0.1% TWEEN-20 and 2% prionex. Following incubation at room temperature for 30 min, the reaction mixture loaded into premium coated capillaries and analyzed in the Monolith NT.115 machine (Nanotemper). Thermophoresis was recorded for each sample and binding curve was generated and K_{d} was calculated using the included software. For reactions where significant ligand specific fluorescence quenching occurred (verified by manufacturer's SDS Denaturation-test protocol), the initial fluorescence quenching data was used directly to calculate the binding curve and K_{d}.

### Example 9—Ubiquitination Assays

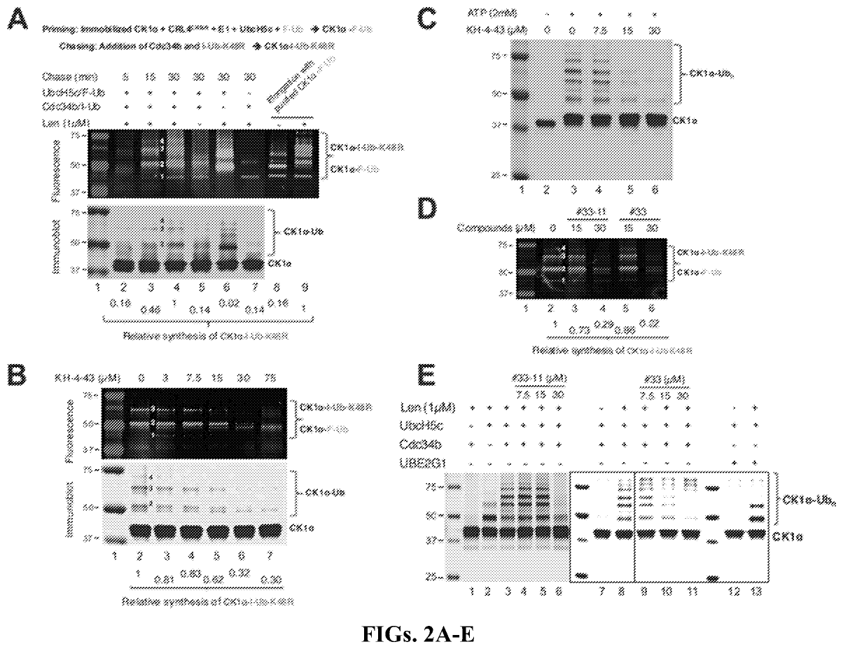

Ubiquitination of CK1α by CRL4^{CBRN} (FIGS. 2A-E)

To prepare Flag-CK1α as a substrate for ubiquitination experiments described in FIGS. 2A-E, the expression vector (purchased from Addgene) was introduced to HEK-293T cells by transfection using Lipofectamine RNAIMAX (ThermoFisher) according to manufacturer's protocols. Cell extracts were prepared and the concentration of Flag-CK1α was determined using immunoblot analysis with purified GST-CK1α (Sigma) as a standard. Typically, 50-100 μl of extracts was used to attach to ˜15 μl of M2 beads (Sigma) at 4° C. for 2 hours. Unbound materials were removed by washing (first three times with a washing buffer containing 50 mM Tris-HCl, pH8.0, 1% Triton X-100, 0.5M NaCl, and then twice with buffer A plus 50 mM NaCl) and the resulting beads were used in the ubiquitination assay (15 μl). The concentration of Flag-CK1α was determined to be ˜100 nM.

Ubiquitination Experiments (FIGS. 2A, 2B, and 2D)

Immobilized, Flag-CK1α-containing beads were initially incubated with a reaction mixture (10 μl) containing 50 mM Tris-HCl (pH7.4), 0.5 mM DTT, 0.1 mg/ml of BSA, 5 mM MgCl₂, 100 nM CRL4^{CRBN}, 1 μM Lenalidomide (Selleckchem), 1 μM Fluorescein-Ub, 100 nM E1, and 1 μM UbcH5c. ATP (20 mM) was added last to initiate the reaction. The reaction was incubated on a thermomixer (1,300 rpm; Eppendorf) at 37° C. for 5 min. The resulting beads were then added with a second reaction mixture (5 μl) containing 50 mM Tris-HCl (pH7.4), 0.5 mM DTT, 0.1 mg/ml of BSA, 5 mM MgCl₂, 4 μM I-Ub-Q31C/K48R, and 1 μM Cdc34b, in the presence or absence of E3 inhibitors as specified. The reaction was incubated on a thermomixer at 37° C. for times as indicated. The concentrations of enzymes, Ubs, and compounds stated above were adjusted based on a final volume of 15 μl. The beads were then washed using conditions as described above. Materials eluted from the beads were separated by 4-20% SDS-PAGE followed by detection with fluorescence scanning (Typhoon 9500) or immunoblot using anti-CK1α antibody (Abcam).

Ubiquitination Experiments (FIGS. 2C and E)

Immobilized, Flag-CK1α-containing beads were incubated with a reaction mixture (15 μl) containing 50 mM Tris-HCl (pH 7.4), 0.5 mM DTT, 0.1 mg/ml of BSA, 5 mM MgCl₂, 100 nM CRL4^{CRBN}, 1 μM Lenalidomide, 10 μM human Ub, 100 nM E1, 1 μM UbcH5c, and 1 μM Cdc34b, in the presence or absence of E3 inhibitors as specified. ATP (20 mM) was added last to initiate the reaction. The incubation was on a thermomixer at 37° C. for 30 min. The beads were then washed using conditions as described above. Materials eluted from the beads were separated by 4-20% SDS-PAGE followed by detection with immunoblot using anti-CK1α antibody.

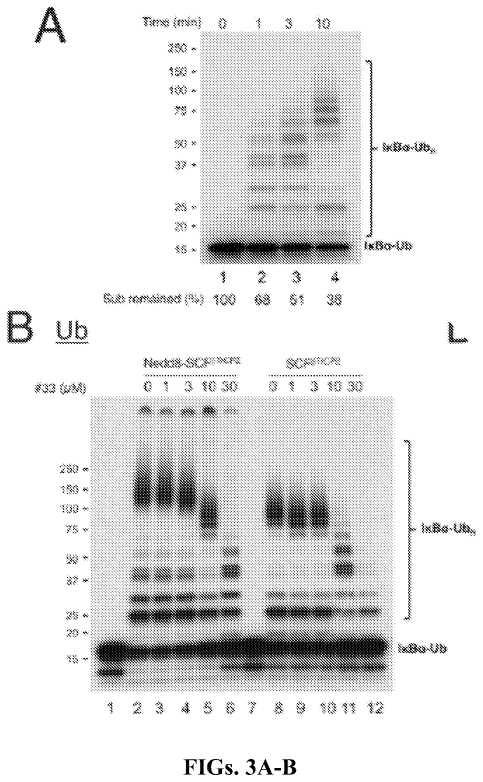

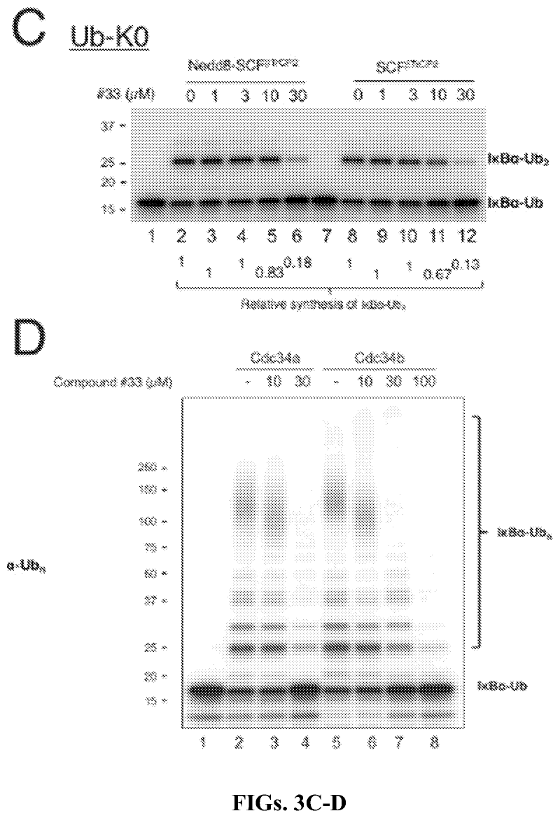

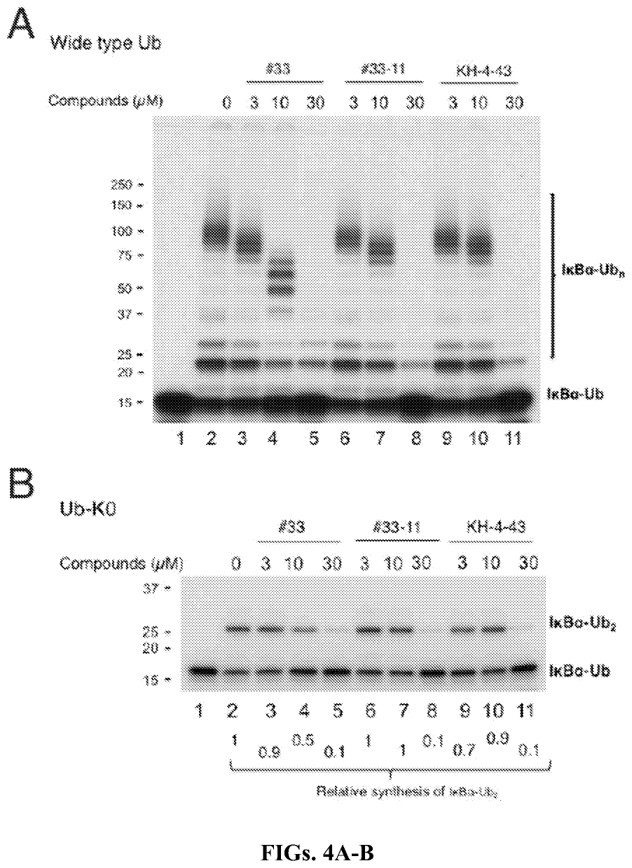

Ubiquitination of ³²P-IκBα-Ub by SCF^{βTrCP} (FIGS. 3A-D and FIGS. 4A-B)

Substrate ³²P-IκBα-Ub was prepared using the previously published protocol (Wu et al., “Priming and Extending: an UbcH5/Cdc34 E2 Handoff Mechanism for Polyubiquitination on a SCF Substrate. Mol. Cell 37:784-796(2010), which is hereby incorporated by reference in its entirety). To assemble Nedd8-SCF^{βTrCP} or SCF^{βTrCP}, Nedd8-ROC1-CUL1 or ROC1-CUL1 (30-100 nM) was mixed with Skp1-βTrCP (30-100 nM) in a reaction mixture containing 50 mM Tris-HCl (pH7.4), 0.5 mM DTT, 0.1 mg/ml of BSA, 5 mM MgCl₂, and 2 mM ATP. The mixture was incubated at room temperature for 10 min. 1.2 pmol of ³²P-IκBα-Ub was added and the mixture was incubated for additional 10 min at room temperature. Compounds, where indicated, were added and the mixture was incubated for another 10 min at room temperature. The E2 charging reaction was assembled in a separate mixture (5 μl) that contained 50 mM Tris-HCl, pH7.4, 5 mM MgCl₂, 2 mM ATP, 0.5 mM DTT, 0.1 mg/ml BSA, Ub (40 μM), E1 (0.1 μM), and Cdc34a or Cdc34b (1 μM). After combining E3-substrate and E2-Ub (in a final volume of 10 μl), the final mixture was incubated at 37° C. for times as indicated. The reaction products were visualized by autoradiography and quantified by phospho-imaging after separation by 4-12% SDS-PAGE.

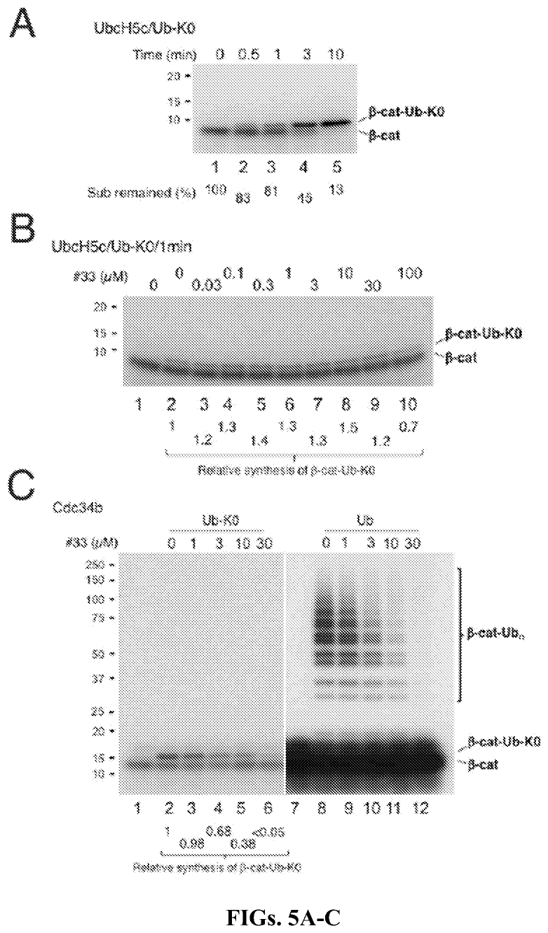

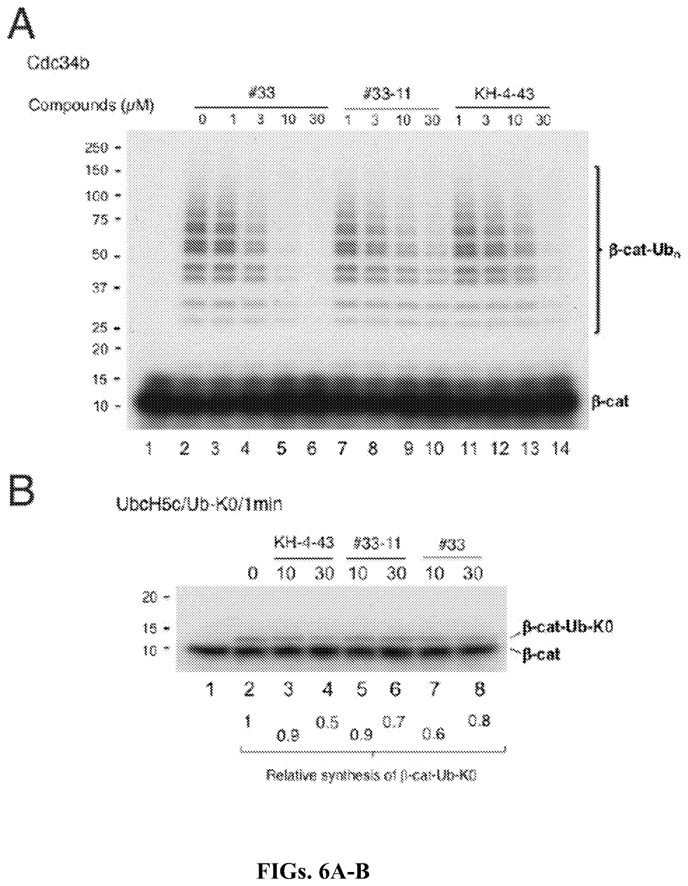

Ubiquitination of ³²P-β-catenin by SCF^{βTrCP} (FIGS. 5A-C and FIGS. 6A-B)

Substrate ³²P-β-catenin was prepared using the previously published protocol (Kovacev et al., A Snapshot at Ubiquitin Chain Elongation: Lysine 48-Tetra-ubiquitin Slows Down Ubiquitination,” J. Biol. Chem. 289(10): 7068-7081(2014), which is hereby incorporated by reference in its entirety). ³²P-β-catenin (2 pmol) was added to the mix containing Nedd8-SCF^{βTrCP} (30 nM) that was assembled as described above. The mixture was incubated at room temperature for 10 min followed by the addition of Na-glutamate to a final concentration of 70 mM (10 μl). Compounds, in amounts as indicated, were added and the mix was incubated for additional 10 min at room temperature. The E2-Ub charging mix was prepared as described above with 1 μM UbcH5c or Cdc34. After mixing the E3-substrate-compound and E2-Ub, the reaction (10 μl) was incubated 37° C. for times as indicated. The reaction products were visualized by autoradiography and quantified by phospho-imaging after separation by 4-12% SDS-PAGE.

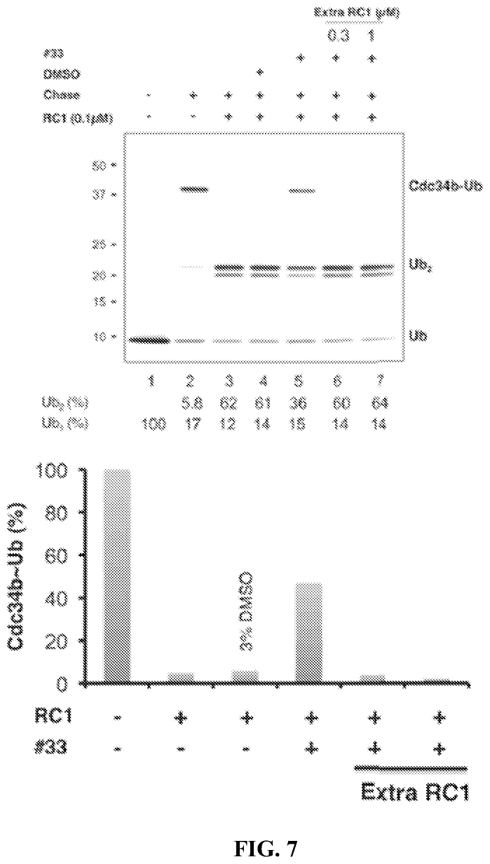

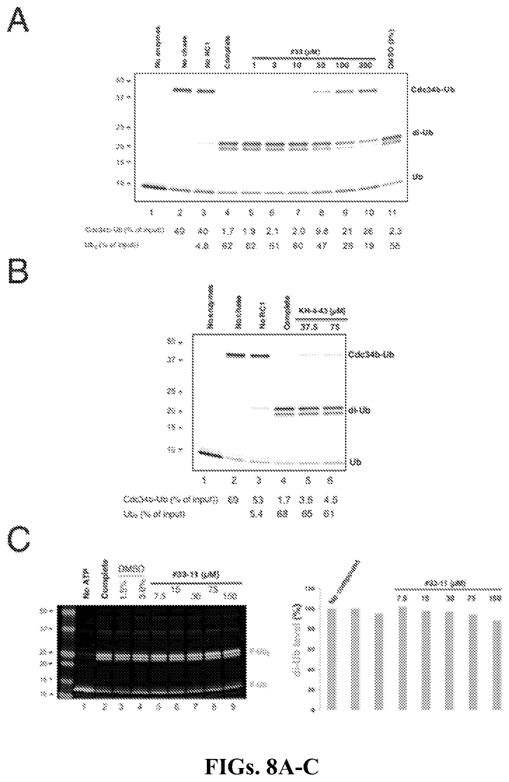

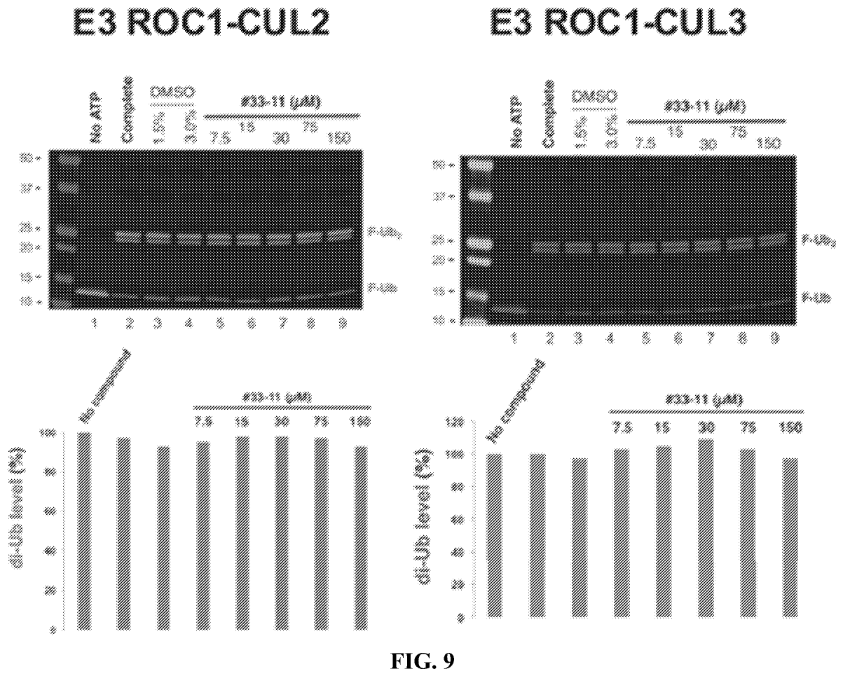

di-Ub Synthesis (FIG. 7, FIGS. 8A-C, FIG. 9)

For the experiment shown in FIG. 7 and FIGS. 8A-B, the di-Ub synthesis reaction was carried out by preassembling the donor Ub mix. The donor Ub mixture (5 μl) contained 50 mM Tris HCl (pH 7.4), 5 mM MgCl₂, 2 mM ATP, 0.5 mM DTT, 0.1 mg/mL BSA, 1 μM Ub-NC-I555, 0.1 μM E1, and 2 μM Cdc34b. The reaction was incubated for 5 min at 37° C. Compounds, in the amounts indicated, were added followed by the addition of ROC1-CUL1 (or related E3 subcomplex) in amounts as indicated and 10 μM PK-Ub (1-74) (as a receptor). The final volume was adjusted to 10 μl. The reaction was incubated for 10 min at 37° C.

For the experiments shown in FIG. 8C and FIG. 9, the reaction was assembled in a 10-μl mixture containing 50 mM Tris HCl (pH 7.4), 5 mM MgCl₂, 0.5 mM DTT, 0.1 mg/mL BSA, 0.05 μM Ub-NC-I555, 2,5 μM PK-Ub (1-74), 0.1 μM E1, 1 μM Cdc34b, and 80 nM ROC1-CUL1 with or without compound. The reaction was initiated by the addition of 2 mM ATP. The products were separated by 4-20% SDS/PAGE and were visualized and quantified on a Typhoon FLA 9500 laser scanner (GE).

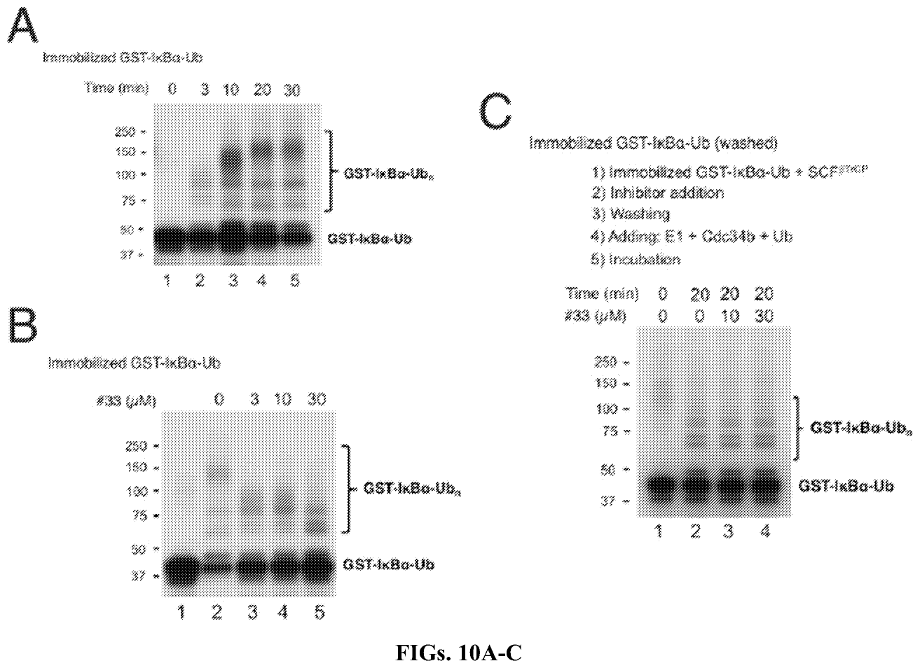

Ubiquitination of Immobilized ³²P-GST-IκBα-Ub by SCF^{βTrCP} (FIGS. 10A-C)

To prepare immobilized, radioactive labeled, phosphorylated GST-IκBα-Ub for ubiquitination experiments, GST-I20-Ub-I23 PK-IκBa (9 μg) was first incubated with Flag-IKKβ (S177E, S181E) (0.5 μg) in a mixture containing 50 mM Tris-HCL pH7.4, 12 mM MgCl₂, 25 μM ATP, γ-[³²P]-ATP, 2 mM NAF, and 50 mM NaCl. The mixture was incubated for 30 min at 37° C. For immobilization, the reaction mixture was mixed with glutathione beads (20 μl) with shacking for 1 hour at 4° C. Unbound materials were removed by washing the beads. Additional glutathione beads (200 μl) were added and stored −20° C.

To perform ubiquitination with immobilized, radioactive labeled, phosphorylated GST-IκBα-Ub, the above beads (10 μl) were incubated with a reaction mixture (10 μl) containing 50 mM Tris-HCl (pH7.4), 0.5 mM DTT, 0.1 mg/ml of BSA, 5 mM MgCl₂, 2 mM ATP, 60 nM Nedd8-ROC1-CUL1, and 60 nM Skp1-βTrCP, on a thermomixer (1,300 rpm; Eppendorf) at room temperature for 10 min. The E2 charging reaction was assembled in a mixture (5 μl) that contained 50 mM Tris-HCl, pH7.4, 5 mM MgCl₂, 2 mM ATP, 0.5 mM DTT, 0.1 mg/ml BSA, Ub (40 μM), E1 (0.1 μM), and Cdc34b (1 μM). After combining E3-substrate and E2-Ub (in a final volume of 15 μl), the final mixture was incubated on a thermomixer (1,300 rpm) at 37° C. for times as indicated. The reaction products were visualized by autoradiography after separation by 4-12% SDS-PAGE.

To perform washout experiments, immobilized GST-IκBα-Ub was first incubated with Nedd8-SCF^{βTrCP2} to form the substrate-E3 complex as described above. Indicated amounts of compound #33 were then added for a brief incubation of 5 min at room temperature on a thermomixer (1,300 rpm). The mixture was then washed once with buffer A plus 50 mM NaCl (200 μl). The resulting beads reacted with pre-charged Cdc34b-Ub mix that contained 50 mM Tris-HCl, pH 7.4, 5 mM MgCl₂, 2 mM ATP, 0.5 mM DTT, 0.1 mg/ml BSA, Ub (40 μM), E1 (0.1 μM), and Cdc34b (1 μM). The final mixture was incubated on a thermomixer (1,300 rpm) at 37° C. for 20 min. The reaction products were analyzed as described above.

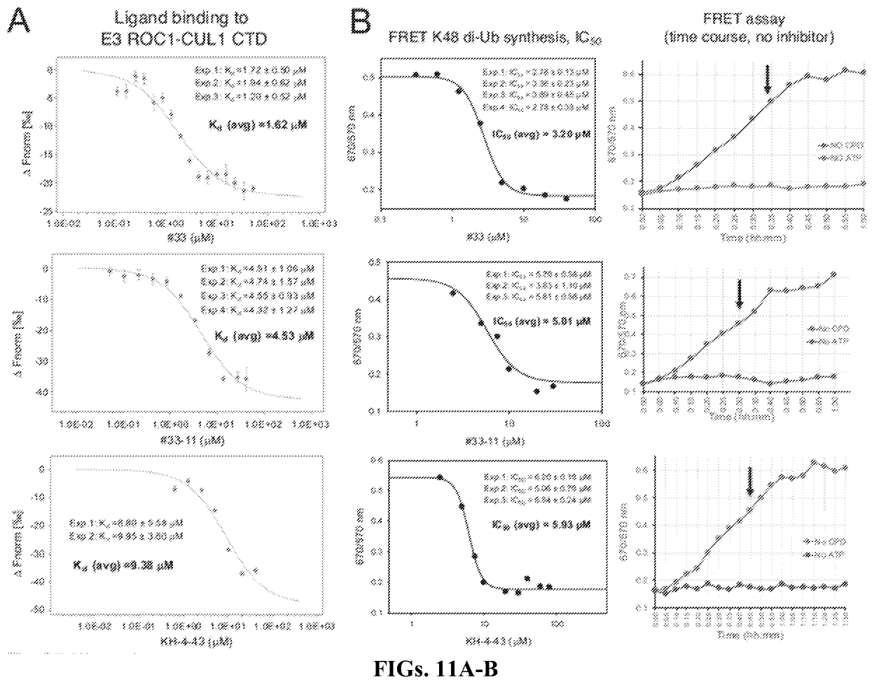

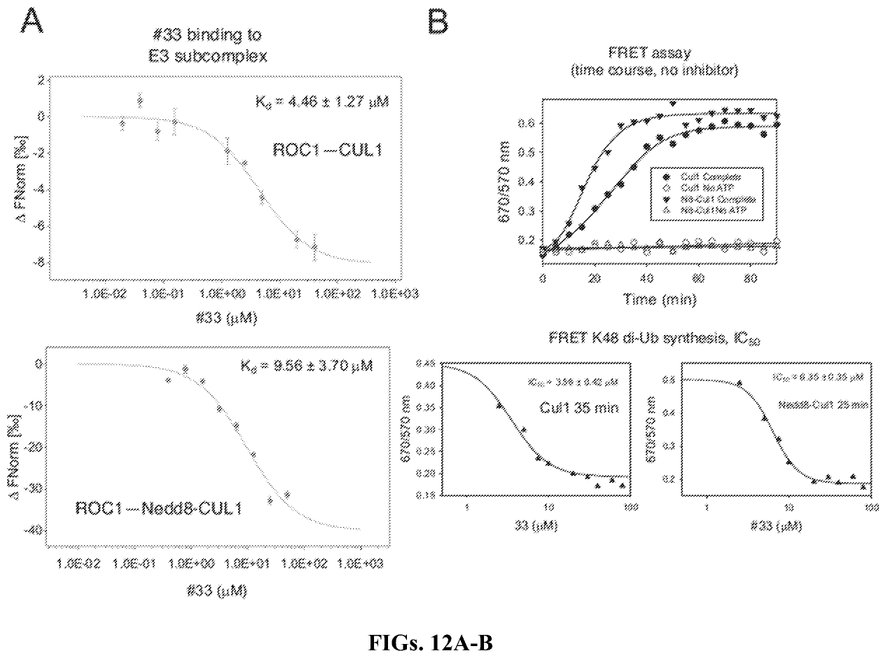

FRET-K48 di-Ub Synthesis (FIGS. 11B and 12B)

The reaction mixture (15 μl) was assembled onto a 384-well microtiter plate. Each well contained 33 mM Tris-HCl (pH 7.4), 1.7 mM MgCl₂, 0.33 mM DTT, BSA (0.07 mg/ml), Ub E1 (14 nM), E2 Cdc34 (124 nM), E3 ROC1-CUL1 CTD complex (1 μM), Ub C31-iFluor 555 (donor, 0.93 μM), and Ub C₆₄-iFluor 647 (receptor, 1.62 μM), in the presence or absence of compound. ATP (0.66 mM) was then added to the mix followed by a brief centrifugation to settle down the mixture. The resulting plate was incubated at 30° C. in the Synergy-H₁ reader and the fluorescence signal was monitored. Ubiquitination was quantified based on the ratio of acceptor:donor fluorescence (excitation 515 nm; donor emission 570 nm, acceptor emission 670 nm).

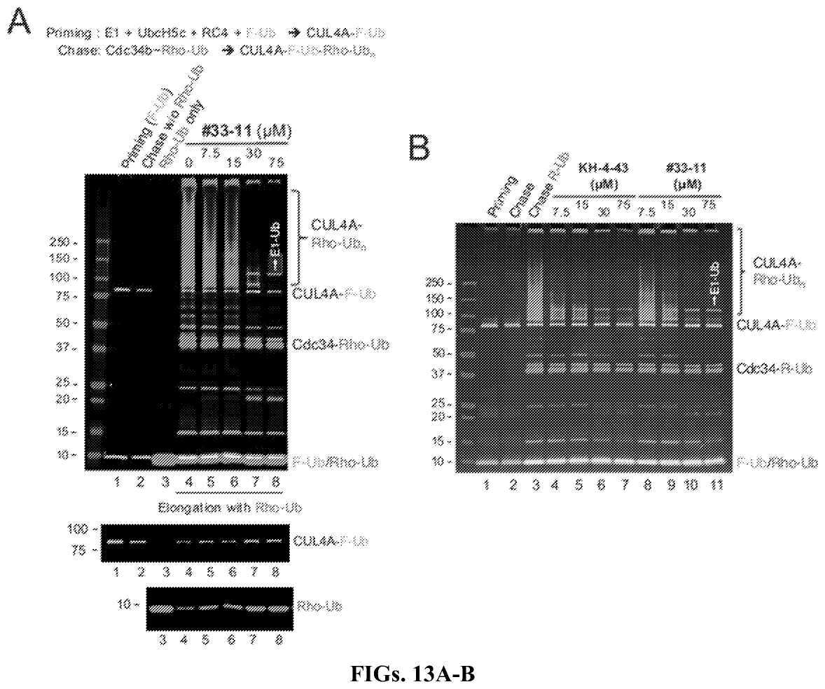

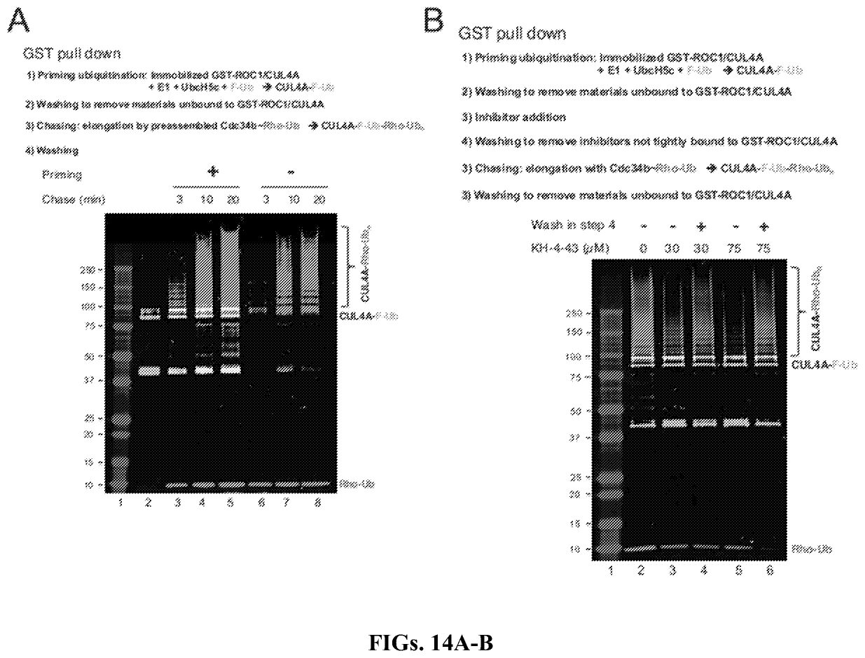

Ubiquitin Chain Elongation by E3 ROC1-CUL4A Detected by Fluorescence (FIGS. 13A-B)

First, a mono-ubiquitination reaction mixture (5 μl) was prepared to contain 50 mM Tris-HCl (pH 7.4), 0.5 mM DTT, 0.1 mg/ml of BSA, 5 mM MgCl₂, 2 mM ATP, 2 μM Fluorescein-Ub (F-Ub), 0.2 μM E1, 2 μM E2 UbcH5c, and 5.2 μM E3 ROC1-CUL4A complex. The reaction was incubated for 5 min at 37° C. A separate, Cdc34b-Ub thiol ester-generating reaction mixture (5 μl) was prepared to contain 50 mM Tris-HCl (pH 7.4), 0.5 mM DTT, 0.1 mg/ml of BSA, 5 mM MgCl₂, 2 mM ATP, 1 μM Rhodamine-Ub (R-Ub), 3 μM unlabeled Ub, 0.1 μM E1, and 1 μM E2 Cdc34b. The reaction was incubated for 5 min at room temperature. In the second step (chase), the completed Cdc34b-Ub thiol ester reaction mixture was combined with one third of the completed mono-ubiquitination mixture. The final volume was adjusted to 10 μl with a mix that contained 50 mM Tris-HCl (pH 7.4), 0.5 mM DTT, 0.1 mg/ml of BSA, 5 mM MgCl₂, and 2 mM ATP. Compounds were added where indicated. The chase reaction was incubated for 10 min at 37° C. The reaction products were separated by 4-20% SDS-polyacrylamide gel electrophoresis.

Ubiquitination of Immobilized GST-ROC1-CUL4A (FIGS. 14A-B)

To prepare immobilized GST-ROC-CUL4A for ubiquitination experiments, bacterial extracts (70 μl) containing GST-ROC-CUL4A were adsorbed to glutathione beads (20 μl). Unbound materials were removed by washing the beads three times with washing buffer (50 mM Tris-HCl, pH 8.0, 1% Triton X-100, 0.5 M NaCl, and then twice with buffer A plus 50 mM NaCl) and then twice with buffer A plus 50 mM NaCl. The concentration of bound GST-ROC1-CUL4A was determined to be ˜3 μg (translated to 2.5 μM in a 10 μl reaction).

To perform ubiquitination with immobilized GST-ROC1-CUL4A, the above beads (20 μl) were incubated with a reaction mixture (10 μl) containing 50 mM Tris-HCl (pH 7.4), 0.5 mM DTT, 0.1 mg/ml of BSA, 5 mM MgCl₂, 2 mM ATP, 1 μM Fluorescein-Ub, 0.1 μM E1, and 1 μM UbcH5c, on a thermomixer (1,300 rpm) at 30° C. for 5 min. The beads were washed once with the washing buffer as described above, and once with buffer A plus 50 mM NaCl. The washed beads were incubated with a second mixture (10 μl) that contained 50 mM Tris-HCl, pH 7.4, 5 mM MgCl₂, 2 mM ATP, 0.5 mM DTT, 0.1 mg/ml BSA, human Ub (3 μM), Rhodamine Ub (1 μM), E1 (0.1 μM), and Cdc34b (1 μM). The incubation was on a thermomixer (1,300 rpm) at 37° C. for times as indicated. The reaction products were visualized by fluorescence scanning (Typhoon 9500) after separation by 4-12% SDS-PAGE.

To perform washout experiments, immobilized GST-ROC1-CUL4A was first mono-ubiquitinated with the Fluorescein Ub/E1/UbcH5c mix and then washed as described above. Indicated amounts of compound KH-4-43 were added. Where indicated, the mixture was washed once with the washing buffer as described above, and once with buffer A plus 50 mM NaCl. The resulting beads were then incubated with the Rhodamine Ub/E1/Cdc34b mix as described above in a final volume of 10 μl. The final mixture was incubated on a thermomixer (1,300 rpm) at 37° C. for 10 min. The reaction products were analyzed as described above.

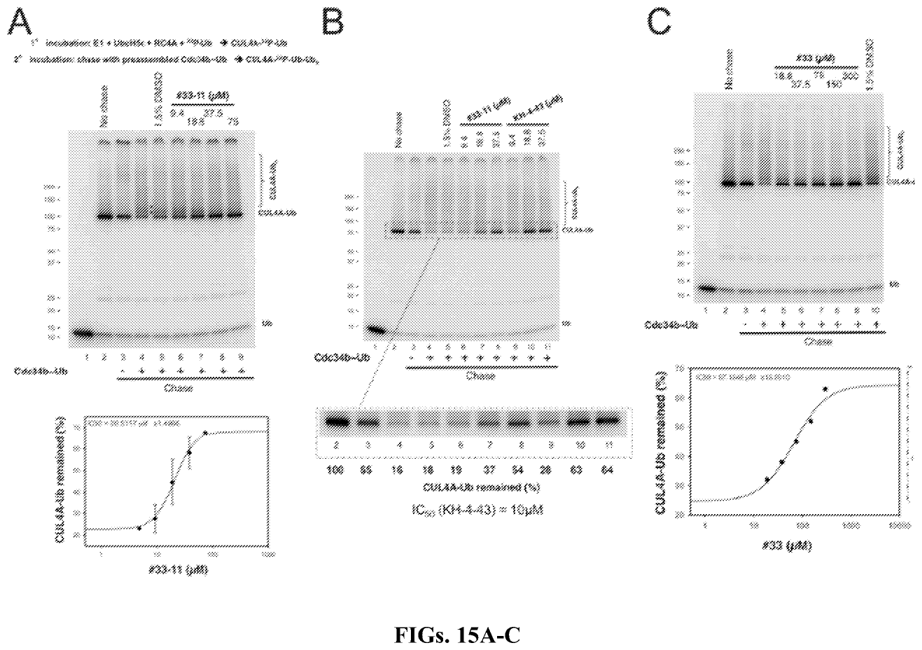

Ubiquitin Chain Elongation by E3 ROC1-CUL4A Detected by Autoradiography (FIGS. 15A-C)

The reaction scheme was identical to the one described above for FIGS. 12A-B. The exceptions were that 2 μM ³²P-PK-Ub or 100 μM Ub was used in lieu of F-Ub or R-Ub, respectively. The reaction products, separated by SDS-PAGE, were visualized and analyzed by phospho-imaging using Typhoon 9500 scanner.

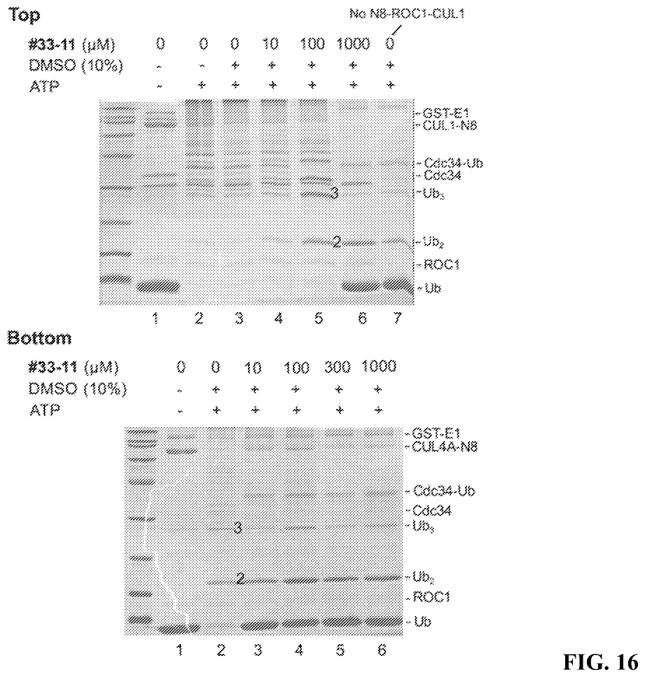

Ub Chain Assembly Measured by Coomassie Stain (FIG. 16)

Reaction mixture contained 2 mM ATP, 10 mM MgCl₂, 0.01 μg/μl of GST-E1 (Ub), 0.01 μg/μl of Cdc34, 0.1 μg/μl of Nedd8-ROC1-CUL1 or Nedd8-ROC1-CUL4A, and 1 μg/μl of Ub. Where indicated, DMSO and compound were added. The reaction was incubated at 37° C. for 60 min. Following separation of reaction products by SDS-PAGE, proteins were visualized by Coomassie stain.

### Example 10—Compound Treatment and Immunoblot Analysis

Cells (6×10⁵) were seeded 24 hours before compound treatment. Compound was added to the culture media and cells were cultured with the compound for 48 hours. To harvest the treated cells, the cells were pelleted at 180×g for 5 min with a Beckman CS-6KR centrifuge at 4° C. and washed with 5 ml of phosphate-buffered saline. The washed cells were pelleted and re-suspended in 0.1 ml per dish of lysis buffer (50 mM Tris-HCl [pH 7.4], 150 mM NaCl, 2 mM MgCl₂, 10% glycerol, 1% Triton X-100, 1 mM DTT, 50 units/ml of Benzonase, 1 mM phenylmethylsulfonyl fluoride, 2 μg of antipain per ml, and 2 μg of leupeptin per ml). The resulting suspension was sonicated with three repetitive 20-s treatments. The mixture was agitated for 30 min at 4° C. and then centrifugated (at 16,873×g at 4° C. for 30 min). Supernatants were saved. Extracts were run on 4-20% Tris-glycine gels, then transferred to membranes using standard Western blotting procedures. After blocking, the membranes were incubated with primary antibodies, which were typically diluted in solution (10 ml) containing PBS and 0.5% gelatin. The incubation was typically carried out overnight at 4° C. on a shaker. Following wash with PBS and 0.05% TWEEN-20, the membrane was incubated with secondary antibodies (1:10,000 dilution in PBS and 0.05% TWEEN-20) that carry a fluorophore. Antibody-reactive protein(s) was visualized and quantified using the LI-COR Odyssey IR Imaging System (LI-COR Biosciences, Lincoln, NE).

### Example 11—Compound Treatment and Annexin V Flow Cytometry

Cells (5×10⁴) were seeded 24 hours before compound treatment. Compound was added to the culture media and cells were cultured with the compound for 48 hours. The treated cells were then transferred into 5 ml flow cytometry tubes and the well was washed once with PBS to collect any remaining cells. The cells were then pelleted at 500×g for 5 min with a Beckman CS-6KR centrifuge at 4° C. After removal of supernatant, the cells pellet was washed with Annexin V binding buffer (10 mM Hepes-NaOH, pH 7.4, 150 mM NaCl, 5 mM KCl, 1 mM MgCl₂, and 1.8 mM CaCl₂). Cells were pelleted once again and incubated in the dark with Annexin V-FITC (2 μg/ml) in binding buffer for 5 min then analyzed using LSRFortessa flow cytometer (BD).

### Example 12—CellTiter-Glo® 2.0 Assay

For cell viability assay, 10,000 cells (per well) were seeded in 96 well plates in 100 microliters of media and incubated overnight. Serial dilutions of compounds tested were then added to wells in triplicate. The cells were incubated for an additional 48 hours. For analysis, 100 microliters of CellTiter-Glo reagent was added per well and the plate was placed on a shaker for 15 minutes. The luminescence of each well was then read using a Synergy multi-mode plate reader (BioTek). For determining relative luminescence, the background (average reading of wells containing only media and CellTiter-Glo reagent, no cells) was first subtracted from the reading of each sample. Then, the background subtracted values was divided by the average value of DMSO treated control wells. The average of the triplicates was plotted using SigmaPlot 10 software (Systat). Then EC₅₀ was determined using the four parameter logistics regression standard curve.

### Example 13—ADME Studies

ADME studies on compounds #33-11 and KH-4-43 for “absorption, distribution, metabolism, and excretion” were carried out at CRO, BioDuro. Kinetic solubility, plasma and microsome stability in rat, mouse and human, as well as Caco-2 Permeability were examined.

### Example 14—Mouse IP PK

Mouse IP PK experiments on compounds #33-11 and KH-4-43 were carried out at CRO, BioDuro.

### Example 15—Mouse Tolerance/Toxicity Study

Mouse MTD experiments on compound #33-11 were carried out at CRO, BioDuro (FIG. 10A). Female NU/NU mice at age of 6-8 week were used. Mice were obtained from Beijing Vital River Laboratory Animal Technology Co., Ltd. #33-11 (50 or 100 mg/kg) was formulated in 100% DMSO and the injection volume was 50 μl. Three treatment groups include G1 (vehicle only), G2 (#33-11, 50 mg/kg), and G3 (#33-11, 100 mg/kg). Each group had three mice. Compound was administrated by IP daily (QD) for seven days. Body weight was measured every day. The treated mice were observed daily for features including appearance (hair coat, discharges, injury or lesion and eye), as well as behavior/condition (gait, activity, Central Nervous System, respiration and feces).

### Example 16—Tumor Mouse Xenograft Assay

Mouse MV4-11 xenograft model studies were carried out at CRO, BioDuro.

Animals and Materials

Female NU/NU mice at age of 6-8 weeks with body weight no less than 20 grams at study starting point. Mice were obtained from Beijing Vital River Laboratory Animal Technology Co., Ltd. KH-4-43 (25 or 50 mg/kg) was formulated in 100% DMSO and the injection volume was 50 μl. Sorafenib (3 mg/kg) was formulated in PBS by vortexing and sonication. The MV-4-11 tumor cells were cultured in IMDM medium supplemented with 10% heat inactivated fetal bovine serum, 100 U/ml penicillin, and 100 μg/ml streptomycin at 37° C. in an atmosphere of 5% CO₂ in air. The tumor cells were routinely sub-cultured twice weekly. The cells growing in an exponential growth phase were harvested and counted for tumor inoculation.

Tumor Inoculation and Grouping

Each mouse was inoculated subcutaneously at the right flank with the MV-4-11 tumor cells (1×10⁷) in 0.1 ml growth media mixed with 50% matri-gel for tumor development. The tumor cell inoculation day is Day 0 for this study. When the tumor volume reached approximately 150 mm³, animals were randomly grouped into 5 groups based on the animal body weight and tumor volume. Each group had 10 tumor-bearing mice.

Dosing

The first dosing day was day 19 and last dosing day was day 42 with the tumor cell inoculation at day 0. KH-4-43 was administrated by IP, daily (QD) or every two days (Q2D) (Table 4). The Sorafenib treated group was administrated by PO, QD.

Tumor Measurement and the Endpoints

Tumor volume was measured three times weekly in two dimensions using a caliper, and the volume was expressed in mm³ using the formula:

V=0.5a×b² (Eq.1)

where a and b are the long and short diameters of the tumor, respectively. The tumor volume was then used for calculations of T/C values. The T/C value (in percent) was the ratio of T and C, and it is an indication of antitumor effectiveness; T and C were the mean volume of the treated and control groups, respectively, on a given day. TGI value was calculated as follows:

TGI (%)=(Vc−Vt)/(Vc−Vo)*100 (Eq.2)

where Vc and Vt are the median of the control and treated groups at the end of the study and Vo at the start.

Tumor tissues were weighed at the end of study. At the endpoint, 100 μl blood and tumors from Group 3 (G3) and Group 4 (G4) were collected as shown in the Table 3, for further pharmacokinetics or biomarker analysis. Plasma were isolated from all blood samples.

TABLE 3
Blood and Tumors from Group 3 and Group 4

| plasma | 7.5 min | 30 min | 2 hours | 8 hours | 24 hours |
|---|---|---|---|---|---|
| G3-KH-4-43 - | 10 | 10 | 10 | 10 | 10 |
| 50 mk/kg (Q2D) | animals | animals | animals | animals | animals |
| tumor | 2 hours | 8 hours | 24 hours | | |
| G4-KH-4-43 - | 3 | 3 | 3 | | |
| 50 mk/kg (QD) | animals | animals | animals | | |

Mouse Handling, Care, and Observations

All the procedures related to animal handling, care, and the treatment were performed according to the guidelines approved by the Institutional Animal Care and Use Committee (IACUC) of BioDuro following the guidance of the Association for Assessment and Accreditation of Laboratory Animal Care (AAALAC). At the time of routine monitoring, the animals were checked for any effects of tumor growth and treatments on normal behavior such as mobility, food and water consumption (by looking only), and body weight gain/loss (body weights were measured every other day), eye/hair matting and any other abnormal effect. Death and observed clinical signs were recorded.

Statistical Analysis

A two-way ANOVA was performed to compare body weight and tumor volume. A one-way ANOVA was performed to compare tumor weight on the terminated day. All the data was analyzed using GraphPad Prism 5. P<0.05 was statistically significant.

### Example 17—COMT Assay

COMT assay with compound #33-11 was carried out. Reaction contained 50 mM Tris-HCl (pH 7.5), 5 mM MgCl₂, 0.002% TWEEN20, 0.005% BSA, 1 mM DTT, 1% DMSO, 20 nM of human recombinant full-length s-COMT, 0.5 mM of substrate COMT-S01, and 1 mM S-Adenosyl-L-[methyl-³H] methionine (3H-SAM) as methyl donor. An increasing amount of #33-11 or Tolcapone (positive control) was mixed with all reaction components without ³H-SAM and incubated for 15 min at room temperature. 3H-SAM was added to initiate the reaction. The reaction was incubated for 2 hours at 30° C. and then terminated by the addition of 50 mM EDTA (final conc.). The reaction mixture was transferred into FlashPlate and read by TopCount. Data was analyzed using Excel and GraphPad Prism software for IC₅₀ curve fits.

### Example 18—Results of Examples 1-17

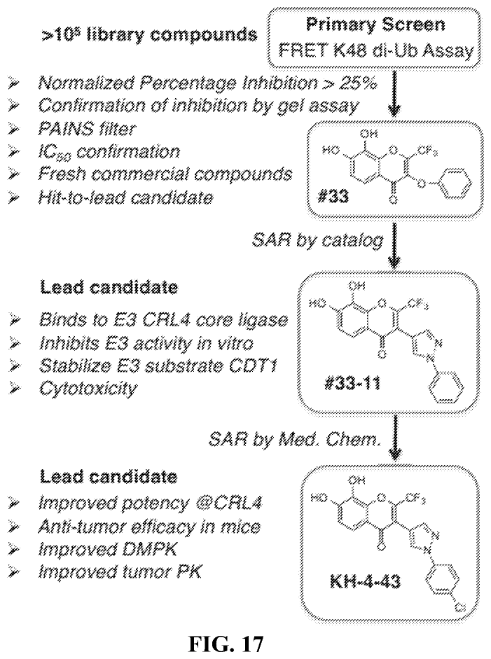

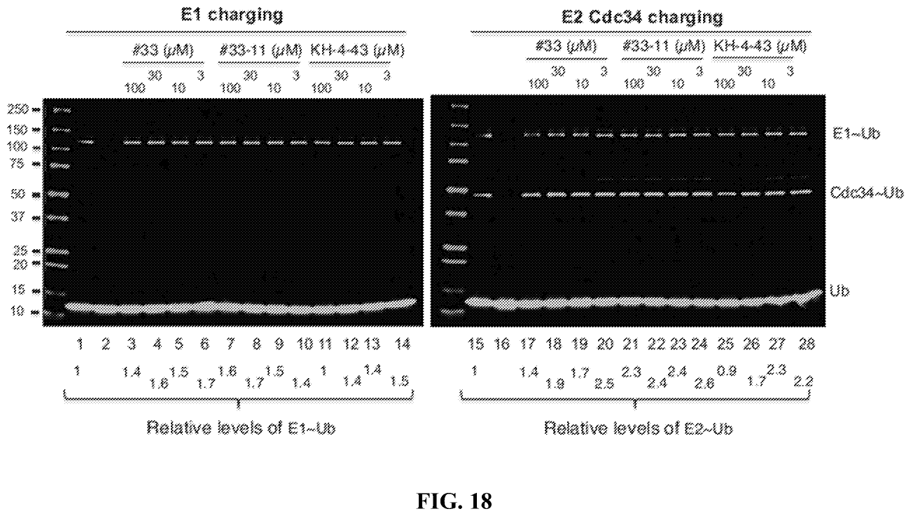

The novel high throughput screen (HTS) of >10⁵ compounds and subsequent follow-up characterization studies have identified hit compound #33 (FIG. 17) as an inhibitor of E3 CRL1/SCF using multiple assays including reconstituted substrate ubiquitination (FIGS. 3A-D and FIGS. 5A-C) and K48 di-Ub synthesis (FIG. 7). Collectively the results revealed the inhibitory effects of #33 on the ubiquitination of E3 SCF^{βTrCP2} substrates IκBα (FIGS. 3A-D) and β-catenin (FIGS. 5A-C), and on di-Ub chain assembly catalyzed by the E3 subcomplex ROC1-CUL1 CTD (FIG. 7), without significantly affecting Ub thiol ester formation with E1 and E2 Cdc34a (FIG. 18). Compound #33 exhibited ability to: 1) inhibit ubiquitination of IκBα by SCF^{βTrCP2} with E2 Cdc34a or Cdc34b equally (FIG. 3D); 2) block the activity of SCF^{βTrCP2} in either Nedd8-modified or unmodified form, with the unmodified E3 appearing slightly more sensitive to the compound (FIGS. 3B-3C); and 3) inhibit ubiquitination reactions by E2 Cdc34 more potently than those by E2 UbcH5c (FIGS. 5B and 5C). In addition, the results of immobilization experiments showed that the inhibitory effects of #33 were diminished by washing the pre-assembled E3-inhibitor complex (FIGS. 10A-C), suggesting that the compound acts in a reversible manner.

Subsequent structural-activity relationship (SAR) studies using both SAR-by-catalog and medicinal chemistry have led to the discovery of lead E3 CRL4 inhibitors #33-11 and KH-4-43 (FIG. 17), respectively.

KH-4-43/#33-11 Bind to E3 ROC1-CUL4A CTD

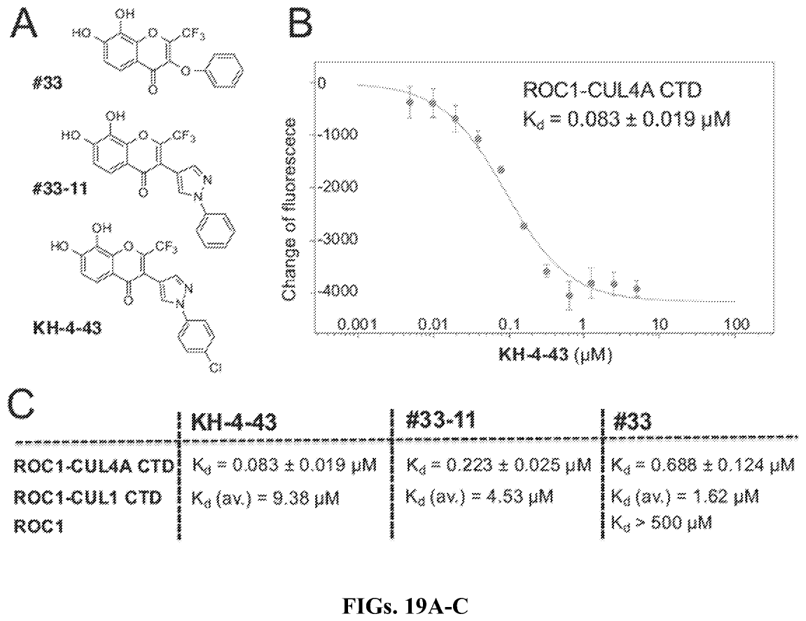

The ligand-target interactions were analyzed using Micro-Scale Thermophoresis (MST), which measures the motion of molecules along microscopic temperature gradients and detects changes in their hydration shell, charge or size (Seidel et al., “Microscale Thermophoresis Quantifies Biomolecular Interactions Under Previously Challenging Conditions,” Methods 59(3):301-315 (2013), which is hereby incorporated by reference in its entirety). MST allows analysis of binding interaction events directly in solution without the need of immobilization to a surface. KH-4-43, #33-11 and #33 directly bind to the purified E3 ROC1-CUL4A CTD complex with a K_{d} of 83, 223, or 688 nM, respectively (FIGS. 19B-C). By comparison, KH-4-43, #33-11 and #33 bind to the purified, highly related E3 ROC1-CUL1 CTD complex with a K_{d} of 9.4, 4.5, or 1.6 μM, respectively (FIG. 11A and FIG. 19C). Thus, while #33 binds to ROC1-CUL4A-CTD and ROC1-CUL1-CTD with comparable affinity (˜0.7 vs 1.6 μM in K_{d}), KH-4-43 and #33-11 bind to ROC1-CUL4A-CTD about 100- or 20-fold more effectively than ROC1-CUL1-CTD. In addition, #33 exhibits virtually no binding activity to ROC1 alone with a K_{d} of >500 μM (FIGS. 19A-C). Thus, the KH-4-43/#33-11-based scaffold binds selectively to ROC1-CUL4A CTD. The MST binding data also suggest that the pyrazole ring substitution of #33-11/KH-4-43 enhances the selective binding to ROC1-CUL4A CTD over the oxygen atom linker of #33 to pendant phenyl group (FIG. 19A).

Furthermore, 1) #33-11/KH-4-43 showed little effects on Ub thiol ester formation with E1/E2 Cdc34 (FIG. 18); 2) in keeping with observations that #33 binds to ROC1-CUL1 CTD more effectively than #33-11/KH-4-43 (FIG. 11A), #33 was found more potent than #33-11/KH-4-43 in inhibiting FRET K48 di-Ub synthesis catalyzed by the E3 subcomplex ROC1-CUL1 (FIG. 111B) (this effect was further confirmed by the results of gel-based di-Ub synthesis experiments (FIG. 8)); and 3) #33 binds to ROC1-CUL1 more effectively than ROC1-CUL1-Nedd8 (FIG. 12A), and showed ability to inhibit ubiquitination by ROC1-CUL1 more potently than that by ROC1-CUL1-Nedd8 (FIG. 12B). These results are consistent with FIGS. 2B-C, supporting that modification of CUL1 by Nedd8 renders the E3 slightly less sensitive to compound #33.

KH-4-43/#33-11 Inhibit Ubiquitination by E3 CRL4 in vitro

Previous studies have established lenalidomide-dependent ubiquitination of CK1α by E3 CRL4^{CRBN} in vivo and in vitro (Kronke et al., “Lenalidomide Induces Ubiquitination and Degradation of CK1α in del(5q) MDS,” Nature 523(7559):183-188 (2015); Petzold et al., “Structural Bbasis of Lenalidomide-Induced CK1α Degradation by the CRL4^{CRBN} Ubiquitin Ligase,” Nature 532(7597):127-130 (2016), which are hereby incorporated by reference in their entirety). To evaluate and quantify inhibitory effects of KH-4-43 on CRL4, a novel biochemical assay was developed to monitor CK1α ubiquitination with UbcH5c as a priming E2, and Cdc34b as an elongating E2 (FIG. 2A, top scheme). This approach utilizes a two-step reaction employing two Ub molecules labeled with distinct fluorophores, Fluorescein-Ub (F-Ub) and iFluor555-Ub-Q31C-K48R (I-Ub-K48R; ref. 5), respectively. After a brief incubation of the immobilized Flag-CK1α with CRL4^{CRBN}, lenalidomide, F-Ub, E1 and E2 UbcH5c, E2 Cdc34b and I-Ub-K48R were then added for further incubation followed by an extensive washing step to remove materials unbound to Flag-CK1α. Ub-K48R was used because it allows E2 Cdc34b to attach only one Ub moiety to a receptor Ub that is linked to CK1α, thereby facilitating quantification. Fluorescence imaging showed time dependent accumulation of CK1α-I-Ub-K48R conjugates (FIG. 2A, upper, lanes 2-4; red-colored) that in size, corresponded to the substrate modified by two or three Ub moieties. These products were significantly diminished in the absence of lenalidomide (lane 5) or UbcH5c/F-Ub (lane 7), demonstrating the dependency on both lenalidomide, which is required for E3-substrate interactions (Petzold et al., “Structural Bbasis of Lenalidomide-Induced CK1α Degradation by the CRL4^{CRBN} Ubiquitin Ligase,” Nature 532(7597):127-130 (2016), which is hereby incorporated by reference in its entirety), and UbcH5c, which is necessary for priming the ubiquitination (Wu et al., “Priming and Extending: An UbcH5/Cdc34 E2 Handoff Mechanism for Polyubiquitination on a SCF Substrate,” Mol. Cell 37:784-796 (2010), which is hereby incorporated by reference in its entirety). Omission of E2 Cdc34b/I-Ub-K48R abolished the formation of CK1α-I-Ub-K48R conjugates, but instead accumulated CK1α-F-Ub (lane 6, green-colored). To unequivocally determine whether CK1α-F-Ub can be utilized by CRL4^{CRBN}/Cdc34b to form CK1α-I-Ub-K48R, preformed CK1α-F-Ub was purified and subjected to ubiquitination reaction with freshly added E3/E2/Ub reagents. The results showed lenalidomide-dependent formation of CK1α-I-Ub-K48R (red), concomitantly with the disappearance of CK1α-F-Ub (green) (FIG. 2A, lanes 8 and 9). The reaction products revealed by fluorescence scanning were verified by immunoblot analysis (FIG. 2A, bottom). Collectively, these data demonstrated that CK1α is ubiquitinated by sequential actions of UbcH5c and Cdc34b: the substrate is first mono-ubiquitinated by UbcH5c followed by Ub chain elongation by Cdc34b. This mechanism closely resembles the ubiquitination of IκBα by E3 SCF^{βTrCP} (Wu et al., “Priming and Extending: An UbcH5/Cdc34 E2 Handoff Mechanism for Polyubiquitination on a SCF Substrate,” Mol. Cell 37:784-796 (2010), which is hereby incorporated by reference in its entirety).

Using the above assay, the effects of KH-4-43 were determined, which was added in the elongation phase of the reaction along with E2 Cdc34b/I-Ub-K48R. The results of fluorescence imaging or immunoblot analysis revealed the ability of KH-4-43 to inhibit the formation of CK1α-I-Ub-K48R conjugates in a dose-dependent fashion (FIG. 2B). To confirm this effect, KH-4-43 was subjected to CK1α ubiquitination with non-fluorescent, wild type Ub to allow Ub chain formation, and both UbcH5c and Cdc34b added simultaneously. The reaction products were detected by immunoblot. As shown, this reaction scheme supported Ub chain formation on CK1α that required ATP (FIG. 2C, lanes 2 and 3), UbcH5c and Cdc34b (FIG. 2E, lanes 1-3), as well as lenalidomide (FIG. 2E, lanes 7 and 8). KH-4-43 was able to block the formation of Ub chains on CK1α in a concentration-dependent manner (FIG. 2C).

The effects of compounds #33-11 and #33 on the ubiquitination of CK1α by CRL4^{CRBN} were examined using both the fluorescence assay with I-Ub-K48R (FIG. 2D) and straight immunoblot experiment with the wild type Ub (FIG. 2E). Significant inhibitory effects by #33-11 and #33 were observed at 30 μM. Comparison of the effects by KH-4-43, #33-11, and #33 suggests that KH-4-43 was the most potent among three compounds, exhibiting inhibition at lower dose ranges of 7.5 and 15 μM (FIGS. 2B-E). The immobilization procedure used in these experiments inevitably causes significant variations, which preclude precise determination of the quantitative difference between these compounds. Despite this limitation, the above results unequivocally establish that KH-4-43/#33-11/#33 inhibit the ubiquitination of CK1α by CRL4^{CRBN} in vitro.

Recent studies have shown that UBE2G1 works as an elongating E2 in the ubiquitination of E3 CRL4^{CRBN} substrates include IKZFs and GSPT1 (Lu et al., “UBE2G1 Governs the Destruction of Cereblon Neomorphic Substrates,” Elife 7, pii: e40958 (2018); Sievers et al., “Genome-Wide Screen Identifies Cullin-RING Ligase Machinery Required for Lenalidomide-Dependent CRL4CRBN Activity,” Blood 132(12):1293-1303 (2018), which are hereby incorporated by reference in their entirety). When UBE2G1 was used in lieu of Cdc34b for the ubiquitination of CK1α by CRL4^{CRBN}, UBE2G1 was found to support ubiquitination poorly (FIG. 2E, lanes 3, 8, and 13). Data quantification suggests that in this case, Cdc34b was ˜4-fold more effective than UBE2G1 in converting the UbcH5c-primed mono-Ub to longer Ub chains.

To more precisely determine the compounds' inhibitory effects on E3 CRL4, a newly developed “CUL4A-Ub elongation assay” was employed that measures ubiquitination catalyzed by the CRL4 core ligase complex ROC1-CUL4A (FIG. 13A). Both KH-4-43 and #33-11 were able to inhibit this ubiquitination reaction, with KH-4-43 appearing more potent than #33-11 (FIG. 13B). In addition, the results of immobilization experiments showed that the inhibitory effects of compound KH-4-43 were diminished by washing the preformed ROC1-CUL4A/inhibitor complex (FIGS. 14A-B), suggesting that the compound acts reversibly. To aid quantitative analysis, a ³²P-Ub chain elongation by ROC1-CUL4A assay was developed (FIGS. 15A-C). The results revealed the order of compound's inhibitory strength against ROC1-CUL4A as: KH-4-43 (IC₅₀ of 10 μM), #33-11 (IC₅₀ of 21 μM), and #33 (IC₅₀ of 67 μM).

The results of additional experiments shed lights into the properties of KH-4-43/#33-11/#33. First, side-by-side comparison found #33-11/KH-4-43 less active than #33 in inhibiting the ubiquitination of E3 SCF^{βTrCP2} substrates IκBα (FIGS. 4A-B) and β-catenin (FIGS. 6A-B). Consistent with these observations, #33-11/KH-4-43 were less potent than #33 in inhibiting di-Ub chain assembly by ROC1-CUL1 (FIGS. 8A-C and FIG. 11B) and showed no inhibition of reactions by ROC1-CUL2/ROC1-CUL3 (FIG. 9). In addition, #33-11 inhibited ubiquitination mediated by Nedd8-ROC1-CUL4A more effectively than Nedd8-ROC1-CUL1 (FIG. 16). CUL5 is the only major canonical cullin that was not tested, however, CUL5 is highly similar to CUL2. It is thus clear that #33-11 and KH-4-43 are less effective than #33 in inhibiting ubiquitination by the CUL1-based E3 CRL1/SCF. By contrast, KH-4-43/#33-11 effectively inhibit ubiquitination by CRL4 (FIGS. 2A-E, FIGS. 13A-B, and FIGS. 15A-C). Combining with the binding data that show KH-4-43 and #33-11 bind to ROC1-CUL4A CTD 100 or 20-fold, respectively, more effectively than to ROC1-CUL1 CTD (FIGS. 19A-C and FIG. 11A), it can be concluded that KH-4-43/#33-11 inhibit E3 CRL4 more specifically.

Compound #33 appears to be a more promiscuous inhibitor of E3 CRL as it inhibits ubiquitination by CRL1/SCF (FIGS. 3A-D, FIGS. 5A-C, and FIG. 7) and by CRL4 (FIGS. 2D-E). These properties are consistent with the ability of #33 to bind to both ROC1-CUL4A CTD and ROC1-CUL1 CTD with comparable K_{d} of ˜0.7 and 1.6 μM, respectively. The discrepancy between binding K_{d} measurement and ubiquitination inhibition assessment is noted. For example, KH-4-43 shows difference in K_{d} by two orders of magnitude in binding to ROC1-CUL4A CTD vs. ROC1-CUL1 CTD (FIGS. 19A-C). However, KH-4-43 inhibits ubiquitination by CRL4 and CRL1/SCF in the similar dose range of 10 and 30 μM, respectively (FIGS. 2B-C, FIGS. 4A-B, and FIGS. 6A-B).

Stabilization of E3 CRL4 Substrate CDT1

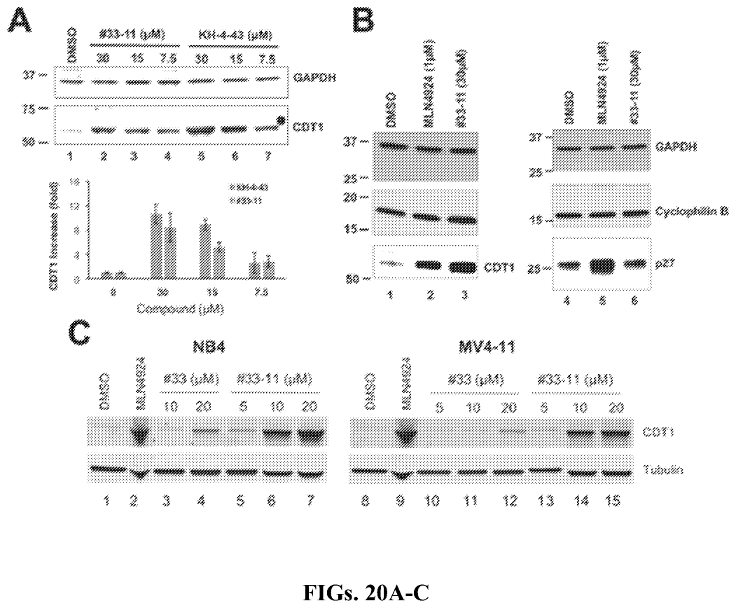

Treatment of Acute Myeloid Leukemia (AML) MV4-11 cells with KH-4-43/#33-11 caused accumulation of the E3 CRL4 substrate CDT1 (Higa et al., “Radiation-Mediated Proteolysis of Cdt1 by CUL4-ROC1 and CSN Complexes Constitutes a New Checkpoint,” Nat. Cell Biol. 5:1008-1015 (2003); Hu et al., “Targeted Ubiquitination of CDT1 by the DDB1-CUL4A-ROC1 Ligase in Response to DNA Damage,” Nat. Cell Biol. 6:1003-1009 (2004), which are hereby incorporated by reference in their entirety), with KH-4-43 exhibiting more pronounced stabilization effects (FIG. 20A). CDT1 is a DNA replication initiation factor and its accumulation induces DNA damage response to trigger apoptosis.

Compound #33-11 caused accumulation of CDT1 to levels comparable to those observed with MLN4924 (FIG. 20B, lanes 1-3), which is a Nedd8 inhibitor that unselectively blocks all CRL activity (Soucy et al., “An Inhibitor of NEDD8-Activating Enzyme as a New Approach to Treat Cancer,” Nature 458:732-736 (2009), which is hereby incorporated by reference in its entirety). In contrast, #33-11 did not change the levels of p27 (FIG. 20B, lane 6), which is a substrate by E3 CRL1/SCF^{Skp2 15} and was stabilized by MLN4924 (FIG. 20B, lane 5). These findings support the higher inhibitory activity of #33-11 towards E3 CRL4 over CRL1. Moreover, #33-11 was found to be more effective than #33 in causing stabilization of CDT1 in AML MV4-11 and NB-4 cells (FIG. 20C), consistent with the observation that #33-11 is more potent than #33 in CRL4 binding and inhibition (FIGS. 19A-C). The effects of KH-4-43/#33-11 on the stability of CRL2 substrate Hif-α (Kamura et al., “Activation of HIF1alpha Ubiquitination by a Reconstituted von-Hippel-Lindau (VHL) Tumor Suppressor Complex,” Proc. Natl. Acad. Sci. USA 97:10430-10435 (2000), which is hereby incorporated by reference in its entirety) and CRL3 substrate Nrf2 (Kobayashi et al., “Oxidative Stress Sensor Keap1 Functions as an Adaptor for Cul3-Based E3 Ligase to Regulate Proteasomal Degradation of Nrf2,” Mol. Cell. Biol. 24:7130-7139 (2004), which is hereby incorporated by reference in its entirety) cannot be examined in MV4-11, NB-4, or MOLT-4 cells due to the lack of expression in these cells.

Cytotoxicity by #33-11/KH-4-43 Against a Subset of Tumor Cells

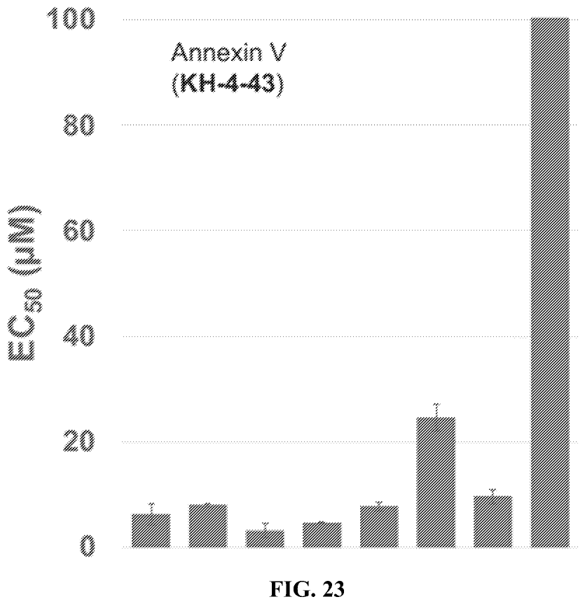

To evaluate the cytotoxic effects of #33-11/KH-4-43, flow cytometry with Annexin V staining was employed to detect cells undergoing apoptosis (Martin and Henry, “Distinguishing Between Apoptosis, Necrosis, Necroptosis and Other Cell Death Modalities,” Methods 61(2):87-89 (2013), which are hereby incorporated by reference in their entirety) (FIG. 1A and FIG. 23) and ATP-based cell viability assay (FIG. 1B). The results showed a significant variation among the 36 tumor cell lines in response to the compounds. As shown, a subset of tumor lines was relatively sensitive to the treatment of #33-11/KH-4-43 including AML (NB4/MV4-11/THP-1/ML2), acute lymphoblastic leukemia (ALL; MOLT-4/CCRF-CEM), T lymphoma (Jurkat), pancreas (CAPAN-2 and K8082), as well as ovary (OVCAR-3). While a panel of breast, liver, and lung tumor cell lines were more resistant with EC₅₀>100 μM (FIG. 1A). Cytotoxicity was observed in both the ATP-based cell viability assay and Annexin V staining, despite the former assay appearing to be more sensitive. KH-4-43 was evidently more toxic than #33-11 in multiple cell lines tested, exhibiting EC₅₀ of 1.8, 3.0, 3.9, and 4.8 μM in NB-4, MV4-11, OVCAR-3, and CAPAN-2 cells, respectively (FIG. 1B). Moreover, apoptosis was confirmed because the compound induced effects were reversed by zVAD-fmkm (an apoptosis inhibitor). To assess requirement of time exposure by #33-11/KH-4-43 on cytotoxicity, washout experiments were performed (FIG. 1C). The results showed that exposure between 6 to 24 hours was required for either KH-4-43 or #33-11 to achieve maximal toxic effects on MV4-11 cells. These effects were similar to what was observed with the proteasome drug bortezomib (Brignole et al., “Effect of Bortezomib on Human Neuroblastoma Cell Growth, Apoptosis, and Angiogenesis,” J. Natl. Cancer Inst. 98(16):1142-1157 (2006), which are hereby incorporated by reference in their entirety). Together these findings suggest that #33-11 and KH-4-43 are not generically toxic as they have tumor specific effects indicating that this approach has clinical potential.

#33-11 Cytotoxicity in a Subset of Tumor Lines was Correlated with Low CUL4 Abundance

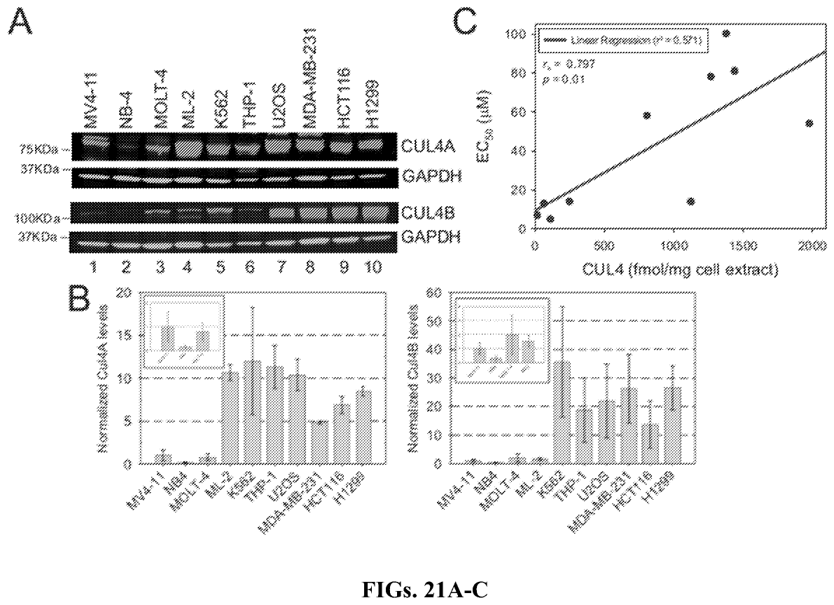

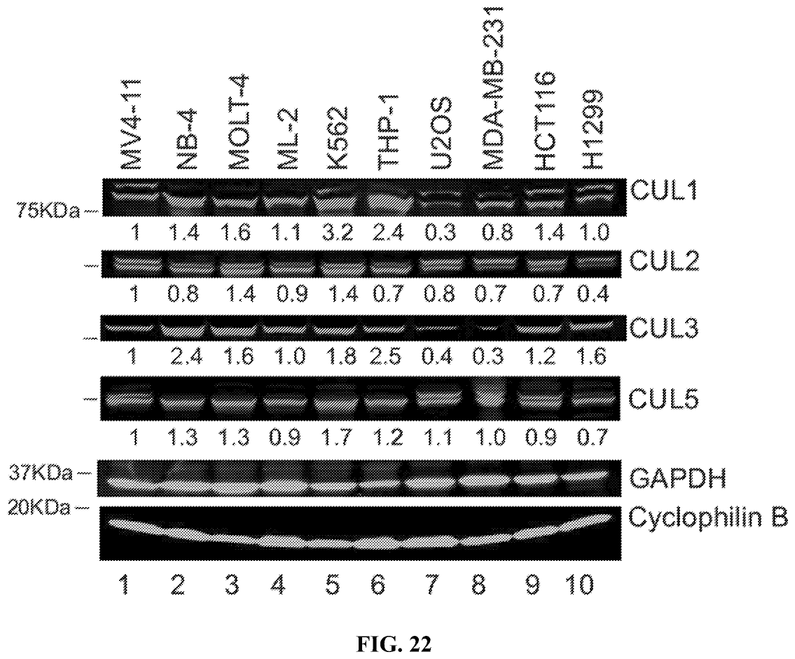

CUL4A and CUL4B are highly homologous except that CUL4B contains a distinct N-terminal extension that comprises a nuclear localization sequence, thereby shifting its cellular localization, making it a predominantly nuclear protein (Zou et al., “Characterization of Nuclear Localization Signal in the N Terminus of CUL4B and its Essential Role in Cyclin E Degradation and Cell Cycle Progression,” J. Biol. Chem. 284:33320-33332 (2009); Reichermeier et al., “PIKES Analysis Reveals Response to Degraders and Key Regulatory Mechanisms of the CRL4 Network,” Mol. Cell 77:1092-1106 (2020), which are hereby incorporated by reference in their entirety). To determine if the cytotoxicity of #33-11 was related to the protein levels of CUL4A and CUL4B, immunoblots were employed to analyze their abundance in a panel of 10 tumor cell lines (FIG. 21A). The relative protein abundance is shown revealed differences in protein levels of both CUL4A and CUL4B amongst tumor cell lines (FIG. 21). Using quantitative immunoblots with purified recombinant CUL4A and CUL4B proteins as standards, the concentrations of both CUL4A and CUL4B in these 10 tumor lines were estimated (Table 4). A scatter-plot of CUL4 protein level versus observed EC₅₀ for #33-11 in the ten cell lines revealed a trend indicating that CUL4 expression impacts sensitivity to #33-11 as indicated by Spearman Rank Test, Rₛ=0.797, p-value=0.01 using a two-sided test (FIG. 21C). As shown, AML NB-4/MV4-11/ML-2, ALL MOLT-4, and HCT116/U2OS/H1299 appeared to fit into this regression line, suggesting that these tumor lines exhibit an inverse relationship between CUL4 abundance and drug sensitivity. In all, these findings suggest that a subset of tumor cell lines: 1) express CUL4 at strikingly low levels in comparison to others, by a factor as large as 70-fold; and 2) appear to respond to #33-11 for apoptosis at levels more profoundly than those with more abundant CUL4. The difference in CUL4 abundance appears to be specific, because this subset of low-CUL4 expressing cells do not under-express related cullin proteins (CUL1/CUL2/CUL3/CUL5) (FIG. 22).

TABLE 4
Estimated Concentrations of CUK4A/CUL4B in a Panel of Tumor Cell Lines

| | | CUL4A | CUL4B | CUL4A/4B | EC₅₀, μM |
|---|---|---|---|---|---|
| Tumor Lines | Type | (fmol/mg) | (fmol/mg) | (fmol/mg) | (#33-11) |
| NB-4 | AML | ~3 | ~15 | ~18 | ~7 |
| MV4-11 | AML | ~16 | ~50 | ~66 | ~13 |
| MOLT-4 | ALL | ~13 | ~100 | ~113 | ~5 |
| ML-2 | AML | ~174 | ~77 | ~251 | ~14 |
| HCT116 | Colon | ~110 | ~700 | ~810 | ~58 |
| THP-1 | AML | ~185 | ~940 | ~1,125 | ~14 |
| U2OS | Bone | ~170 | ~1,100 | ~1,270 | ~78 |
| K562 | CML | ~200 | ~1,780 | ~1,980 | ~54 |
| MDA-MB-231 | Breast | ~80 | ~1,300 | ~1,380 | >100 |
| H1299 | Lung | ~140 | ~1,300 | ~1,440 | ~81 |

The estimated concentrations of CUL4A and CUL4B in indicated tumor lines are expressed as fmols per mg of total cellular proteins. The sensitivity of these tumor cells in response to #33-11 for apoptosis is expressed as EC₅₀ based on data shown in FIG. 1A. Italics marks those cell lines exhibiting invense correlation between CUL4 concentration and drug sensitivity based on analysis shown in FIG. 21C.

However, it cannot be generalized that increased sensitivity to CUL4 inhibition is inversely correlated with CUL4 abundance in all tumor cell types, as AML THP-1 has high levels of CUL4 (˜1,125 fmol/mg), but is sensitive to #33-11 with EC₅₀ of 14 μM (Table 4; FIG. 21B). In addition, both K8082 and OVCAR-3 were more sensitive to KH-4-43 than K8484 and SK-OV-3, respectively (FIG. 23 and FIG. 1), but exhibited no significant difference in CUL4 abundance.

Taken together, these findings suggest that the low abundance of CUL4 protein might be correlated with their sensitivity to #33-11 in a tumor cell-specific manner. The CUL4B protein is expressed at levels significantly higher than CUL4A in most tumor lines examined (Table 4). In recently published proteomics studies (Reichermeier et al., “PIKES Analysis Reveals Response to Degraders and Key Regulatory Mechanisms of the CRL4 Network,” Mol. Cell 77:1092-1106 (2020); Wang et al., “A Deep Proteome and Transcriptome Abundance Atlas of 29 Healthy Human Tissues,” Mol. Syst. Biol. 15(2):e8503 (2019), which are hereby incorporated by reference in their entirety), CUL4B was also found more abundant than CUL4A.

Reduction in CUL4 Levels Sensitized U2OS Cells to #33-11

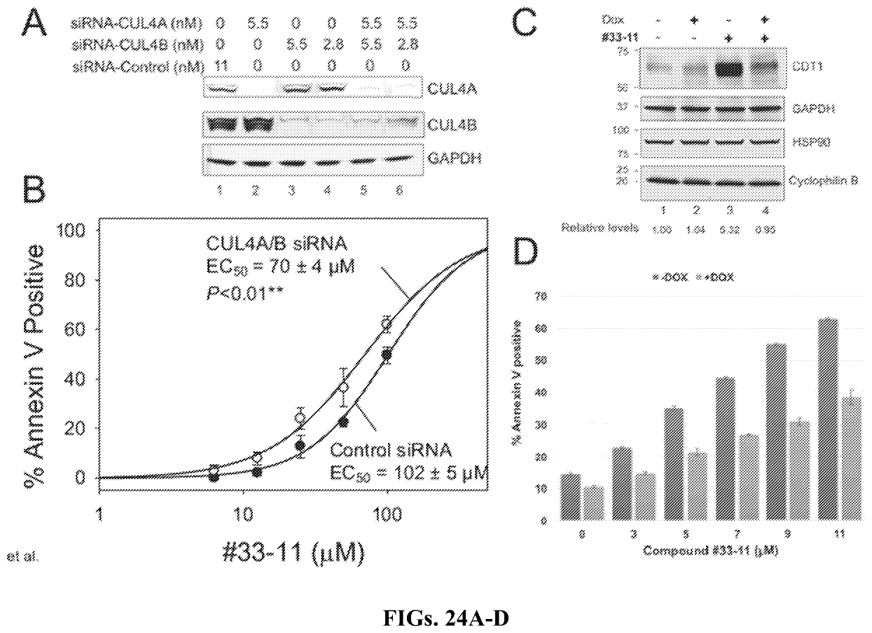

To evaluate whether decreased CUL4 expression sensitizes cells to #33-11, siRNAs targeting CUL4A, CUL4B, or both were used. U2OS cells expressing these siRNAs showed loss of CUL4A, CUL4B, or both as predicted (FIG. 24A). The siRNA-transfected cells were then treated with #33-11 and followed by flow cytometry analysis using Annexin V staining to quantify apoptosis. The results showed that combined depletion of both CUL4A and CUL4B enhanced the apoptotic response of the cells to #33-11 for apoptosis in a statistically significant manner (FIG. 6B; p=0.0047). These findings suggest that at least in some tumor cell type, reducing CUL4 levels sensitized cells to #33-11 for apoptosis.

Depletion of CDT1 Partially Overcomes #33-11-Induced Cytotoxicity

These results suggest that aberrant accumulation of CDT1, as a result of inactivation of E3 CRL4 by #33-11, triggers apoptosis. If this were true, shRNA-mediated CDT1 knockdown would reverse the cytotoxic effects by #33-11. To test this, Dharmacon SMARTvector Inducible Lentiviral shRNA system (GE) was employed to create an inducible AML MV4-11 cell line to regulate the abundance of CDT1 by Doxycycline (DOX). In the absence of compound, DOX treatment appeared to have little effect on CDT1 expression (FIG. 24C, lanes 1 and 2), presumably because this cell line expresses CDT1 protein at very low levels (also see Fv. 20). Consistent with previous observations (FIGS. 20A-C), #33-11 induced accumulation of CDT1 (FIG. 24C, lane 3). However, the #33-11-induced CDT1 accumulation was abolished by DOX treatment (FIG. 24C, lane 4). Under these conditions, the DOX-treated cells exhibited a significant decrease in #33-11-induced apoptosis as compared to the control (FIG. 24D). These findings further support that #33-11 is specifically impacting the cellular E3 CRL4/CDT1 pathway.

AML-Xenograft Mouse Model

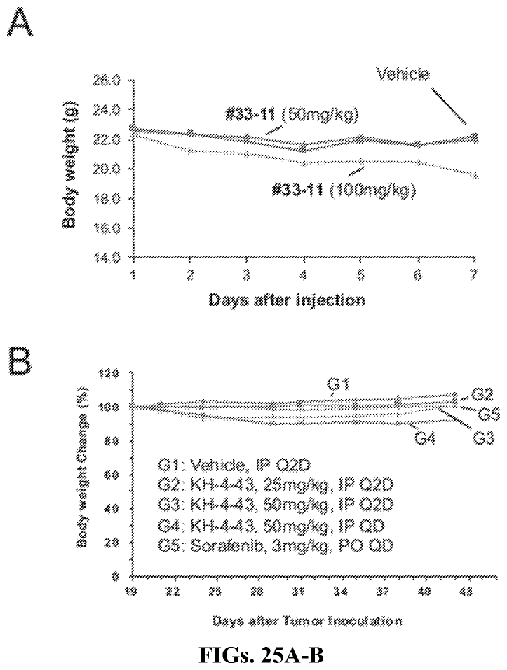

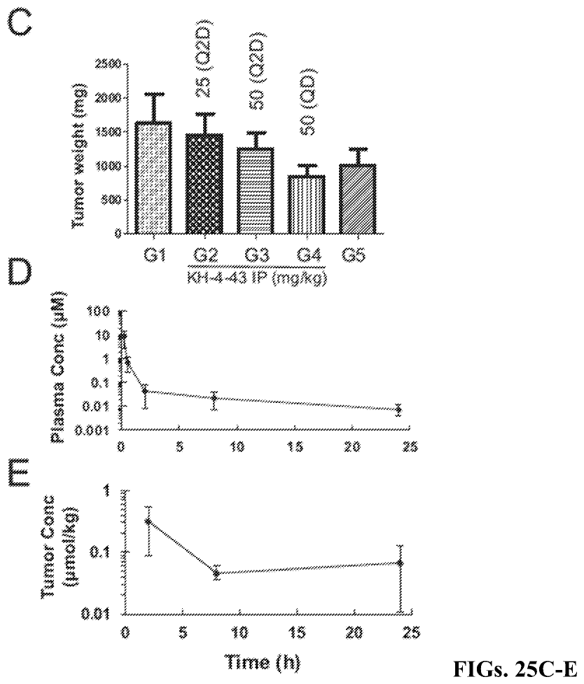

The anti-tumor activity of KH-4-43/#33-11 in mice was next determined. To access toxicity, female BALB/c nude mice (n=3) received vehicle only (40 μl DMSO), 50 mg/kg or 100 mg/kg of #33-11 by intraperitoneal (IP) injection and were monitored for 7 days. Data showed no significant body weight loss by either vehicle alone or #33-11 50 mg/kg (FIG. 25A), with the #33-11 100 mg/kg group showing >10%, but <20%, body weight loss. The results of necropsy revealed no adverse effects with either vehicle alone or the #33-11 50 mg/kg group. However, non-lethal abnormalities were observed in the #33-11 100 mg/kg group, including light red ascites, small intestine enlargement with yellow liquid, a yellow lump on the liver (4 mm*3 mm), intestinal adhesion, and slight adhesion of abdominal viscera. These data reveal a preliminary no adverse effect level (NOAEL) at 50 mg/kg and thus limited structure-based (#33-11) and target-based (E3 CRL4 inhibition) toxicity as well as useful safety window (i.e., <50 mg/kg dose) for our therapeutic disease model approach.

AML MV4-11 contains receptor FLT3 internal tandem repeat domain insertions that lead to FLT3 constitutive activation and ligand-independent growth. FLT3 mutations are found in ⅓ of AML patients and MV4-11 has been used as a model for AML xenograft studies (Auclair et al., “Antitumor Activity of Sorafenib in FLT3-Driven Leukemic Cells,” Leukemia 21(3):439-445 (2007), which is hereby incorporated by reference in its entirety). Studies to test anti-tumor activity in mouse MV4-11 xenograft model with lead KH-4-43 were conducted. MV4-11 tumors (n=10 per group) were implanted in female BALB/c nude mice by inoculation subcutaneously at the right flank (10⁷ MV4-11 cells). Treatment was initiated at day 19 when all mice had the tumor volume ˜150 mm³. For a positive control, group 5 mice (G5) were treated with sorafenib (Auclair et al., “Antitumor Activity of Sorafenib in FLT3-Driven Leukemic Cells,” Leukemia 21(3):439-445 (2007), which is hereby incorporated by reference in its entirety) by daily oral administration (QD) at 3 mg/kg. For a negative control, G1 received vehicle only (40 μl DMSO) by IP injection. G2 and G3 were IP injected every 2 days (Q2D) with KH-4-43 at 25 or 50 mg/kg, respectively. G4 was IP injected daily (QD) with KH-4-43 at 50 mg/kg. Each treatment was for a total of 23 days of dosing. Animal body weight and tumor volume were measured twice a week. Given the guideline that >20% body weight loss is considered toxic, KH-4-43 at the tested dose levels is judged to be safe in the earlier dose ranging study (FIG. 25B).

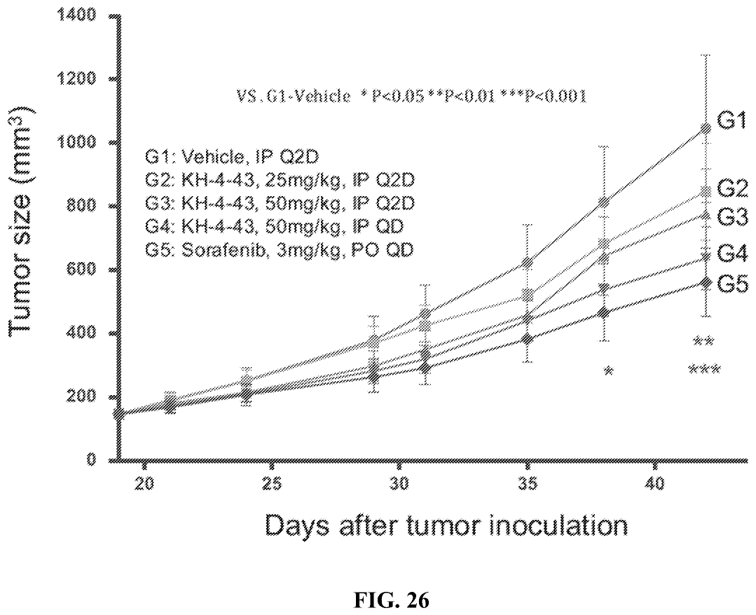

The changes of tumor volume and weight in groups G1-G5 at indicated time points after tumor inoculation were subjected to statistical analysis using two-way and one-way ANOVA, respectively. Tumor volume changes among 5 groups are shown in FIG. 26. G2 (KH-4-43, 25 mg/kg, Q2D), G3 (KH-4-43, 50 mg/kg, Q2D), G4 (KH-4-43, 50 mg/kg, QD), and G5 (Sorafenib, 3 mg/kg, QD) exhibited tumor inhibition values of 18.60%, 25.15%, 38.90%, and 46.02%, respectively. At day 42, G4 (P<0.01) and G5 (p<0.001) showed significant difference in tumor size in comparison to the vehicle control (G1). Consistent with this, G2, G3, and G4 exhibited reduction of tumor weight compared with the control (FIG. 25C). End-of-study PK analyses has detected KH-4-43 in plasma (FIG. 25D) and in tumor (FIG. 25E). In summary, KH-4-43 inhibited tumor growth in a dose-dependent manner and daily dosing at 50 mg/kg reached statistically significant tumor growth inhibition effect in these MV4-11 xenografts. KH-4-43 exhibited improved anti-tumor efficacy in comparison to #33-11. While #33-11 showed a trend of tumor growth inhibition in an earlier AML MV4-11 xenograft model study, the inhibitory effects did not reach statistically significant levels. Moreover, KH-4-43 was measured in plasma with area under the curve over 24 hours (AUC) of ˜3.3 μM-hr and slightly higher in tumor of ˜4.7 μM-hr of the MV4-11 xenograft mice, respectively, which is 6-fold higher AUC than that found in tumors of mice dosed with #33-11 (0.75 μM-hr) (FIGS. 25A-E). Overall, the results of mouse model studies helped establish proof-of-concept evidence that compound KH-4-43 possesses modest anti-tumor activity in vivo.

Analog SAR, ADME/PK, and Other Properties

Both commercial (21) and synthetic (56) analogs were used to develop SAR at various positions of the E3 CRL inhibitor scaffold. The results of MST binding experiments have revealed that #33-7, a related analog without the 8-position hydroxyl group (—OH) of hit #33, is inactive in blocking ubiquitination, with drastically reduced binding activity to ROC1-CUL1 CTD (K_{d} decreases by >30 fold) (Table 5). #33-3 lacking both the 7- and 8-hydroxyl groups was completely inactive in both blocking ubiquitination and E3 binding (Table 5). On the other hand, #33-2, an analog with the replacement of the 2-position trifluoromethyl (—CF₃) group with simple methyl (—CH₃), exhibited a 7-fold drop in IC₅₀ and an 8-fold reduction of K_{d}(Table 5). These results strongly suggest that #33's hydroxyl group (—OH) at the 8-position is essential, and its trifluoromethyl group at the 2-position is required for maximal inhibitory activity.

TABLE 5
#33 Analogs and Their SAR Studies

| Compound | Structure | E3 inhibition | E3 binding |
|---|---|---|---|
| 33 | 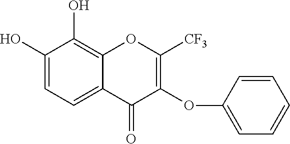 [SMILES: O=c1c(Oc2ccccc2)c(C(F)(F)F)oc2c(O)c(O)ccc12] | IC₅₀ = 2.8 μM | K_{d} = 1.84 μM |
| 33-2 | 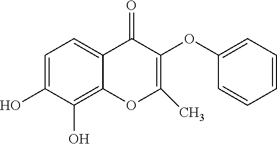 [SMILES: Cc1oc2c(O)c(O)ccc2c(=O)c1Oc1ccccc1] | IC₅₀ = 8.9 μM | K_{d} = 15 μM |
| 33-3 | 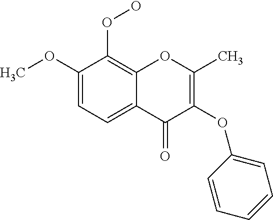 [SMILES: COc1ccc2c(=O)c(Oc3ccccc3)c(C)oc2c1OO] | No inhibition | K_{d} > 500 μM |
| 33-4 | 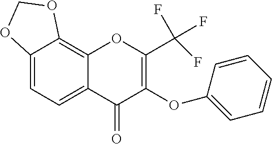 [SMILES: O=c1c(Oc2ccccc2)c(C(F)(F)F)oc2c3c(ccc12)OCO3] | IC₅₀ = 80 μM | K_{d} = 19 μM |
| 33-5 | 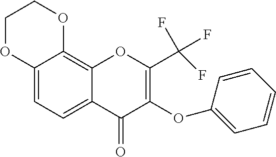 [SMILES: O=c1c(Oc2ccccc2)c(C(F)(F)F)oc2c3c(ccc12)OCCO3] | No inhibition | K_{d} = 48 μM |
| 33-7 | 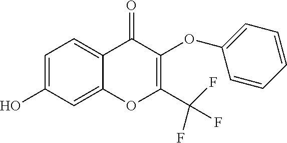 [SMILES: O=c1c(Oc2ccccc2)c(C(F)(F)F)oc2cc(O)ccc12] | No inhibition | K_{d} = 57 μM |

E3 activity was measured using the FRET-K48-di-Ub synthesis assay with ROC1-CUL1 CTD as shown in FIG. 7A. IC₅₀ values are indicated. No inhibition was observed with up to 80 μM of #33-3, #33-5, or #33-7. E3-ligand binding was determined using MST with E3 complex ROC1-CUL1 CTD as described in FIGS. 19A-C.

To further SAR studies, KH-4-43 and #33-11 were compared with their related structural analogs KH-4-119, KH-3-141, KH-3-115, MM-007, MM-008, and MM-009 (Table 6). KH-4-43 and #33-11 bound to ROC1-CUL4A (full length) with a K_{d} of 0.55 and 1.59 μM, respectively. Lower binding with ROC1-CUL4A than ROC1-CUL4A CTD was observed (FIGS. 191B-C). With ROC1-CUL4A possessing free CUL4A N-terminus, which binds to the DDB1/DCAF sub-complex in the context of holo-enzyme, it is possible that the unoccupied CUL4A N-terminus may negatively impact the binding activity of K1-4-43/#33-11 to ROC1-CUL4A. KH-4-119, a related analog with the 8-position methoxy group (—OMe), has no detectable binding activity to ROC1-CUL4A (Table 6). In addition, KH-4-119 (7-OH, 8-OMe analog) showed very weak activity in blocking ubiquitination in vitro using a biochemical assay (FIGS. 15A-C), and is incapable of inducing apoptosis in cancer cells using Annexin V flow cytometry assay (FIG. 1A). Moreover, KH-3-115 lacking the pyrazole N-pendant phenyl group of #33-11 exhibited >20-fold drop in K_{d}, and is weak in inhibiting ubiquitination and inactive in inducing apoptosis (Table 6). Finally, substitution of the pendant phenyl group with carbon chains of varying length (MM-007, MM-008, and MM-009, respectively) significantly weakened analogs in E3 binding/inhibition and apoptosis induction. These results confirm the importance of the hydroxyl group (—OH) at the 8-position, and suggest that the pendant phenyl group is required for improved E3 binding, E3 inhibitory activity and apoptosis.

TABLE 6
SAR Studies of #33-11/KH-4-43 Analogs

| | | E3 binding | E3 CRL4 | |
|---|---|---|---|---|
| | | ROC1- | Relative | Apoptosis |
| Compound | Structure | CUL4A | Inhibition | EC₅₀ |
| KH-4-43 | 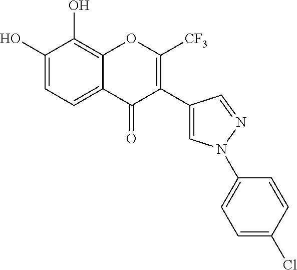 [SMILES: O=c1c(-c2cnn(-c3ccc(Cl)cc3)c2)c(C(F)(F)F)oc2c(O)c(O)ccc12] | Kd 0.55 ± 0.12 μM | 100% | 8 μM |
| #33-11 | 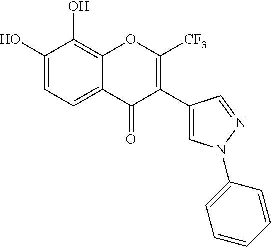 [SMILES: O=c1c(-c2cnn(-c3ccccc3)c2)c(C(F)(F)F)oc2c(O)c(O)ccc12] | Kd 1.59 ± 0.24 μM | 37% | 13 μM |
| KH-4-119 | 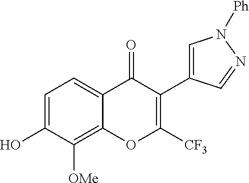 [SMILES: COc1c(O)ccc2c(=O)c(-c3cnn(-c4ccccc4)c3)c(C(F)(F)F)oc12] | No detectable binding | 13% | Inactive |
| KH-3-141 | 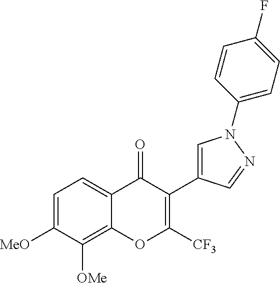 [SMILES: COc1ccc2c(=O)c(-c3cnn(-c4ccc(F)cc4)c3)c(C(F)(F)F)oc2c1OC] | No detectable binding | 0% | Inactive |
| KH-3-115 | 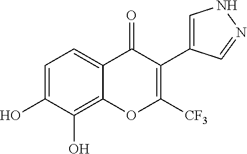 [SMILES: O=c1c(-c2cn[nH]c2)c(C(F)(F)F)oc2c(O)c(O)ccc12] | Kd 37.7 ± 18.4 μM | 13% | Inactive |
| MM-007 | 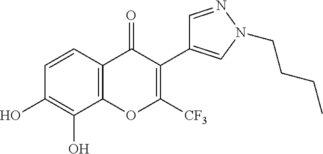 [SMILES: CCCCn1cc(-c2c(C(F)(F)F)oc3c(O)c(O)ccc3c2=O)cn1] | Kd 38.0 ± 7.3 μM | 2% | 28 μM |
| MM-008 | 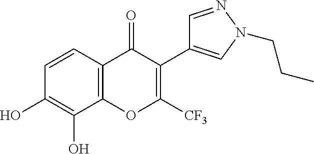 [SMILES: CCCn1cc(-c2c(C(F)(F)F)oc3c(O)c(O)ccc3c2=O)cn1] | Kd 26.4 ± 6.7 μM | 0% | 38 μM |
| MM-009 | 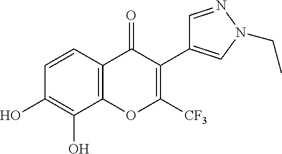 [SMILES: CCn1cc(-c2c(C(F)(F)F)oc3c(O)c(O)ccc3c2=O)cn1] | Kd >400 μM | 0% | 66 μM |

E3-ligand binding was determined using MST with E3 complex ROC1-CUL4A as described in FIGS. 19A-C. E3 CRL4 inhibition was determined using the assay described in FIGS. 15A-C. Values are expressed as relative inhibition in reference to KH-4-43 (1000%). Apoptosis experiments were carried out as described in FIG. 1A. At 30 μM, KH-4-119, KH-3-141, and KH-3-115 were unable to induce any detectable level of apoptosis.

Table 7 shows the results of in vitro ADMIE/DMIPK (Absorption, Distribution, Metabolism, and Excretion/Drug metabolism and pharmacokinetics) assays as well as i.p. PK in mice with #33-11/KH-4-43. Both #33-11/KH-4-43 have many viable ADME properties, as well as some minor liabilities expected of early stage lead compounds. For example, #33-11 has high plasma stability in three species and stability in rat and human liver microsomes. KH-4-43 shows excellent in vitro stability in mouse, rat and human microsomes with low clearance (<10 and long in vitro half-life >5 hrs) as a result of the 4-chloro substituent, which blocks a potential site of metabolism in 4-phenyl position of #33-11 (see FIG. 19A for chemical structure comparison). KH-4-43 is also highly permeable in CACO-2 assay and is not a substrate of PGP-transporter (MDR1). KH-4-43 performed well in the in vivo mouse PK study, when i.p. dosed at 100 mg/kg. KH-4-43 was well tolerated and provided a Cmax of ˜35 μM with higher clearance in the first 3 hrs (α-phase), though it provided sizable AUC (0-24 hr of 19.2 μM-hr), long terminal half-life of ˜9.6 hrs (β-phase elimination) and 24 hr C-trough concentration of 89 nM. Taken together, #33-11/KH-4-43 have important properties for a new lead, but also reveal areas for improvement, especially on initial (α-phase) mouse PK clearance parameter.

TABLE 7
In Vitro/In Vivo DMPK Parameters and S-COMT Activity
Preliminary In vitro DMPK

| | Mouse | Rat | Human |
|---|---|---|---|
| Plasma Stability: t_{1/2} (min) | | | |
| #33-11 | 231 | 231 | >500 |
| KH-4-43 | ND | ND | ND |
| Liver microsomes: t_{1/2} (min) | | | |
| #33-11 | 11 | 112 | 315 |
| KH-4-43 | 321 | >500 | >500 |
| CACO-2 permeability: | | #33-11 | KH-4-43 |
| Papp (×10⁻⁶ cm/s; n = 2) | A to B | 31.4 | 51.2 |
| | B to A | 61.3 | 52.7 |
| A:B/B:A | | 1.95 | 1.0 |
| Permeability | | high | high |
| PGP substrate | | probable | Not a |
| | | | substrate |
| Kinetic solubility (pH 7.4): | | | |

| | |
|---|---|
| #33-11 | 14.6 mM; moderate solubility |
| KH-4-43 | ND |

Preliminary in vivo i.p. mouse PK (100 mg/kg in DMSO)

| | T_{1/2} | Clp | AUC | Cmax | Ctrough |
|---|---|---|---|---|---|
| | (hr) | (ml/min/kg) | (μM-hr) | (μM) | (μM) |
| #33-11 | 7.2 | 231 | 19.6 | 36.9 | 0.16 |
| KH-4-43 | 9.6 | 206 | 19.2 | 35.6 | 0.09 |

Catechol O-methyltransferase assay (1 μM; RBC-DA1 as substrate)

| | |
|---|---|
| 0.25 μM Sub: | IC₅₀ (Tolcapone) = 0.378 nM; IC₅₀ |
| | (#33-11) = 105.00 nM |
| 1 μM Sub: | IC₅₀ (Tolcapone) = 0.555 nM; IC₅₀ |
| | (#33-11) = 540.00 nM |
| 3 μM Sub: | IC₅₀ (Tolcapone) = 0.825 nM; IC₅₀ |
| | (#33-11) = 807.00 nM |

#33-11 and KH-4-43 both contain a catechol moiety (FIG. 19A), which might be deactivated rapidly in vivo by the enzyme Catechol 0-Methyl Transferase (COMT) (Eisenhofer et al., “Catecholamine Metabolism: A Contemporary View With Implications for Physiology and Medicine,” Pharmacological Reviews 56(3):331-349 (2004), which is hereby incorporated by reference in its entirety). However, side-by-side comparison between #33-11 and Tolcapone, a potent inhibitor of COMT (Antonini et al., “COMT Inhibition With Tolcapone in the Treatment Algorithm of Patients With Parkinson's Disease (PD): Relevance for Motor and Non-Motor Features,” Neuropsychiatric Disease and Treatment 4(1):1-9 (2008), which is hereby incorporated by reference in its entirety), showed that at a range of substrate concentrations, #33-11 is 277- to 978-fold less active than Tolcapone (Table 7). These findings suggest that #33-11 is not a substrate of COMT.

The presence of the catechol moiety in #33-11/KH-4-43 (FIG. 19A) also raises perceived concerns about its stability due to redox cycling. However, the data presented in this application have refuted these perceptions; ADME/PK analysis has shown that #33-11/KH-4-43 are stable in mouse, rat and human microsomes and exhibit high plasma stability (Table 7). In addition, anti-tumor activity by KH-4-43 in mouse xenograft was have observed (FIGS. 25A-E, FIG. 26) and detected this compound in both the plasma (FIG. 25D) and the tumor (FIG. 25E) of the experimental mice. Importantly, the results of SAR studies demonstrated the importance of not only the hydroxyl group (—OH) at the 8-position, but also the pendant pyrazole N-phenyl group required for improved E3 binding, E3 inhibitory activity and apoptosis (Table 6). These findings are at odds with any hypothesis that the observed inhibition by KH-4-43/#33-11/#33 is mediated by non-specific effects of the catechol group alone on E3 CRL. Specifically, KH-3-115, which possesses the catechol group, is inactive in apoptosis assay and weakly active E3 binding and CRL4 assay (Table 6). Finally, the results of immobilization experiments (FIGS. 10 and 14) suggest that KH-4-43/#33 act reversibly, which would be inconsistent with a possibility that this compound exerts inhibitory effects on E3 CRL4 by using the catechol group to mediate covalent interactions. There is a long history of chromones (contained in #33-11/KH-4-43) in drug discovery including several that are marketed drugs (Silva et al., “Chromones: A Promising Ring System for New Antiinflammatory Drugs,” Chem. Med. Chem. 11:2252-2260 (2016), which is hereby incorporated by reference in its entirety). Indeed, the chromone scaffold has been referred to as a “privileged structure” for drug discovery (Keri et al., “Chromones as a Privileged Scaffold in Drug Discovery: A Review,” Eur. J. Med. Chem. 78:340-374 (2014), which is hereby incorporated by reference in its entirety), indicating it is a useful starting scaffold to optimize (Matos et al., “Synthesis and Pharmacological Activities of Nonflavonoid Chromones: A Patent Review (from 2005 to 2015),” Expert Opin. Ther. Patents 25(11):1285-1304 (2015), which is hereby incorporated by reference in its entirety). Of note, multiple approved chromone drugs include Cromolyn, Nedocromil, Diosmin and more recently Alvocidib as well as Flavoxate. There are also 17 FDA-approved catechol drugs (Yang et al., “How Many Drugs Are Catecholics?,” Molecules 12(4):878-884 (2007), which is hereby incorporated by reference in its entirety). Taken together, the data presented in this application demonstrate that #33-11/KH-4-43 are starting points for further medicinal chemistry optimization.

### Example 19—Discussion of Examples 1-18

Novel Scaffold Against E3 CRL4

According to Structural Genomics Consortium, chemical probes are required to minimally have in vitro potency of the target protein at <100 nM, possess >30× selectivity relative to other sequence-related proteins of the same target family, and have demonstrated on-target effects at <1 μM (Arrowsmith et al., “The Promise and Peril of Chemical Probes,” Nat. Chem. Biol. 11:536-541 (2015), which is hereby incorporated by reference in its entirety). KH-4-43 appears close to this criteria because it has binding K_{d} to E3 ROC1-CUL4A CTD or highly related ROC1-CUL1 CTD at 83 nM or 9.4 μM, respectively (FIGS. 19A-C), which represents a difference of 2 orders of magnitude. KH-4-43 exhibits cytotoxicity in a subset of tumor cell lines with EC₅₀ approaching ˜2 μM (FIGS. 1A-C). The results of RNAi-mediated sensitization (CUL4 depletion; FIGS. 24A-B) and rescue (CDT1 depletion; FIGS. 24C-D) experiments suggest on-target effects by the KH-4-43 related analog #33-11.

Preliminary SAR suggests that the #33-11/KH-4-43 scaffolds (FIG. 19A) are highly amenable to medicinal chemistry optimization with several substituents available for modification and building blocks that have been easily incorporated into the scaffold. The C-3 position likely contributes significantly to the binding specificity for E3 CRL4A. #33-11 differs from hit #33 (FIG. 19A) only at C-3 (N-Ph pyrazole in #33-11 vs. phenyl ether in #33). Chromone substituent change from aryl ether of #33 to the N-phenyl pyrazole group of #33-11 as the C-3 linker, enhances the binding affinity by 20-fold (FIGS. 19A-C and FIG. 11A). SAR also has underscored the significance of the hydroxyl group (—OH) at the 8-position and the pendant phenyl group (Tables 5-6). Future SAR optimization by exploring various phenyl substituents and replacement of pyrazole by other heteroaryl (or aryl) group will lead to further increase of affinity for CRL4.

Ligand-Target Interactions and Specificity

An effective drug typically exerts its effects via interactions with receptors, thereby initiating biochemical and physiologic changes that characterize the drug's response. The binding of compounds #33-11/KH-4-43 to E3 CUL4's CTD (FIGS. 19A-C) presumably antagonize the E3's ability to interact with E2 conjugating enzyme(s), hence resulting in biochemical changes including inhibition of ubiquitination (FIGS. 2A-E, FIGS. 13A-B, and FIGS. 15A-C) and stabilization of E3 CRL4's substrate CDT1 (FIGS. 20A-C). The physiologic response by a subset of tumor cell lines to #33-11 and KH-4-43 is apoptosis (FIG. 1A and FIG. 23). The cytotoxicity has been validated in the mouse tumor suppression in vivo model (FIGS. 25A-E and FIG. 26).

Despite conservation between Cullin 1-5, KH-4-43/#33-11 exhibit specificity in targeting E3 CRL4's core ligase. First, biophysical experiments showed that KH-4-43/#33-11 bind to core ligase ROC1-CUL4A CTD with ˜100-200 nM affinity, 20-100-fold higher than the related ROC1-CUL1 CTD complex (FIGS. 19A-C). Second, biochemical assays showed that KH-4-43 is more potent than #33 in inhibiting the ubiquitination of CK1α by E3 CRL4^{CRBN} (FIGS. 2A-E). Third, by contrast, KH-4-43 is less effective than #33 in inhibiting the ubiquitination of SCF^{βTρXII} substrates IκBα (FIGS. 4A-B) and β-catenin (FIGS. 6A-B). Compound #33-11 exhibits little inhibitory activity toward di-Ub synthesis reactions catalyzed by E3 subcomplexes including ROC1-CUL1 (FIG. 8C) or ROC1-CUL2/ROC1-CUL3 (FIG. 9). Fourth, cell-based experiments revealed that #33-11/KH-4-43 induces accumulation of E3 CRL4 substrate CDT1, but not E3 CRL1 substrate p27 (FIGS. 20A-C). Fifth, RNAi experiments showed that depletion of CUL4 sensitized U2OS cells for #33-11-induced apoptosis (FIGS. 24A-B) and knockdown of E3 CRL4 substrate CDT1 partially overcame #33-11's cytotoxicity (FIGS. 24C-D), providing physiological evidence that #33-11 targets the CRL4/CDT1 pathway specifically.

Thus, these data demonstrate that it is possible for a small molecule agent to selectively target a specific cullin CTD. Despite a common globular CTD domain adopted by the cullin 1-5, however, different cullin CTDs have divergent folds (Angers et al., “Molecular Architecture and Assembly of the DDB1-CUL4A Ubiquitin Ligase Machinery,” Nature 443(7111):590-593 (2006); Zheng et al., “Structure of the Cul1-Rbx1-Skp1-F boxSkp2 SCF Ubiquitin Ligase Complex,” Nature 416(6882):703-709 (2002); Cardote et al., “Crystal Structure of the Cul2-Rbx1-EloBC-VHL Ubiquitin Ligase Complex,” Structure 25(6):901-911 (2017); Duda et al., “Structural Insights Into NEDD8 Activation of Cullin-RING Ligases: Conformational Control of Conjugation,” Cell 134:995-1006 (2008), which are hereby incorporated by reference in their entirety), and different total areas of interface with ROC1/Rbx1 that result in significant divergent orientation of the ROC1/Rbx1 RING domain among CRLs (Cardote et al., “Crystal Structure of the Cul2-Rbx1-EloBC-VHL Ubiquitin Ligase Complex,” Structure 25(6):901-911 (2017), which is hereby incorporated by reference in its entirety). It is therefore conceivable that a small molecule (such as #33-11/KH-4-43) may bind to a specific site within a cullin CTD that impact the cullin-E2 interaction, and/or alter the ROC1/Rbx1 orientation, leading to selective inhibition of ubiquitination.

E3 CUL4 Inhibitors Possess Tumor Inhibitory Potential

The observed anti-tumor activity by our E3 CRL4 lead inhibitors is consistent with previous studies that have shown oncogenic potential of both CUL4A and CUL4B (Cheng et al., “The Emerging Role for Cullin 4 Family of E3 Ligases in Tumorigenesis,” Biochim. Biophys. Acta Rev. Cancer 1871(1):138-159 (2019), which is hereby incorporated by reference in its entirety). In mouse tumor models both overexpression or silencing approaches have established a tumor-promoting role for either CUL4A or CUL4B in multiple cancer types including lung, breast, colon, and hepatocellular carcinomas. In addition, a large body of investigations have linked CUL4 overexpression to human cancers including breast, ovary, stomach, colon, pancreas, lung, and bile ducts.

The most successful cancer therapies are chemical entities that preferentially target a protein or enzyme that carries a tumor specific vulnerability, such as mutation or other alteration that is specific to cancer cells and not found in normal host tissue. This is best exemplified by the clinical success of Gleevec, which is an inhibitor with exceptional affinity for the oncogenic, tumor-specific fusion kinase BCR-Abl that drives tumorigenesis in chronic myelogenous leukemia (Takimoto and Calvo, “Principles of Oncologic Pharmacotherapy,” Archived 15 May 2009 at the Wayback Machine in Pazdur R, Wagman L D, Camphausen K A, Hoskins W J (Eds.) “Cancer Management: A Multidisciplinary Approach,” Archived 4 Oct. 2013 at the Wayback Machine. 11 ed. (2008), which is hereby incorporated by reference in its entirety). Here, a different type of vulnerability in a subset of tumor cells are suggested, which are characterized by low expression of the E3 component CUL4 (FIGS. 21A-C) and sensitivity to our CRL4 lead inhibitor #33-11/KH-4-43 (FIGS. 1A-C; Table 4). To support the key role played by CUL4 abundance in these drug effects of certain tumor cells, the fact that reduction of the CUL4 levels by means of siRNA-mediated depletion sensitized U2OS cells to #33-11 treatment for apoptosis was demonstrated (FIGS. 24A-B). Moreover, lead inhibitors #33-11/KH-4-43 exhibited in vivo anti-tumor activity in AML MV4-11 xenograft mouse model studies (FIG. 26). The major impact of low target expression in enhancing druggability has been previously recognized as clinically significant. A well-known example, leukemic del(5q) MDS cells are haplo-insufficient for CK1-α and these cells are sensitized to lenalidomide therapy that specifically targets CK1-α for degradation (Krönke et al., “Lenalidomide Induces Ubiquitination and Degradation of CK1α in del(5q) MDS,” Nature 523(7559):183-188 (2015), which is hereby incorporated by reference in its entirety). If validated, the low-target-expression-driven-drug sensitivity mechanism may be advantageous as compared to targeting amplified or mutated oncoproteins because targeting these oncogenic drivers frequently leads to selection events such as secondary mutations which result in drug resistance (Orlando et al., “Oncogene Addiction as a Foundation of Targeted Cancer Therapy: The Paradigm of the MET Receptor Tyrosine Kinase,” Cancer Lett. 443:189-202 (2019), which is hereby incorporated by reference in its entirety).

### Example 20—Synthesis of E3 Ligase Inhibitors

Materials and Methods

¹H spectra were acquired on a Bruker DRX-600 spectrometer at 600 MHz for 1H. Thin layer chromatography (TLC) was performed on silica coated aluminum sheets (thickness 200 μm) or alumina coated (thickness 200 μm) aluminum sheets supplied by Sorbent Technologies, and column chromatography was carried out on Teledyne ISCO combiflash equipped with a variable wavelength detector and a fraction collector using a RediSep Rf high performance silica flash columns by Teledyne ISCO. LCMS/HPLC analysis for purity determination and HRMS was conducted on an Agilent Technologies G1969A high-resolution API-TOF mass spectrometer attached to an Agilent Technologies 1200 HPLC system. Samples were ionized by electrospray ionization (ESI) in positive mode. Chromatography was performed on a 2.1×150 mm Zorbax 300SBC18 5-μm column with water containing 0.1% formic acid as solvent A and acetonitrile containing 0.1% formic acid as solvent B at a flow rate of 0.4 mL/min. The gradient program was as follows: 1% B (0-1 min), 1-99% B (1-4 min), and 99% B (4-8 min). The temperature of the column was held at 50° C. for the entire analysis. The purity of all the compounds was ≥95%. The chemicals and reagents were purchased from Combi-Blocks, Sigma-Aldrich and Acros Organics. All solvents were purchased in anhydrous from Acros Organics and used without further purification.

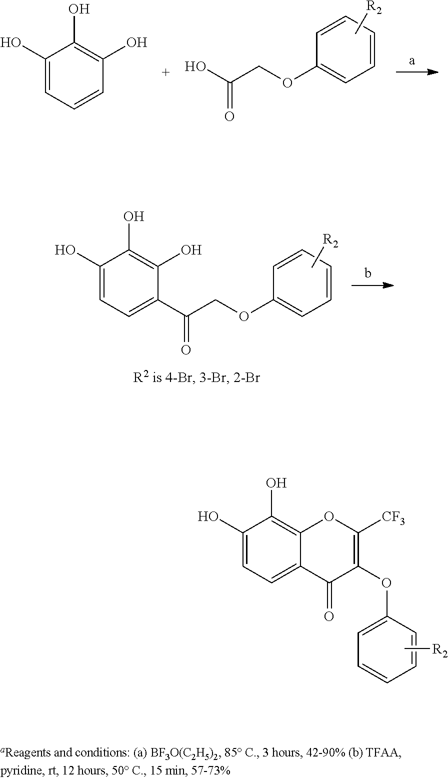

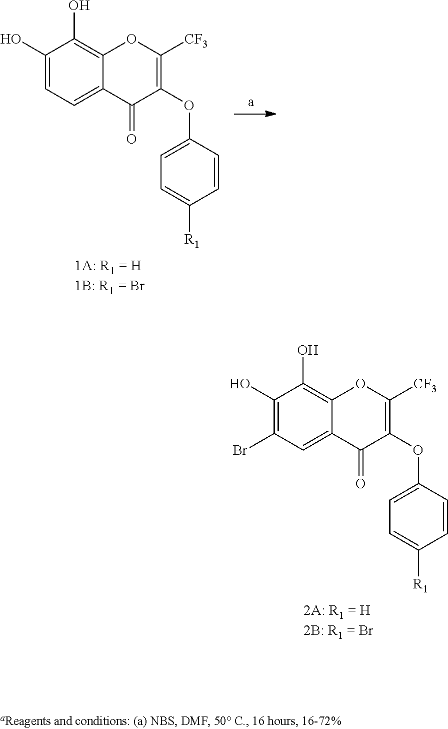

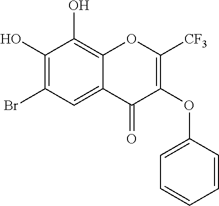 [SMILES: O=c1c(Oc2ccccc2)c(C(F)(F)F)oc2c(O)c(O)c(Br)cc12]

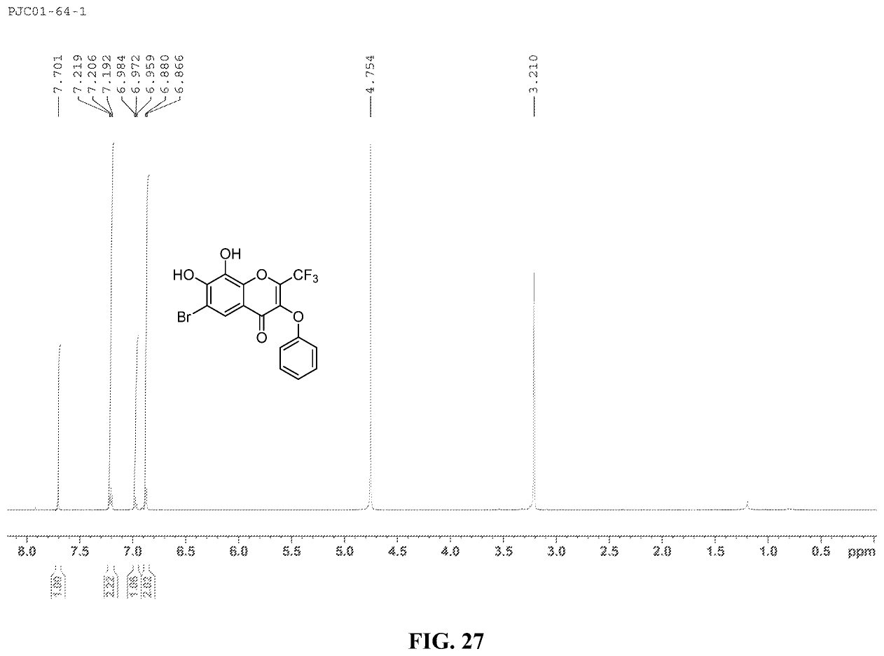

6-Bromo-7,8-dihydroxy-3-phenoxy-2-(trifluoromethyl)-4H-chromen-4-one: A solution of 7,8-dihydroxy-3-phenoxy-2-(trifluoromethyl)-4H-chromen-4-one (0.0458 g, 1 eq, 0.135 mmol) and N-bromosuccinimide (0.0506 mg, 2.10 eq, 0.284 mmol) in DMF (1 mL) was stirred at 50° C. for 16 hours. After 16 hours, the reaction mixture was extracted with ethyl acetate and water. The ethyl acetate layer was collected, dried with magnesium sulfate and filtered to obtain a filtrate. The filtrate was purified by the normal phase chromatography (100% hexane to 20% ethyl acetate in hexane) to afford 6-bromo-7,8-dihydroxy-3-phenoxy-2-(trifluoromethyl)-4H-chromen-4-one (yield: 0.0358 g; 72%). ¹H NMR (MeOD-d₄, 600 MHz) δ 7.70 (s, 1H), 7.21 (t, J=7.81, 8.11 Hz, 2H), 6.97 (t, J=7.34, 7.40 Hz, 1H), 6.87 (d, J=8.26 Hz, 2H); HRMS (ESI): m/z [M+H]⁺ calculated for C₁₆H₈BrF₃O₅ 416.9580; found 416.9606; purity >95%. FIG. 27 shows the characterization of the product.

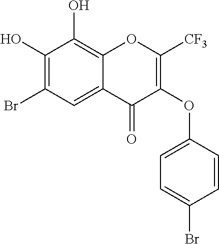 [SMILES: O=c1c(Oc2ccc(Br)cc2)c(C(F)(F)F)oc2c(O)c(O)c(Br)cc12]

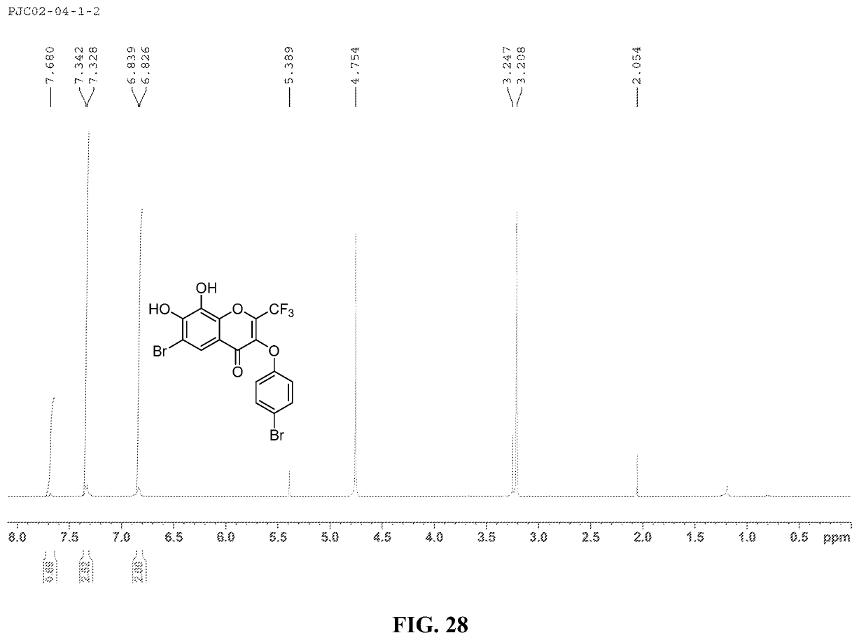

6-Bromo-3-(4-bromophenoxy)-7,8-dihydroxy-2-(trifluoromethyl)-4H-chromen-4-one was prepared using the procedure described above (yield: 0.0082 mg; 16.4%). ¹H NMR (MeOD-d₄, 600 MHz) δ 7.68 (s, 1H), 7.34 (d, J=7.98 Hz, 2H), 6.83 (d, J=7.87 Hz, 2H); HRMS (ESI): m/z [M+H]⁺ calculated for C₁₆H₇Br₂F₃O₅ 496.8665; found 496.8653; purity >95%. FIG. 28 shows the characterization of the product.

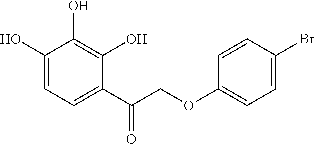 [SMILES: O=C(COc1ccc(Br)cc1)c1ccc(O)c(O)c1O]

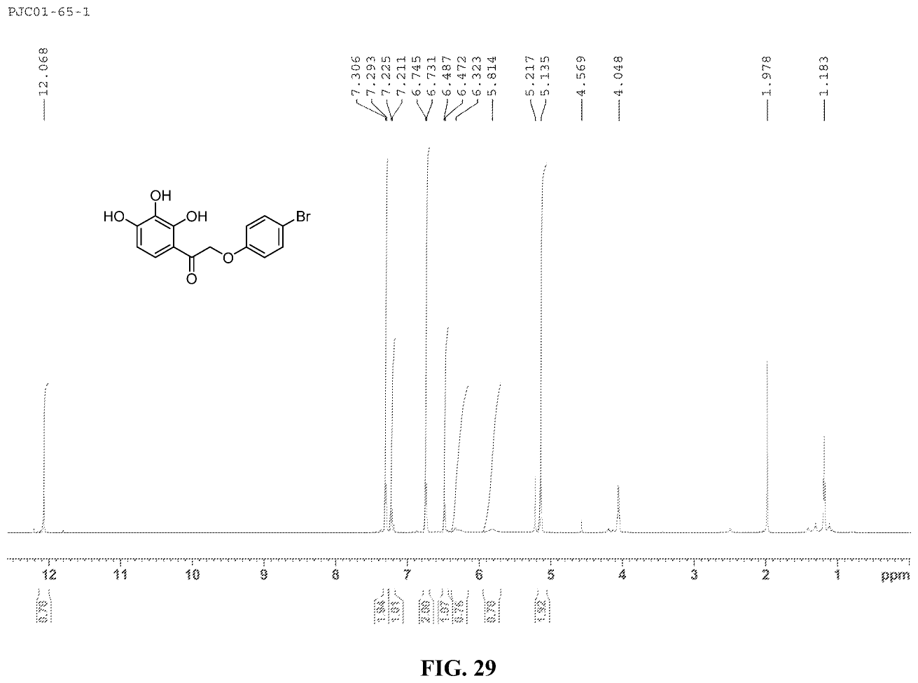

2-(4-Bromophenoxy)-1-(2,3,4-trihydroxyphenyl)ethan-1-one: A solution of benzene-1,2,3-triol (0.05 g, 1.0 eq, 0.4 mmol) and 2-(4-bromophenoxy)acetic acid (0.1 g, 1.0 eq, 0.4 mmol) in boron trifluoride etherate (2 g, 2 mL, 20 eq, 9 mmol) was stirred at 85° C. for 3 hour. After 3 hours, the reaction was quenched with saturated sodium carbonate and extracted with water and ethyl acetate. The ethyl acetate layer was collected, dried with magnesium sulfate and filtered to obtain a filtrate. The filtrate was purified by the normal phase chromatography (100% hexane to 50% ethyl acetate in hexane) to afford 2-(4-bromophenoxy)-1-(2,3,4-trihydroxyphenyl)ethan-1-one (yield: 0.0892 g; 90%). ¹H NMR (Chloroform-d, 600 MHz) δ 12.07 (s, 1H), 7.30 (d, J=8.10 Hz, 2H), 7.22 (d, J=8.72 Hz, 1H), 6.74 (d, J=8.14 Hz, 2H), 6.48 (d, J=8.72 Hz, 1H), 6.32 (broad s, 1H), 5.81 (broad s, 1H), 5.14 (s, 2H); LCMS (ESI): m/z [M+H]⁺=338.9885. FIG. 29 shows the characterization of the product.

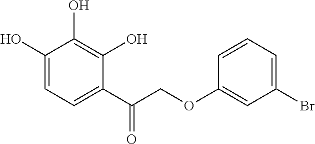 [SMILES: O=C(COc1cccc(Br)c1)c1ccc(O)c(O)c1O]

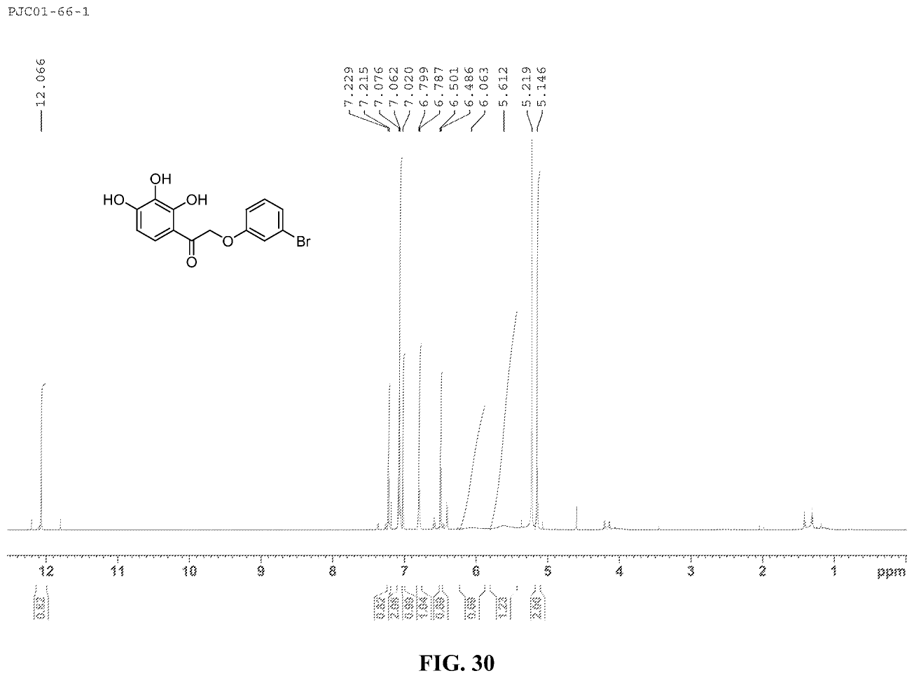

2-(3-Bromophenoxy)-1-(2,3,4-trihydroxyphenyl)ethan-1-one was prepared using the procedure described above (yield: 0.1259 g; 42%). ¹H NMR (Chloroform-d, 600 MHz) δ 12.07 (s, 1H), 7.22 (d, J=8.75 Hz, 1H), 7.09-7.05 (quart, J=7.57, 8.04, 8.16 Hz, 1H), 7.05 (d, J=8.16 Hz, 1H), 7.02 (s, 1H), 6.79 (d, J=7.35 Hz, 1H), 6.49 (d, J=8.88 Hz, 1H), 6.06 (broad s, 1H), 5.61 (broad s, 1H), 5.15 (s, 2H); LCMS (ESI): m/z [M+H]⁺=338.9867. FIG. 30 shows the characterization of the product.

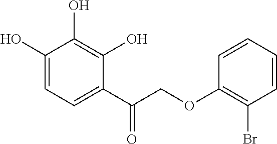 [SMILES: O=C(COc1ccccc1Br)c1ccc(O)c(O)c1O]

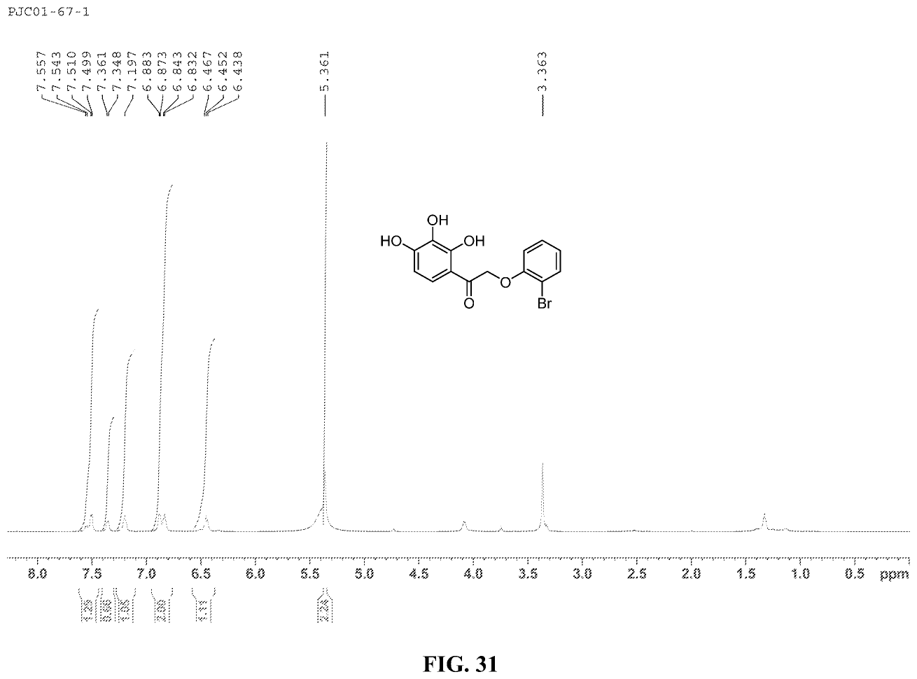

2-(2-Bromophenoxy)-1-(2,3,4-trihydroxyphenyl)ethan-1-one was prepared using the procedure described above (yield: 0.1293 g; 43%). ¹H NMR (MeOD-d₄, 600 MHz) δ 7.56-7.50 (dd, J=6.63, 8.29, 20.02 Hz, 1H), 7.35 (d, J=8.05 Hz, 1H), 7.20 (broad s, 1H), 6.88 (d, J=5.80 Hz, 1H), 6.84-6.83 (m, 1H), 6.47-6.44 (m, 1H), 5.36 (s, 2H); LCMS (ESI): m/z [M+H]+=338.9891. FIG. 31 shows the characterization of the product.

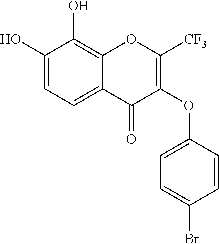 [SMILES: O=c1c(Oc2ccc(Br)cc2)c(C(F)(F)F)oc2c(O)c(O)ccc12]

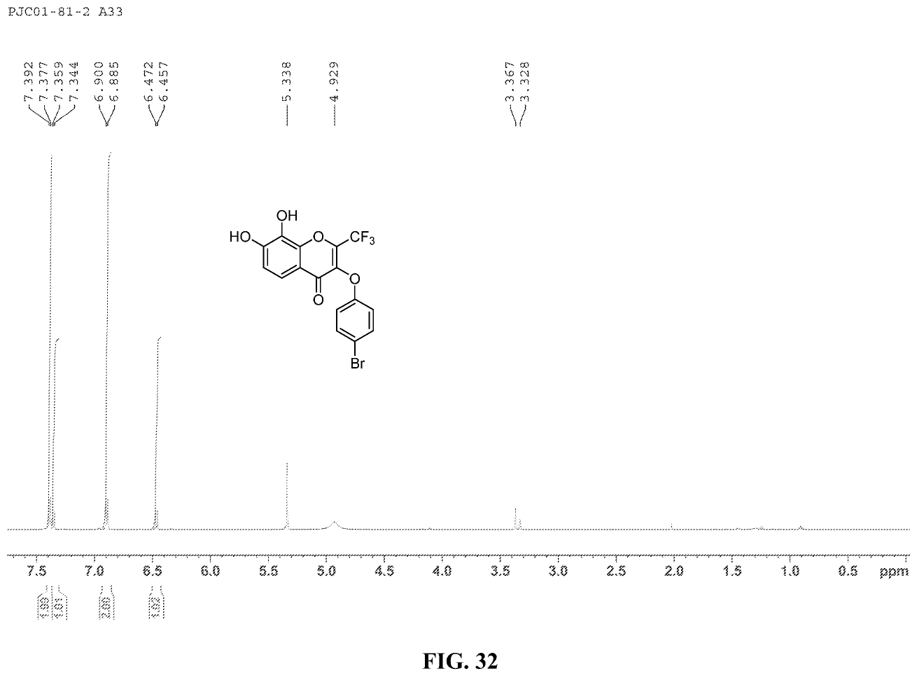

3-(4-Bromophenoxy)-7,8-dihydroxy-2-(trifluoromethyl)-4H-chromen-4-one: A solution of 2-(4-bromophenoxy)-1-(2,3,4-trihydroxyphenyl)ethan-1-one (0.0644 g, 1.0 eq, 0.190 mmol), pyridine (1.00 mL, 65.1 eq, 12.4 mmol) and 2,2,2-trifluoroacetic anhydride (0.040 mL, 1.5 eq, 0.29 mmol) was stirred at room temperature for 12 hours. After 12 hours, the reaction was then stirred at 50° C. for 15 minutes. After 15 minutes, the reaction was then extracted with ethyl acetate and water. The ethyl acetate extraction was dried with magnesium sulfate and filtered. The filtrate was purified by the normal phase chromatography (100% hexane to 20% ethyl acetate in hexane) to afford 3-(4-bromophenoxy)-7,8-dihydroxy-2-(trifluoromethyl)-4H-chromen-4-one (yield: 0.0579 g; 73%). ¹H NMR (MeOD-d₄, 600 MHz) δ 7.38 (d, J=8.05 Hz, 2H), 7.35 (d, J=8.87 Hz, 1H), 6.89 (d, J=8.84 Hz, 2H), 6.47 (d, J=8.85 Hz, 1H); HRMS (ESI): m/z [M+H]⁺ calculated for C₁₆HgBrF₃O₅ 418.9560; found 418.9593; purity >95%. FIG. 32 shows the characterization of the product.

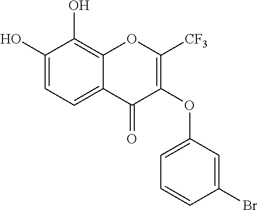 [SMILES: O=c1c(Oc2cccc(Br)c2)c(C(F)(F)F)oc2c(O)c(O)ccc12]

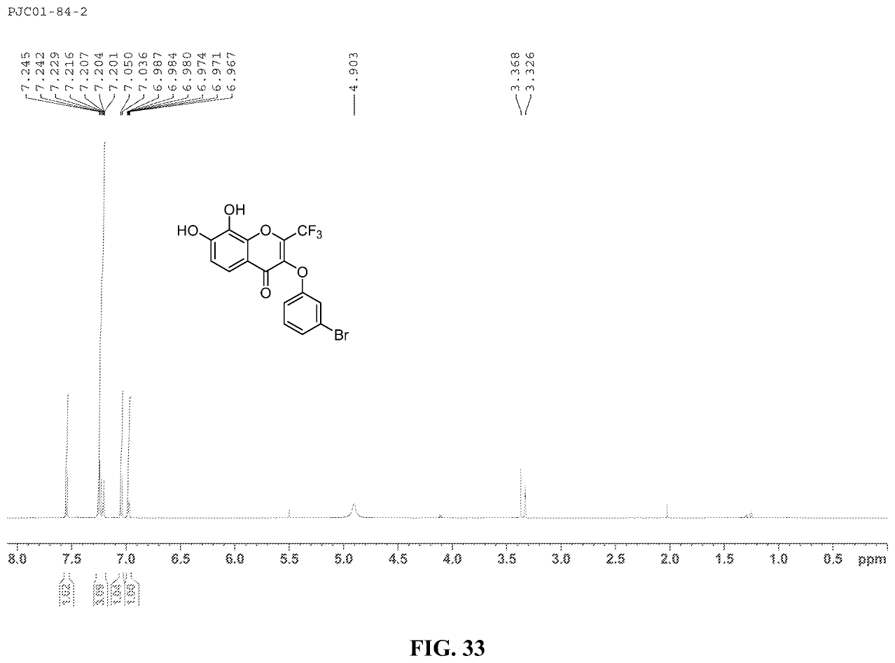

3-(3-Bromophenoxy)-7,8-dihydroxy-2-(trifluoromethyl)-4H-chromen-4-one was prepared using the procedure described above (yield: 0.0415 g; 57%). ¹H NMR (MeOD-d₄, 600 MHz) δ 7.55 (d, J=8.78 Hz, 1H), 7.26-7.24 (m, 1H), 7.22-7.20 (m, 1H), 7.04 (d, J=8.78 Hz, 1H), 6.99-6.97 (td, J=1.70, 1.72, 2.10, 2.26, 3.73 Hz, 1H); HRMS (ESI): m/z [M+H]⁺ calculated for C₁₆HgBrF₃O₅ 416.9580; found 416.9605; purity >95%. FIG. 33 shows the characterization of the product.

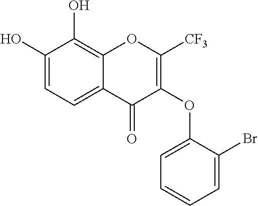 [SMILES: O=c1c(Oc2ccccc2Br)c(C(F)(F)F)oc2c(O)c(O)ccc12]

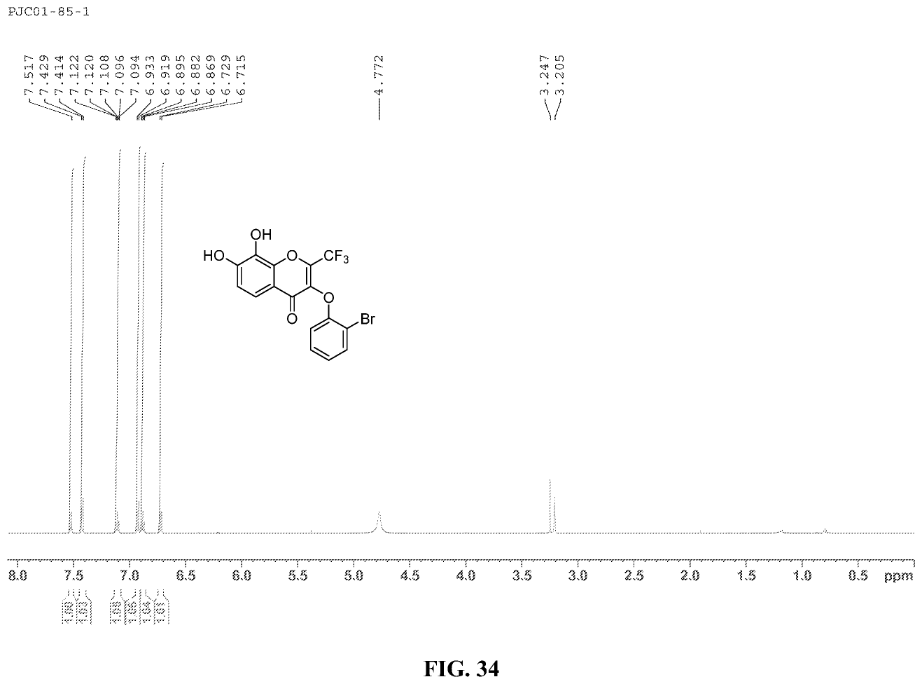

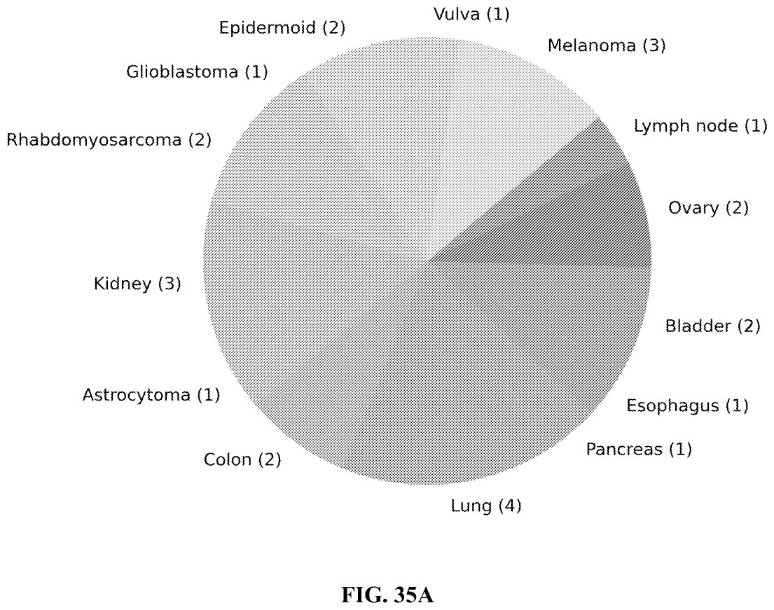

3-(2-Bromophenoxy)-7,8-dihydroxy-2-(trifluoromethyl)-4H-chromen-4-one was prepared using the procedure described above (yield: 0.0381 g; 60%). ¹H NMR (MeOD-d₄, 600 MHz) δ 7.53-7.52 (dd, J=1.13, 1.16, 6.79 Hz, 1H), 7.42 (d, J=8.78 Hz, 1H), 7.12-7.09 (dt, J=1.22, 1.23, 7.05, 7.35 Hz, 1H), 6.93 (d, J=8.78 Hz, 1H), 6.88 (t, J=7.16, 7.85 Hz, 1H), 6.72 (d, J=8.23 Hz, 11H); HRMS (ESI): m/z [M+H]⁺ calculated for C₁₆1H₈BrF₃O₅ 416.9580; found 416.9607; purity >95%. FIG. 34 shows the characterization of the product.

Physiochemical data for these compounds is provided in Table 8.

TABLE 8
Physicochemical Data

| Structure | MW | Sample amount | CLogP | tPSA |
|---|---|---|---|---|
| 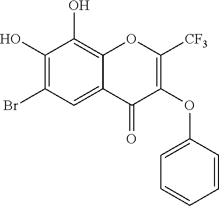 [SMILES: O=c1c(Oc2ccccc2)c(C(F)(F)F)oc2c(O)c(O)c(Br)cc12] | 417.13 | 1.1 mg | 4.42 | 75.99 |
| 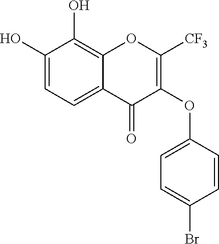 [SMILES: O=c1c(Oc2ccc(Br)cc2)c(C(F)(F)F)oc2c(O)c(O)ccc12] | 417.13 | 3.2 mg | 4.42 | 75.99 |
| 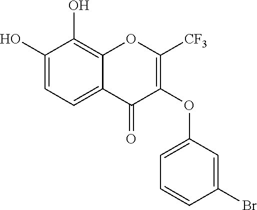 [SMILES: O=c1c(Oc2cccc(Br)c2)c(C(F)(F)F)oc2c(O)c(O)ccc12] | 417.13 | 1.1 mg | 4.42 | 75.99 |
| 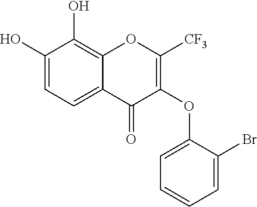 [SMILES: O=c1c(Oc2ccccc2Br)c(C(F)(F)F)oc2c(O)c(O)ccc12] | 417.13 | 3.2 mg | 4.42 | 75.99 |
| 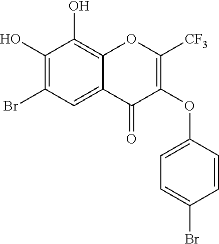 [SMILES: O=c1c(Oc2ccc(Br)cc2)c(C(F)(F)F)oc2c(O)c(O)c(Br)cc12] | 496.03 | 1.2 mg | 5.29 | 75.99 |

Activity data for these compounds is provided in Table 9. E3 activity was measured using the FRET-K48-di-Ub synthesis assay with ROC1-CUL1 CTD as shown in FIG. 11B. IC₅₀ values are indicated. E3-ligand binding was determined using MST with E3 complex ROC1-CUL1 CTD as described in FIG. 19.

TABLE 9
Activity

| | IC₅₀ (FRET CUL1 | E3 Binding Kd (MST with |
|---|---|---|
| Structure | assay) | CUL1 CTD) |
| 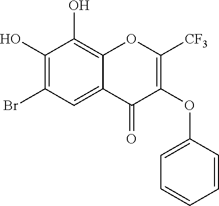 [SMILES: O=c1c(Oc2ccccc2)c(C(F)(F)F)oc2c(O)c(O)c(Br)cc12] | 3.1 μM (original #33 compound 3.3 μM) | 1.89 ± 0.96 μM (#33: 1.62 μM) |
| 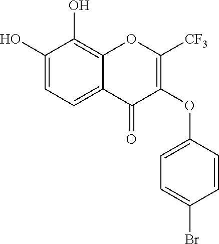 [SMILES: O=c1c(Oc2ccc(Br)cc2)c(C(F)(F)F)oc2c(O)c(O)ccc12] | 3.5 μM | 1.42 ± 0.26 μM |
| 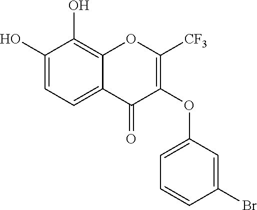 [SMILES: O=c1c(Oc2cccc(Br)c2)c(C(F)(F)F)oc2c(O)c(O)ccc12] | 2.4 μM | 1.90 ± 0.28 μM |
| 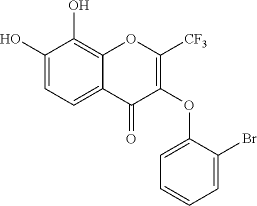 [SMILES: O=c1c(Oc2ccccc2Br)c(C(F)(F)F)oc2c(O)c(O)ccc12] | 2.7 μM | 1.95 ± 0.44 μM |
| 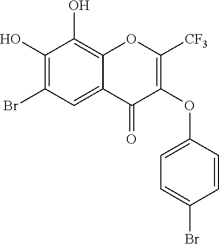 [SMILES: O=c1c(Oc2ccc(Br)cc2)c(C(F)(F)F)oc2c(O)c(O)c(Br)cc12] | 8.3 μM | 1.19 ± 0.36 μM |

### Example 21—Xenograft Method, Ovcar8

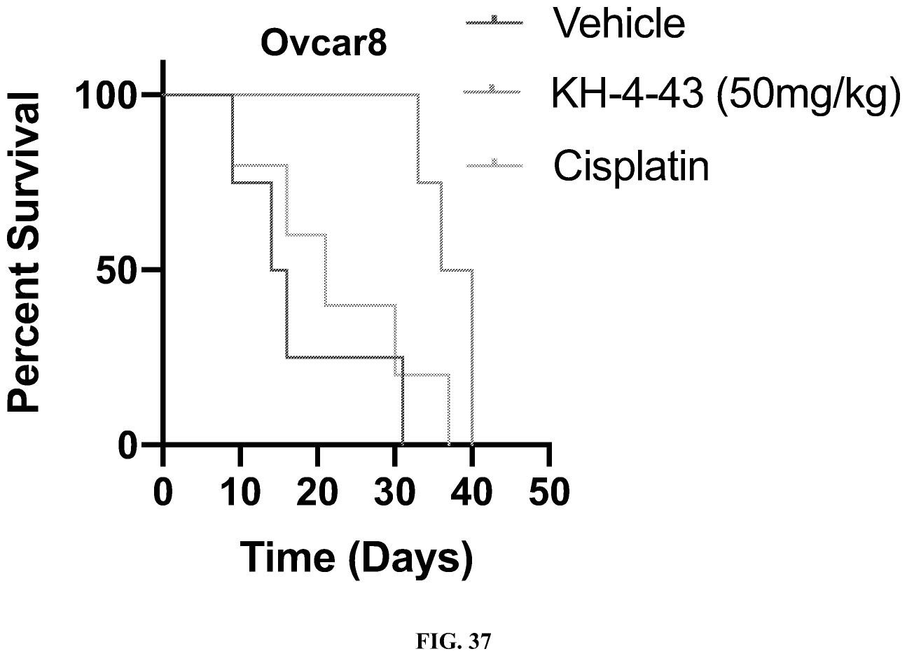

Xenografts of OVCAR8 cells in immune-compromised Nude mice were used to validate findings from the drug screening in vivo (FIG. 37). For the xenografts, 10⁶ cells were injected into the flank of 10 week old mice. Once tumor reached 0.6 cm diameter, animals were randomized into treatment cohorts as indicated (Vehicle, Cisplatin, or KH443). Tumor size was measured with calipers twice a week. The ellipsoid volume (½×L×W×H) was used to estimate tumor volume. Mice were euthanized when they reach humane endpoints. The overall survival (Mantel-Cox) was used to assess survival differences between the groups.

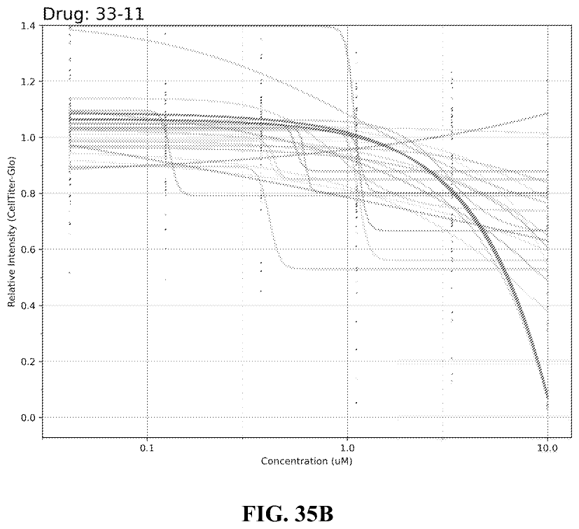

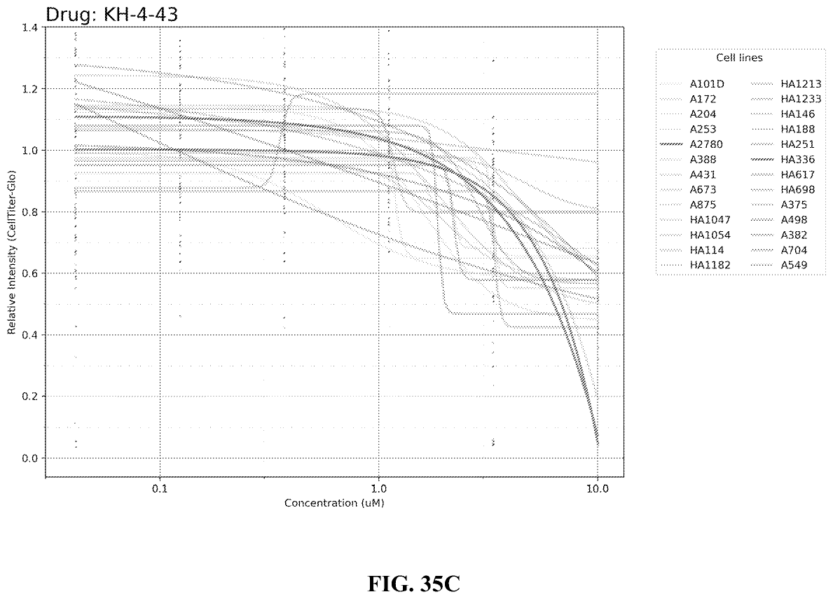

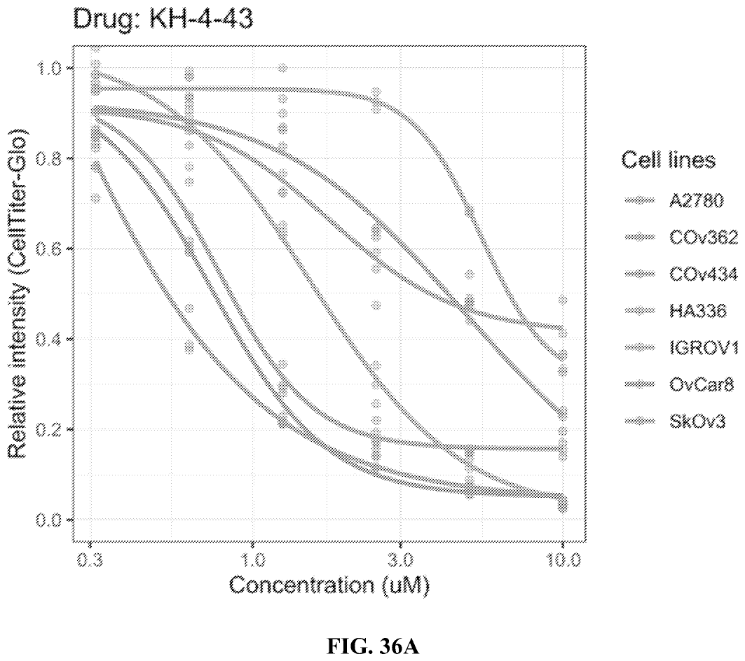

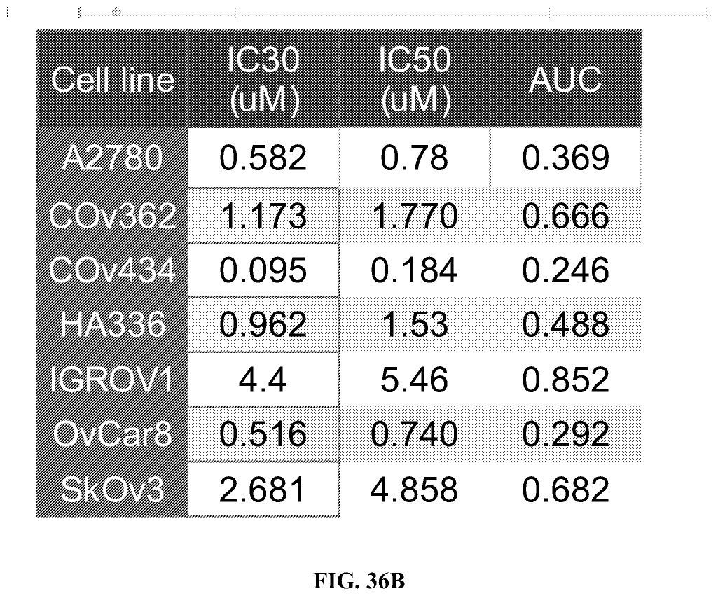

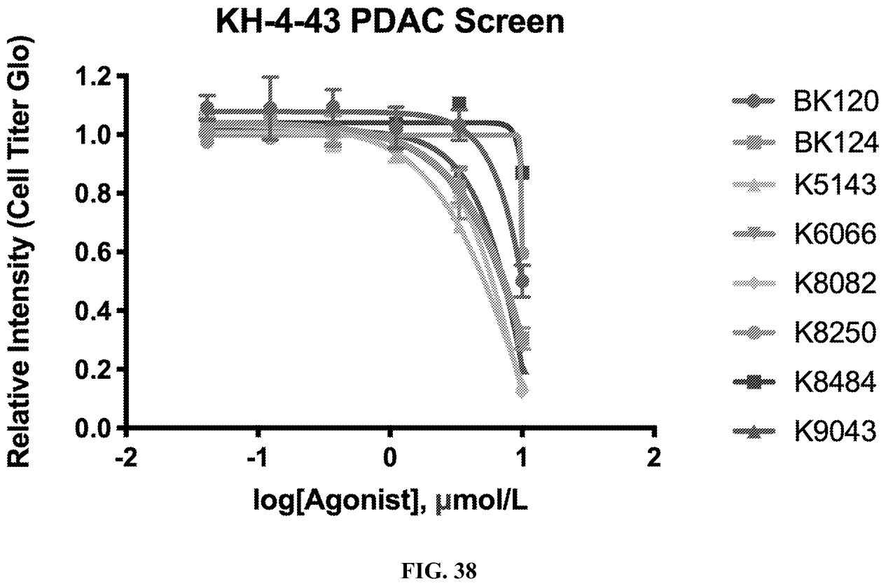

Preclinical evidence in multiple cell lines (FIGS. 35A-C, FIGS. 36A-B, and FIG. 38) and xenografts (FIG. 37) show that KH-4-43 and #33-11 are active anti-cancer agents in several tumor types, including both ovarian and pancreatic cancers.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A compound of formula (I) having the following structure: 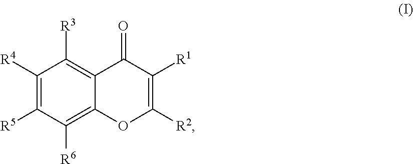
or a stereoisomer, pharmaceutically acceptable salt, oxide, or solvate thereof, wherein
R¹ is H, 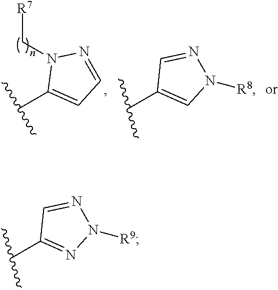
R² is H or CF₃;
R³, R⁴, R⁵, and R⁶ are each independently selected from the group consisting of H, OH, OAc, alkoxy, OTf, and ONa;
R⁷, R⁸, and R⁹ are each independently selected from H, C₁-C₆ alkyl, pyridine, or 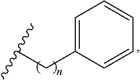
wherein the benzene ring of 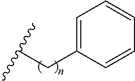
is optionally substituted one or more times with C₁-C₆ alkyl, NH₂, CF₃, halogen, CN, C(O)NH₂, and alkoxy; and
n is 0 or 1;
with the proviso that when R³ and R⁴ are H, R⁵ and R⁶ are OH, and R² is CF₃, R¹ is not

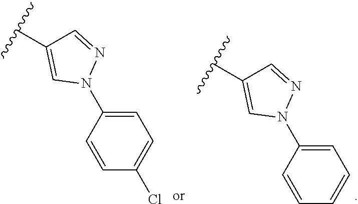 [SMILES: *c1cnn(-c2ccc(Cl)cc2)c1 ; *c1cnn(-c2ccccc2)c1]

2. The compound according to claim 1, wherein R¹ is H.

3. The compound according to claim 1, wherein R¹ is 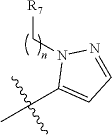
and R⁷ is

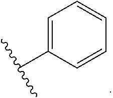 [SMILES: *c1ccccc1]

4. The compound according to claim 1, wherein R¹ is 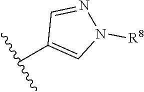
and R⁸ is substituted

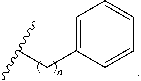

5. The compound according to claim 1, wherein R¹ is

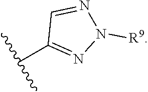

6. The compound according to claim 1, wherein R¹ is 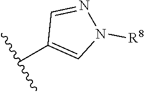
and R⁸ is C₁-C₆ alkyl.

7. The compound according to claim 1, wherein R² is H.

8. The compound according to claim 7, wherein R² is CF₃.

9. The compound according to claim 1, wherein R³ and R⁴ are both H.

10. The compound according to claim 1, wherein R⁵ and R⁶ are both OH.

11. The compound according to claim 10 selected from the group consisting of

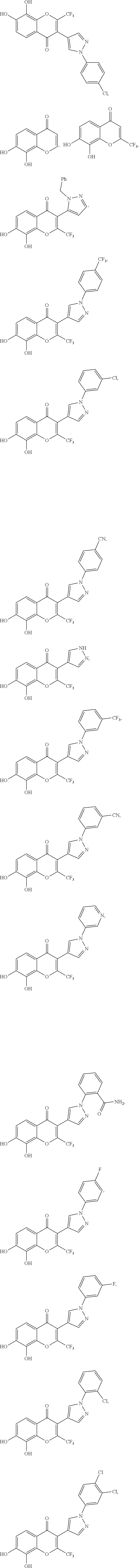

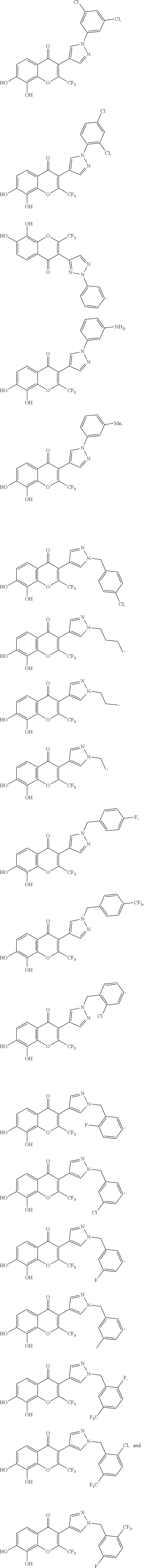

12. The compound according to claim 10 selected from the group consisting of

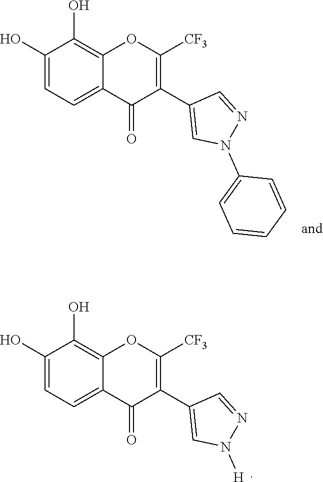 [SMILES: O=c1c(-c2cnn(-c3ccccc3)c2)c(C(F)(F)F)oc2c(O)c(O)ccc12 ; O=c1c(-c2cn[nH]c2)c(C(F)(F)F)oc2c(O)c(O)ccc12]

13. The compound according to claim 1, wherein R⁵ and R⁶ are both OAc.

14. The compound according to claim 13, wherein the compound is

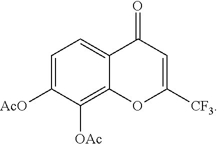 [SMILES: CC(=O)Oc1ccc2c(=O)cc(C(F)(F)F)oc2c1OC(C)=O]

15. The compound according to claim 1, wherein R⁵ and R⁶ are both alkoxy.

16. The compound according to claim 15 selected from the group consisting of

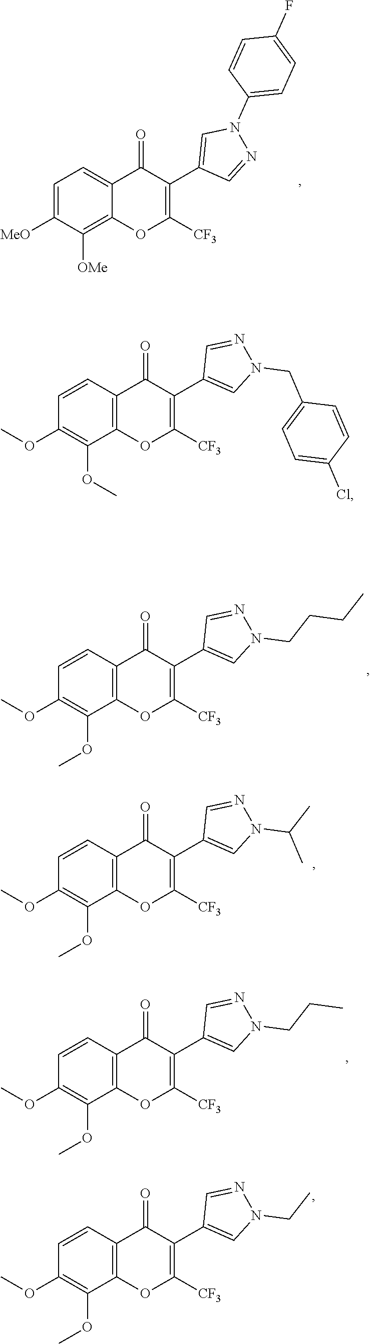

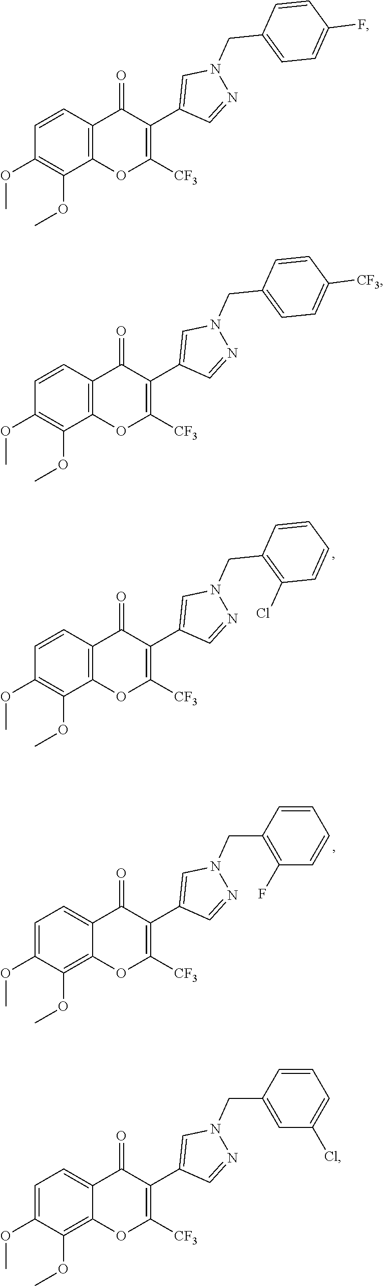 [SMILES: COc1ccc2c(=O)c(-c3cnn(Cc4ccc(F)cc4)c3)c(C(F)(F)F)oc2c1OC ; COc1ccc2c(=O)c(-c3cnn(Cc4ccc(C(F)(F)F)cc4)c3)c(C(F)(F)F)oc2c1OC ; COc1ccc2c(=O)c(-c3cnn(Cc4ccccc4Cl)c3)c(C(F)(F)F)oc2c1OC ; COc1ccc2c(=O)c(-c3cnn(Cc4ccccc4F)c3)c(C(F)(F)F)oc2c1OC ; COc1ccc2c(=O)c(-c3cnn(Cc4cccc(Cl)c4)c3)c(C(F)(F)F)oc2c1OC]

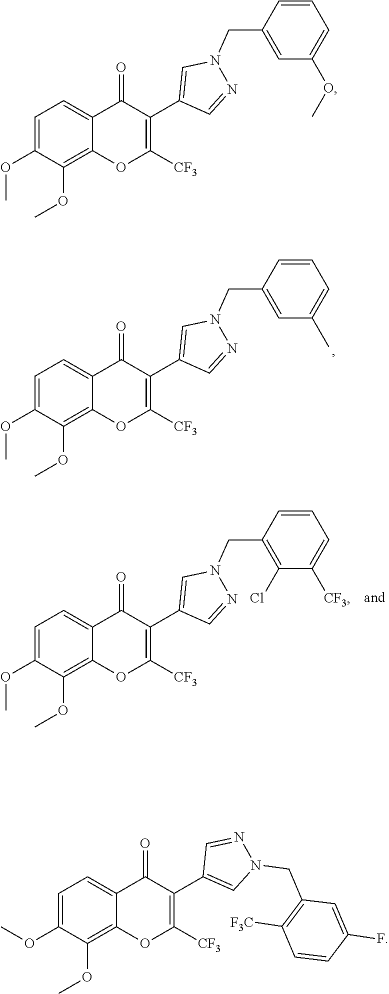

17. The compound according to claim 15 having a structure as follows:

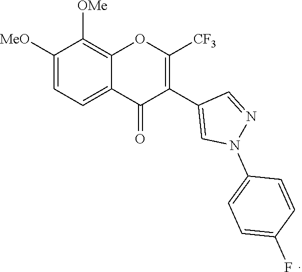 [SMILES: COc1ccc2c(=O)c(-c3cnn(-c4ccc(F)cc4)c3)c(C(F)(F)F)oc2c1OC]

18. The compound according to claim 1, wherein R⁵ and R⁶ are both ONa.

19. The compound according to claim 18, wherein the compound is

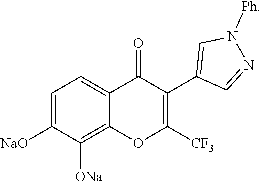

20. The compound according to claim 1, wherein R⁵ and R⁶ are different substituents.

21. The compound according to claim 20 selected from the group consisting of

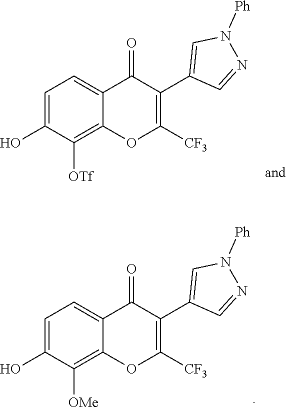 [SMILES: O=c1c(-c2cnn(-c3ccccc3)c2)c(C(F)(F)F)oc2c(OS(=O)(=O)C(F)(F)F)c(O)ccc12 ; COc1c(O)ccc2c(=O)c(-c3cnn(-c4ccccc4)c3)c(C(F)(F)F)oc12]

22. A method of inhibiting the catalytic activity of a Cullin-RING E3 Ubiquitin (Ub) Ligase (CRL) in a cell, said method comprising:
contacting a cell with a compound of formula (II) having the following structure: 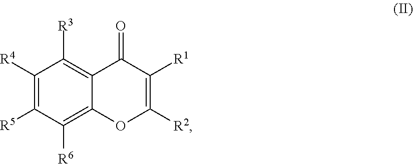
or a stereoisomer, pharmaceutically acceptable salt, oxide, or solvate thereof, wherein
R¹ and R² are each independently H, CF₃, 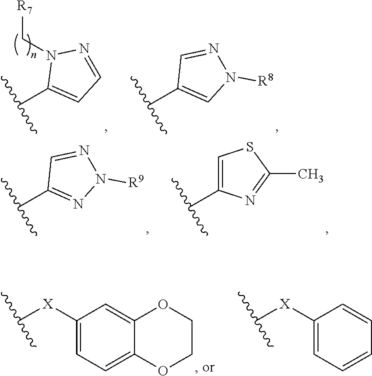
wherein the benzene ring of 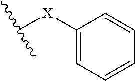
is optionally substituted with NO₂ or halogen;
R³, R⁴, R⁵, and R⁶ are each independently selected from the group consisting of H, halogen, OH, C₁-C₆ alkyl, OAc, NaO, OTf, and alkoxy, wherein two adjacent alkoxy groups may be taken together to form a heterocycle;
R⁷, R⁸, and R⁹ are each independently selected from H, C₁-C₆ alkyl, pyridine, or 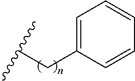
wherein the benzene ring of 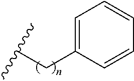
is optionally substituted one or more times with C₁-C₆ alkyl, NH₂, CF₃, halogen, CN, C(O)NH₂, and alkoxy;
X is either present or absent, and when present is CH₂ or O; and
n is 0 or 1;
under conditions effective to inhibit activity of the CRL in the cell.

23. The method according to claim 22, wherein R³, R⁴, R⁵, and R⁶ are each independently selected from the group consisting of H, OH, C₁-C₆ alkyl, OAc, NaO, OTf, and alkoxy, wherein two adjacent alkoxy groups may be taken together to form a heterocycle.

24. The method according to claim 22, wherein R¹ is 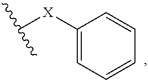
wherein the benzene ring is optionally substituted, and wherein X is O.

25. The method according to claim 24, wherein the compound is selected from the group consisting of

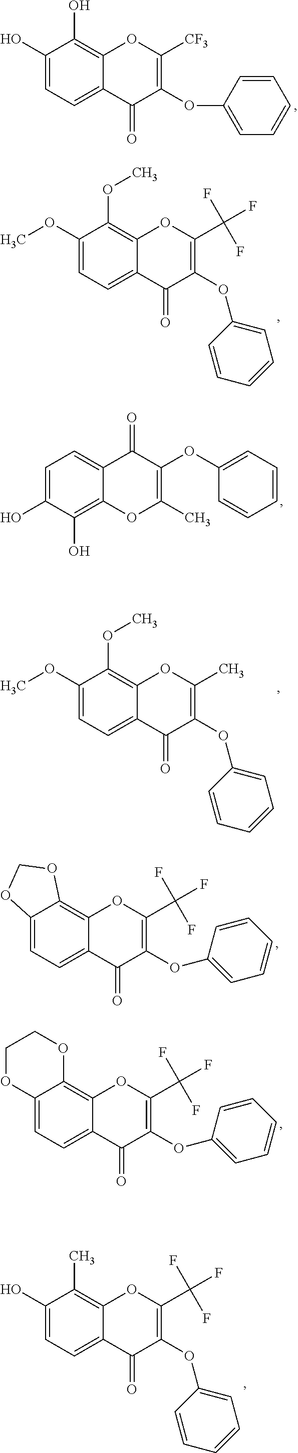

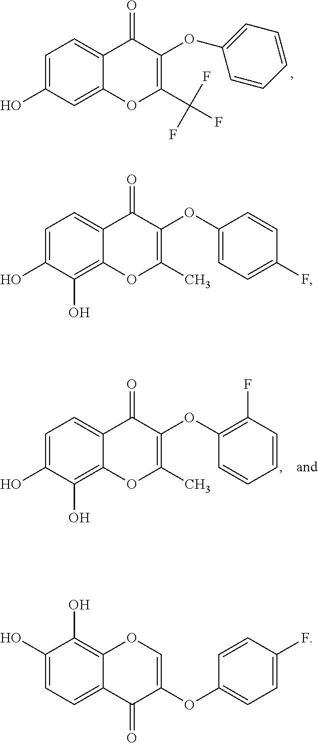 [SMILES: O=c1c(Oc2ccccc2)c(C(F)(F)F)oc2cc(O)ccc12 ; Cc1oc2c(O)c(O)ccc2c(=O)c1Oc1ccc(F)cc1 ; Cc1oc2c(O)c(O)ccc2c(=O)c1Oc1ccccc1F ; O=c1c(Oc2ccc(F)cc2)coc2c(O)c(O)ccc12]

26. The method according to claim 24, wherein the compound is selected from the group consisting of

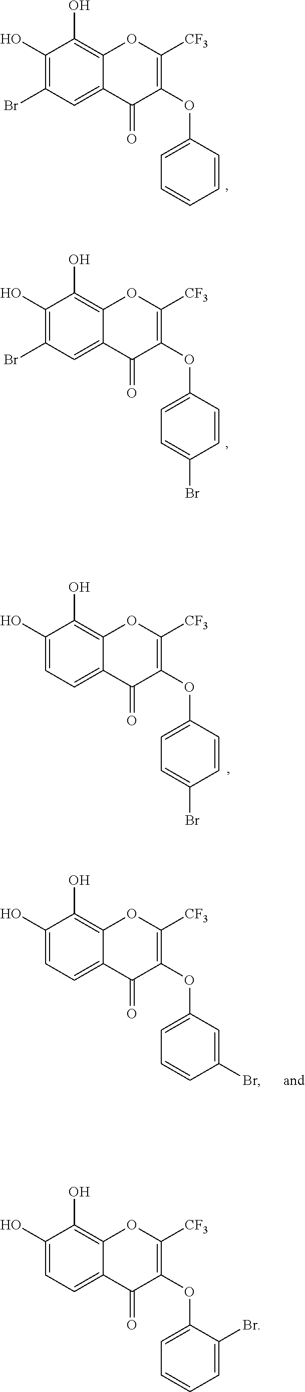 [SMILES: O=c1c(Oc2ccccc2)c(C(F)(F)F)oc2c(O)c(O)c(Br)cc12 ; O=c1c(Oc2ccc(Br)cc2)c(C(F)(F)F)oc2c(O)c(O)c(Br)cc12 ; O=c1c(Oc2ccc(Br)cc2)c(C(F)(F)F)oc2c(O)c(O)ccc12 ; O=c1c(Oc2cccc(Br)c2)c(C(F)(F)F)oc2c(O)c(O)ccc12 ; "and" ; O=c1c(Oc2ccccc2Br)c(C(F)(F)F)oc2c(O)c(O)ccc12]

27. The method according to claim 22, wherein R² is CF₃.

28. The method according to claim 22, wherein R³ and R⁴ are both H.

29. The method according to claim 28, wherein the compound is selected from the group consisting of

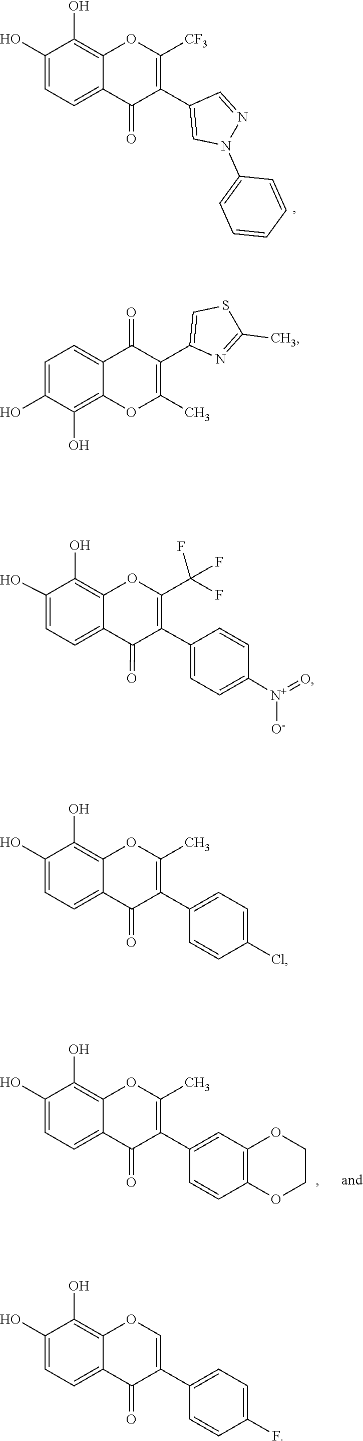 [SMILES: O=c1c(-c2cnn(-c3ccccc3)c2)c(C(F)(F)F)oc2c(O)c(O)ccc12 ; Cc1nc(-c2c(C)oc3c(O)c(O)ccc3c2=O)cs1 ; O=c1c(-c2ccc([N+](=O)[O-])cc2)c(C(F)(F)F)oc2c(O)c(O)ccc12 ; Cc1oc2c(O)c(O)ccc2c(=O)c1-c1ccc(Cl)cc1 ; Cc1oc2c(O)c(O)ccc2c(=O)c1-c1ccc2c(c1)OCCO2 ; O=c1c(-c2ccc(F)cc2)coc2c(O)c(O)ccc12]

30. The method according to claim 28, wherein the compound is selected from the group consisting of

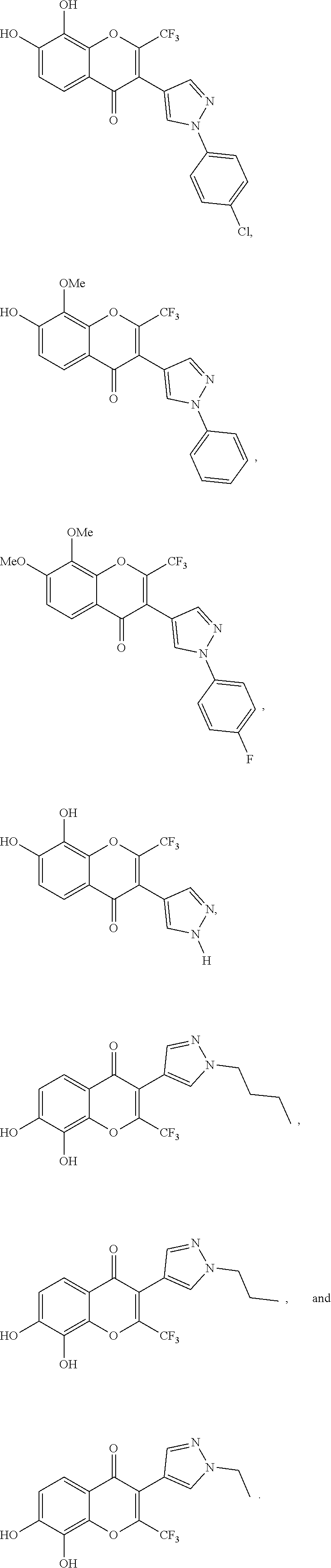

31. The method according to claim 22, wherein R⁵ and R⁶ are both OH.

32. A method of treating a tumor, said method comprising:
contacting a tumor with a compound of claim 22 under conditions effective to treat the tumor.

33. The method according to claim 32, wherein said method is carried out ex vivo.

34. The method according to claim 32, wherein said method is carried out in vivo.

35. The method according to claim 32, wherein the tumor comprises cells that are low-CUL4-expressing cells.

36. A method of treating a subject for cancer, said method comprising:
administering to a subject in need of treatment for cancer a compound of formula (II) having the following structure: 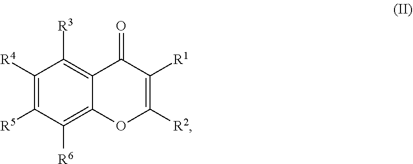
or a stereoisomer, pharmaceutically acceptable salt, oxide, or solvate thereof, wherein
R¹ and R² are each independently H, CF₃, 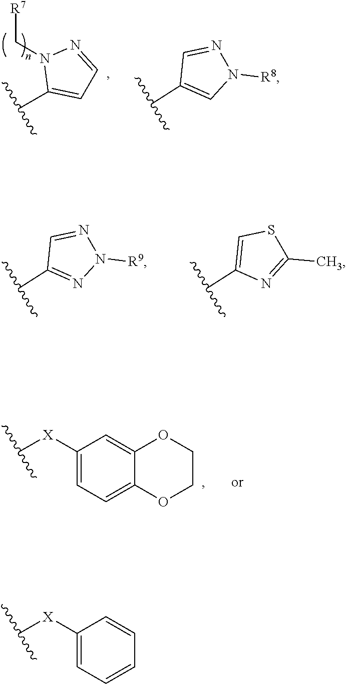
wherein the benzene ring of 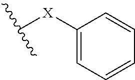
is optionally substituted with NO₂ or halogen;
R³, R⁴, R⁵, and R⁶ are each independently selected from the group consisting of H, halogen, OH, C₁-C₆ alkyl, OAc, NaO, OTf, and alkoxy, wherein two adjacent alkoxy groups may be taken together to form a heterocycle;
R⁷, R⁸, and R⁹ are each independently selected from H, C₁-C₆ alkyl, pyridine, or 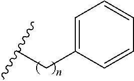
wherein the benzene ring of 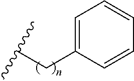
is optionally substituted one or more times with C₁-C₆ alkyl, NH₂, CF₃, halogen, CN, C(O)NH₂, and alkoxy;
X is either present or absent, and when present is C or O; and
n is 0 or 1;
under conditions effective to treat the subject for cancer.

37. The method according to claim 36, wherein R³, R⁴, R⁵, and R⁶ are each independently selected from the group consisting of H, OH, C₁-C₆ alkyl, OAc, NaO, OTf, and alkoxy, wherein two adjacent alkoxy groups may be taken together to form a heterocycle.

38. The method according to claim 36, wherein the compound is selected from the group consisting of

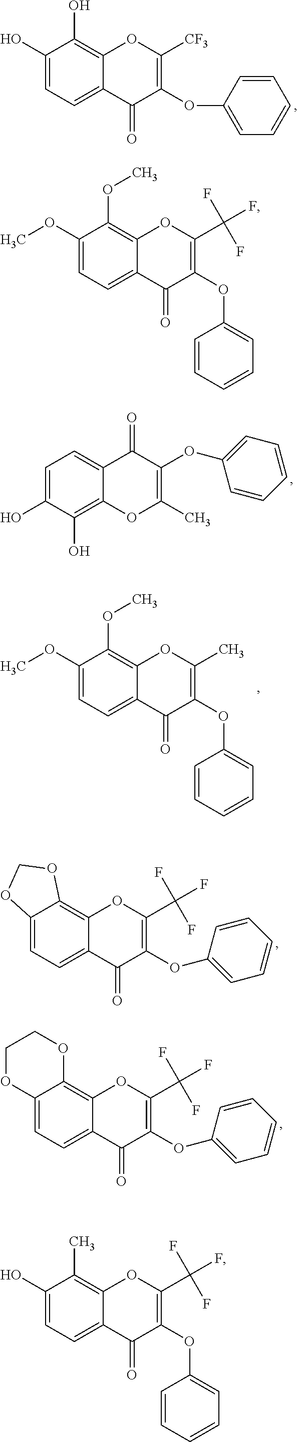

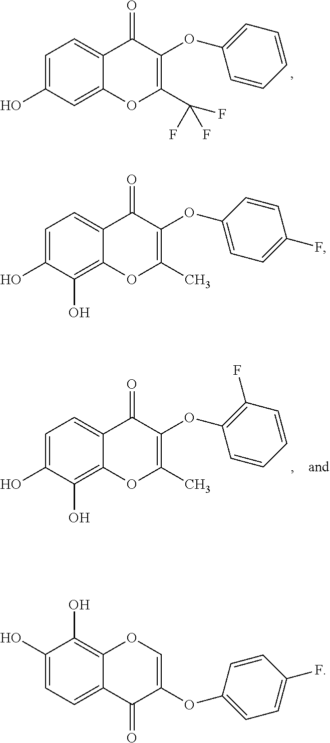 [SMILES: O=c1c(Oc2ccccc2)c(C(F)(F)F)oc2cc(O)ccc12 ; Cc1oc2c(O)c(O)ccc2c(=O)c1Oc1ccc(F)cc1 ; Cc1oc2c(O)c(O)ccc2c(=O)c1Oc1ccccc1F ; O=c1c(Oc2ccc(F)cc2)coc2c(O)c(O)ccc12]

39. The method according to claim 36, wherein the compound is selected from the group consisting of

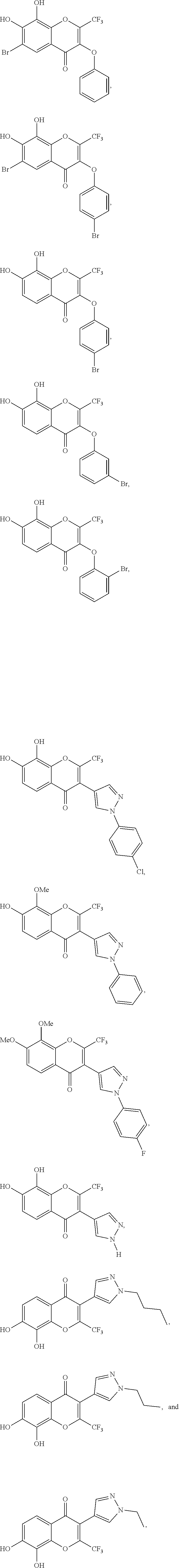

40. The method according to claim 36, wherein said administering is carried out orally, transdermally, parenterally, subcutaneously, intravenously, intramuscularly, or intraperitoneally.

41. The method according to claim 36, wherein the subject is a mammalian subject.

42. The method according to claim 36, wherein the subject is a human subject.

43. A compound having a structure selected from the group consisting of

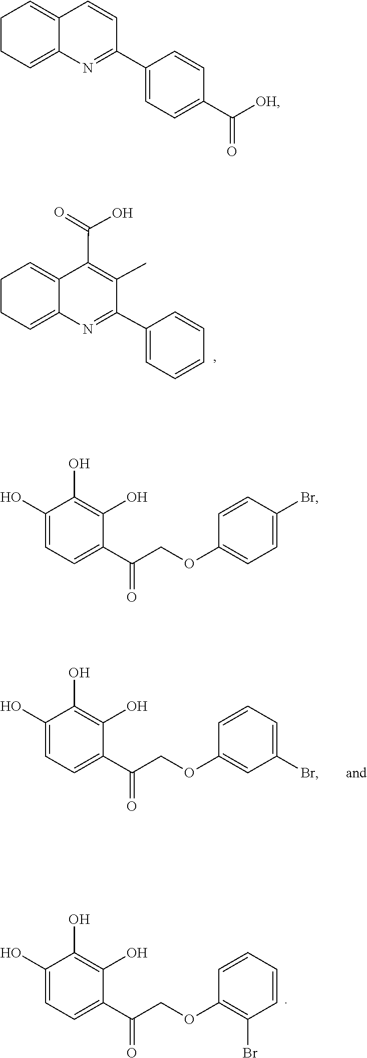 [SMILES: O=C(O)c1ccc(-c2ccc3c(n2)=CCCC=3)cc1 ; Cc1c(-c2ccccc2)nc2c(c1C(=O)O)=CCCC=2 ; O=C(COc1ccc(Br)cc1)c1ccc(O)c(O)c1O ; O=C(COc1cccc(Br)c1)c1ccc(O)c(O)c1O ; "and" ; O=C(COc1ccccc1Br)c1ccc(O)c(O)c1O]

44. The compound according to claim 43, wherein the compound is selected from

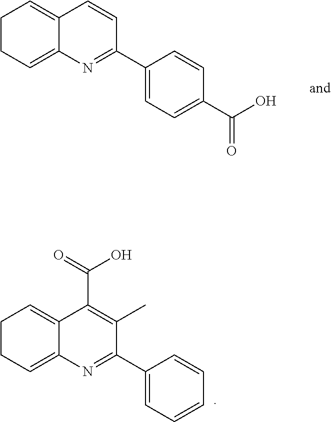 [SMILES: O=C(O)c1ccc(-c2ccc3c(n2)=CCCC=3)cc1 ; Cc1c(-c2ccccc2)nc2c(c1C(=O)O)=CCCC=2]

45. A method of inhibiting the catalytic activity of a Cullin-RING E3 Ubiquitin (Ub) Ligase (CRL) in a cell, said method comprising:
contacting a cell with a compound selected from the group consisting of 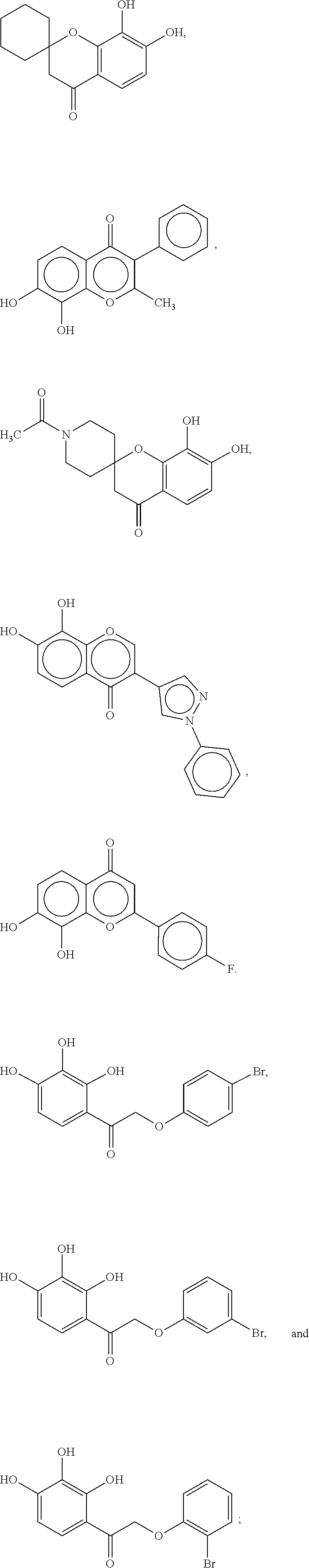
under conditions effective to inhibit activity of the CRL in the cell.

46. The method according to claim 45, wherein the compound is selected from the group consisting of

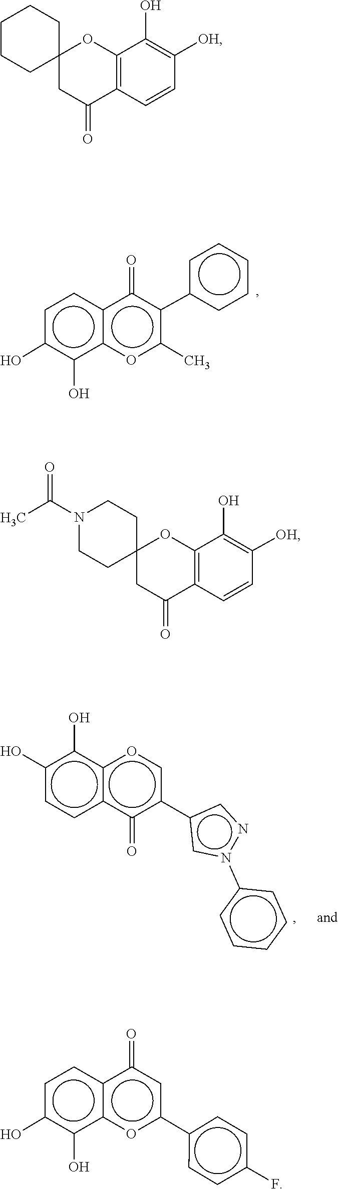

47. A method of treating a subject for cancer, said method comprising: administering to a subject in need of treatment for cancer a compound selected from the group consisting of 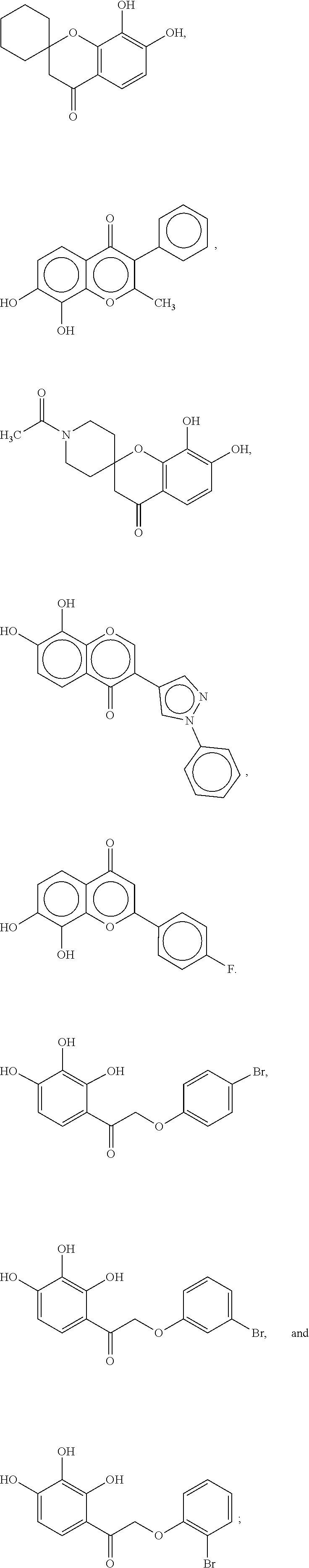
under conditions effective to treat the subject for cancer.


SEQUENCE LISTING (11 sequences)

SEQ ID NO: 1 - DNA, 30 bp, Artificial
  Primer
gccgcgcggcagccataagagacccaagcc

SEQ ID NO: 2 - DNA, 30 bp, Artificial
  Primer
ggcttgggtctcttatggctgccgcgcggc

SEQ ID NO: 3 - RNA, 19 nt, Artificial
  CUL4A siRNA
gcacagauccuuccguuua

SEQ ID NO: 4 - RNA, 19 nt, Artificial
  CUL4A siRNA
gaacagcgaucguaaucaa

SEQ ID NO: 5 - RNA, 19 nt, Artificial
  CUL4A siRNA
gcauguggauucaaaguua

SEQ ID NO: 6 - RNA, 19 nt, Artificial
  CUL4A siRNA
gcgaguacaucaagacuuu

SEQ ID NO: 7 - RNA, 19 nt, Artificial
  CUL4B siRNA
uaaauaaccuccuugauga

SEQ ID NO: 8 - RNA, 19 nt, Artificial
  CUL4B siRNA
cagaagucauuaauugcua

SEQ ID NO: 9 - RNA, 19 nt, Artificial
  CUL4B siRNA
cggaaagagugcaucugua

SEQ ID NO: 10 - RNA, 19 nt, Artificial
  CUL4B siRNA
gcuauuggccgacauaugu

SEQ ID NO: 11 - DNA, 19 bp, Homo sapiens
gatgctggggagtcctgca